(19) United States
(12) Patent Application Publication
(10) Pub. No.: US 2023/0332119 A1
(43) Pub. Date: Oct. 19, 2023

(54) COMPOSITIONS COMPRISING A CAS12I2 VARIANT POLYPEPTIDE AND USES THEREOF

(71) Applicant: Arbor Biotechnologies, Inc., Cambridge, MA (US)

(72) Inventors: Shaorong CHONG, Arlington, MA (US); Brendan Jay HILBERT, Natick, MA (US); Quinton Norman WESSELLS, Cambridge, MA (US); Noah Michael JAKIMO, Cambridge, MA (US); Roy ZIBLAT, Newton, MA (US); Jason Michael CARTE, Cambridge, MA (US); Tia Marie DITOMMASO, Newton, MA (US); Jeffrey Raymond HASWELL, Needham, MA (US); Anthony James GARRITY, Hingham, MA (US); Colin Alexander MCGAW, Boston, MA (US); David A. SCOTT, San Francisco, CA (US); Derek Michael CERCHIONE, Cambridge, MA (US)

(21) Appl. No.: 17/916,270

(22) PCT Filed: Mar. 31, 2021
(86) PCT No.: PCT/US2021/025257

§ 371 Date: Sep. 30, 2022

Related U.S. Application Data
(60) Provisional application No. 63/147,850, filed on Feb. 10, 2021.
(60) Provisional application No. 63/147,968, filed on Feb. 10, 2021.
(60) Provisional application No. 63/085,752, filed on Sep. 30, 2020.
(60) Provisional application No. 63/085,792, filed on Sep. 30, 2020.
(60) Provisional application No. 63/085,862, filed on Sep. 30, 2020.
(60) Provisional application No. 63/085,895, filed on Sep. 30, 2020.
(60) Provisional application No. 63/063,819, filed on Aug. 10, 2020.
(60) Provisional application No. 63/063,831, filed on Aug. 10, 2020.
(60) Provisional application No. 63/063,859, filed on Aug. 10, 2020.
(60) Provisional application No. 63/063,879, filed on Aug. 10, 2020.
(60) Provisional application No. 63/026,277, filed on May 18, 2020.
(60) Provisional application No. 63/026,523, filed on May 18, 2020.
(60) Provisional application No. 63/026,562, filed on May 18, 2020.
(60) Provisional application No. 63/026,606, filed on May 18, 2020.
(60) Provisional application No. 63/012,007, filed on Apr. 17, 2020.
(60) Provisional application No. 63/002,999, filed on Mar. 31, 2020.
(60) Provisional application No. 63/003,064, filed on Mar. 31, 2020.
(60) Provisional application No. 63/003,090, filed on Mar. 31, 2020.

(51) Int. Cl. C12N 9/22; C12N 15/63; C12N 15/10; C12N 15/90

(52) U.S. Cl. CPC C12N 9/22; C12N 15/63; C12N 15/102; C12N 15/907; C12N 2310/20

(57) ABSTRACT

The present invention relates to variant Cas12i2 polypeptides, methods of producing the variant Cas12i2 polypeptides, processes for characterizing the variant Cas12i2 polypeptides, cells comprising the variant Cas12i2 polypeptides, and methods of using the variant Cas12i2 polypeptides. The invention further relates to complexes comprising the variant Cas12i2 polypeptides, methods of producing the complexes, processes for characterizing the complexes, cells comprising the complexes, and methods of using the complexes.

### SEQUENCE LISTING

The instant application contains a Sequence Listing which has been submitted electronically in ASCII format and is hereby incorporated by reference in its entirety. Said ASCII copy, created on Mar. 31, 2021, is named 51451-008WO5_Sequence_Listing_03_30_2021_ST25, and is 1,589,401 bytes in size.

### BACKGROUND

Clustered Regularly Interspaced Short Palindromic Repeats (CRISPR) and CRISPR-associated (Cas) genes, collectively known as CRISPR-Cas or CRISPR/Cas systems, are adaptive immune systems in archaea and bacteria that defend particular species against foreign genetic elements.

### SUMMARY OF THE INVENTION

It is against the above background that the present invention provides certain advantages and advancements over the prior art.

Although the invention disclosed herein is not limited to specific advantages or functionalities, the invention provides a variant Cas12i2 polypeptide comprising a sequence having at least 95% identity to a sequence set forth in any one of SEQ ID NOs: 3 to 146 or any one of SEQ ID NOs: 495 to 512.

In one aspect of the variant Cas12i2 polypeptide, the variant Cas12i2 polypeptide comprises the sequence set forth in SEQ ID NO: 4.

In another aspect of the variant Cas12i2 polypeptide, the variant Cas12i2 polypeptide comprises the sequence set forth in SEQ ID NO: 5.

In another aspect of the variant Cas12i2 polypeptide, the variant Cas12i2 polypeptide comprises the sequence set forth in SEQ ID NO: 495.

In another aspect of the variant Cas12i2 polypeptide, the variant Cas12i2 polypeptide comprises the sequence set forth in SEQ ID NO: 496.

In another aspect of the variant Cas12i2 polypeptide, the variant Cas12i2 polypeptide further comprises one or more substitution of Table 2.

In another aspect of the variant Cas12i2 polypeptide, the variant Cas12i2 polypeptide is a variant of a Cas12i2 polypeptide comprising the sequence set forth in SEQ ID NO: 2.

The invention yet further provides a composition comprising a variant Cas12i2 polypeptide as described herein, wherein the composition further comprises an RNA guide or a nucleic acid encoding the RNA guide, wherein the RNA guide comprises a direct repeat sequence and a spacer sequence.

In one aspect of the composition, the direct repeat sequence comprises a nucleotide sequence with at least 95% sequence identity to any one of SEQ ID NO: 492-494.

In another aspect of the composition, the direct repeat sequence comprises a nucleotide sequence set forth in any one of SEQ ID NO: 492-494.

In another aspect of the composition, the spacer sequence comprises between about 11 and about 50 nucleotides.

In another aspect of the composition, the spacer sequence comprises between about 15 and about 35 nucleotides.

In another aspect of the composition, the spacer sequence binds to a target nucleic acid sequence, wherein the target nucleic acid sequence is adjacent to a 5′-NTTN-3′ sequence.

In another aspect of the variant Cas12i2 polypeptide or the composition, the variant Cas12i2 polypeptide further comprises at least one nuclear localization signal (NLS), at least one nuclear export signal (NES), or at least one NLS and at least one NES.

In another aspect of the variant Cas12i2 polypeptide or the composition, the variant Cas12i2 polypeptide further comprises a peptide tag, a fluorescent protein, a base-editing domain, a DNA methylation domain, a histone residue modification domain, a localization factor, a transcription modification factor, a light-gated control factor, a chemically inducible factor, or a chromatin visualization factor.

In another aspect of the composition, the composition is present in a delivery system comprising a nanoparticle, a liposome, an exosome, a microvesicle, or a gene-gun.

The invention yet further provides a nucleic acid molecule encoding a variant Cas12i2 polypeptide as described herein.

The invention yet further provides a cell comprising a composition or variant Cas12i2 polypeptide as described herein.

In one aspect of the cell, the cell is a eukaryotic cell or a prokaryotic cell.

In another aspect of the cell, the cell is a mammalian cell or a plant cell.

In another aspect of the cell, the cell is a human cell.

The invention yet further provides a composition or formulation comprising a variant Cas12i2 polypeptide as described herein, and optionally an RNA guide and/or a cell.

The invention yet further provides a composition comprising a variant Cas12i2 polypeptide and an RNA guide, wherein the Cas12i2 variant polypeptide and the RNA guide form a complex, and wherein the variant Cas12i2 polypeptide exhibits increased complex formation with the RNA guide as compared to a parent polypeptide.

The invention yet further provides a composition comprising a variant Cas12i2 polypeptide and an RNA guide, wherein the Cas12i2 variant polypeptide and the RNA guide form a complex, and wherein the Cas12i2 variant polypeptide exhibits increased binding affinity to the RNA guide, as compared to a parent polypeptide.

The invention yet further provides a composition comprising a variant Cas12i2 polypeptide and an RNA guide, wherein the Cas12i2 variant polypeptide and the RNA guide form a complex, and the Cas12i2 variant polypeptide and the RNA guide exhibit increased protein-RNA interactions, as compared to a parent polypeptide and the RNA guide.

In one aspect of the composition, the variant Cas12i2 polypeptide exhibits increased complex formation, increased binding affinity to the RNA guide and/or increased stability over a range of temperatures, e.g., 20° C. to 65° C.

In another aspect of the composition, the variant Cas12i2 polypeptide exhibits increased complex formation, increased binding affinity to the RNA guide and/or increased stability over a range of incubation times.

In another aspect of the composition, the variant Cas12i2 polypeptide exhibits increased complex formation, increased binding affinity to the RNA guide and/or increased stability in a buffer having a pH in a range of about 7.3 to about 8.6.

In another aspect of the composition, the variant Cas12i2 polypeptide exhibits increased complex formation, increased binding affinity to the RNA guide and/or increased stability when a Tₘ value of the variant binary complex is at least 8° C. greater than the Tₘ value of the parent complex.

In another aspect of the composition, the parent polypeptide comprises the amino acid sequence of SEQ ID NO: 2.

In another aspect of the composition, the variant Cas12i2 polypeptide exhibits equivalent to or greater enzymatic activity than the parent polypeptide.

In another aspect of the composition, the equivalent to or greater enzymatic activity occurs at a temperature range from about 20° C. to about 90° C.

In another aspect of the composition, the variant Cas12i2 polypeptide exhibits increased stability and/or protein-RNA interactions.

In another aspect of the composition, the variant Cas12i2 polypeptide further lacks enzymatic activity.

In another aspect of the composition, the variant Cas12i2 polypeptide further exhibits increased enzymatic activity.

In another aspect of the composition, the variant Cas12i2 polypeptide exhibits increased RNA guide complex formation, RNA guide binding activity and/or RNA guide binding specificity.

In another aspect of the composition, the variant Cas12i2 polypeptide exhibits altered on-target binding.

In another aspect of the composition, the variant Cas12i2 polypeptide exhibits altered off-target binding.

The invention yet further provides a composition comprising a variant Cas12i2 polypeptide and an RNA guide, wherein the Cas12i2 variant polypeptide and the RNA guide form a variant binary complex, and wherein the variant binary complex exhibits decreased complex dissociation than a complex formed by a parent polypeptide and the RNA guide.

The invention yet further provides a composition comprising a variant Cas12i2 polypeptide and an RNA guide, wherein the Cas12i2 variant polypeptide and the RNA guide form a variant binary complex, and wherein the RNA guide exhibits decreased dissociation from the variant Cas12i2 polypeptide than an RNA guide of a parent complex.

The invention yet further provides a composition comprising a variant Cas12i2 polypeptide and an RNA guide, wherein the Cas12i2 variant polypeptide and the RNA guide form a variant binary complex, and wherein the variant binary complex exhibits increased stability than a complex formed by a parent polypeptide and the RNA guide.

In one aspect of the composition, the variant binary complex exhibits increased ternary complex formation, increased binding affinity to the target nucleic acid and/or increased stability over a range of temperatures, e.g., 20° C. to 65° C.

In another aspect of the composition, the variant binary complex exhibits increased stability over a range of incubation times.

In another aspect of the composition, the variant binary complex exhibits increased stability in a buffer having a pH in a range of about 7.3 to about 8.6.

In another aspect of the composition, the variant binary complex exhibits increased ternary complex formation, increased binding affinity to the target nucleic acid and/or increased stability hen a Tₘ value of the variant binary complex is at least 8° C. greater than the Tₘ value of the parent complex.

In another aspect of the composition, the parent polypeptide comprises the amino acid sequence of SEQ ID NO: 2.

In another aspect of the composition, the variant Cas12i2 polypeptide exhibits equivalent or greater enzymatic activity than the parent polypeptide.

In another aspect of the composition, the equivalent or greater enzymatic activity occurs at a temperature range from about 20° C. to about 90° C.

In another aspect of the composition, the variant Cas12i2 polypeptide exhibits increased stability and/or protein-RNA interactions.

In another aspect of the composition, the variant Cas12i2 polypeptide further lacks enzymatic activity.

In another aspect of the composition, the variant Cas12i2 polypeptide further exhibits increased enzymatic activity.

In another aspect of the composition, the variant Cas12i2 polypeptide exhibits increased RNA guide complex formation, RNA guide binding activity and/or RNA guide binding specificity.

In another aspect of the composition, the variant Cas12i2 polypeptide exhibits altered on-target binding.

In another aspect of the composition, the variant Cas12i2 polypeptide exhibits altered off-target binding.

The invention yet further provides a method of complexing a variant Cas12i2 polypeptide as described herein with an RNA guide, e.g., RNA guide, as described herein.

The invention yet further provides a composition comprising a variant Cas12i2 polypeptide, an RNA guide and a target nucleic acid, wherein the variant Cas12i2 polypeptide and the RNA guide form a variant binary complex, and wherein the variant binary complex exhibits increased ternary complex formation with the target nucleic acid as compared to a parent binary complex.

The invention yet further provides a composition comprising a variant Cas12i2 polypeptide, an RNA guide and a target nucleic acid, wherein the Cas12i2 variant polypeptide and the RNA guide form a variant binary complex, and wherein the variant binary complex exhibits increased binding affinity to the target nucleic acid, as compared to a parent binary complex.

The invention yet further provides a composition comprising a variant Cas12i2 polypeptide, an RNA guide and target nucleic acid, wherein the Cas12i2 variant polypeptide and the RNA guide form a variant binary complex, and wherein the variant binary complex exhibit increased protein-RNA interactions as compared to a parent binary complex.

The invention yet further provides a composition comprising a variant Cas12i2 polypeptide, an RNA guide and target nucleic acid, wherein the Cas12i2 variant polypeptide and the RNA guide form a variant binary complex, and wherein the variant binary complex exhibit increased protein-DNA interactions as compared to a parent binary complex.

In one aspect of the composition, the variant binary complex exhibits increased ternary complex formation, increased binding affinity to the target nucleic acid and/or increased stability over a range of temperatures, e.g., 20° C. to 65° C.

In another aspect of the composition, the variant binary complex exhibits increased ternary complex formation, increased binding affinity to the target nucleic acid and/or increased stability over a range of incubation times.

In another aspect of the composition, the variant binary complex exhibits increased ternary complex formation, increased binding affinity to the target nucleic acid and/or increased stability in a buffer having a pH in a range of about 7.3 to about 8.6.

In another aspect of the composition, the variant binary complex exhibits increased ternary complex formation, increased binding affinity to the target nucleic acid and/or increased stability when a Tₘ value of the binary complex is at least 8° C. greater than the Tₘ value of the parent binary complex.

In another aspect of the composition, the parent polypeptide comprises the amino acid sequence of SEQ ID NO: 2.

In another aspect of the composition, the variant binary complex exhibits equivalent to or greater enzymatic activity than the parent binary complex.

In another aspect of the composition, the equivalent to or greater enzymatic activity occurs at a temperature range from about 20° C. to about 90° C.

In another aspect of the composition, the variant binary complex exhibits increased stability and/or protein-RNA interactions.

In another aspect of the composition, the variant binary complex exhibits increased stability and/or protein-DNA interactions.

In another aspect of the composition, the variant binary complex further lacks enzymatic activity.

In another aspect of the composition, the variant binary complex further exhibits increased enzymatic activity.

In another aspect of the composition, the variant Cas12i2 polypeptide exhibits increased RNA guide complex formation, RNA guide binding activity and/or RNA guide binding specificity.

In another aspect of the composition, the variant binary complex exhibits increased target nucleic acid complex formation, target nucleic acid activity and/or target nucleic acid specificity.

In another aspect of the composition, the variant binary complex exhibits altered on-target binding.

In another aspect of the composition, the variant binary complex exhibits altered off-target binding

The invention yet further provides a composition comprising a variant Cas12i2 polypeptide, an RNA guide and target nucleic acid, wherein the Cas12i2 variant polypeptide and the RNA guide form a variant binary complex, wherein the variant binary complex and target nucleic acid form a variant ternary complex, and wherein the variant ternary complex exhibits decreased complex dissociation than a parent ternary complex.

The invention yet further provides a composition comprising a variant Cas12i2 polypeptide, an RNA guide and target nucleic acid, wherein the Cas12i2 variant polypeptide and the RNA guide form a variant binary complex, wherein the variant binary complex and target nucleic acid form a variant ternary complex, and wherein the target nucleic acid exhibits decreased dissociation from the variant ternary complex than a parent ternary complex.

The invention yet further provides a composition comprising a variant Cas12i2 polypeptide, an RNA guide and target nucleic acid, wherein the Cas12i2 variant polypeptide and the RNA guide form a variant binary complex, wherein the variant binary complex and target nucleic acid form a variant ternary complex, and wherein the variant ternary complex exhibits increased stability than a parent ternary complex.

In one aspect of the composition, the variant ternary complex exhibits increased stability over a range of temperatures, e.g., 20° C. to 65° C.

In another aspect of the composition, the variant ternary complex exhibits increased stability over a range of incubation times.

In another aspect of the composition, the variant ternary complex exhibits increased stability in a buffer having a pH in a range of about 7.3 to about 8.6.

In another aspect of the composition, the variant ternary complex exhibits increased stability hen a Tₘ value of the variant ternary complex is at least 8° C. greater than the Tₘ value of the parent ternary complex.

In another aspect of the composition, the parent polypeptide comprises the amino acid sequence of SEQ ID NO: 2.

In another aspect of the composition, the variant Cas12i2 polypeptide exhibits equivalent or greater enzymatic activity than the parent polypeptide.

In another aspect of the composition, the equivalent or greater enzymatic activity occurs at a temperature range from about 20° C. to about 90° C.

In another aspect of the composition, the variant Cas12i2 polypeptide exhibits increased stability and/or protein-RNA interactions.

In another aspect of the composition, the variant binary complex exhibits increased stability and/or protein-DNA interactions.

In another aspect of the composition, the variant ternary complex exhibits increased stability.

In another aspect of the composition, the variant binary complex further lacks enzymatic activity.

In another aspect of the composition, the variant binary complex further exhibits increased enzymatic activity.

In another aspect of the composition, the variant Cas12i2 polypeptide exhibits increased RNA guide complex formation, RNA guide binding activity and/or RNA guide binding specificity.

In another aspect of the composition, the variant binary complex exhibits increased target nucleic acid complex formation, target nucleic acid binding activity and/or target nucleic acid binding specificity.

In another aspect of the composition, the variant binary complex exhibits altered on-target binding.

In another aspect of the composition, the variant binary complex exhibits altered off-target binding.

The invention yet further provides a method of complexing a variant binary complex as described herein with a target nucleic acid, e.g., DNA, as described herein.

The invention yet further provides a composition comprising a variant Cas12i2 polypeptide and an RNA guide, wherein the variant Cas12i2 polypeptide and the RNA guide form a variant binary complex, and wherein the variant binary complex exhibits increased binding affinity to a target nucleic acid as compared to a parent binary complex.

The invention yet further provides a composition comprising a variant Cas12i2 polypeptide and an RNA guide, wherein the Cas12i2 variant polypeptide and the RNA guide form a variant binary complex, and wherein the variant binary complex exhibits increased target binding affinity to a target locus of a target nucleic acid as compared to a parent binary complex.

In one aspect of the composition, the variant binary complex exhibits increased ternary complex formation and/or increased stability over a range of temperatures, e.g., 20° C. to 65° C.

In another aspect of the composition, the variant binary complex exhibits increased ternary complex formation and/or increased stability over a range of incubation times.

In another aspect of the composition, the variant binary complex exhibits increased ternary complex formation and/or increased stability in a buffer having a pH in a range of about 7.3 to about 8.6.

In another aspect of the composition, the variant binary complex exhibits increased ternary complex formation and/or increased stability when a Tₘ value of the binary complex is at least 8° C. greater than the Tₘ value of the parent binary complex.

In another aspect of the composition, the parent polypeptide comprises the amino acid sequence of SEQ ID NO: 2.

In another aspect of the composition, the variant binary complex exhibits equivalent to or greater enzymatic activity than the parent binary complex.

In another aspect of the composition, the equivalent to or greater enzymatic activity occurs at a temperature range from about 20° C. to about 90° C.

In another aspect of the composition, the variant binary complex exhibits increased stability and/or protein-RNA interactions.

In another aspect of the composition, the variant binary complex exhibits increased stability and/or protein-DNA interactions.

In another aspect of the composition, the variant binary complex further lacks enzymatic activity.

In another aspect of the composition, the variant binary complex further exhibits increased enzymatic activity.

In another aspect of the composition, the variant Cas12i2 polypeptide exhibits increased RNA guide complex formation, RNA guide binding activity and/or RNA guide binding specificity.

In another aspect of the composition, the variant binary complex exhibits increased target nucleic acid complex formation, target nucleic acid activity and/or target nucleic acid specificity.

In another aspect of the composition, the variant binary complex exhibits altered on-target binding.

In another aspect of the composition, the variant binary complex exhibits altered off-target binding.

The invention yet further provides a composition comprising a plurality of variant Cas12i2 polypeptides and two or more distinct RNA guides, wherein the distinct RNA guides individually form variant binary complexes with separate variant Cas12i2 polypeptides, and wherein the variant binary complexes specifically bind with two or more target loci of a target nucleic acid.

The invention yet further provides a composition comprising a plurality of variant Cas12i2 polypeptides and two or more distinct RNA guides, wherein the distinct RNA guides individually form variant binary complexes with separate variant Cas12i2 polypeptides, and wherein the variant binary complexes exhibit increased on-target binding of two or more target loci of a target nucleic acid as compared to parent binary complexes.

The invention yet further provides a composition comprising a plurality of variant Cas12i2 polypeptides and two or more distinct RNA guides, wherein the distinct RNA guides individually form variant binary complexes with separate variant Cas12i2 polypeptides, and wherein the variant binary complexes exhibit increased on-target binding with two or more target loci of a target nucleic acid as compared to parent binary complexes.

The invention yet further provides a composition comprising a plurality of variant Cas12i2 polypeptides and two or more distinct RNA guides, wherein the distinct RNA guides individually form variant binary complexes with separate variant Cas12i2 polypeptides, and wherein the variant binary complexes exhibit on-target ternary complex formation with two or more target loci of a target nucleic acid.

The invention yet further provides a composition comprising a plurality of variant Cas12i2 polypeptides and two or more distinct RNA guides, wherein the distinct RNA guides individually form variant binary complexes with separate variant Cas12i2 polypeptides, and wherein the variant binary complexes exhibit increased ternary complex formation with two or more target loci of a target nucleic acid as compared to parent binary complexes.

In one aspect of the composition, the variant binary complex exhibits increased ternary complex formation with the target nucleic acid and/or increased stability over a range of temperatures, e.g., 20° C. to 65° C.

In another aspect of the composition, the variant binary complex exhibits increased ternary complex formation with the target nucleic acid and/or increased stability over a range of incubation times.

In another aspect of the composition, the variant binary complex exhibits increased ternary complex formation with the target nucleic acid and/or increased stability in a buffer having a pH in a range of about 7.3 to about 8.6.

In another aspect of the composition, the variant binary complex exhibits increased ternary complex formation with the target nucleic acid and/or increased stability when a Tₘ value of the binary complex is at least 8° C. greater than the Tₘ value of the parent binary complex.

In another aspect of the composition, the parent polypeptide comprises the amino acid sequence of SEQ ID NO: 2.

In another aspect of the composition, the variant Cas12i2 polypeptide exhibits equivalent or greater enzymatic activity than the parent polypeptide.

In another aspect of the composition, the equivalent or greater enzymatic activity occurs at a temperature range from about 20° C. to about 90° C.

In another aspect of the composition, the variant Cas12i2 polypeptide exhibits increased stability and/or protein-RNA interactions.

In another aspect of the composition, the variant binary complex exhibits increased stability and/or protein-DNA interactions.

In another aspect of the composition, the variant binary complex further lacks enzymatic activity.

In another aspect of the composition, the variant binary complex further exhibits increased enzymatic activity.

In another aspect of the composition, the variant Cas12i2 polypeptide exhibits increased RNA guide complex formation, RNA guide binding activity and/or RNA guide binding specificity.

In another aspect of the composition, the variant binary complex exhibits increased target nucleic acid ternary complex formation, target nucleic acid binding affinity and/or target nucleic acid binding specificity.

In another aspect of the composition, the variant binary complex exhibits altered on-target binding.

In another aspect of the composition, the variant binary complex exhibits altered off-target binding.

The invention yet further provides a method of complexing a variant binary complex described herein with a target nucleic acid, e.g., DNA, as described herein.

The invention yet further provides a composition comprising a variant Cas12i2 polypeptide and an RNA guide, wherein the Cas12i2 variant polypeptide and the RNA guide form a variant binary complex, and wherein the variant binary complex exhibits increased on-target binding affinity to a target locus of a target nucleic acid as compared to a parent binary complex.

The invention yet further provides a composition comprising a variant Cas12i2 polypeptide and an RNA guide, wherein the variant Cas12i2 polypeptide and the RNA guide form a variant binary complex, and wherein the variant binary complex exhibits decreased binding affinity to a non-target locus of a target nucleic acid as compared to a parent binary complex.

The invention yet further provides a composition comprising a variant Cas12i2 polypeptide and an RNA guide, wherein the variant Cas12i2 polypeptide and the RNA guide form a variant binary complex, and wherein the variant binary complex exhibits increased activity at an on-target locus of a target nucleic acid as compared to a parent binary complex.

The invention yet further provides a composition comprising a variant Cas12i2 polypeptide and an RNA guide, wherein the variant Cas12i2 polypeptide and the RNA guide form a variant binary complex, and wherein the variant binary complex exhibits decreased activity at a non-target locus of a target nucleic acid as compared to a parent binary complex.

In one aspect of the composition, the variant binary complex exhibits increased ternary complex formation at the target locus and/or increased stability over a range of temperatures, e.g., 20° C. to 65° C.

In another aspect of the composition, the variant binary complex exhibits increased ternary complex formation at the target locus and/or increased stability over a range of incubation times.

In another aspect of the composition, the variant binary complex exhibits increased ternary complex formation at the target locus and/or increased stability in a buffer having a pH in a range of about 7.3 to about 8.6.

In another aspect of the composition, the variant binary complex exhibits increased ternary complex formation at the target locus and/or increased stability when a Tₘ value of the binary complex is at least 8° C. greater than the Tₘ value of the parent binary complex.

In another aspect of the composition, the parent binary complex comprises a parent polypeptide that comprises the amino acid sequence of SEQ ID NO: 2.

In another aspect of the composition, the variant Cas12i2 polypeptide exhibits equivalent or greater enzymatic activity than the parent polypeptide.

In another aspect of the composition, the equivalent or greater enzymatic activity occurs at a temperature range from about 20° C. to about 90° C.

In another aspect of the composition, the variant binary complex exhibits increased stability and/or protein-RNA interactions.

In another aspect of the composition, the variant binary complex exhibits increased stability and/or protein-DNA interactions.

In another aspect of the composition, the variant Cas12i2 polypeptide exhibits increased binary complex formation, RNA guide binding activity and/or RNA guide binding specificity.

The invention yet further provides a composition comprising a plurality of variant Cas12i2 polypeptides and two or more distinct RNA guides, wherein the distinct RNA guides individually form a plurality of variant binary complexes with separate variant Cas12i2 polypeptides, and wherein the plurality of variant binary complexes exhibit increased on-target binding to two or more target loci of a target nucleic acid as compared to a plurality of parent binary complexes.

The invention yet further provides a composition comprising a plurality of variant Cas12i2 polypeptides and two or more distinct RNA guides, wherein the distinct RNA guides individually form a plurality of variant binary complexes with separate variant Cas12i2 polypeptides, and wherein the plurality of variant binary complexes exhibit decreased off-target binding to two or more non-target loci of a target nucleic acid as compared to a plurality of parent binary complexes.

The invention yet further provides a composition comprising a plurality of variant Cas12i2 polypeptides and two or more distinct RNA guides, wherein the distinct RNA guides individually form a plurality of variant binary complexes with separate variant Cas12i2 polypeptides, and wherein the plurality of variant binary complexes exhibit increased on-target activity at two or more target loci of a target nucleic acid as compared to a plurality of parent binary complexes.

The invention yet further provides a composition comprising a plurality of variant Cas12i2 polypeptides and two or more distinct RNA guides, wherein the distinct RNA guides individually form a plurality of variant binary complexes with separate variant Cas12i2 polypeptides, and wherein the plurality of variant binary complexes exhibit decreased off-target activity at two or more non-target loci of a target nucleic acid as compared to a plurality of parent binary complexes.

In one aspect of the composition, the plurality of variant binary complexes exhibit increased ternary complex formation at the target loci of the target nucleic acid and/or increased stability over a range of temperatures, e.g., 20° C. to 65° C.

In another aspect of the composition, the plurality of variant binary complexes exhibit increased ternary complex formation at the target loci of the target nucleic acid and/or increased stability over a range of incubation times.

In another aspect of the composition, the plurality of variant binary complexes exhibit increased ternary complex formation at the target loci the target nucleic acid and/or increased stability in a buffer having a pH in a range of about 7.3 to about 8.6.

In another aspect of the composition, the plurality of variant binary complexes exhibit increased ternary complex formation at the target loci of the target nucleic acid and/or increased stability when a Tₘ value of the binary complex is at least 8° C. greater than the Tₘ value of the parent binary complex.

In another aspect of the composition, the plurality of parent binary complexes comprise a parent polypeptide that comprises the amino acid sequence of SEQ ID NO: 2.

In another aspect of the composition, the variant Cas12i2 polypeptide exhibits equivalent or greater enzymatic activity than the parent polypeptide.

In another aspect of the composition, the equivalent or greater enzymatic activity occurs at a temperature range from about 20° C. to about 90° C.

In another aspect of the composition, the variant Cas12i2 polypeptide exhibits increased stability and/or protein-RNA interactions.

In another aspect of the composition, the plurality of variant binary complexes exhibit increased stability and/or protein-DNA interactions.

In another aspect of the composition, the variant Cas12i2 polypeptide exhibits increased binary complex formation, RNA guide binding activity and/or RNA guide binding specificity.

The invention yet further provides a method of complexing a variant binary complex as described herein with a target nucleic acid, e.g., DNA, as described herein.

The invention yet further provides a method of complexing a plurality of variant binary complexes described herein with a target nucleic acid, e.g., DNA, as described herein.

In one aspect of a composition or Cas12i2 polypeptide described herein, the variant Cas12i2 polypeptide comprises at least one of a D581, G624, F626, D835, L836, P868, S879, D911, 1926, V1020, V1030, E1035, and S1046 substitution of amino acid sequence of SEQ ID NO: 2.

In another aspect of the composition or Cas12i2 polypeptide, the variant Cas12i2 polypeptide comprises at least one of a D581G, D581R, G624R, F626G, F626R, D835G, D835R, L836G, L836R, P868G, P868R, P868T, S879G, S879R, D911G, D911R, 1926G, 1926R, V1020 G, V1020R, V1030G, V1030R, E1035G, E1035R, S1046G, and S1046R substitution of amino acid sequence of SEQ ID NO: 2.

In another aspect of the composition or Cas12i2 polypeptide, the variant Cas12i2 polypeptide comprises at least one of a D581R, G624R, F626R, P868T, D911R, 1926R, V1030G, E1035R, and S1046G substitution of amino acid sequence of SEQ ID NO: 2.

In another aspect of the composition or Cas12i2 polypeptide, the variant Cas12i2 polypeptide comprises at least one substitution listed in Table 1.

In another aspect of the composition or Cas12i2 polypeptide, the variant Cas12i2 polypeptide comprises any one amino acid sequence of SEQ ID NO: 3 to 146 or any one amino acid sequence of SEQ ID NOs: 495 to 512.

In another aspect of the composition or Cas12i2 polypeptide, the variant Cas12i2 polypeptide comprises at least one of an epitope peptide, nuclear localization signal, and nuclear export signal.

In another aspect of the composition, the RNA guide comprises a DNA targeting sequence.

In another aspect of the composition, the DNA targeting sequence is an RNA guide.

In another aspect of the composition, the DNA targeting sequence is between 13 to 30 nucleotides.

In another aspect of the composition, the RNA guide comprises a direct repeat sequence linked to a DNA targeting sequence.

In another aspect of the composition, the composition further comprises a target nucleic acid.

In another aspect of the composition, the target nucleic acid is present in a cell.

In another aspect of the composition, the variant Cas12i2 polypeptide and RNA guide are encoded in a vector, e.g., expression vector.

The invention yet further provides a cell comprising a composition as described herein.

The invention yet further provides a method of expressing a vector as described herein.

The invention yet further provides a method of producing a composition as described herein.

The invention yet further provides a method of delivering a composition as described herein.

The invention yet further provides a kit or system comprising a composition as described herein or one or more component thereof.

In one aspect of a composition described herein, the RNA guide comprises or consists of, or about, 43 nucleotides.

In another aspect of the composition, the RNA guide is a tracr-less RNA guide.

In another aspect of the composition or Cas12i2 polypeptide, the variant Cas12i2 polypeptide further exhibits about 40× greater enzymatic activity than parent polypeptide.

In another aspect of the composition or Cas12i2 polypeptide, the variant Cas12i2 polypeptide exhibits increased on-target specificity as compared to the parent polypeptide.

In another aspect of the composition or Cas12i2 polypeptide, the variant Cas12i2 polypeptide exhibits decreased off-target specificity as compared to the parent polypeptide.

In another aspect of the composition, the variant Cas12i2 polypeptide selectively induces a deletion adjacent to a 5′-NTTN-3′ sequence, wherein N is any nucleotide.

In another aspect of the composition, the deletion is downstream of the 5′-NTTN-3′ sequence.

In another aspect of the composition, the parent polypeptide does not induce the deletion.

In another aspect of the composition, the length of the deletion is greater than the length of a Cas9 polypeptide-induced deletion.

In another aspect of the composition, the deletion is in a gene of a cell.

In another aspect of the composition, the deletion is up to about 40 nucleotides in length.

In another aspect of the composition, the deletion is from about 4 nucleotides to 40 nucleotides in length.

In another aspect of the composition, the deletion is from about 4 nucleotides to 25 nucleotides in length.

In another aspect of the composition, the deletion is from about 10 nucleotides to 25 nucleotides in length.

In another aspect of the composition, the deletion is from about 10 nucleotides to 15 nucleotides in length.

In another aspect of the composition, the deletion starts within about 5 nucleotides to about 15 nucleotides of the 5′-NTTN-3′ sequence.

In another aspect of the composition, the deletion starts within about 5 nucleotides to about 10 nucleotides of the 5′-NTTN-3′ sequence.

In another aspect of the composition, the deletion starts within about 10 nucleotides to about 15 nucleotides of the 5′-NTTN-3′ sequence.

In another aspect of the composition, the deletion starts within about 5 nucleotides to about 15 nucleotides downstream of the 5′-NTTN-3′ sequence.

In another aspect of the composition, the deletion starts within about 5 nucleotides to about 10 nucleotides downstream of the 5′-NTTN-3′ sequence.

In another aspect of the composition, the deletion starts within about 10 nucleotides to about 15 nucleotides downstream of the 5′-NTTN-3′ sequence.

In another aspect of the composition, the deletion ends within about 20 nucleotides to about 30 nucleotides of the 5′-NTTN-3′ sequence.

In another aspect of the composition, the deletion ends within about 20 nucleotides to about 25 nucleotides of the 5′-NTTN-3′ sequence.

In another aspect of the composition, the deletion ends within about 25 nucleotides to about 30 nucleotides of the 5′-NTTN-3′ sequence.

In another aspect of the composition, the deletion ends within about 20 nucleotides to about 30 nucleotides downstream of the 5′-NTTN-3′ sequence.

In another aspect of the composition, the deletion ends within about 20 nucleotides to about 25 nucleotides downstream of the 5′-NTTN-3′ sequence.

In another aspect of the composition, the deletion ends within about 25 nucleotides to about 30 nucleotides downstream of the 5′-NTTN-3′ sequence.

In another aspect of the composition, the deletion starts within about 5 nucleotides to about 15 nucleotides downstream of the 5′-NTTN-3′ sequence and ends within about 20 nucleotides to about 30 nucleotides downstream of the 5′-NTTN-3′ sequence.

In another aspect of the composition, the deletion starts within about 5 nucleotides to about 15 nucleotides downstream of the 5′-NTTN-3′ sequence and ends within about 20 nucleotides to about 25 nucleotides downstream of the 5′-NTTN-3′ sequence.

In another aspect of the composition, the deletion starts within about 5 nucleotides to about 15 nucleotides downstream of the 5′-NTTN-3′ sequence and ends within about 25 nucleotides to about 30 nucleotides downstream of the 5′-NTTN-3′ sequence.

In another aspect of the composition, the deletion starts within about 5 nucleotides to about 10 nucleotides downstream of the 5′-NTTN-3′ sequence and ends within about 20 nucleotides to about 30 nucleotides downstream of the 5′-NTTN-3′ sequence.

In another aspect of the composition, the deletion starts within about 5 nucleotides to about 10 nucleotides downstream of the 5′-NTTN-3′ sequence and ends within about 20 nucleotides to about 25 nucleotides downstream of the 5′-NTTN-3′ sequence.

In another aspect of the composition, the deletion starts within about 5 nucleotides to about 10 nucleotides downstream of the 5′-NTTN-3′ sequence and ends within about 25 nucleotides to about 30 nucleotides downstream of the 5′-NTTN-3′ sequence.

In another aspect of the composition, the deletion starts within about 10 nucleotides to about 15 nucleotides downstream of the 5′-NTTN-3′ sequence and ends within about 20 nucleotides to about 30 nucleotides downstream of the 5′-NTTN-3′ sequence.

In another aspect of the composition, the deletion starts within about 10 nucleotides to about 15 nucleotides downstream of the 5′-NTTN-3′ sequence and ends within about 20 nucleotides to about 25 nucleotides downstream of the 5′-NTTN-3′ sequence.

In another aspect of the composition, the deletion starts within about 10 nucleotides to about 15 nucleotides downstream of the 5′-NTTN-3′ sequence and ends within about 25 nucleotides to about 30 nucleotides downstream of the 5′-NTTN-3′ sequence.

In another aspect of the composition, the 5′-NTTN-3′ sequence is 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′, wherein Y is C or T, B is any nucleotide except for A, D is any nucleotide except for C, and R is A or G.

In another aspect of the composition, the 5′-NTTN-3′ sequence is 5′-CTTT-3′, 5′-CTTC-3′, 5′-GTTT-3′, 5′-GTTC-3′, 5′-TTTC-3′, 5′-GTTA-3′, or 5′-GTTG-3′.

In another aspect of the composition, the deletion is in an exon of the gene, e.g., B2M, TRAC, PDCD1.

In another aspect of the composition, the deletion overlaps with a mutation in the gene.

In another aspect of the composition, the deletion overlaps with an insertion in the gene.

In another aspect of the composition, the deletion removes a repeat expansion of the gene.

In another aspect of the composition, the deletion disrupts one or both alleles of the gene.

In another aspect of the composition, the deletion is induced in a eukaryotic cell or a prokaryotic cell.

In another aspect of the composition, the deletion is induced in an animal cell, a plant cell, or a fungal cell or the cell is derived from an animal cell, a plant cell, or a fungal cell.

In another aspect of the composition, the deletion is induced in a mammalian cell or derived from a mammalian cell.

In another aspect of the composition, the deletion is induced in a human cell or derived from a human cell.

In another aspect of the composition, the deletion is induced in a primary cell.

In another aspect of the composition, the deletion is induced in a cell line.

In another aspect of the composition, the deletion is induced in a T cell.

In another aspect of the composition, the deletion is induced in a stem cell (e.g., a totipotent/omnipotent stem cell, a pluripotent stem cell, a multipotent stem cell, an oligopotent stem cell, or an unipotent stem cell), a differentiated cell, or a terminally differentiated cell.

In another aspect of the composition, 2 or more (e.g., multiplexed targeted deletions) deletions are induced.

The invention yet further provides a method of obtaining a deletion in a cell, wherein the method comprises contacting a variant Cas12i2 polypeptide or complex as described herein with DNA in the cell.

The invention yet further provides a composition or formulation comprising a variant Cas12i2 polypeptide as described herein, an RNA guide, and a cell.

The invention yet further provides a method of producing a composition as described herein.

The invention yet further provides a method of complexing a variant Cas12i2 polypeptide as described herein with an RNA guide, such as an RNA guide described herein.

The invention yet further provides a method of complexing a variant binary complex as described herein with a target nucleic acid.

The invention yet further provides a method of delivering a composition as described herein.

The invention yet further provides a composition comprising a variant Cas12i2 polypeptide, wherein the variant Cas12i2 polypeptide comprises a substitution that increases interactions between the variant Cas12i2 polypeptide and a nucleic acid, as compared to a parent polypeptide.

In one aspect of the composition, the variant Cas12i2 polypeptide comprises the sequence set forth in SEQ ID NO: 4.

In another aspect of the composition, the variant Cas12i2 polypeptide comprises the sequence set forth in any one of SEQ ID NO: 3, SEQ ID NO: 5, SEQ ID NO: 495, and SEQ ID NO: 496.

In another aspect of the composition, the interactions are electrostatic interactions.

In another aspect of the composition, the interactions are non-specific interactions.

In another aspect of the composition, the interactions are aromatic, hydrophobic, Van der Waals, and/or cation-pi interactions.

In another aspect of the composition, the substitution is in or adjacent to a nucleic acid interface.

In another aspect of the composition, the nucleic acid is an RNA guide comprising a direct repeat sequence and a spacer sequence.

In another aspect of the composition, the direct repeat sequence comprises a nucleotide sequence with at least 95% sequence identity to any one of SEQ ID NO: 492-494.

In another aspect of the composition, the direct repeat sequence comprises a nucleotide sequence set forth in any one of SEQ ID NO: 492-494.

In another aspect of the composition, the substitution increases interactions between the variant Cas12i2 polypeptide and the direct repeat sequence.

In another aspect of the composition, the substitution increases binary complex formation, as compared to a parent polypeptide.

In another aspect of the composition, a binary complex comprising the variant Cas12i2 polypeptide exhibits increased stability, as compared to a parent binary complex.

In another aspect of the composition, the substitution is an arginine, lysine, glutamine, asparagine, histidine, tyrosine, or serine substitution.

In another aspect of the composition, the substitution is in an RNA binding interface.

In another aspect of the composition, the substitution is a substitution in the Wedge domain or Rec2 domain.

In another aspect of the composition, the substitution is a substitution listed in Table 4.

In another aspect of the composition, the variant Cas12i2 polypeptide comprises a sequence having at least 95% identity to a sequence set forth in any one of SEQ ID NOs: 3 to 146 or in any one of SEQ ID NOs: 495-512.

In another aspect of the composition, the variant Cas12i2 polypeptide comprises a sequence having at least 95% identity to a sequence set forth in any one of SEQ ID NOs: 3 to 146 or in any one of SEQ ID NOs: 495 to 512 and further having a substitution listed in Table 4.

In another aspect of the composition, the variant Cas12i2 polypeptide comprises a sequence set forth in any one of SEQ ID NOs: 3 to 146 or in any one of SEQ ID NOs: 495 to 512.

In another aspect of the composition, the variant Cas12i2 polypeptide comprises a sequence set forth in any one of SEQ ID NOs: 3 to 146 or in any one of SEQ ID NOs: 495 to 512 and further has a substitution listed in Table 4.

In another aspect of the composition, the direct repeat sequence is set forth in SEQ ID NO: 492 and the variant Cas12i2 polypeptide comprises a sequence set forth in SEQ ID NO: 4.

In another aspect of the composition, the direct repeat sequence is set forth in SEQ ID NO: 492 and the variant Cas12i2 polypeptide comprises the sequence set forth in any one of SEQ ID NO: 3, SEQ ID NO: 5, SEQ ID NO: 495, and SEQ ID NO: 496.

In another aspect of the composition, the nucleic acid is a target nucleic acid.

In another aspect of the composition, the nucleic acid is double-stranded DNA.

In another aspect of the composition, the substitution increases interactions between the variant Cas12i2 polypeptide and double-stranded DNA.

In another aspect of the composition, the double-stranded DNA comprises a PAM sequence.

In another aspect of the composition, the substitution increases ternary complex formation, as compared to a parent polypeptide.

In another aspect of the composition, a ternary complex comprising the variant Cas12i2 polypeptide exhibits increased stability, as compared to a parent ternary complex.

In another aspect of the composition, the substitution is an arginine, lysine, glutamine, asparagine, histidine, or serine substitution.

In another aspect of the composition, the substitution is in a double-stranded DNA binding interface.

In another aspect of the composition, the substitution is a substitution in the Rec1 domain, PI domain, or Wedge domain.

In another aspect of the composition, the substitution is a substitution listed in Table 5.

In another aspect of the composition, the variant Cas12i2 polypeptide comprises a sequence having at least 95% identity to a sequence set forth in any one of SEQ ID NOs: 3-146 or in any one of SEQ ID NOs: 495-512.

In another aspect of the composition, the variant Cas12i2 polypeptide comprises a sequence having at least 95% identity to a sequence set forth in any one of SEQ ID NOs: 3-146 or in any one of SEQ ID NOs: 495-512 and further having a substitution listed in Table 5.

In another aspect of the composition, the variant Cas12i2 polypeptide comprises a sequence set forth in any one of SEQ ID NOs: 3-146 or in any one of SEQ ID NOs: 495-512.

In another aspect of the composition, the variant Cas12i2 polypeptide comprises a sequence set forth in any one of SEQ ID NOs: 3-146 or in any one of SEQ ID NOs: 495-512 and further has a substitution listed in Table 5.

In another aspect of the composition, the nucleic acid is single-stranded DNA.

In another aspect of the composition, the single-stranded DNA comprises a non-target strand.

In another aspect of the composition, the single-stranded DNA comprises a target strand.

In another aspect of the composition, the substitution increases ternary complex formation, as compared to a parent polypeptide.

In another aspect of the composition, a ternary complex comprising the variant Cas12i2 polypeptide exhibits increased stability, as compared to a parent ternary complex.

In another aspect of the composition, the substitution is an arginine, lysine, glutamine, asparagine, histidine, or alanine substitution.

In another aspect of the composition, the substitution is in a single-stranded DNA binding interface.

In another aspect of the composition, the substitution is a substitution in the PI domain, Rec1 domain, Wedge domain, RuvC domain, Rec2 domain, or Nuc domain.

In another aspect of the composition, the substitution is a substitution listed in Table 6.

In another aspect of the composition, the variant Cas12i2 polypeptide comprises a sequence having at least 95% identity to a sequence set forth in any one of SEQ ID NOs: 3 to 146 or in any one of SEQ ID NOs: 495 to 512.

In another aspect of the composition, the variant Cas12i2 polypeptide comprises a sequence having at least 95% identity to a sequence set forth in any one of SEQ ID NOs: 3 to 146 or in any one of SEQ ID NOs: 495 to 512 and further having a substitution listed in Table 6.

In another aspect of the composition, the variant Cas12i2 polypeptide comprises a sequence set forth in any one of SEQ ID NOs: 3 to 146 or in any one of SEQ ID NOs: 495 to 512.

In another aspect of the composition, the variant Cas12i2 polypeptide comprises a sequence set forth in any one of SEQ ID NOs: 3 to 146 or in any one of SEQ ID NOs: 495 to 512 and further has a substitution listed in Table 6.

In another aspect of the composition, the substitution increases interactions between the variant Cas12i2 polypeptide and a DNA/RNA hybrid molecule.

In another aspect of the composition, the DNA/RNA hybrid molecule is a heteroduplex comprising a spacer sequence of an RNA guide and a target strand.

In another aspect of the composition, the substitution stabilizes the heteroduplex.

In another aspect of the composition, the substitution increases ternary complex formation, as compared to a parent polypeptide.

In another aspect of the composition, a ternary complex comprising the variant Cas12i2 polypeptide exhibits increased stability, as compared to a parent ternary complex.

In another aspect of the composition, the substitution is an arginine, lysine, glutamine, asparagine, histidine, or serine substitution.

In another aspect of the composition, the substitution is a substitution in the Rec1 domain, PI domain, Rec2 domain, or RuvC2 motif.

In another aspect of the composition, the substitution is a substitution listed in Table 7.

In another aspect of the composition, the variant Cas12i2 polypeptide comprises a sequence having at least 95% identity to a sequence set forth in any one of SEQ ID NOs: 3-146 or in any one of SEQ ID NOs: 495-512.

In another aspect of the composition, the variant Cas12i2 polypeptide comprises a sequence having at least 95% identity to a sequence set forth in any one of SEQ ID NOs: 3-146 or in any one of SEQ ID NOs: 495-512 and further having a substitution listed in Table 7.

In another aspect of the composition, the variant Cas12i2 polypeptide comprises a sequence set forth in any one of SEQ ID NOs: 3-146 or in any one of SEQ ID NOs: 495-512.

In another aspect of the composition, the variant Cas12i2 polypeptide comprises a sequence set forth in any one of SEQ ID NOs: 3-146 or in any one of SEQ ID NOs: 495-512 and further has a substitution listed in Table 7.

In another aspect of the composition, the substitution increases interactions between the variant Cas12i2 polypeptide and bases of (a) a double-stranded DNA duplex and/or (b) a heteroduplex comprising a spacer sequence of an RNA guide and a target strand.

In another aspect of the composition, the double-stranded DNA duplex comprises a PAM sequence.

In another aspect of the composition, the interactions are aromatic, hydrophobic, Van der Waals, and/or cation-pi interactions.

In another aspect of the composition, the substitution stabilizes the R-loop.

In another aspect of the composition, the substitution increases ternary complex formation, as compared to a parent polypeptide.

In another aspect of the composition, a ternary complex comprising the variant Cas12i2 polypeptide exhibits increased stability, as compared to a parent ternary complex.

In another aspect of the composition, the substitution is an arginine, lysine, tryptophan, phenylalanine, tyrosine, methionine, histidine, glutamine, threonine, or valine substitution.

In another aspect of the composition, the substitution is a substitution in the Wedge domain, Rec1 domain, or RuvC domain.

In another aspect of the composition, the substitution is a substitution listed in Table 8.

In another aspect of the composition, the variant Cas12i2 polypeptide comprises a sequence having at least 95% identity to a sequence set forth in any one of SEQ ID NOs: 3-146 or in any one of SEQ ID NOs: 495-512.

In another aspect of the composition, the variant Cas12i2 polypeptide comprises a sequence having at least 95% identity to a sequence set forth in any one of SEQ ID NOs: 3-146 or in any one of SEQ ID NOs: 495-512 and further having a substitution listed in Table 8.

In another aspect of the composition, the variant Cas12i2 polypeptide comprises a sequence set forth in any one of SEQ ID NOs: 3-146 or in any one of SEQ ID NOs: 495-512.

In another aspect of the composition, the variant Cas12i2 polypeptide comprises a sequence set forth in any one of SEQ ID NOs: 3-146 or in any one of SEQ ID NOs: 495-512 and further has a substitution listed in Table 8.

In another aspect of the composition, the parent polypeptide comprises the amino acid sequence of SEQ ID NO: 2.

In another aspect of the composition, the variant Cas12i2 polypeptide exhibits increased enzymatic activity, as compared to a parent polypeptide.

In another aspect of the composition, the composition further comprises an RNA guide comprising a direct repeat sequence and a spacer sequence.

In another aspect of the composition, the direct repeat sequence comprises a nucleotide sequence with at least 95% sequence identity to any one of SEQ ID NO: 492-494.

In another aspect of the composition, the direct repeat sequence comprises a nucleotide sequence set forth in any one of SEQ ID NO: 492-494.

In another aspect of the composition, the direct repeat sequence is set forth in SEQ ID NO: 492 and the variant Cas12i2 polypeptide comprises a sequence set forth in SEQ ID NO: 4. 239.

In another aspect of the composition, the direct repeat sequence is set forth in SEQ ID NO: 492 and the variant Cas12i2 polypeptide comprises the sequence set forth in any one of SEQ ID NO: 3, SEQ ID NO: 5, SEQ ID NO: 495, and SEQ ID NO: 496.

The invention yet further provides a cell comprising a composition described herein.

In one aspect of the cell, the composition does not substantially affect viability of the cell.

The invention yet further provides a composition comprising a variant Cas12i2 polypeptide, wherein the variant Cas12i2 polypeptide comprises a substitution that increases flexibility of the variant Cas12i2 polypeptide during DNA binding, as compared to a parent polypeptide.

In one aspect of the composition, the variant Cas12i2 polypeptide comprises the sequence set forth in SEQ ID NO: 4.

In another aspect of the composition, the Cas12i2 polypeptide comprises the sequence set forth in any one of SEQ ID NO: 3, SEQ ID NO: 5, SEQ ID NO: 495, and SEQ ID NO: 496.

In another aspect of the composition, the substitution increases binding of the variant Cas12i2 polypeptide to DNA.

In another aspect of the composition, the substitution increases binding of the variant Cas12i2 polypeptide to double-stranded DNA.

In another aspect of the composition, the substitution increases binding of the variant Cas12i2 polypeptide to single-stranded DNA.

In another aspect of the composition, the substitution increases ternary complex formation, as compared to a parent polypeptide.

In another aspect of the composition, a ternary complex comprising the variant Cas12i2 polypeptide exhibits increased stability, as compared to a parent ternary complex.

In another aspect of the composition, the substitution is a substitution of a bulky amino acid to an amino acid with a smaller side chain.

In another aspect of the composition, the substitution is an alanine, valine, glycine, or serine substitution.

In another aspect of the composition, the substitution is in the Helical II domain of the variant Cas12i2 polypeptide.

In another aspect of the composition, the substitution is a substitution listed in Table 9.

In another aspect of the composition, the variant Cas12i2 polypeptide comprises a sequence having at least 95% identity to a sequence set forth in any one of SEQ ID NOs: 3-146 or in any one of SEQ ID NOs: 495-512.

In another aspect of the composition, the variant Cas12i2 polypeptide comprises a sequence having at least 95% identity to a sequence set forth in any one of SEQ ID NOs: 3-146 or in any one of SEQ ID NOs: 495-512 and further having a substitution listed in Table 9.

In another aspect of the composition, the variant Cas12i2 polypeptide comprises a sequence set forth in any one of SEQ ID NOs: 3-146 or in any one of SEQ ID NOs: 495-512.

In another aspect of the composition, the variant Cas12i2 polypeptide comprises a sequence set forth in any one of SEQ ID NOs: 3-146 or in any one of SEQ ID NOs: 495-512 and further has a substitution listed in Table 9.

The invention yet further provides a composition comprising a variant Cas12i2 polypeptide, wherein the variant Cas12i2 polypeptide comprises a substitution that stabilizes a domain-domain interface that forms during ternary complex formation, as compared to a parent polypeptide.

In one aspect of the composition, the variant Cas12i2 polypeptide comprises the sequence set forth in SEQ ID NO: 4.

In another aspect of the composition, the variant Cas12i2 polypeptide comprises the sequence set forth in any one of SEQ ID NO: 3, SEQ ID NO: 5, SEQ ID NO: 495, and SEQ ID NO: 496.

In another aspect of the composition, the domain-domain interface forms when single-stranded DNA contacts an active site of the variant Cas12i2 polypeptide.

In another aspect of the composition, the domain-domain interface is a Helical II domain-Nuc domain interface.

In another aspect of the composition, the substitution increases ternary complex formation, as compared to a parent polypeptide.

In another aspect of the composition, a ternary complex comprising the variant Cas12i2 polypeptide exhibits increased stability, as compared to a parent ternary complex.

In another aspect of the composition, the substitution is an aspartic acid, glutamic acid, arginine, or lysine substitution.

In another aspect of the composition, the substitution is a substitution listed in Table 10.

In another aspect of the composition, the variant Cas12i2 polypeptide comprises a sequence having at least 95% identity to a sequence set forth in any one of SEQ ID NOs: 3-146 or in any one of SEQ ID NOs: 495-512.

In another aspect of the composition, the variant Cas12i2 polypeptide comprises a sequence having at least 95% identity to a sequence set forth in any one of SEQ ID NOs: 3-146 or in any one of SEQ ID NOs: 495-512 and further having a substitution listed in Table 10.

In another aspect of the composition, the variant Cas12i2 polypeptide comprises a sequence set forth in any one of SEQ ID NOs: 3-146 or in any one of SEQ ID NOs: 495-512.

In another aspect of the composition, the variant Cas12i2 polypeptide comprises a sequence set forth in any one of SEQ ID NOs: 3-146 or in any one of SEQ ID NOs: 495-512 and further has a substitution listed in Table 10.

In another aspect of the composition, the parent polypeptide comprises the amino acid sequence of SEQ ID NO: 2.

In another aspect of the composition, the variant Cas12i2 polypeptide exhibits increased enzymatic activity, as compared to a parent polypeptide.

In another aspect of the composition, the composition further comprises an RNA guide comprising a direct repeat sequence and a spacer sequence.

In another aspect of the composition, the direct repeat sequence comprises a nucleotide sequence with at least 95% sequence identity to any one of SEQ ID NO: 492-494.

In another aspect of the composition, the direct repeat sequence comprises a nucleotide sequence set forth in any one of SEQ ID NO: 492-494.

In another aspect of the composition, the direct repeat sequence is set forth in SEQ ID NO: 492 and the variant Cas12i2 polypeptide comprises a sequence set forth in SEQ ID NO: 4.

In another aspect of the composition, the direct repeat sequence is set forth in SEQ ID NO: 492 and the variant Cas12i2 polypeptide comprises the sequence set forth in any one of SEQ ID NO: 3, SEQ ID NO: 5, SEQ ID NO: 495, and SEQ ID NO: 496.

The invention yet further provides a cell comprising the composition as described herein.

In one aspect of the cell, the composition does not substantially affect viability of the cell.

The invention yet further provides a composition comprising a variant Cas12i2 polypeptide, wherein the variant Cas12i2 polypeptide comprises a substitution that increases on-target specificity of the variant Cas12i2 polypeptide, as compared to a parent polypeptide.

In one aspect of the composition, the variant Cas12i2 polypeptide comprises the sequence set forth in SEQ ID NO: 4.

In another aspect of the composition, the variant Cas12i2 polypeptide comprises the sequence set forth in any one of SEQ ID NO: 3, SEQ ID NO: 5, SEQ ID NO: 495, and SEQ ID NO: 496.

In another aspect of the composition, the substitution increases on-target DNA binding.

In another aspect of the composition, the substitution decreases off-target DNA binding.

In another aspect of the composition, the substitution increases on-target ternary complex formation, as compared to a parent polypeptide.

In another aspect of the composition, an on-target ternary complex comprising the variant Cas12i2 polypeptide exhibits increased stability, as compared to a parent ternary complex.

In another aspect of the composition, the substitution is a substitution of an amino acid contacting the spacer sequence of an RNA guide.

In another aspect of the composition, the substitution is a substitution of a bulky amino acid to an amino acid with a smaller side chain.

In another aspect of the composition, the substitution is an alanine, serine, valine, glutamine, or asparagine substitution.

In another aspect of the composition, the substitution is a substitution in the Wedge domain, Rec1 domain, Rec2 domain, or RuvC domain.

In another aspect of the composition, the substitution is a substitution in the Helical II domain.

In another aspect of the composition, the substitution is a substitution listed in Table 11.

In another aspect of the composition, the variant Cas12i2 polypeptide comprises a sequence having at least 95% identity to a sequence set forth in any one of SEQ ID NOs: 3-146 or in any one of SEQ ID NOs: 495-512.

In another aspect of the composition, the variant Cas12i2 polypeptide comprises a sequence having at least 95% identity to a sequence set forth in any one of SEQ ID NOs: 3-146 or in any one of SEQ ID NOs: 495-512 and further having a substitution listed in Table 11.

In another aspect of the composition, the variant Cas12i2 polypeptide comprises a sequence set forth in any one of SEQ ID NOs: 3-146 or in any one of SEQ ID NOs: 495-512.

In another aspect of the composition, the variant Cas12i2 polypeptide comprises a sequence set forth in any one of SEQ ID NOs: 3-146 or in any one of SEQ ID NOs: 495-512 and further has a substitution listed in Table 11.

In another aspect of the composition, the substitution decreases a catalysis rate (Kcat) of the variant Cas12i2 polypeptide.

In another aspect of the composition, the substitution is an alanine, serine, threonine, valine, leucine, methionine, asparagine, or isoleucine substitution.

In another aspect of the composition, the substitution is a substitution in the Wedge domain, Rec1 domain, Rec2 domain, or RuvC domain.

In another aspect of the composition, the substitution is a substitution in the RuvC domain.

In another aspect of the composition, the substitution is a substitution listed in Table 12.

In another aspect of the composition, the variant Cas12i2 polypeptide comprises a sequence having at least 95% identity to a sequence set forth in any one of SEQ ID NOs: 3-146 or in any one of SEQ ID NOs: 495-512.

In another aspect of the composition, the variant Cas12i2 polypeptide comprises a sequence having at least 95% identity to a sequence set forth in any one of SEQ ID NOs: 3-146 or in any one of SEQ ID NOs: 495-512 and further having a substitution listed in Table 12.

In another aspect of the composition, the variant Cas12i2 polypeptide comprises a sequence set forth in any one of SEQ ID NOs: 3-146 or in any one of SEQ ID NOs: 495-512.

In another aspect of the composition, the variant Cas12i2 polypeptide comprises a sequence set forth in any one of SEQ ID NOs: 3-146 or in any one of SEQ ID NOs: 495-512 and further has a substitution listed in Table 12.

In another aspect of the composition, the variant Cas12i2 polypeptide exhibits increased on-target enzymatic activity, as compared to a parent polypeptide.

In another aspect of the composition, the variant Cas12i2 polypeptide exhibits decreased off-target enzymatic activity, as compared to a parent polypeptide.

In another aspect of the composition, the variant Cas12i2 polypeptide exhibits off-target editing that is no more than 10% of the on-target editing.

In another aspect of the composition, the variant Cas12i2 polypeptide exhibits off-target editing that is no more than 5% of the on-target editing.

In another aspect of the composition, the composition further comprises an RNA guide comprising a direct repeat sequence and a spacer sequence.

In another aspect of the composition, the direct repeat sequence comprises a nucleotide sequence with at least 95% sequence identity to any one of SEQ ID NO: 492-494.

In another aspect of the composition, the direct repeat sequence comprises a nucleotide sequence set forth in any one of SEQ ID NO: 492-494.

In another aspect of the composition, the direct repeat sequence is set forth in SEQ ID NO: 492 and the variant Cas12i2 polypeptide comprises a sequence set forth in SEQ ID NO: 4.

In another aspect of the composition, the direct repeat sequence is set forth in SEQ ID NO: 492 and the variant Cas12i2 polypeptide comprises the sequence set forth in any one of SEQ ID NO: 3, SEQ ID NO: 5, SEQ ID NO: 495, and SEQ ID NO: 496.

The invention yet further provides a cell comprising the composition as described herein.

In one aspect of the cell, the composition does not substantially affect viability of the cell.

In another aspect of the composition, the variant Cas12i2 polypeptide exhibits a higher ratio of on-target binding to off-target binding, as compared to a Cas9 polypeptide.

In another aspect of the composition, the variant Cas12i2 polypeptide exhibits a higher ratio of on-target activity to off-target activity, as compared to a Cas9 polypeptide.

In another aspect of the composition, the variant Cas12i2 polypeptide exhibits a higher ratio of on-target editing to off-target editing, as compared to a Cas9 polypeptide.

In another aspect of the composition, the variant Cas12i2 polypeptide exhibits less off-target binding, as compared to a Cas9 polypeptide.

In another aspect of the composition, the variant Cas12i2 polypeptide exhibits less off-target activity, as compared to a Cas9 polypeptide.

In another aspect of the composition, the variant Cas12i2 polypeptide exhibits less off-target editing, as compared to a Cas9 polypeptide.

In another aspect of the composition, off-target binding, off-target activity, and/or off-target editing by the variant Cas12i2 polypeptide is at least 10% less than off-target binding, off-target activity, and/or off-target editing by a Cas9 polypeptide.

In another aspect of the composition, off-target binding, off-target activity, and/or off-target editing by the variant Cas12i2 polypeptide is at least 20% less than off-target binding, off-target activity, and/or off-target editing by a Cas9 polypeptide.

In another aspect of the composition, off-target binding, off-target activity, and/or off-target editing by the variant Cas12i2 polypeptide is at least 30% less than off-target binding, off-target activity, and/or off-target editing by a Cas9 polypeptide.

In another aspect of the composition, off-target binding, off-target activity, and/or off-target editing by the variant Cas12i2 polypeptide is at least 40% less than off-target binding, off-target activity, and/or off-target editing by a Cas9 polypeptide.

In another aspect of the composition, off-target binding, off-target activity, and/or off-target editing by the variant Cas12i2 polypeptide is at least 50% less than off-target binding, off-target activity, and/or off-target editing by a Cas9 polypeptide.

In yet another aspect, the invention provides a cell comprising a composition described herein.

In one aspect of the cell, the composition does not substantially affect viability of the cell.

### Definitions

The present invention will be described with respect to particular embodiments and with reference to certain Figures, but the invention is not limited thereto but only by the claims. Terms as set forth hereinafter are generally to be understood in their common sense unless indicated otherwise.

As used herein, the term “activity” refers to a biological activity. In some embodiments, the activity refers to effector activity. In some embodiments, activity includes enzymatic activity, e.g., catalytic ability of an effector. For example, activity can include nuclease activity. In some embodiments, activity includes binding activity, e.g., binding activity of an effector to an RNA guide and/or target nucleic acid.

As used herein, the term “adjacent to” refers to a nucleotide or amino acid sequence in close proximity to another nucleotide or amino acid sequence. In some embodiments, a nucleotide sequence is adjacent to another nucleotide sequence if no nucleotides separate the two sequences. In some embodiments, a nucleotide sequence is adjacent to another nucleotide sequence if a small number of nucleotides separate the two sequences (e.g., about 1, 2, 3, 4, 5, 6, 7, 8, 9, 10, 11, 12, 13, 14, 15, 16, 17, 18, 19, or 20 nucleotides). In some embodiments, a first sequence is adjacent to a second sequence if the two sequences are separated by about 5, 6, 7, 8, 9, 10, 11, 12, 13, 14, or 15 nucleotides. In some embodiments, the term “adjacent to” is used to refer a protein residue that interacts with another protein residue. In some embodiments, the term “adjacent to” is used to refer a protein residue that interacts with a nucleotide or nucleic acid. In some embodiments, the term “adjacent to” is used to refer to a protein domain or motif that interacts with another protein domain or motif. In some embodiments, the term “adjacent to” is used to refer to a protein domain or motif that interacts with a nucleotide or nucleic acid sequence. As used herein, the term “adjacent to” is used to refer to the positioning of an indel (insertion/deletion) in a modified cell of the disclosure.

As used herein, the term “complex” refers to a grouping of two or more molecules. In some embodiments, the complex comprises a polypeptide and a nucleic acid molecule interacting with (e.g. binding to, coming into contact with, adhering to) one another.

As used herein, the term “binary complex” refers to a grouping of two molecules (e.g., a polypeptide and a nucleic acid molecule). In some embodiments, a binary complex refers to a grouping of a polypeptide and a targeting moiety (e.g., an RNA guide). In some embodiments, a binary complex refers to a ribonucleoprotein (RNP). As used herein, the term “variant binary complex” refers to the grouping of a variant Cas12i2 polypeptide and RNA guide. As used herein, the term “parent binary complex” refers to the grouping of a parent polypeptide and RNA guide or a reference polypeptide and RNA guide.

As used herein, the term “ternary complex” refers to a grouping of three molecules (e.g., a polypeptide and two nucleic acid molecules). In some embodiments, a “ternary complex” refers to a grouping of a polypeptide, an RNA molecule, and a DNA molecule. In some embodiments, a ternary complex refers to a grouping of a polypeptide, a targeting moiety (e.g., an RNA guide), and a target nucleic acid (e.g., a target DNA molecule). In some embodiments, a “ternary complex” refers to a grouping of a binary complex (e.g., a ribonucleoprotein) and a third molecule (e.g., a target nucleic acid). As used herein, the terms “variant ternary complex,” “variant Cas12i2 ternary complex,” and “Cas12i2 variant ternary complex” refer to the grouping of a variant Cas12i2 polypeptide, RNA guide, and target nucleic acid (e.g., a variant Cas12i2 ribonucleoprotein and target nucleic acid). As used herein, the terms “parent ternary complex,” “parent Cas12i2 ternary complex,” and “Cas12i2 parent ternary complex” refer to the grouping of a parent polypeptide, RNA guide, and target nucleic acid (e.g., a parent ribonucleoprotein and target nucleic acid) or a reference polypeptide, RNA guide, and target nucleic acid (e.g., a reference ribonucleoprotein and target nucleic acid).

As used herein, the term “deletion” refers to a loss or removal of nucleotides in a nucleic acid sequence. The deletion can be in a genome of an organism. The deletion can be in a cell. The deletion can be a DNA sequence. The deletion can be an RNA sequence. The deletion can be a frameshift mutation or a non-frameshift mutation. A Cas12i2-induced deletion described herein can refer to a deletion of up to about 100 nucleotides, such as from about 4 nucleotides and 100 nucleotides, from about 4 nucleotides and 50 nucleotides, from about 4 nucleotides and 40 nucleotides, from about 4 nucleotides and 25 nucleotides, from about 10 nucleotides and 25 nucleotides, from about 10 nucleotides and 15 nucleotides, from a nucleic acid molecule. In some embodiments, a Cas12i2-induced deletion described herein occurs downstream of a 5′-NTTN-3′ sequence. In some embodiments, the term “Cas12i2-induced” refers to a deletion that results from a DNA break induced by a Cas12i2 polypeptide. In some embodiments, the term “Cas12i2-induced” refers to a deletion that results from a DNA break induced by a Cas12i2 polypeptide and repaired by a cell's DNA repair machinery.

As used herein, the term “domain” refers to a distinct functional and/or structural unit of a polypeptide. In some embodiments, a domain may comprise a conserved amino acid sequence.

As used herein, the terms “editing efficiency” and “indel activity” refer to the ability of an enzyme (e.g., a variant Cas12i2 polypeptide) to introduce an indel (insertion/deletion) into a sequence. For example, in some embodiments, an enzyme that introduces an indel into each of ten target loci exhibits an editing efficiency of 100%. An enzyme that introduces an indel into five out of ten target loci exhibits an editing efficiency of 50%. In another example, an enzyme that introduces an indel at a target locus in 50% of a plurality of cells exhibits an editing efficiency of 50%. As used herein, the terms “editing efficiency,” “indel activity,” and “on-target editing” refer to the ability of an enzyme (e.g., a variant Cas12i2 polypeptide) to selectively introduce an indel at a target locus. In some embodiments, editing efficiency at a target locus is compared to editing efficiency at a non-target locus. In some embodiments, editing at a target locus is compared to editing at a non-target locus.

As used herein, the term “effector activity” refers to a biological activity. In some embodiments, effector activity includes enzymatic activity, e.g., catalytic ability of an effector. For example, effector activity can include nuclease activity.

As used herein, the term “interface” refers to one or more residues of a variant Cas12i2 polypeptide (e.g., a domain/motif or a portion of a domain/motif) in contact with (e.g., that interact with or are adjacent to) a nucleic acid molecule or a distinct domain/motif or a portion of a distinct domain/motif of the variant Cas12i2 polypeptide. In some aspects, an interface is a buried surface area between adjacent domains or motifs. In some aspects, an interface is a surface area between the a polypeptide and a ligand (e.g., DNA or RNA) where the polypeptide and ligand make contact. As used herein, the term “nucleic acid interface” refers to residues of the variant Cas12i2 polypeptide that are in close proximity to (e.g., are adjacent to) or interact with a nucleic acid sequence (e.g., a DNA sequence or an RNA sequence). As used herein, the term “RNA binding interface” refers to the residues of the variant Cas12i2 polypeptide that are in close proximity to (e.g., are adjacent to) or interact with an RNA guide (e.g., the direct repeat of the RNA guide). As used herein, the term “double-stranded DNA binding interface” refers to the residues of the variant Cas12i2 polypeptide that are in close proximity to (e.g., are adjacent to) and/or interact with double-stranded DNA. As used herein, the term “single-stranded DNA binding interface” refers to the residues of the variant Cas12i2 polypeptide that are in close proximity to (e.g., are adjacent to) and/or interact with single-stranded DNA. As used herein, the term “domain-domain interface” refers to a domain in close-proximity to (e.g., adjacent to) a separate domain. In some embodiments, a domain-domain interface (e.g., a Helical II domain-Nuc domain interface) forms upon complex formation (e.g., ternary complex formation).

As used herein, the terms “parent,” “parent polypeptide,” and “parent sequence” refer to an original polypeptide (e.g., starting polypeptide) to which an alteration is made to produce a variant Cas12i2 polypeptide of the present invention. In some embodiments, the parent is a polypeptide having an identical amino acid sequence of the variant with one or more variations at one or more specified positions. In exemplary embodiments, variations refer to amino acid changes within the polypeptide sequence. The parent may be a naturally occurring (wild-type) polypeptide. In a particular embodiment, the parent is a polypeptide with at least 60%, at least 61%, at least 62%, at least 63%, at least 64%, at least 65%, at least 70%, at least 72%, at least 73%, at least 74%, at least 75%, at least 80%, at least 81%, at least 82%, at least 83%, at least 84%, at least 85%, at least 90%, at least 91%, at least 92%, at least 93%, at least 94%, at least 95%, at least 96%, at least 97%, at least 98%, at least 99% or 100% identity to a polypeptide of SEQ ID NO: 2. In some embodiments, variations may include structural changes suck as linkages, fusions, or other changes that do not alter the original amino acid sequence of the parent. In some embodiments, the parent polypeptide sequence includes amino acid variations and structural changes.

As used herein, a “plurality of variant binary complexes” refers to a plurality of binary complexes comprising a plurality of variant Cas12i2 polypeptides. A plurality of binary complexes comprising a plurality of parent polypeptides (e.g., wild-type Cas12i2 polypeptides parent of SEQ ID NO: 2) are referred to herein as a “plurality of parent binary complexes.” A plurality of Cas12i2 polypeptides, e.g., a plurality of variant Cas12i2 polypeptides, and two or more distinct targeting moieties, e.g., two or more distinct RNA guides, individually form two or more binary complexes (e.g., a plurality of binary complexes). In such embodiments, complex formation may be accomplished simultaneously in a single composition or independently in separate compositions.

As used herein, the term “protospacer adjacent motif” or “PAM” refers to a DNA sequence adjacent to a target sequence to which a binary complex comprising a polypeptide (e.g., an enzyme such as Cas12i2 or a variant thereof) and a targeting moiety (e.g., an RNA guide) binds. In some embodiments, a PAM is required for enzyme activity. In the case of a double-stranded target, the targeting moiety (e.g., the RNA guide) binds to a first strand of the target, and a PAM sequence as described herein is present in the second, complementary strand. For example, in some embodiments, the RNA guide binds to the target strand (e.g., the spacer-complementary strand), and the PAM sequence as described herein is present in the non-target strand (i.e., the non-spacer-complementary strand). In some embodiments, the target strand (i.e., the spacer-complementary strand) comprises a 5′-NAAN-3′ sequence.

As used herein, the terms “reference composition,” “reference molecule,” “reference sequence,” “reference,” and “reference complex” refer to a control, such as a negative control or a parent (e.g., a parent sequence, a parent protein, a wild-type protein, or a complex comprising a parent sequence). For example, a reference molecule refers to a Cas12i2 polypeptide to which a variant Cas12i2 polypeptide is compared. Likewise, a reference RNA guide refers to a targeting moiety to which a modified RNA guide is compared. The variant or modified molecule may be compared to the reference molecule on the basis of sequence (e.g., the variant or modified molecule may have X % sequence identity or homology with the reference molecule), thermostability, or activity (e.g., the variant or modified molecule may have X % of the activity of the reference molecule). For example, where the reference molecule is a Cas12i2 polypeptide, a variant or modified molecule may be characterized as having no more than 10% of an activity of the reference Cas12i2 polypeptide or may be characterized as having at least 10% greater of an activity of the reference Cas12i2 polypeptide. Examples of reference Cas12i2 polypeptides include naturally occurring unmodified Cas12i2 polypeptides, e.g., naturally occurring Cas12i2 polypeptide from archaea or other bacterial species. In certain embodiments, the reference Cas12i2 polypeptide is a naturally occurring Cas12i2 polypeptide having the closest sequence identity or homology with the variant Cas12i2 polypeptide to which it is being compared. In certain embodiments, the reference Cas12i2 polypeptide is a parental molecule having a naturally occurring or known sequence on which a mutation has been made to arrive at the variant Cas12i2 polypeptide.

As used herein, the terms “RNA guide” or “RNA guide sequence” refer to any RNA molecule that facilitates the targeting of a polypeptide described herein to a target nucleic acid. For example, an RNA guide can be a molecule that recognizes (e.g., binds to) a target nucleic acid. An RNA guide may be designed to be complementary to a specific nucleic acid sequence. An RNA guide comprises a DNA targeting sequence and a direct repeat (DR) sequence. The terms CRISPR RNA (crRNA), pre-crRNA and mature crRNA are also used herein to refer to an RNA guide.

As used herein, the term “substantially identical” refers to a sequence, polynucleotide, or polypeptide, that has a certain degree of identity to a reference sequence.

As used herein, the term “targeting moiety” refers to a molecule or component (e.g., nucleic acid and/or RNA guide) that facilitates the targeting of another molecule or component to a target nucleic acid. In some embodiments, the targeting moiety specifically interacts or associates with the target nucleic acid.

As used herein, the terms “target nucleic acid,” “target sequence,” “target substrate,” and “on-target locus” refer to a nucleic acid sequence to which a targeting moiety (e.g., RNA guide) specifically binds. In some embodiments, the DNA targeting sequence of an RNA guide binds to a target nucleic acid. Binding of a binary complex to a target locus is referred to herein as “on-target binding.”

As used herein, the terms “non-target” and “off-target” refer to a nucleic acid sequence other than the sequence to which a targeting moiety specifically binds or is intended to specifically bind. A non-target locus is an unintended target of a targeting moiety (e.g., an RNA guide). Binding of a binary complex to a non-target locus is referred to herein as “off-target binding.” In some embodiments, a non-target locus is a locus on a target nucleic acid. In some embodiments, a non-target locus is a locus on a nucleic acid other than the target nucleic acid (e.g., a non-target nucleic acid).

As used herein, the terms “upstream” and “downstream” refer to relative positions within a single nucleic acid (e.g., DNA) sequence in a nucleic acid molecule. As used herein, the terms “upstream” and “downstream” refer to the positioning of two sequences relative to one another. “Upstream” and “downstream” relate to the 5′ to 3′ direction, respectively, in which RNA transcription occurs. A first sequence is upstream of a second sequence when the 3′ end of the first sequence occurs before the 5′ end of the second sequence. A first sequence is downstream of a second sequence when the 5′ end of the first sequence occurs after the 3′ end of the second sequence. As used herein, the terms “upstream” and “downstream” are used to refer to the relative positioning of a deletion in reference to a 5‘-NTTN’-3′ sequence. As used herein, the 5′-NTTN-3′ sequence is upstream of a Cas12i2-induced deletion, and a Cas12i2-induced deletion is downstream of the 5′-NTTN-3′ sequence.

As used herein, the terms “variant Cas12i2 polypeptide” and “variant effector polypeptide” refer to a polypeptide comprising an alteration, e.g., a substitution, insertion, deletion and/or fusion, at one or more residue positions, compared to a parent polypeptide. As used herein, the terms “variant Cas12i2 polypeptide” and “variant effector polypeptide” refer to a polypeptide comprising an alteration as compared to the polypeptide of SEQ ID NO: 2.

### BRIEF DESCRIPTION OF THE DRAWINGS

FIG. 1 is a schematic showing wild-type Cas12i2 (SEQ ID NO: 2) and Cas12i2 variants set forth in SEQ ID NOs: 3-5, 495, or 496. The RuvC motifs and mutated residues are depicted.

FIG. 2 shows an overlapping PCR method to introduce single mutations and generate linear DNA templates expressing variant Cas12i2 sequences.

FIG. 3A is a DNA EMSA gel showing the ability of RNPs prepared with a) wild-type Cas12i2 (SEQ ID NO: 2), variant Cas12i2 of SEQ ID NO: 3, or variant Cas12i2 of SEQ ID NO: 4 and b) crRNA 1 (SEQ ID NO: 147) to bind an AAVS1 dsDNA target (SEQ ID NO: 150). Bound dsDNA and unbound dsDNA bands are indicated.

FIG. 3B is a DNA EMSA gel showing the ability of RNPs prepared with a) wild-type Cas12i2 (SEQ ID NO: 2), variant Cas12i2 of SEQ ID NO: 3, or variant Cas12i2 of SEQ ID NO: 4 and b) crRNA 2 (SEQ ID NO: 148) to bind a VEGFA dsDNA target (SEQ ID NO: 151). Bound dsDNA and unbound dsDNA bands are indicated.

FIG. 3C is a DNA EMSA gel showing the ability of RNPs prepared with a) wild-type Cas12i2 (SEQ ID NO: 2), variant Cas12i2 of SEQ ID NO: 3, or variant Cas12i2 of SEQ ID NO: 4 and b) crRNA 3 (SEQ ID NO: 149) to bind an EMX1 dsDNA target (SEQ ID NO: 152). Bound dsDNA and unbound dsDNA bands are indicated.

FIG. 3D is a DNA EMSA gel showing the ability of RNPs prepared with a) wild-type Cas12i2 (SEQ ID NO: 2), variant Cas12i2 of SEQ ID NO: 3, or variant Cas12i2 of SEQ ID NO: 4 and b) crRNA 1 (SEQ ID NO: 147) to bind an EMX1 dsDNA target (SEQ ID NO: 152). Unbound dsDNA bands are indicated.

FIG. 3E is a gel showing migration of samples comprising a) crRNA 1 (SEQ ID NO: 147) and DNA target 1 (SEQ ID NO: 150), b) crRNA 2 (SEQ ID NO: 148) and DNA target 2 (SEQ ID NO: 151), c) crRNA 3 (SEQ ID NO: 149) and DNA target 3 (SEQ ID NO: 152), and d) crRNA 1 (SEQ ID NO: 147) and DNA target 3 (SEQ ID NO: 152).

FIG. 4 is a schematic of the fluorescence depletion assay described in Example 10 to measure variant Cas12i2 activity.

FIG. 5A-5T are graphs of GFP Depletion Ratios (Non-target/target) for wild-type Cas12i2 (solid line), variant Cas12i2 of SEQ ID NO: 3 (dotted line), and variant Cas12i2 of SEQ ID NO: 4 (dashed line). The Depletion Ratio values were calculated from measurements taken over a period of 12 hours. Twenty GFP targets are shown: top1 (FIG. 5A), top2 (FIG. 5B), top3 (FIG. 5C), top4 (FIG. 5D), top5 (FIG. 5E), top6 (FIG. 5F), top7 (FIG. 5G), top8 (FIG. 5H), top9 (FIG. 5I), top10 (FIG. 5J), bot1 (FIG. 5K), bot2 (FIG. 5L), bot3 (FIG. 5M), bot4 (FIG. 5N), bot5 (FIG. 5O), bot6 (FIG. 5P), bot7 (FIG. 5Q), bot8 (FIG. 5R), bot9 (FIG. 5S), bot10 (FIG. 5T).

FIG. 6A shows indel measurements of fifteen genetic regions targeted with wild-type Cas12i2, variant Cas12i2 of SEQ ID NO:3, or variant Cas12i2 of SEQ ID NO:4, as assessed by Next Generation Sequencing. FIG. 6B shows the fraction of fifteen genetic regions found to be capable of being targeted with wild-type Cas12i2, variant Cas12i2 of SEQ ID NO:3, or variant Cas12i2 of SEQ ID NO:4, using the data of FIG. 6A.

FIG. 7A compares indel rates on an AAVS1 target using wild-type Cas12i2 (SEQ ID NO: 2) or Cas12i2 variants of SEQ ID NOs: 3-5, 495, or 496, 46, 47, 50-63, 65-68, 79, 84, 87-90, 95-97, 99, 101, 103, 104, 112, 114-118, 123, 130, and 131. FIG. 7B compares indel rates on an EMX1 target using wild-type Cas12i2 or Cas12i2 variants of SEQ ID NOs: 3-5, 495, or 496, 46, 47, 50-63, 65-68, 76, 79, 84, 86-90, 95-97, 99, 101, 103, 104, 112, and 114-124. FIG. 7C compares indel rates on an VEGFA1 target using wild-type Cas12i2 or Cas12i2 variants of SEQ ID NOs: 3-5, 495, or 496, 46, 47, 50-63, 65-68, 76, 79, 84, 86-90, 95-97, 99, 101, 103, 104, 112, and 114-124.

FIG. 8 shows indel activity by variant binary complexes comprising variant Cas12i2 of SEQ ID NO: 4 and several individual crRNAs targeting B2M at various concentrations in primary T cells. Error bars represent standard deviation of the mean of four technical replicates from one representative donor.

FIG. 9 shows B2M expression reduction by variant binary complexes comprising variant Cas12i2 of SEQ ID NO: 4 and several individual crRNAs targeting B2M at various concentrations in primary T cells. Error bars represent standard deviation of the mean of four technical replicates from one representative donor.

FIG. 10 shows viability of cells (via DAPI staining) seven days following introduction of variant Cas12i2 RNPs targeting B2M at various concentrations in primary T cells. Error bars represent standard deviation of the mean of four technical replicates from one representative donor.

FIG. 11A indel activity by variant binary complexes comprising variant Cas12i2 of SEQ ID NO: 4 and several individual crRNAs targeting TRAC at various concentrations in primary T cells. Error bars represent standard deviation of the mean of four technical replicates from one representative donor.

FIG. 11B shows viability of cells (via DAPI staining) seven days following introduction of variant Cas12i2 RNPs targeting TRAC at various concentrations in primary T cells. Error bars represent standard deviation of the mean of four technical replicates from one representative donor.

FIG. 12A shows indel activity by variant binary complexes comprising variant Cas12i2 of SEQ ID NO: 4 and several individual crRNAs targeting PDCD1 at various concentrations in primary T cells. Error bars represent standard deviation of the mean of four technical replicates from one representative donor.

FIG. 12B shows viability of cells (via DAPI staining) seven days following introduction of variant Cas12i2 RNPs targeting PDCD1 at various concentrations in primary T cells. Error bars represent standard deviation of the mean of four technical replicates from one representative donor.

FIG. 13 is a schematic showing how Levenshtein distances (edit distances) are calculated using exemplary on-target and non-target sequences. An on-target sequence and four non-target sequences are shown, each having an edit distance of 1, 2, 3, or 4. Each substituted, inserted, or deleted residue is indicated in bold. The PAM sequence for the target sequence and each non-target sequence is indicated to the left of the dotted line.

FIG. 14A shows on-target indel percentages on eight AAVS1 loci, eight EMX1 loci, and eight VEGFA loci using the Cas12i2 variant of SEQ ID NO: 3 and further shows off-target indel percentages on loci having an edit distance of 1, 2, 3, or 4 as compared to the target loci. FIG. 14B shows on-target indel percentages on eight AAVS1 loci, eight EMX1 loci, and eight VEGFA loci using the Cas12i2 variant of SEQ ID NO: 4 and further shows off-target indel percentages on loci having an edit distance of 1, 2, 3, or 4 as compared to the target loci. FIG. 14C shows on-target indel rates on eight AAVS1 loci, eight EMX1 loci, and eight VEGFA loci using the Cas12i2 variant of SEQ ID NO: 5 and further shows off-target indel rates on loci having an edit distance of 1, 2, 3, or 4 as compared to the target loci.

FIG. 15 is a schematic showing steps of the tagmentation-based tag integration site sequencing (TTISS) method used to analyze Cas12i2 variant specificity and activity in Example 16.

FIG. 16A shows on-target and off-target reads for variant Cas12i2 of SEQ ID NO: 4 and SpCas9 at the target AAVS1_T5. FIG. 16B shows on-target and off-target reads for variant Cas12i2 of SEQ ID NO: 4 and SpCas9 at the target EMX1_T2. FIG. 16C shows on-target and off-target reads for variant Cas12i2 of SEQ ID NO: 4 and SpCas9 at the target EMX1_T4. FIG. 16D shows on-target and off-target reads for variant Cas12i2 of SEQ ID NO: 4 and SpCas9 at the target VEGFA_T6.

FIG. 17A is a graph showing indels induced in EMX1_T6 and VEGFA_T7 by several engineered Cas12i2 variants.

FIG. 17B is a graph showing indels induced in EMX1_T6 and VEGFA_T7 by the Cas12i2 variants of SEQ ID NOs: 3-5 and 495.

FIG. 18 is a graph showing indels induced in AAVS1_T6, AAVS1_T7, EMX1_T2, EMX1_T6, and VEGFA_T5 by the Cas12i2 variants of SEQ ID NOs: 4, 495, and 496.

FIG. 19 is a schematic showing the domain structure of the Cas12i2 polypeptide.

FIG. 20A depicts the location of the D581R substitution in the Cas12i2 structure. The D581R substitution can form an electrostatic contact with DNA at the PAM sequence. D581R can interact with the non-target strand.

FIG. 20B depicts the locations of the I926R and V1030G substitutions in the Cas12i2 structure, which are close to the active site. I926R can interact with single-stranded DNA near the active site and stabilizes the interface with Rec1. V1030G is at the C-terminal portion of the structure.

FIG. 20C depicts direct repeat stabilization of unpaired and non-stacking bases. The direct repeat sequence interacts with Cas12i2 over a large area. Additionally, some RNA guide bases pair with one another. However, some bases are left exposed (stars), e.g., the exposed bases do not pair with other bases and do not have many interactions with Cas12i2. Substitutions that increase interactions between Cas12i2 and the RNA guide, especially at the exposed regions, can stabilize the binary complex and increase enzymatic activity.

FIG. 21A is a schematic showing ternary complex formation. Double-stranded DNA downstream of the PAM melts, and the spacer of an RNA guide binds to the target strand, forming a heteroduplex. The PAM sequence remains as intact double-stranded DNA, resulting in partial exposure of the terminal PAM double-stranded DNA base pair to the environment and the protein. The terminal base pair of the heteroduplex is also exposed. The exposed bases are indicated as the “heteroduplex end” and the “dsDNA duplex end.”

FIG. 21B shows exposed bases at the site of DNA melting and heteroduplex annealing in the Cas12i2 structure. Substitutions are described herein to stabilize the ends of the double-stranded DNA duplex and the heteroduplex. These substitutions can lower the energy barrier to initial target unwinding.

FIG. 22A shows conformational changes required for the binary complex to ternary complex transition. Most changes in Ca position between the binary and ternary structures occur in Helical II domain. Vectors show Ca movement ≥3.0 Å, ˜25° rotation of Helical II around axis. Substitutions are described herein to enhance ternary complex formation.

FIG. 22B shows regions in the Helical II domain where substitutions can increase Helical II domain flexibility. FIG. 22B further shows regions in the Cas12i2 structure where substitutions can stabilize the interface between the Helical II domain and the Nuc domain.

### DETAILED DESCRIPTION

The present disclosure relates to novel variants of the effector of SEQ ID NO: 2 and methods of production and use thereof. The present disclosure further relates to complexes comprising a variant of the effector of SEQ ID NO: 2 and methods of production and use thereof. In some aspects, a composition comprising a complex having one or more characteristics is described herein. In some aspects, a method of delivering a composition comprising the complex is described.

### Compositions

In some aspects, the invention described herein comprises compositions comprising a complex (e.g., a binary complex). In some aspects, the invention described herein comprises compositions comprising a complex comprising a Cas12i2 polypeptide and a targeting moiety. In some embodiments, a composition of the invention includes a variant Cas12i2 polypeptide and an RNA guide, and the variant Cas12i2 polypeptide has increased complex formation with the RNA guide as compared to a parent polypeptide.

In some aspects, a composition of the invention includes a complex comprising a variant Cas12i2 polypeptide and an RNA guide, wherein the Cas12i2 variant polypeptide and the RNA guide have a greater binding affinity, as compared to a parent polypeptide and the RNA guide.

In some aspects, a composition of the invention includes a complex comprising a variant Cas12i2 polypeptide and an RNA guide, wherein the Cas12i2 variant polypeptide and the RNA guide have stronger protein-RNA interactions, as compared to a parent polypeptide and the RNA guide. In some embodiments, the protein-RNA interactions are ionic interactions.

In some aspects, a composition of the invention includes a complex comprising a variant Cas12i2 polypeptide and an RNA guide, wherein the complex is more stable than a complex formed by a parent polypeptide and the RNA guide.

In some aspects, the invention described herein comprises compositions comprising a complex (e.g., a ternary complex). In some embodiments, a composition of the invention includes a variant Cas12i2 polypeptide, an RNA guide, and a target nucleic acid, and the variant Cas12i2 polypeptide has increased complex formation (e.g., ternary complex formation) with the RNA guide and target nucleic acid, as compared to a parent polypeptide.

In some aspects, a composition of the invention includes a binary complex comprising a variant Cas12i2 polypeptide and an RNA guide, and the composition further comprises a target nucleic acid. In some embodiments, the binary complex has increased ternary complex formation with the target nucleic acid, as compared to a parent binary complex.

In some aspects, a composition of the invention includes a variant Cas12i2 polypeptide, an RNA guide, and a target nucleic acid, wherein the Cas12i2 variant polypeptide and the RNA guide have a greater binding affinity to the target nucleic acid, as compared to a parent polypeptide and RNA guide.

In some aspects, a composition of the invention includes a binary complex comprising a variant Cas12i2 polypeptide and an RNA guide, and the composition further comprises a target nucleic acid. In some embodiments, the binary complex has a greater binding affinity to the target nucleic acid, as compared to a parent binary complex.

In some aspects, a composition of the invention includes a ternary complex comprising a variant Cas12i2 polypeptide, an RNA guide, and a target nucleic acid, wherein the ternary complex is more stable than a complex formed by a parent polypeptide, RNA guide, and target nucleic acid.

In some aspects, a composition of the invention includes a binary complex comprising a variant Cas12i2 polypeptide and an RNA guide, and the composition further comprises a target nucleic acid. In some embodiments, the binary complex forms a more stable ternary complex with the target nucleic acid than a ternary complex formed by a parent binary complex and target nucleic acid.

In some aspects, the invention described herein comprises compositions comprising a complex (e.g., a ternary complex). In some embodiments, a composition of the invention includes a variant Cas12i2 polypeptide, an RNA guide, and a target nucleic acid, and the variant Cas12i2 polypeptide and RNA guide form a variant binary complex having greater binding affinity to the target nucleic acid, as compared to a parent binary complex.

In some aspects, a composition of the invention includes a binary complex comprising a variant Cas12i2 polypeptide and an RNA guide, and the composition further comprises a target nucleic acid. In some embodiments, the binary complex has greater target binding affinity to a target locus of the target nucleic acid, as compared to a parent binary complex.

In some aspects, a composition of the invention includes a plurality of variant Cas12i2 polypeptides and two or more distinct RNA guides. In some embodiments, the distinct RNA guides individually form variant binary complexes with separate variant Cas12i2 polypeptides, and the variant binary complexes have greater on-target binding of two or more target loci of a target nucleic acid, as compared to parent binary complexes.

In some aspects, a composition of the invention includes a plurality of variant Cas12i2 polypeptides and two or more distinct RNA guides. In some embodiments, the distinct RNA guides individually form variant binary complexes with separate variant Cas12i2 polypeptides, and the variant binary complexes have greater on-target ternary complex formation with two or more target loci of a target nucleic acid, as compared to parent binary complexes.

In some aspects, a composition of the invention includes a plurality of variant Cas12i2 polypeptides and two or more distinct RNA guides. In some embodiments, the distinct RNA guides individually form variant binary complexes with separate variant Cas12i2 polypeptides, and the variant binary complexes form more stable ternary complexes with two or more target loci of a target nucleic acid, as compared to parent binary complexes.

In some aspects, the invention described herein comprises compositions comprising a complex. In some embodiments, the invention comprises a binary complex comprising a variant Cas12i2 polypeptide and an RNA guide.

In some embodiments, the invention comprises a ternary complex comprising a variant Cas12i2 polypeptide, an RNA guide, and a target locus of a target nucleic acid. In some embodiments, a composition of the invention comprises a plurality of variant Cas12i2 polypeptides and two or more distinct RNA guides, and the distinct RNA guides individually form variant binary complexes with separate variant Cas12i2 polypeptides.

In some aspects, a composition of the invention includes a variant Cas12i2 polypeptide and an RNA guide, and the variant Cas12i2 polypeptide and RNA guide form a variant binary complex having higher on-target binding affinity to a target locus of a target nucleic acid, as compared to a parent binary complex. In some embodiments, a composition of the invention includes a plurality of variant binary complexes having higher on-target binding to two or more target loci of a target nucleic acid, as compared to a plurality of parent binary complexes.

In some aspects, a composition of the invention includes a binary complex comprising a variant Cas12i2 polypeptide and RNA guide, and the binary complex has a lower binding affinity to a non-target locus of a target nucleic acid, as compared to a parent binary complex. In some embodiments, a composition of the invention includes a plurality of variant binary complexes having lower off-target binding to two or more non-target loci of a target nucleic acid, as compared to a plurality of parent binary complexes.

In some aspects, a composition of the invention includes a binary complex comprising a variant Cas12i2 polypeptide and an RNA guide, and the binary complex has higher activity at an on-target locus of a target nucleic acid, as compared to a parent binary complex. In some embodiments, a composition of the invention includes a plurality of variant binary complexes having higher on-target activity at two or more target loci of a target nucleic acid, as compared to a plurality of parent binary complexes.

In some aspects, a composition of the invention includes a binary complex comprising a variant Cas12i2 polypeptide and an RNA guide, and the binary complex has lower activity at a non-target locus of a target nucleic acid, as compared to a parent binary complex. In some embodiments, a composition of the invention includes a plurality of variant binary complexes having lower off-target activity at two or more non-target loci of a target nucleic acid, as compared to a plurality of parent binary complexes.

### Variant Cas12i2 Polypeptide

In some embodiments, the composition of the present invention includes a variant Cas12i2 polypeptide described herein.

In some embodiments, the polypeptide of the present invention is a variant of a parent polypeptide, wherein the parent is encoded by a polynucleotide that comprises a nucleotide sequence such as SEQ ID NO: 1 or comprises an amino acid sequence such as SEQ ID NO: 2.

TABLE 1
Parent sequences.

| SEQ ID | | |
|---|---|---|
| NO | Sequence | Description |
| 1 | ATGAGCAGCGCGATCAAAAGCTACAAGAGCGTTCTGCGTCCGAACGA | Nucleotide |
| | GCGTAAGAACCAACTGCTGAAAAGCACCATTCAGTGCCTGGAAGACG | sequence |
| | GTAGCGCGTTCTTTTTCAAGATGCTGCAAGGCCTGITTGGTGGCATC | encoding |
| | ACCCCGGAGATTGTTCGTTTCAGCACCGAACAGGAGAAACAGCAACA | parent |
| | GGATATCGCGCTGTGGTGCGCGGTTAACTGGTTCCGTCCGGTGAGCC | polypeptide |
| | AAGACAGCCTGACCCACACCATTGCGAGCGATAACCTGGTGGAGAAG | |
| | TTTGAGGAATACTATGGTGGCACCGCGAGCGACGCGATCAAACAGTA | |
| | CTTCAGCGCGAGCATTGGCGAAAGCTACTATTGGAACGACTGCCGTC | |
| | AACAGTACTATGATCTGTGCCGTGAGCTGGGTGTTGAGGTGAGCGAC | |
| | CTGACCCATGATCTGGAGATCCIGTGCCGTGAAAAGTGCCTGGCGGT | |
| | TGCGACCGAGAGCAACCAGAACAACAGCATCATTAGCGTTCTGTTTG | |
| | GCACCGGCGAAAAAGAGGACCGTAGCGTGAAACTGCGTATCACCAAG | |
| | AAAATTCTGGAGGCGATCAGCAACCTGAAAGAAATCCCGAAGAACGT | |
| | TGCGCCGATTCAAGAGATCATTCTGAACGTGGCGAAAGCGACCAAGG | |
| | AAACCTTCCGTCAGGTGTATGCGGGTAACCTGGGTGCGCCGAGCACC | |
| | CTGGAGAAATTTATCGCGAAGGACGGCCAAAAAGAGTTCGATCTGAA | |
| | GAAACTGCAGACCGACCTGAAGAAAGTTATTCGTGGTAAAAGCAAGG | |
| | AGCGTGATTGGTGCTGCCAGGAAGAGCTGCGTAGCTACGTGGAGCAA | |
| | AACACCATCCAGTATGACCTGTGGGCGTGGGGCGAAATGTTCAACAA | |
| | AGCGCACACCGCGCTGAAAATCAAGAGCACCCGTAACTACAACTTTG | |
| | CGAAGCAACGTCTGGAACAGTTCAAAGAGATTCAGAGCCTGAACAAC | |
| | CTGCTGGTTGTGAAGAAGCTGAACGACTTTTTCGATAGCGAATTTTT | |
| | CAGCGGCGAGGAAACCTACACCATCTGCGTTCACCATCTGGGTGGCA | |
| | AGGACCTGAGCAAACTGTATAAGGCGTGGGAGGATGATCCGGCGGAC | |
| | CCGGAAAACGCGATTGTGGTTCTGTGCGACGATCTGAAAAACAACTT | |
| | TAAGAAAGAGCCGATCCGTAACATTCTGCGTTACATCTTCACCATTC | |
| | GTCAAGAATGCAGCGCGCAGGACATCCTGGCGGCGGCGAAGTACAAC | |
| | CAACAGCTGGATCGTTATAAAAGCCAAAAGGCGAACCCGAGCGTTCT | |
| | GGGTAACCAGGGCTTTACCTGGACCAACGCGGTGATCCTGCCGGAGA | |
| | AGGCGCAGCGTAACGACCGTCCGAACAGCCTGGATCTGCGTATTTGG | |
| | CTGTACCTGAAACTGCGTCACCCGGACGGTCGTTGGAAGAAACACCA | |
| | TATCCCGTTCTACGATACCCGTTTCTTCCAAGAAATTTATGCGGCGG | |
| | GCAACAGCCCGGTTGACACCTGCCAGTTTCGTACCCCGCGTTTCGGT | |
| | TATCACCTGCCGAAACTGACCGATCAGACCGCGATCCGTGTTAACAA | |
| | GAAACATGTGAAAGCGGCGAAGACCGAGGCGCGTATTCGTCTGGCGA | |
| | TCCAACAGGGCACCCTGCCGGTGAGCAACCTGAAGATCACCGAAATT | |
| | AGCGCGACCATCAACAGCAAAGGTCAAGTGCGTATTCCGGTTAAGTT | |
| | TGACGTGGGTCGTCAAAAAGGCACCCTGCAGATCGGTGACCGTTTCT | |
| | GCGGCTACGATCAAAACCAGACCGCGAGCCACGCGTATAGCCTGTGG | |
| | GAAGTGGTTAAAGAGGGTCAATACCATAAAGAGCTGGGCTGCTTTGT | |
| | TCGTTTCATCAGCAGCGGTGACATCGTGAGCATTACCGAGAACCGTG | |
| | GCAACCAATTTGATCAGCTGAGCTATGAAGGTCTGGCGTACCCGCAA | |
| | TATGCGGACTGGCGTAAGAAAGCGAGCAAGTTCGTGAGCCTGTGGCA | |
| | GATCACCAAGAAAAACAAGAAAAAGGAAATCGTGACCGTTGAAGCGA | |
| | AAGAGAAGTTTGACGCGATCTGCAAGTACCAGCCGCGTCTGTATAAA | |
| | TTCAACAAGGAGTACGCGTATCTGCTGCGTGATATTGTTCGTGGCAA | |
| | AAGCCTGGTGGAACTGCAACAGATTCGTCAAGAGATCTTTCGTTTCA | |
| | TTGAACAGGACTGCGGTGTTACCCGTCTGGGCAGCCTGAGCCTGAGC | |
| | ACCCTGGAAACCGTGAAAGCGGTTAAGGGTATCATTTACAGCTATTT | |
| | TAGCACCGCGCTGAACGCGAGCAAGAACAACCCGATCAGCGACGAAC | |
| | AGCGTAAAGAGTTTGATCCGGAACTGTTCGCGCTGCTGGAAAAGCTG | |
| | GAGCTGATTCGTACCCGTAAAAAGAAACAAAAAGTGGAACGTATCGC | |
| | GAACAGCCTGATTCAGACCTGCCTGGAGAACAACATCAAGTTCATTC | |
| | GTGGTGAAGGCGACCTGAGCACCACCAACAACGCGACCAAGAAAAAG | |
| | GCGAACAGCCGTAGCATGGATTGGTTGGCGCGTGGTGTTTTTAACAA | |
| | AATCCGTCAACTGGCGCCGATGCACAACATTACCCTGTTCGGTTGCG | |
| | GCAGCCTGTACACCAGCCACCAGGACCCGCTGGTGCATCGTAACCCG | |
| | GATAAAGCGATGAAGTGCCGTTGGGCGGCGATCCCGGTTAAGGACAT | |
| | TGGCGATTGGGTGCTGCGTAAGCTGAGCCAAAACCTGCGTGCGAAAA | |
| | ACATCGGCACCGGCGAGTACTATCACCAAGGTGTTAAAGAGTTCCTG | |
| | AGCCATTATGAACTGCAGGACCTGGAGGAAGAGCTGCTGAAGTGGCG | |
| | TAGCGATCGTAAAAGCAACATTCCGTGCTGGGTGCTGCAGAACCGTC | |
| | TGGCGGAGAAGCTGGGCAACAAAGAAGCGGTGGTTTACATCCCGGTT | |
| | CGTGGTGGCCGTATTTATTTTGCGACCCACAAGGTGGCGACCGGTGC | |
| | GGTGAGCATCGTTTTCGACCAAAAACAAGTGTGGGTTTGCAACGCGG | |
| | ATCATGTTGCGGCGGCGAACATCGCGCTGACCGTGAAGGGTATTGGC | |
| | GAACAAAGCAGCGACGAAGAGAACCCGGATGGTAGCCGTATCAAACT | |
| | GCAGCTGACCAGC | |
| 2 | MSSAIKSYKSVLRPNERKNOLLKSTIQCLEDGSAFFFKMLQGLFGGI | Parent |
| | TPEIVRFSTEQEKQQQDIALWCAVNWFRPVSQDSLTHTIASDNLVEK | polypeptide |
| | FEEYYGGTASDAIKQYFSASIGESYYWNDCRQQYYDLCRELGVEVSD | |
| | LTHDLEILCREKCLAVATESNQNNSIISVLFGTGEKEDRSVKLRITK | |
| | KILEAISNLKEIPKNVAPIQEIILNVAKATKETFRQVYAGNLGAPST | |
| | LEKFIAKDGQKEFDLKKLQTDLKKVIRGKSKERDWCCQEELRSYVEQ | |
| | NTIQYDLWAWGEMFNKAHTALKIKSTRNYNFAKQRLEQFKEIQSLNN | |
| | LLVVKKLNDFFDSEFFSGEETYTICVHHLGGKDLSKLYKAWEDDPAD | |
| | PENAIVVLCDDLKNNFKKEPIRNILRYIFTIRQECSAQDILAAAKYN | |
| | QQLDRYKSQKANPSVLGNQGFTWTNAVILPEKAQRNDRPNSLDLRIW | |
| | LYLKLRHPDGRWKKHHIPFYDTRFFQEIYAAGNSPVDTCQFRTPRFG | |
| | YHLPKLTDQTAIRVNKKHVKAAKTEARIRLAIQQGTLPVSNLKITEI | |
| | SATINSKGQVRIPVKFDVGRQKGTLQIGDRFCGYDQNQTASHAYSLW | |
| | EVVKEGQYHKELGCFVRFISSGDIVSITENRGNQFDQLSYEGLAYPQ | |
| | YADWRKKASKFVSLWQITKKNKKKEIVTVEAKEKFDAICKYQPRLYK | |
| | FNKEYAYLLRDIVRGKSLVELQQIRQEIFRFIEQDCGVTRLGSLSLS | |
| | TLETVKAVKGIIYSYFSTALNASKNNPISDEQRKEFDPELFALLEKL | |
| | ELIRTRKKKQKVERIANSLIQTCLENNIKFIRGEGDLSTTNNATKKK | |
| | ANSRSMDWLARGVFNKIRQLAPMHNITLFGCGSLYTSHQDPLVHRNP | |
| | DKAMKCRWAAIPVKDIGDWVLRKLSQNLRAKNIGTGEYYHQGVKEFL | |
| | SHYELQDLEEELLKWRSDRKSNIPCWVLQNRLAEKLGNKEAVVYIPV | |
| | RGGRIYFATHKVATGAVSIVFDQKQVWVCNADHVAAANIALTVKGIG | |
| | EQSSDEENPDGSRIKLQLTS | |

In an aspect of the invention, a variant polypeptide sequence includes one or more variations.

A nucleic acid sequence encoding the parent polypeptide described herein may be substantially identical to a reference nucleic acid sequence, e.g., SEQ ID NO: 1. In some embodiments, the variant Cas12i2 polypeptide is encoded by a nucleic acid comprising a sequence having least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to the reference nucleic acid sequence, e.g., nucleic acid sequence encoding the parent polypeptide, e.g., SEQ ID NO: 1. The percent identity between two such nucleic acids can be determined manually by inspection of the two optimally aligned nucleic acid sequences or by using software programs or algorithms (e.g., BLAST, ALIGN, CLUSTAL) using standard parameters. One indication that two nucleic acid sequences are substantially identical is that the nucleic acid molecules hybridize to the complementary sequence of the other under stringent conditions (e.g., within a range of medium to high stringency).

In some embodiments, the variant Cas12i2 polypeptide is encoded by a nucleic acid sequence having at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more sequence identity, but not 100% sequence identity, to a reference nucleic acid sequence, e.g., nucleic acid sequence encoding the parent polypeptide, e.g., SEQ ID NO: 1.

In some embodiments, the variant Cas12i2 polypeptide of the present invention comprises a polypeptide sequence having 50%, 60%, 65%, 70%, 75%, 80%, 85%, 90%, 91%, 92%, 93%, 94%, 95%, 96%, 97%, 98%, or 99%, but not 100%, identity to SEQ ID NO: 2. In some embodiments, the variant Cas12i2 polypeptide of the present invention comprises a polypeptide sequence having greater than 50%, 60%, 65%, 70%, 75%, 80%, 85%, 90%, 91%, 92%, 93%, 94%, 95%, 96%, 97%, 98%, or 99%, but not 100%, identity to SEQ ID NO: 2.

In some embodiments, the present invention describes a variant Cas12i2 polypeptide having a specified degree of amino acid sequence identity to one or more reference polypeptides, e.g., a parent polypeptide, e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99%, but not 100%, sequence identity to the amino acid sequence of SEQ ID NO: 2. Homology or identity can be determined by amino acid sequence alignment, e.g., using a program such as BLAST, ALIGN, or CLUSTAL, as described herein.

In some embodiments, the variant Cas12i2 polypeptide comprises an alteration at one or more (e.g., several) amino acids of a parent polypeptide, wherein at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 162, 164, 164, 165, 166, 167, 168, 169, 170, 171, 172, 173, 174, 175, 176, 177, 178, 179, 180, 181, 182, 183, 184, 185, 186, 187, 188, 189, 190, 191, 193, 194, 195, 196, 197, 198, 199, 200, or more are altered.

In some embodiments, the variant Cas12i2 polypeptide comprises one or more of the amino acid substitutions listed in Table 2. In some embodiments, the variant Cas12i2 polypeptide comprises at least one of a D581, G624, F626, D835, L836, P868, S879, D911, 1926, V1020, V1030, E1035, and S1046 substitution. In some embodiments, the variant Cas12i2 polypeptide comprises at least one of a D581R, G624R, F626R, D835R, L836R, P868R, S879R, D911R, 1926R, V1020R, V1030R, E1035R, and 51046R substitution. In some embodiments, the variant Cas12i2 polypeptide comprises at least one of a D581G, F626G, D835G, L836G, P868G, S879G, D911G, 1926G, V1020G, V1030G, E1035G, and 51046G substitution. In some embodiments, the variant Cas12i2 polypeptide comprises at least one of a D581R, G624R, F626R, D835R, L836R, P868T, S879R, D911R, 1926R, V1020G, V1030G, E1035R, and 51046G substitution and at least one additional substitution listed in Table 2. In some embodiments, the variant Cas12i2 polypeptide comprises any one of SEQ ID NOs: 3-146 and 495-512. In some embodiments, the variant Cas12i2 polypeptide comprises any one of SEQ ID NOs: 3-146 and 495-512 and at least one additional substitution listed in Table 2. In some embodiments, the variant Cas12i2 polypeptide comprises the amino acid sequence of SEQ ID NO: 3. In some embodiments, the variant Cas12i2 polypeptide comprises the amino acid sequence of SEQ ID NO: 4. In some embodiments, the variant Cas12i2 polypeptide comprises the amino acid sequence of SEQ ID NO: 5. In some embodiments, the variant Cas12i2 polypeptide comprises the amino acid sequence of SEQ ID NO: 495. In some embodiments, the variant Cas12i2 polypeptide comprises the amino acid sequence of SEQ ID NO: 496.

TABLE 2
TABLE 2. Single amino acid substitutions
in variant Cas12i2 polypeptide.

| | Wild-Type | |
|---|---|---|
| Position | Residue | Substitution(s) |
| 1 | M | |
| 2 | S | R, G, A, K, Q, N, H |
| 3 | S | R, G, A, K, Q, N, H |
| 4 | A | R, G, K, Q, N, H |
| 5 | I | R, G, A, K, Q, N, H, W, F, Y, M |
| 6 | K | R, G, A, Q, N, H |
| 7 | S | P, R, G, A, K, Q, N, H |
| 8 | Y | R, G, A, K, Q, N, H |
| 9 | K | R, G, A, Q, N, H |
| 10 | S | R, G, A, K, Q, N, H |
| 11 | V | R, K, G, A, Q, N, H |
| 12 | L | R, G, A, K, Q, N, H |
| 13 | R | A, G, K, Q, N, H |
| 14 | P | R, G, A, K, Q, N, H |
| 15 | N | R, D, G, A, K, Q, H |
| 16 | E | R, D, G, A, K, Q, N, H |
| 17 | R | G, A, K, Q, N, H |
| 18 | K | R, G, A, Q, N, H |
| 19 | N | R, G, A, K, Q, H |
| 20 | Q | R, K, G, A, N, H |
| 21 | L | R, M, G, A, K, Q, N, H |
| 22 | L | R, G, A, K, Q, N, H |
| 23 | K | R, N, G, A, Q, H |
| 24 | S | R, D, G, A, K, Q, N, H |
| 25 | T | R, G, A, K, Q, N, H |
| 26 | I | R, F, G, A, K, Q, N, H |
| 27 | Q | R, N, G, A, K, H |
| 28 | C | R, W, G, A, K, Q, N, H |
| 29 | L | R, G, A, K, Q, N, H |
| 30 | E | R, D, G, A, K, Q, N, H |
| 31 | D | R, G, A, K, Q, N, H |
| 32 | G | R, A, K, Q, N, H |
| 33 | S | R, G, A, K, Q, N, H |
| 34 | A | R, G, K, Q, N, H |
| 35 | F | R, V, G, A, K, Q, N, H |
| 36 | F | R, G, A, K, Q, N, H |
| 37 | F | R, G, A, K, Q, N, H |
| 38 | K | R, D, G, A, Q, N, H |
| 39 | M | R, G, A, K, Q, N, H |
| 40 | L | R, G, A, K, Q, N, H |
| 41 | Q | R, V, G, A, K, N, H |
| 42 | G | R, A, K, Q, N, H |
| 43 | L | R, G, A, K, Q, N, H |
| 44 | F | R, G, A, K, Q, N, H |
| 45 | G | R, A, K, Q, N, H |
| 46 | G | R, A, K, Q, N, H |
| 47 | I | R, G, A, K, Q, N, H |
| 48 | T | R, G, A, K, Q, N, H |
| 49 | P | R, H, G, A, K, Q, N |
| 50 | E | R, G, A, K, Q, N, H |
| 51 | I | R, G, A, K, Q, N, H |
| 52 | V | R, A, G, K, Q, N, H |
| 53 | R | G, A, K, Q, N, H |
| 54 | F | R, G, A, K, Q, N, H |
| 55 | S | R, G, A, K, Q, N, H |
| 56 | T | R, G, A, K, Q, N, H |
| 57 | E | R, G, A, K, Q, N, H |
| 58 | Q | R, G, A, K, N, H |
| 59 | E | R, G, A, K, Q, N, H |
| 60 | K | R, G, A, Q, N, H |
| 61 | Q | R, S, G, A, K, N, H |
| 62 | Q | R, K, G, A, K, N, H |
| 63 | Q | R, G, A, K, N, H |
| 64 | D | R, G, A, K, Q, N, H |
| 65 | I | R, G, A, K, Q, N, H |
| 66 | A | R, D, G, K, Q, N, H |
| 67 | L | R, G, A, K, Q, N, H |
| 68 | W | R, L, G, A, K, Q, N, H |
| 69 | C | R, G, A, K, Q, N, H |
| 70 | A | R, G, K, Q, N, H |
| 71 | V | R, I, G, A, K, Q, N, H |
| 72 | N | R, G, A, K, Q, H |
| 73 | W | R, G, A, K, Q, N, H |
| 74 | F | R, G, A, K, Q, N, H |
| 75 | R | G, A, K, Q, N, H |
| 76 | P | R, L, G, A, K, Q, N, H |
| 77 | V | R, G, A, K, Q, N, H |
| 78 | S | R, G, A, K, Q, N, H |
| 79 | Q | R, K, G, A, N, H |
| 80 | D | R, G, A, K, Q, N, H |
| 83 | S | R, G, A, K, Q, N, H |
| 82 | L | R, G, A, K, Q, N, H |
| 83 | T | R, G, A, K, Q, N, H |
| 84 | H | R, G, A, K, Q, N |
| 85 | T | R, G, A, K, Q, N, H |
| 86 | I | R, Q, G, A, K, N, H |
| 87 | A | R, T, G, K, Q, N, H |
| 88 | S | R, G, A, K, Q, N, H |
| 89 | D | R, E, G, A, K, Q, N, H |
| 90 | N | R, G, A, K, Q, H |
| 91 | L | R, G, A, K, Q, N, H |
| 92 | V | R, G, A, K, Q, N, H |
| 93 | E | R, G, A, K, Q, N, H |
| 94 | K | R, L, G, A, Q, N, H |
| 95 | F | R, G, A, K, Q, N, H |
| 96 | E | R, G, A, K, Q, N, H |
| 97 | E | R, G, A, K, Q, N, H |
| 98 | Y | R, G, A, K, Q, N, H |
| 99 | Y | R, S, G, A, K, Q, N, H |
| 100 | G | R, A, K, Q, N, H |
| 101 | G | R, H, A, K, Q, N |
| 102 | T | R, E, G, A, K, Q, N, H |
| 103 | A | R, P, G, K, Q, N, H |
| 104 | S | R, G, A, K, Q, N, H |
| 105 | D | R, S, G, A, K, Q, N, H |
| 106 | A | R, F, G, K, Q, N, H |
| 107 | I | R, G, A, K, Q, N, H |
| 108 | K | R, Q, G, A, N, H |
| 109 | Q | R, E, G, A, K, N, H |
| 110 | Y | R, G, A, K, Q, N, H |
| 111 | F | R, G, A, K, Q, N, H |
| 112 | S | R, G, A, K, Q, N, H |
| 113 | A | R, G, K, Q, N, H |
| 114 | S | R, N, G, A, K, Q, N, H |
| 115 | I | R, G, A, K, Q, N, H |
| 116 | G | R, D, A, K, Q, N, H |
| 117 | E | R, G, A, K, Q, N, H |
| 118 | S | R, K, G, A, K, Q, N, H |
| 119 | Y | R, G, A, K, Q, N, H |
| 120 | Y | R, G, A, K, Q, N, H |
| 121 | W | R, G, A, K, Q, N, H |
| 122 | N | R, V, G, A, K, Q, H |
| 123 | D | R, G, A, K, Q, N, H |
| 124 | C | R, G, A, K, Q, N, H |
| 125 | R | G, A, K, Q, N, H |
| 126 | Q | R, L, G, A, K, N, H |
| 127 | Q | R, G, A, K, N, H |
| 128 | Y | R, F, G, A, K, Q, N, H |
| 129 | Y | R, G, A, K, Q, N, H |
| 130 | D | R, G, A, K, Q, N, H |
| 131 | L | R, G, A, K, Q, N, H |
| 132 | C | R, A, G, K, Q, N, H |
| 133 | R | G, A, K, Q, N, H |
| 134 | E | R, N, G, A, K, Q, N, H |
| 135 | L | R, I, G, A, K, Q, N, H |
| 136 | G | R, A, K, Q, N, H |
| 137 | V | R, G, A, K, Q, N, H |
| 138 | E | R, S, G, A, K, Q, N, H |
| 139 | V | R, G, A, K, Q, N, H |
| 140 | S | R, G, A, K, Q, N, H |
| 141 | D | R, G, A, K, Q, N, H |
| 142 | L | R, G, A, K, Q, N, H |
| 143 | T | R, G, A, K, Q, N, H |
| 144 | H | R, G, A, K, Q, N |
| 145 | D | R, G, A, K, Q, N, H |
| 146 | L | R, A, G, K, Q, N, H |
| 147 | E | R, G, A, K, Q, N, H |
| 148 | I | R, T, G, A, K, Q, N, H |
| 149 | L | R, M, G, A, K, Q, N, H |
| 150 | C | R, G, A, K, Q, N, H |
| 151 | R | G, A, K, Q, N, H |
| 152 | E | R, G, A, K, Q, N, H |
| 153 | K | R, G, A, Q, N, H |
| 154 | C | R, L, G, A, K, Q, N, H |
| 155 | L | R, I, G, A, K, 2, N, H |
| 156 | A | R, P, G, K, Q, N, H |
| 157 | V | R, L, G, A, K, Q, N, H |
| 158 | A | R, G, K, Q, N, H |
| 159 | T | R, K, G, A, Q, N, H |
| 160 | E | R, D, G, A, K, Q, N, H, L |
| 161 | S | R, D, G, A, K, Q, N, H |
| 162 | N | R, F, G, A, K, Q, H |
| 163 | Q | R, N, G, A, K, H, M |
| 164 | N | R, A, G, K, Q, H, M, W, F |
| 165 | N | R, G, A, K, Q, H, M, P, F, Y, W |
| 166 | S | R, G, A, K, Q, N, H |
| 167 | I | R, G, A, K, Q, N, H |
| 168 | I | R, G, A, K, Q, N, H |
| 169 | S | R, G, A, K, Q, N, H |
| 170 | V | R, G, A, K, Q, N, H |
| 171 | L | R, G, A, K, Q, N, H |
| 172 | F | R, G, A, K, Q, N, H |
| 173 | G | R, A, K, Q, N, H, S |
| 174 | T | R, G, A, K, Q, N, H |
| 175 | G | R, A, K, Q, N, H |
| 176 | E | R, K, G, A, K, Q, N, H |
| 177 | K | R, G, A, Q, N, H |
| 178 | E | R, G, A, K, Q, N, H |
| 179 | D | R, G, A, K, Q, N, H |
| 180 | R | G, A, K, Q, N, H |
| 181 | S | R, G, A, K, Q, N, H |
| 182 | V | R, G, A, K, Q, N, H |
| 183 | K | R, G, A, Q, N, H |
| 184 | L | R, A, G, K, Q, N, H |
| 185 | R | G, A, K, Q, N, H |
| 186 | I | R, M, G, A, K, Q, N, H |
| 187 | T | R, L, G, A, K, Q, N, H |
| 188 | K | R, G, A, Q, N, H |
| 189 | K | R, G, A, Q, N, H |
| 190 | I | R, G, A, K, Q, N, H |
| 191 | L | R, S, G, A, K, Q, N, H |
| 192 | E | R, N, G, A, K, Q, H |
| 193 | A | R, G, K, Q, N, H |
| 194 | I | R, L, G, A, K, Q, N, H |
| 195 | S | R, G, A, K, Q, N, H |
| 196 | N | R, G, A, K, Q, H |
| 197 | L | R, G, A, K, Q, N, H |
| 198 | K | R, D, G, A, Q, N, H |
| 199 | E | R, K, G, A, Q, N, H |
| 200 | I | R, G, A, K, Q, N, H |
| 201 | P | R, G, A, K, Q, N, H |
| 202 | K | R, G, A, Q, N, H |
| 203 | N | R, T, G, A, K, Q, H |
| 204 | V | R, W, G, A, K, Q, N, H |
| 205 | A | R, E, G, K, Q, N, H |
| 206 | P | R, E, G, A, K, Q, N, H |
| 207 | I | R, Y, G, A, K, Q, N, H |
| 208 | Q | R, G, A, K, N, H |
| 209 | E | R, D, G, A, K, Q, N, H |
| 210 | I | R, L, G, A, K, Q, N, H |
| 211 | I | R, G, A, K, Q, N, H |
| 212 | L | R, G, A, K, Q, N, H |
| 213 | N | R, K, G, A, Q, H |
| 214 | V | R, G, A, K, Q, N, H |
| 215 | A | R, F, G, K, Q, N, H |
| 216 | K | R, G, A, Q, N, H |
| 217 | A | R, G, K, Q, N, H |
| 218 | T | R, G, A, K, Q, N, H |
| 219 | K | R, G, A, Q, N, H |
| 220 | E | R, G, A, K, Q, N, H |
| 221 | T | R, L, G, A, K, Q, N, H |
| 222 | F | R, K, G, A, Q, N, H |
| 223 | R | G, A, K, Q, N, H |
| 224 | Q | R, K, G, A, N, H |
| 225 | V | R, G, A, K, Q, N, H |
| 226 | Y | R, G, A, K, Q, N, H |
| 227 | A | R, G, K, Q, N, H |
| 228 | G | R, A, K, Q, N, H |
| 229 | N | R, S, G, A, K, Q, H |
| 230 | L | R, G, A, K, Q, N, H, S |
| 231 | G | R, A, K, Q, N, H |
| 232 | A | R, G, K, Q, N, H, S |
| 233 | P | R, G, A, K, Q. N, H, S |
| 234 | S | R, G, A, K, Q, N, H, P |
| 235 | T | R, S, G, A, K, Q, N, H |
| 236 | L | R, G, A, K, Q, N, H |
| 237 | E | R, V, G, A, K, Q, N, H, S |
| 238 | K | R, G, A, Q, N, H |
| 239 | F | R, D, G, A, K, Q, N, H |
| 240 | I | R, G, A, K, Q, N, H |
| 241 | A | R, K, G, Q, N, H |
| 242 | K | R, G, A, Q, N, H |
| 243 | D | R, G, A, K, Q, N, H |
| 244 | G | R, A, K, Q, N, H |
| 245 | Q | R, G, A, K, N, H |
| 246 | K | R, G, A, Q, N, H |
| 247 | E | R, G, A, K, Q, N, H |
| 248 | F | R, G, A, K, Q, N, H |
| 249 | D | R, G, A, K, Q, N, H |
| 250 | L | R, G, A, K, Q, N, H |
| 251 | K | R, G, A, Q, N, H |
| 252 | K | R, G, A, Q, N, H |
| 253 | L | R, G, A, K, Q, N, H |
| 254 | Q | R, I, G, A, K, N, H |
| 255 | T | R, G, A, K, Q, N, H |
| 256 | D | R, K, G, A, Q, N, H |
| 257 | L | R, F, G, A, K, Q, N, H |
| 258 | K | R, G, A, Q, N, H |
| 259 | K | R, G, A, Q, N, H |
| 260 | V | R, D, G, A, K, Q, N, H |
| 261 | I | R, A, G, K, Q, N, H |
| 262 | R | G, A, K, Q, N, H |
| 263 | G | R, K, A, K, Q, N, H |
| 264 | K | R, G, A, Q, N, H |
| 265 | S | R, G, A, K, Q, N, H |
| 266 | K | R, G, A, Q, N, H |
| 267 | E | R, G, A, K, Q, N, H |
| 268 | R | G, A, K, Q, N, H |
| 269 | D | R, G, A, K, Q, N, H |
| 270 | W | R, L, G, A, K, Q, N, H |
| 271 | C | R, P, G, A, K, Q, N, H |
| 272 | C | R, N, G, A, K, Q, H |
| 273 | Q | R, G, A, K, N, H |
| 274 | E | R, G, A, K, Q, N, H |
| 275 | E | R, K, G, A, Q, N, H |
| 276 | L | R, G, A, K, Q, N, H |
| 277 | R | K, G, A, Q, N, H |
| 278 | S | R, E, G, A, K, Q, N, H |
| 279 | Y | R, G, A, K, Q, N, H |
| 280 | V | R, I, G, A, K, Q, N, H |
| 281 | E | R, A, G, K, Q, N, H |
| 282 | Q | R, S, G, A, K, N, H |
| 283 | N | R, G, A, K, Q, H |
| 284 | T | R, I, G, A, K, Q, N, H |
| 285 | I | R, G, A, K, Q, N, H |
| 286 | Q | R, P, G, A, K, N, H |
| 287 | Y | R, F, G, A, K, Q, N, H |
| 288 | D | R, G, A, K, Q, N, H, S |
| 289 | L | R, Q, G, A, K, N, H, M |
| 290 | W | R, N, G, A, K, Q, H |
| 291 | A | R, S, G, K, Q, N, H |
| 292 | W | R, G, A, K, Q, N, H |
| 293 | G | R, S, A, K, Q, N, H |
| 294 | E | R, A, G, K, Q, N, H |
| 295 | M | R, G, A, K, Q, N, H |
| 296 | F | R, L, G, A, K, Q, N, H |
| 297 | N | R, G, A, K, Q, H |
| 298 | K | R, N, G, A, Q, H |
| 299 | A | R, G, K, Q, N, H |
| 300 | H | R, G, A, K, Q, N |
| 301 | T | R, G, A, K, Q, N, H |
| 302 | A | R, G, K, Q, N, H |
| 303 | L | R, I, G, A, K, Q, N, H |
| 304 | K | R, Q, G, A, N, H |
| 305 | I | R, S, G, A, K, Q, N, H |
| 306 | K | R, G, A, Q, N, H |
| 307 | S | R, N, G, A, K, Q, H |
| 308 | T | R, S, G, A, K, Q, N, H |
| 309 | R | G, A, K, Q, N, H |
| 310 | N | R, G, A, K, Q, H |
| 311 | Y | R, G, A, K, Q, N, H |
| 312 | N | R, L, G, A, K, Q, H |
| 313 | F | R, Y, G, A, K, Q, N, H |
| 314 | A | R, T, G, K, Q, N, H |
| 315 | K | R, G, A, Q, N, H |
| 316 | Q | R, E, G, A, K, N, H |
| 317 | R | K, G, A, Q, N, H |
| 318 | L | R, G, A, K, Q, N, H |
| 319 | E | R, G, A, K, Q, N, H |
| 320 | Q | R, G, A, K, N, H |
| 321 | F | R, G, A, K, Q, N, H |
| 322 | K | R, N, G, A, Q, H |
| 323 | E | R, G, A, K, Q, N, H |
| 324 | I | R, G, A, K, Q, N, H |
| 325 | Q | R, G, A, K, N, H |
| 326 | S | R, G, A, K, Q, N, H |
| 327 | L | R, G, A, K, Q, N, H, V |
| 328 | N | R, D, G, A, K, Q, H, S |
| 329 | N | R, G, A, K, Q, H, S |
| 330 | L | R, G, A, K, Q, N, H |
| 331 | L | R, G, A, K, Q, N, H |
| 332 | V | R, A, G, K, Q, N, H |
| 333 | V | R, G, A, K, Q, N, H |
| 334 | K | R, G, A, Q, N, H |
| 335 | K | R, I, G, A, Q, N, H |
| 336 | L | R, G, A, K, Q, N, H |
| 337 | N | R, G, A, K, Q, H |
| 338 | D | R, G, A, K, Q, N, H |
| 339 | F | R, G, A, K, Q, N, H |
| 340 | F | R, G, A, K, Q, N, H |
| 341 | D | R, G, A, K, Q, N, H |
| 342 | S | G, R, A, K, Q, N, H |
| 343 | E | R, G, A, K, Q, N, H |
| 344 | F | R, G, A, K, Q, N, H |
| 345 | F | R, G, A, K, Q, N, H |
| 346 | S | R, G, A, K, Q, N, H |
| 347 | G | R, A, K, Q, N, H |
| 348 | E | R, G, A, K, Q, N, H, S |
| 349 | E | R, N, G, A, K, Q, H, S |
| 350 | T | R, G, A, K, Q, N, H |
| 351 | Y | R, F, G, A, K, Q, N, H, S |
| 352 | T | R, V, G, A, K, Q, N, H |
| 353 | I | R, V, G, A, K, Q, N, H |
| 354 | C | R, G, A, K, Q, N, H |
| 355 | V | R, K, G, A, Q, N, H |
| 356 | H | R, G, A, K, Q, N |
| 357 | H | R, G, A, K, Q, N, S |
| 358 | L | R, G, A, K, Q, N, H |
| 359 | G | R, A, K, Q, N, H |
| 360 | G | R, A, K, Q, N, H |
| 361 | K | R, G, A, Q, N, H |
| 362 | D | R, G, A, K, Q, N, H |
| 363 | L | R, G, A, K, Q, N, H |
| 364 | S | R, G, A, K, Q, N, H |
| 365 | K | R, E, G, A, Q, N, H |
| 366 | L | R, G, A, K, Q, N, H |
| 367 | Y | R, F, G, A, K, Q, N, H |
| 368 | K | R, G, A, Q, N, H |
| 369 | A | R, I, G, K, Q, N, H |
| 370 | W | R, G, A, K, Q, N, H |
| 371 | E | R, G, A, K, Q, N, H |
| 372 | D | R, E, G, A, K, Q, N, H |
| 373 | D | R, L, G, A, K, Q, N, H |
| 374 | P | R, G, A, K, Q, N, H |
| 375 | A | R, G, K, Q, N, H |
| 376 | D | R, G, A, K, Q, N, H |
| 377 | P | R, M, G, A, K, Q, N, H |
| 378 | E | R, D, G, A, K, Q, N, H |
| 379 | N | R, G, A, K, Q, H |
| 380 | A | R, G, K, Q, N, H |
| 381 | I | R, G, A, K, Q, N, H |
| 382 | V | R, G, A, K, Q, N, H |
| 383 | V | R, G, A, K, Q, N, H, D, E |
| 384 | L | R, Y, G, A, K, Q, N, H |
| 385 | C | R, G, A, K, Q, N, H |
| 386 | D | R, G, A, K, Q, N, H, E, D |
| 387 | D | R, G, A, K, Q, N, H, E |
| 388 | L | R, C, G, A, K, Q, N, H |
| 389 | K | R, G, A, Q, N, H |
| 390 | N | R, D, G, A, K, Q, H |
| 391 | N | R, K, G, A, Q, H |
| 392 | F | R, G, A, K, Q, N, H |
| 393 | K | R, S, G, A, Q, N, H |
| 394 | K | R, G, A, Q, N, H |
| 395 | E | R, G, A, K, Q, N, H, S |
| 396 | P | R, G, A, K, Q, N, H |
| 397 | I | R, G, A, K, Q, N, H, V |
| 398 | R | G, A, K, Q, N, H |
| 399 | N | R, G, A, K, Q, H, S |
| 400 | I | R, L, G, A, K, Q, N, H |
| 401 | L | R, G, A, K, Q, N, H |
| 402 | R | G, A, K, Q, N, H, S |
| 403 | Y | R, G, A, K, Q, N, H |
| 404 | I | R, G, A, K, Q, N, H |
| 405 | F | R, Y, G, A, K, Q, N, H |
| 406 | T | R, G, A, K, Q, N, H |
| 407 | I | R, Y, G, A, K, Q, N, H |
| 408 | R | G, A, K, Q, N, H |
| 409 | Q | R, D, G, A, K, N, H |
| 410 | E | R, K, G, A, Q, N, H |
| 411 | C | R, I, G, A, K, Q, N, H |
| 412 | S | R, G, A, K, Q, N, H |
| 413 | A | R, G, K, Q, N, H |
| 414 | Q | R, K, G, A, N, H |
| 415 | D | R, G, A, K, Q, N, H |
| 416 | I | R, F, G, A, K, Q, N, H |
| 417 | L | R, G, A, K, Q, N, H |
| 418 | A | R, G, K, Q, N, H |
| 419 | A | R, D, G, K, Q, N, H |
| 420 | A | R, G, K, Q, N, H |
| 421 | K | R, G, A, Q, N, H |
| 422 | Y | R, G, A, K, Q, N, H |
| 423 | N | R, G, A, K, Q, H |
| 424 | Q | R, G, A, K, N, H |
| 425 | Q | R, L, G, A, K, N, H |
| 426 | L | R, G, A, K, Q, N, H |
| 427 | D | R, E, G, A, K, Q, N, H |
| 428 | R | K, G, A, Q, N, H |
| 429 | Y | R, N, G, A, K, Q, H |
| 430 | K | R, G, A, Q, N, H |
| 431 | S | R, G, A, K, Q, N, H |
| 432 | Q | R, K, G, A, K, N, H |
| 433 | K | R, G, A, Q, N, H, S |
| 434 | A | R, I, G, K, Q, N, H |
| 435 | N | R, H, G, A, K, Q |
| 436 | P | R, G, A, K, Q, N, H |
| 437 | S | R, T, G, A, K, Q, N, H |
| 438 | V | R, G, A, K, Q, N, H |
| 439 | L | R, G, A, K, Q, N, H |
| 440 | G | R, A, K, Q, N, H |
| 441 | N | R, G, A, K, Q, H |
| 442 | Q | R, T, G, A, K, N, H |
| 443 | G | R, A, K, Q, N, H |
| 444 | F | R, G, A, K, Q, N, H |
| 445 | T | R, N, G, A, K, Q, H |
| 446 | W | R, G, A, K, Q, N, H |
| 447 | T | R, G, A, K, Q, N, H, S, M |
| 448 | N | R, G, A, K, Q, H |
| 449 | A | R, S, G, K, Q, N, H |
| 450 | V | R, T, G, A, K, Q, N, H |
| 451 | I | R, G, A, K, Q, N, H |
| 452 | L | R, T, G, A, K, Q, N, H |
| 453 | P | R, G, A, K, Q, N, H |
| 454 | E | R, P, G, A, K, Q, N, H |
| 455 | K | R, N, G, A, Q, H |
| 456 | A | R, G, K, Q, N, H |
| 457 | Q | R, G, A, K, N, H |
| 458 | R | V, G, A, K, Q. N, H |
| 459 | N | R, K, G, A, Q, H |
| 460 | D | R, G, A, K, Q, N, H |
| 461 | R | G, A, K, Q, N, H |
| 462 | P | R, G, A, K, Q, N, H |
| 463 | N | R, A, G, K, Q, H |
| 464 | S | R, G, A, K, Q, N, H |
| 465 | L | R, S, G, A, K, Q, N, H |
| 466 | D | R, G, A, K, Q, N, H |
| 467 | L | R, G, A, K, Q, N, H |
| 468 | R | M, G, A, K, Q, N, H |
| 469 | I | R, G, A, K, Q, N, H |
| 470 | W | R, G, A, K, Q, N, H |
| 471 | L | R, V, G, A, K, Q, N, H |
| 472 | Y | R, T, G, A, K, Q, N, H |
| 473 | L | R, M, G, A, K, Q, N, H |
| 474 | K | R, T, G, A, Q, N, H |
| 475 | L | R, V, G, A, K, Q, N, H |
| 476 | R | G, A, K, Q, N, H |
| 477 | H | R, G, A, K, Q, N |
| 478 | P | R, D, G, A, K, Q, N, H |
| 479 | D | R, N, G, A, K, Q, H |
| 480 | G | R, A, K, Q, N, H |
| 481 | R | G, A, K, Q, N, H |
| 482 | W | R, G, A, K, Q, N, H |
| 483 | K | R, G, A, Q, N, H |
| 484 | K | R, G, A, Q, N, H |
| 485 | H | R, G, A, K, Q, N |
| 486 | H | R, G, A, K, Q, N |
| 487 | I | R, G, A, K, Q, N, H |
| 488 | P | R, G, A, K, Q, N, H |
| 489 | F | R, G, A, K, Q, N, H |
| 490 | Y | R, H, G, A, K, Q, N |
| 491 | D | R, N, G, A, K, Q, N, H |
| 492 | T | R, S, G, A, K, Q, N, H |
| 493 | R | G, A, K, Q, N, H |
| 494 | F | R, Y, G, A, K, Q, N, H |
| 495 | F | R, Y, G, A, K, Q, N, H |
| 496 | Q | R, E, G, A, K, N, H, S |
| 497 | E | R, G, A, K, Q, N, H |
| 498 | I | R, G, A, K, Q, N, H |
| 499 | Y | R, G, A, K, Q, N, H |
| 500 | A | R, G, K, Q, N, H |
| 501 | A | R, Y, G, K, Q, N, H |
| 502 | G | R, A, K, Q, N, H |
| 503 | N | R, G, A, K, Q, H |
| 504 | S | R, G, G, A, K, Q, N, H |
| 505 | P | R, L, G, A, K, Q, N, H |
| 506 | V | R, P, G, A, K, Q, N, H |
| 507 | D | R, T, G, A, K, Q, N, H |
| 508 | T | R, G, A, K, Q, N, H |
| 509 | C | R, G, A, K, Q, N, H |
| 510 | Q | R, G, A, K, N, H |
| 511 | F | R, P, G, A, K, Q, N, H |
| 512 | R | G, A, K, Q, N, H |
| 513 | T | R, G, A, K, Q, N, H |
| 514 | P | R, K, G, A, K, Q, N, H |
| 515 | R | G, A, K, Q, N, H |
| 516 | F | R, G, A, K, Q, N, H |
| 517 | G | R, A, K, Q, N, H |
| 518 | Y | R, G, A, K, Q, N, H |
| 519 | H | R, G, A, K, Q, N |
| 520 | L | R, G, A, K, Q, N, H |
| 521 | P | R, G, A, K, Q, N, H |
| 522 | K | R, G, A, Q, N, H |
| 523 | L | R, I, G, A, K, Q, N, H |
| 524 | T | R, S, G, A, K, Q, N, H |
| 525 | D | R, G, A, K, Q, N, H |
| 526 | Q | R, G, A, K, N, H |
| 527 | T | R, G, A, K, Q, N, H |
| 528 | A | R, G, K, Q, N, H |
| 529 | I | R, G, A, K, Q, N, H |
| 530 | R | G, A, K, Q, N, H |
| 531 | V | R, L, G, A, K, Q, N, H |
| 532 | N | R, K, G, A, Q, H |
| 533 | K | R, G, A, Q, N, H |
| 534 | K | R, G, A, Q, N, H |
| 535 | H | R, G, A, K, Q, N |
| 536 | V | R, G, A, K, Q, N, H |
| 537 | K | R, G, A, Q, N, H |
| 538 | A | R, G, K, Q, N, H |
| 539 | A | R, G, K, Q, N, H |
| 540 | K | R, G, A, Q, N, H |
| 541 | T | R, G, A, K, Q, N, H, Y |
| 542 | E | R, G, A, K, Q, N, H |
| 543 | A | R, G, K, Q, N, H |
| 544 | R | G, A, K, Q, N, H |
| 545 | I | R, G, A, K, Q, N, H |
| 546 | R | G, A, K, Q, N, H |
| 547 | L | R, G, A, K, Q, N, H |
| 548 | A | R, N, G, K, Q, N, H |
| 549 | I | R, G, A, K, Q, N, H |
| 550 | Q | R, G, A, K, N, H |
| 551 | Q | R, G, A, K, N, H |
| 552 | G | R, N, A, K, Q, N, H |
| 553 | T | R, V, G, A, K, Q, N, H |
| 554 | L | R, A, G, A, K, Q, N, H |
| 555 | P | R, G, A, K, Q, N, H |
| 556 | V | R, G, A, K, Q, N, H |
| 557 | S | R, G, A, K, Q, N, H |
| 558 | N | R, G, A, K, Q, H |
| 559 | L | R, F, G, A, K, Q, N, H |
| 560 | K | R, D, G, A, Q, N, H |
| 561 | I | R, G, A, K, Q, N, H |
| 562 | T | R, G, A, K, Q, N, H |
| 563 | E | R, G, A, K, Q, N, H |
| 564 | I | R, T, G, A, K, Q, N, H |
| 565 | S | R, N, G, A, K, Q, H |
| 566 | A | R, F, G, K, Q, N, H |
| 567 | T | R, G, A, K, Q, N, H |
| 568 | I | R, V, G, A, K, Q, N, H |
| 569 | N | R, G, A, K, Q, H |
| 570 | S | R, G, A, K, Q, N, H |
| 571 | K | R, G, A, Q, N, H |
| 572 | G | R, N, A, K, Q, N, H |
| 573 | Q | R, D, G, A, K, N, H |
| 574 | V | R, I, G, A, K, Q, N, H |
| 575 | R | T, H, K, N, G, A. Q |
| 576 | I | R, H, K, N, G, A, Q |
| 577 | P | R, S, H, K, N, G, A, Q, T, |
| | | V, Y, F, L |
| 578 | V | R, H, K, N, G, A, Q |
| 579 | K | R, H, N, G, A, Q |
| 580 | F | R, V, H, K, N, G, A, Q |
| 581 | D | R, H, K, N, G, A, Q |
| 582 | V | R, G, H, K, N, G, A, Q |
| 583 | G | R, H, K, N, A, Q |
| 584 | R | G, K, H, K, N, A, Q |
| 585 | Q | R, G, Y, H, K, N, A |
| 586 | K | R, G, H, N, A, Q |
| 587 | G | R, H, K, NA, Q |
| 588 | T | R, G, K, H, N A, Q |
| 589 | L | R, G, H, K, N A, Q |
| 590 | Q | R, G, H, K, N, A |
| 591 | I | R, G, H, K, N, A, Q |
| 592 | G | R, H, K, N, A, Q |
| 593 | D | R, G, H, K, N, A, Q |
| 594 | R | G, H, K, N, A, Q |
| 595 | F | R, G, I, H, K, N, A, Q |
| 596 | C | R, G, M, H, K, N, A, Q |
| 597 | G | R, H, K, N, A, Q |
| 598 | Y | R, G, H, K, N, A, Q |
| 599 | D | A |
| 600 | Q | R, G, K, N, H, A, D, C, E, I, L, |
| | | M, F, P, S, T, W, Y, V |
| 601 | N | R, G, K, H, A, D, C, Q, E, I, L, |
| | | M, F, P, S, T, W, Y, V |
| 602 | Q | R, G, K, N, H, A, D, C, E, I, L, |
| | | M, F, P, S, T, W, Y, V |
| 603 | T | R, G, K, N, H, A, D, C, Q, E, I, |
| | | L, M, F, P, S, W, Y, V |
| 604 | A | R, G, K, N, H, D, C, Q, E, I, L, |
| | | M, F, P, S, T, W, Y, V |
| 605 | S | R, G, K, N, H, A, D, C, Q, E, I, |
| | | L, M, F, P, T, W, Y, V |
| 606 | H | R, G, K, N, A, D, C, Q, E, I, L, |
| | | M, F, P, S, T, W, Y, V |
| 607 | A | R, G, K, N, H, D, C, Q, E, I, L, |
| | | M, F, P, S, T, W, Y, V |
| 608 | Y | R, G, H, K, N, A, Q |
| 609 | S | R, G, H, K, N, A, Q |
| 610 | L | R, G, I, H, K, N, A, Q |
| 611 | W | R, G, H, K, N, A, Q |
| 612 | E | R, G, H, K, N, A, Q |
| 613 | V | R, G, H, K, N, A, Q |
| 614 | V | R, G, H, K, N, A, Q |
| 615 | K | R, G, E, H, N, A, Q |
| 616 | E | R, G, H, K, N, A, Q |
| 617 | G | R, H, K, N, A, Q |
| 618 | Q | R, G, T, H, K, N, A |
| 619 | Y | R, G, S, H, K, N, A, Q |
| 620 | H | R, G, K, N, A, Q |
| 621 | K | R, G, H, N, A, Q |
| 622 | E | R, G, H, K, N, A, Q |
| 623 | L | R, G, N, H, K, A, Q |
| 624 | G | R, H, K, N, A, Q |
| 625 | C | R, G, H, K, N, A, Q, A, Q |
| 626 | F | R, G, W, H, K, N, A, Q |
| 627 | V | R, G, H, K, N, A, Q |
| 628 | R | G, H, K, N, A, Q |
| 629 | F | R, G, L, H, K, N, A, Q |
| 630 | I | R, G, V, H, K, N, A, Q |
| 631 | S | R, G, E, H, K, N, A, Q |
| 632 | S | R, G, D, H, K, N, A, Q |
| 633 | G | R, H, K, N, A, Q |
| 634 | D | R, G, K, H, K, N, A, Q |
| 635 | I | R, G, H, K, N, A, Q |
| 636 | V | R, G, T, H, K, N, A, Q |
| 637 | S | R, G, H, K, N, A, Q |
| 638 | I | R, G, H, K, N, A, Q |
| 639 | T | R, G, H, K, N, A, Q |
| 640 | E | R, G, H, K, N, A, Q |
| 641 | N | R, G, H, K, A, Q |
| 642 | R | G, H, K, N, A, Q |
| 643 | G | R, H, K, N, A, Q |
| 644 | N | R, G, H, K, A, Q |
| 645 | Q | R, G, H, K, N, A |
| 646 | F | R, G, V, H, K, N, A, Q, Y |
| 647 | D | R, G, H, K, N, A, Q |
| 648 | Q | R, G, H, K, N, A, |
| 649 | L | R, G, H, K, N, A, Q |
| 650 | S | R, G, H, K, N, A, Q |
| 651 | Y | R, G, H, K, N, A, Q |
| 652 | E | R, G, D, H, K, N, A, Q |
| 653 | G | R, H, K, N, A, Q |
| 654 | L | R, G, H, K, N, A, Q |
| 655 | A | R, G, H, K, N, Q |
| 656 | Y | R, G, H, K, N, A, Q |
| 657 | P | R, G, H, K, N, A, Q |
| 658 | Q | R, G, H, K, N, A |
| 659 | Y | R, G, F, H, K, N, A, Q |
| 660 | A | R, G, H, K, N, Q |
| 661 | D | R, G, E, H, K, N, A, Q |
| 662 | W | R, G, H, K, N, A, Q |
| 663 | R | G, H, K, N, A, Q |
| 664 | K | R, G, H, N, A, Q |
| 665 | K | R, G, H, N, A, Q |
| 666 | A | R, G, H, K, N, Q |
| 667 | S | R, G, H, K, N, A, Q |
| 668 | K | R, G, H, N, A, Q |
| 669 | F | R, G, H, K, N, A, Q |
| 670 | V | R, G, L, H, K, N, A, Q |
| 671 | S | R, G, H, K, N, A, Q |
| 672 | L | R, G, S, H, K, N, A, Q |
| 673 | W | R, G, H, K, N, A, Q |
| 674 | Q | R, G, H, K, N, A |
| 675 | I | R, G, H, K, N, A, Q |
| 676 | T | R, G, H, K, N, A, Q |
| 677 | K | R, G, H, N, A, Q |
| 678 | K | R, G, H, N, A, Q |
| 679 | N | R, G, K, N, A, Q |
| 680 | K | R, G, H, N, A, Q |
| 681 | K | R, G, H, N, A, Q |
| 682 | K | R, G, H, N, A, Q |
| 683 | E | R, G, H, K, N, A, Q |
| 684 | I | R, G, D, H, K, N, A, Q |
| 685 | V | R, G, H, K, N, A, Q |
| 686 | T | R, G, H, K, N, A, Q |
| 687 | V | R, G, K, H, N, A, Q |
| 688 | E | R, G, H, K, N, A, Q |
| 689 | A | R, G, H, K, N, Q |
| 690 | K | R, G, H, N, A, Q |
| 691 | E | R, G, H, K, N, A, Q |
| 692 | K | R, G, H, N, A, Q |
| 693 | F | R, G, H, K, N, A, Q |
| 694 | D | R, G, K, H, N, A, Q |
| 695 | A | R, G, H, K, N, Q |
| 696 | I | R, G, H, K, N, A, Q |
| 697 | C | R, G, H, K, N, A, Q |
| 698 | K | R, G, W, H, N, A, Q |
| 699 | Y | R, G, H, K, N, A, Q |
| 700 | Q | R, G, H, K, N, A |
| 701 | P | R, G, H, K, N, A, Q |
| 702 | R | G, H, K, N, A, Q |
| 703 | L | R, G, H, K, N, A, Q |
| 704 | Y | R, G, H, K, N, A, Q |
| 705 | K | R, G, H, N, A, Q |
| 706 | F | R, G, W, H, K, N, A, Q |
| 707 | N | R, G, H, K, A, Q |
| 708 | K | R, G, H, N, A, Q |
| 709 | E | R, G, H, K, N, A, Q |
| 710 | Y | R, G, H, K, N, A, Q |
| 711 | A | R, G, H, K, N, Q |
| 712 | Y | R, G, H, K, N, A, Q |
| 713 | L | R, G, H, K, N, A, Q |
| 714 | L | R, G, H, K, N, A, Q |
| 715 | R | G, L, H, K, N, A, Q |
| 716 | D | R, G, H, K, N, A, Q |
| 717 | I | R, G, H, K, N, A, Q |
| 718 | V | R, G, M, H, K, N, A, Q |
| 719 | R | G, K, H, N, A, Q |
| 720 | G | R, H, K, N, A, Q |
| 721 | K | R, G, N, H, A, Q |
| 722 | S | R, G, H, K, N, A, Q |
| 723 | L | R, G, H, K, N, A, Q |
| 724 | V | R, G, H, K, N, A, Q |
| 725 | E | R, G, N, H, K, A, Q |
| 726 | L | R, G, H, K, N, A, Q |
| 727 | Q | R, G, H, K, N, A |
| 728 | Q | R, G, H, K, N, A |
| 729 | I | R, G, F, H, K, N, A, Q |
| 730 | R | G, H, K, N, A, Q |
| 731 | Q | R, G, A, H, K, N, A |
| 732 | E | R, G, H, K, N, A, Q |
| 733 | I | R, G, H, K, N, A, Q |
| 734 | F | R, G, H, K, N, A, Q |
| 735 | R | G, H, K, N, A, Q |
| 736 | F | R, G, H, K, N, A, Q |
| 737 | I | R, G, H, K, N, A, Q |
| 738 | E | R, G, H, K, N, A, Q |
| 739 | Q | R, G, H, K, N, A |
| 740 | D | R, G, H, K, N, A, Q |
| 741 | C | R, G, F, H, K, N, A, Q, S |
| 742 | G | R, H, K, N, A, Q |
| 743 | V | R, G, H, K, N, A, Q |
| 744 | T | R, G, H, K, N, A, Q |
| 745 | R | G, H, K, N, A, Q |
| 746 | L | R, G, H, K, N, A, Q |
| 747 | G | R, H, K, N, A, Q |
| 748 | S | R, G, H, K, N, A, Q |
| 749 | L | R, G, H, K, N, A, Q |
| 750 | S | R, G, H, K, N, A, Q |
| 751 | L | R, G, H, K, N, A, Q |
| 752 | S | R, G, H, K, N, A, Q |
| 753 | T | R, G, S, H, K, N, A, Q |
| 754 | L | R, G, H, K, N, A, Q |
| 755 | E | R, G, H, K, N, A, Q |
| 756 | T | R, G, H, K, N, A, Q |
| 757 | V | R, G, L, H, K, N, A, Q |
| 758 | K | R, G, H, N, A, Q |
| 759 | A | R, G, N, H, K, Q |
| 760 | V | R, G, H, K, N, A, Q |
| 761 | K | R, G, H, N, A, Q |
| 762 | G | R, S, H, K, N, A, Q |
| 763 | I | R, G, L, H, K, N, A, Q |
| 764 | I | R, G, H, K, N, A, Q |
| 765 | Y | R, G, S, H, K, N, A, Q |
| 766 | S | R, G, H, K, N, A, Q |
| 767 | Y | R, G, H, K, N, A, Q |
| 768 | F | R, G, H, K, N, A, Q |
| 769 | S | R, G, H, K, N, A, Q |
| 770 | T | R, G, L, H, K, N, A, Q |
| 771 | A | R, G, N, H, K, Q |
| 772 | L | R, G, H, K, N, A, Q |
| 773 | N | R, G, H, K, A, Q |
| 774 | A | R, G, N, H, K, Q |
| 775 | S | R, G, H, K, N, A, Q |
| 776 | K | R, G, H, N, A, Q |
| 777 | N | R, G, E, H, K, A, Q |
| 778 | N | R, G, H, K, A, Q |
| 779 | P | R, G, H, K, N, A, Q |
| 780 | I | R, G, H, K, N, A, Q |
| 781 | S | R, G, H, K, N, A, Q |
| 782 | D | R, G, H, K, N, A, Q |
| 783 | E | R, G, H, K, N, A, Q |
| 784 | Q | R, G, D, H, K, N, A |
| 785 | R | G, K, Q, H, N, A, Q |
| 786 | K | R, G, E, H, N, A, Q |
| 787 | E | R, G, H, K, N, A, Q |
| 788 | F | R, G, H, K, N, A, Q |
| 789 | D | R, G, H, K, N, A, Q |
| 790 | P | R, G, H, K, N, A, Q |
| 791 | E | R, G, H, K, N, A, Q |
| 792 | L | R, G, H, K, N, A, Q |
| 793 | F | R, G, H, K, N, A, Q |
| 794 | A | R, G, H, K, N, Q |
| 795 | L | R, G, H, K, N, A, Q |
| 796 | L | R, G, M, H, K, N, A, Q |
| 797 | E | R, G, V, H, K, N, A, Q |
| 798 | K | R, G, H, N, A, Q |
| 799 | L | R, G, I, H, K, N, A, Q |
| 800 | E | R, G, H, K, N, A, Q, S |
| 801 | L | R, G, H, K, N, A, Q |
| 802 | I | R, G, K, H, N, A, Q |
| 803 | R | G, H, K, N, A, Q |
| 804 | T | R, G, H, K, N, A, Q |
| 805 | R | G, N, H, K, A, Q |
| 806 | K | R, G, H, N, A, Q |
| 807 | K | R, G, H, N, A, Q |
| 808 | K | R, G, H, N, A, Q |
| 809 | Q | R, G, E, H, K, N, A, Q |
| 810 | K | R, G, H, N, A, Q |
| 811 | V | R, G, H, K, N, A, Q |
| 812 | E | R, G, S, H, K, N, A, Q |
| 813 | R | G, H, K, N, A, Q |
| 814 | I | R, G, H, K, N, A, Q |
| 815 | A | R, G, S, H, K, N, Q |
| 816 | N | R, G, S, H, K, A, Q |
| 817 | S | R, G, H, K, N, A, Q |
| 818 | L | R, G, H, K, N, A, Q |
| 819 | I | R, G, L, H, K, N, A, Q |
| 820 | Q | R, G, H, K, N, A |
| 821 | T | R, G, I, H, K, N, A, Q |
| 822 | C | R, G, A, H, K, N, A, Q |
| 823 | L | R, G, H, K, N, A, Q |
| 824 | E | R, G, H, K, N, A, Q |
| 825 | N | R, G, H, K, A, Q |
| 826 | N | R, G, H, K, A, Q |
| 827 | I | R, G, V, H, K, N, A, Q |
| 828 | K | R, G, H, N, A, Q |
| 829 | F | R, G, H, K, N, A, Q |
| 830 | I | R, G, H, K, N, A, Q |
| 831 | R | G, V, H, K, N, A, Q |
| 832 | G | R, V, H, K, N, A, Q |
| 833 | E | A |
| 834 | G | R, K, N, H, A, D, C, Q, E, I, |
| | | L, M, F, P, S, T, W, Y, V |
| 835 | D | R, G, K, N, H, A, C, Q, E, I, |
| | | L, M, F, P, S, T, W, Y, V |
| 836 | L | R, G, K, N, H, A, D, C, Q, E, |
| | | I, M, F, P, S, T, W, Y, V |
| 837 | S | R, G, K, N, H, A, D, C, Q, E, |
| | | I, L, M, F, P, T, W, Y, V |
| 838 | T | R, G, K, N, H, A, D, C, Q, E, |
| | | I, L, M, F, P, S, W, Y, V |
| 839 | T | R, G, K, N, H, A, D, C, Q, E, |
| | | I, L, M, F, P, S, W, Y, V |
| 840 | N | R, G, K, H, A, D, C, Q, E, I, |
| | | L, M, F, P, S, T, W, Y, V |
| 841 | N | R, G, K, H, A, D, C, Q, E, I, |
| | | L, M, F, P, S, T, W, Y, V |
| 842 | A | R, G, K, N, H, D, C, Q, E, I, |
| | | L, M, F, P, S, T, W, Y, V |
| 843 | T | R, G, K, N, H, A, D, C, Q, E, |
| | | I, L, M, F, P, S, W, Y, V |
| 844 | K | R, G, N, H, A, D, C, Q, E, I, |
| | | L, M, F, P, S, T, W, Y, V |
| 845 | K | R, G, N, H, A, D, C, Q, E, I, |
| | | L, M, F, P, S, T, W, Y, V |
| 846 | K | R, G, N, H, A, D, C, Q, E, I, |
| | | L, M, F, P, S, T, W, Y, V |
| 847 | A | R, G, K, N, H, D, C, Q, E, I, |
| | | L, M, F, P, S, T, W, Y, V |
| 848 | N | R, G, K, H, A, D, C, Q, E, I, |
| | | L, M, F, P, S, T, W, Y, V |
| 849 | S | R, G, K, N, H, A, D, C, Q, E, |
| | | I, L, M, F, P, T, W, Y, V |
| 850 | R | G, K, N, H, A, D, C, Q, E, |
| | | I, L, M, F, P, S, T, W, Y, V |
| 851 | S | R, G, K, N, H, A, D, C, Q, E, |
| | | I, L, M, F, P, T, W, Y, V |
| 852 | M | R, G, K, N, H, A, D, C, Q, E, |
| | | I, L, F, P, S, T, W, Y, V |
| 853 | D | R, G, K, N, H, A, C, Q, E, I, |
| | | L, M, F, P, S, T, W, Y, V |
| 854 | W | R, G, K, N, H, A, D, C, Q, E, |
| | | I, L, M, F, P, S, T, Y, V |
| 855 | L | R, G, K, N, H, A, D, C, Q, E, |
| | | I, M, F, P, S, T, W, Y, V |
| 856 | A | R, G, K, N, H, D, C, Q, E, I, |
| | | L, M, F, P, S, T, W, Y, V |
| 857 | R | G, K, N, H, A, D, C, Q, E, I, |
| | | L, M, F, P, S, T, W, Y, V |
| 858 | G | R, K, N, H, A, D, C, Q, E, I, |
| | | L, M, F, P, S, T, W, Y, V |
| 859 | V | R, G, K, N, H, A, D, C, Q, E, |
| | | I, L, M, F, P, S, T, W, Y |
| 860 | F | R, G, K, N, H, A, D, C, Q, E, |
| | | I, L, M, P, S, T, W, Y, V |
| 861 | N | R, G, K, H, A, D, C, Q, E, I, |
| | | L, M, F, P, S, T, W, Y, V |
| 862 | K | R, G, N, H, A, D, C, Q, E, I, |
| | | L, M, F, P, S, T, W, Y, V |
| 863 | I | R, G, K, N, H, A, D, C, Q, |
| | | E, L, M, F, P, S, T, W, Y, V |
| 864 | R | G, K, N, H, A, D, C, Q, E, I, |
| | | L, M, F, P, S, T, W, Y, V |
| 865 | Q | R, G, K, N, H, A, D, C, E, I, |
| | | L, M, F, P, S, T, W, Y, V |
| 866 | L | R, G, K, N, H, A, D, C, Q, E, |
| | | I, M, F, P, S, T, W, Y, V |
| 867 | A | R, G, K, N, H, D, C, Q, E, I, |
| | | L, M, F, P, S, T, W, Y, V |
| 868 | P | R, G, K, N, H, A, D, C, Q, E, |
| | | I, L, M, F, S, T, W, Y, V |
| 869 | M | R, G, K, N, H, A, D, C, Q, E, |
| | | I, L, F, P, S, T, W, Y, V |
| 870 | H | R, G, K, N, A, D, C, Q, E, I, |
| | | L, M, F, P, S, T, W, Y, V |
| 871 | N | R, G, K, H, A, D, C, Q, E, I, |
| | | L, M, F, P, S, T, W, Y, V |
| 872 | I | R, G, H, K, N, A, Q |
| 873 | T | R, G, H, K, N, A, Q |
| 874 | L | R, G, H, K, N, A, Q |
| 875 | F | R, G, H, K, N, A, Q |
| 876 | G | R, H, K, N, A, Q |
| 877 | C | R, G, K, N, H, A, D, Q, E, I, |
| | | L, M, F, P, S, T, W, Y, V |
| 878 | G | R, K, N, H, A, D, C, Q, E, I, |
| | | L, M, F, P, S, T, W, Y, V |
| 879 | S | R, G, K, N, H, A, D, C, Q, E, |
| | | I, L, M, F, P, T, W, Y, V |
| 880 | L | R, G, K, N, H, A, D, C, Q, E, |
| | | I, M, F, P, S, T, W, Y, V |
| 881 | Y | R, G, K, N, H, A, D, C, Q, E, |
| | | I, L, M, F, P, S, T, W, V |
| 882 | T | R, G, K, N, H, A, D, C, Q, E, |
| | | I, L, M, F, P, S, W, Y, V |
| 883 | S | R, G, K, N, H, A, D, C, Q, E, |
| | | I, L, M, F, P, T, W, Y, V |
| 884 | H | R, G, K, N, A, D, C, Q, E, I, |
| | | L, M, F, P, S, T, W, Y, V |
| 885 | Q | R, G, K, N, H, A, D, C, E, I, |
| | | L, M, F, P, S, T, W, Y, V |
| 886 | D | R, G, H, K, N, A, Q |
| 887 | P | R, G, H, K, N, A, Q |
| 888 | L | R, G, F, H, K, N, A, Q |
| 889 | V | R, G, H, K, N, A, Q |
| 890 | H | R, G, K, N, A, Q |
| 891 | R | G, H, K, N, A, Q |
| 892 | N | R, G, H, K, A, Q |
| 893 | P | R, G, H, K, N, A, Q |
| 894 | D | R, G, H, K, N, A, Q |
| 895 | K | R, G, H, N, A, Q |
| 896 | A | R, G, H, K, N, Q |
| 897 | M | R, G, K, H, N, A, Q |
| 898 | K | R, E, H, N, A, Q |
| 899 | C | R, G, A, H, K, N, Q |
| 900 | R | G, H, K, N, A, Q |
| 901 | W | R, G, F, Y, H, K, N, A, Q |
| 902 | A | R, G, H, K, N, Q |
| 903 | A | R, G, H, K, N, Q |
| 904 | I | R, G, V, H, K, N, A, Q |
| 905 | P | R, G, H, K, N, A, Q |
| 906 | V | R, G, P, H, K, N, A, Q |
| 907 | K | R, G, S, H, N, A, Q |
| 908 | D | R, G, H, K, N, A, Q |
| 909 | I | R, G, H, K, N, A, Q |
| 910 | G | R, K, H, K, N, A, Q |
| 911 | D | R, G, E, H, K, N, A, Q |
| 912 | W | R, G, Y, H, K, N, A, Q |
| 913 | V | R, G, H, K, N, A, Q |
| 914 | L | R, G, H, K, N, A, Q |
| 915 | R | G, H, K, N, A, Q |
| 916 | K | R, G, H, N, A, Q |
| 917 | L | R, G, F, H, K, N, A, Q |
| 918 | S | R, G, H, K, N, A, Q |
| 919 | Q | R, G, H, K, N, A |
| 920 | N | R, G, W, H, K, A, Q |
| 921 | L | R, G, H, K, N, A, Q |
| 922 | R | G, H, K, N, A, Q |
| 923 | A | R, G, H, K, N, Q |
| 924 | K | R, G, H, N, A, Q |
| 925 | N | R, G, T, H, K, A, Q, D, E |
| 926 | I | R, G, S, H, K, N, A, Q |
| 927 | G | R, T, H, K, N, A, Q |
| 928 | T | R, G, H, K, N, A, Q |
| 929 | G | R, H, K, N, A, Q |
| 930 | E | R, G, H, K, N, A, Q |
| 931 | Y | R, G, L, H, K, N, A, Q |
| 932 | Y | R, G, H, K, N, A, Q |
| 933 | H | R, G, K, N, A, Q |
| 934 | Q | R, G, E, H, K, N, A |
| 935 | G | R, A, H, K, N, A, Q |
| 936 | V | R, G, L, H, K, N, A, Q |
| 937 | K | R, G, H, N, A, Q |
| 938 | E | R, G, H, K, N, A, Q |
| 939 | F | R, G, H, K, N, A, Q |
| 940 | L | R, G, H, K, N, A, Q |
| 941 | S | R, G, H, K, N, A, Q |
| 942 | H | R, G, K, N, A, Q |
| 943 | Y | R, G, H, K, N, A, Q |
| 944 | E | R, G, H, K, N, A, Q |
| 945 | L | R, G, H, K, N, A, Q |
| 946 | Q | R, G, D, H, K, N, A |
| 947 | D | R, G, H, K, N, A, Q |
| 948 | L | R, G, D, H, K, N, A, Q |
| 949 | E | R, G, H, K, N, A, Q |
| 950 | E | R, G, L, H, K, N, A, Q |
| 951 | E | R, G, P, H, K, N, A, Q |
| 952 | L | R, G, K, H, K, N, A, Q |
| 953 | L | R, G, M, H, K, N, A, Q |
| 954 | K | R, G, H, N, A, Q |
| 955 | W | R, G, F, H, K, N, A, Q |
| 956 | R | G, H, K, N, A, Q |
| 957 | S | R, G, H, K, N, A, Q |
| 958 | D | R, G, L, H, K, N, A, Q |
| 959 | R | G, K, H, N, A, Q |
| 960 | K | R, G, H, K, N, A, Q |
| 961 | S | R, G, H, K, N, A, Q |
| 962 | N | R, G, H, K, A, Q |
| 963 | I | R, G, H, K, N, A, Q |
| 964 | P | R, G, H, K, N, A, Q |
| 965 | C | R, G, H, K, N, A, Q |
| 966 | W | R, G, H, K, N, A, Q |
| 967 | V | R, G, H, K, N, A, Q |
| 968 | L | R, G, H, K, N, A, Q |
| 969 | Q | R, G, H, K, N, A |
| 970 | N | R, G, H, K, A, Q |
| 971 | R | G, H, K, N, A, Q |
| 972 | L | G, I, H, K, N, A, Q |
| 973 | A | R, G, L, H, K, N, Q |
| 974 | E | R, G, H, K, N, A, Q |
| 975 | K | R, G, D, H, N, A, Q |
| 976 | L | R, G, H, K, N, A, Q |
| 977 | G | R, H, K, N, A, Q |
| 978 | N | R, G, H, K, A, Q |
| 979 | K | R, G, H, N, A, Q |
| 980 | E | R, G, H, K, N, A, Q |
| 981 | A | R, G, H, K, N, Q |
| 982 | V | R, G, H, K, N, A, Q |
| 983 | V | R, G, H, K, N, A, Q |
| 984 | Y | R, G, I, H, K, N, A, Q |
| 985 | I | R, G, H, K, N, A, Q |
| 986 | P | R, G, H, K, N, A, Q |
| 987 | V | R, G, H, K, N, A, Q |
| 988 | R | G, H, K, N, A, Q |
| 989 | G | R, H, K, N, A, Q |
| 990 | G | R, H, K, N, A, Q |
| 991 | R | G, H, K, N, A, Q |
| 992 | I | R, G, H, K, N, A, Q |
| 993 | Y | R, G, H, K, N, A, Q |
| 994 | F | R, G, L, H, K, N, A, Q |
| 995 | A | R, G, S, H, K, N, A, Q |
| 996 | T | R, G, H, K, N, A, Q |
| 997 | H | R, G, K, N, A, Q |
| 998 | K | R, G, P, H, N, A, Q |
| 999 | V | R, G, H, K, N, A, Q |
| 1000 | A | R, G, T, H, K, N, Q |
| 1001 | T | R, G, H, K, N, A, Q |
| 1002 | G | R, D, H, K, N, A, Q |
| 1003 | A | R, G, S, H, K, N, Q |
| 1004 | V | R, G, S, H, K, N, A, Q |
| 1005 | S | R, G, K, H, N, A, Q |
| 1006 | I | R, G, H, K, N, A, Q |
| 1007 | V | R, G, H, K, N, A, Q |
| 1008 | F | R, G, H, K, N, A, Q |
| 1009 | D | R, G, N, H, K, A, Q |
| 1010 | Q | R, G, H, K, N, A |
| 1011 | K | R, G, H, N, A, Q |
| 1012 | Q | R, G, E, H, K, N, A |
| 1013 | V | R, G, H, K, N, A, Q |
| 1014 | W | R, G, H, K, N, A, Q |
| 1015 | V | R, G, H, K, N, A, Q |
| 1016 | C | R, G, N, H, K, A, Q |
| 1017 | N | R, G, H, K, A, Q |
| 1018 | A | R, G, S, H, K, N, Q |
| 1019 | D | A |
| 1020 | H | R, G, K, N, A, Q |
| 1021 | V | R, G, H, K, N, A, Q |
| 1022 | A | R, G, H, K, N, Q, S |
| 1023 | A | R, G, H, K, N, Q |
| 1024 | A | R, G, V, H, K, N, Q |
| 1025 | N | R, G, H, K, A, Q |
| 1026 | I | R, G, H, K, N, A, Q, V |
| 1027 | A | R, G, V, H, K, N, Q |
| 1028 | L | R, G, H, K, N, A, Q |
| 1029 | T | R, G, H, K, N, A, Q |
| 1030 | V | R, G, H, K, N, A, Q |
| 1031 | K | R, G, H, N, A, Q |
| 1032 | G | R, H, K, N, A, Q |
| 1033 | I | R, G, H, K, N, A, Q |
| 1034 | G | R, H, K, N, A, Q |
| 1035 | E | R, G, I, H, K, N, A, Q |
| 1036 | Q | R, G, H, K, N, A |
| 1037 | S | R, G, H, K, N, A, Q |
| 1038 | S | R, G, E, H, K, N, A, Q |
| 1039 | D | R, G, H, K, N, A, Q |
| 1040 | E | R, G, H, K, N, A, Q |
| 1041 | E | R, G, H, K, N, A, Q |
| 1042 | N | R, G, H, K, A, Q |
| 1043 | P | R, G, H, K, N, A, Q |
| 1044 | D | R, G, K, H, N, A, Q |
| 1045 | G | R, H, K, N, A, Q |
| 1046 | S | R, G, K, H, K, N, A, Q |
| 1047 | R | G, K, H, K, N, A, Q |
| 1048 | I | R, G, H, K, N, A, Q |
| 1049 | K | R, G, H, N, A, Q |
| 1050 | L | R, G, H, K, N, A, Q |
| 1051 | Q | R, G, H, K, N, A, Q |
| 1052 | L | R, G, H, K, N, A, Q |
| 1053 | T | R, G, H, K, N, A, Q |
| 1054 | S | R, G, H, K, N, A, Q |

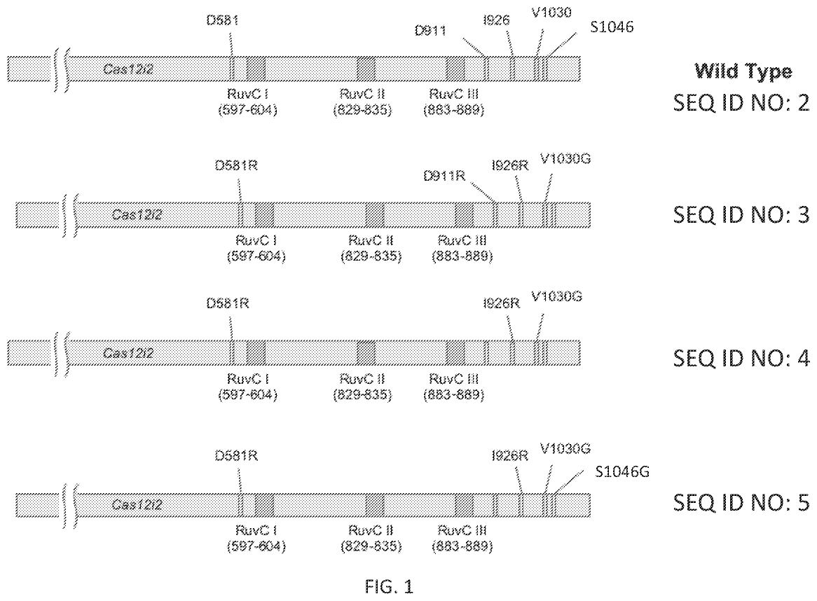

In some embodiments, the variant Cas12i2 polypeptide comprises one or more of the amino acid substitutions listed in Table 2. In some embodiments, the variant Cas12i2 polypeptide comprises a sequence set forth in any one of SEQ ID NOs: 3-5, 495, or 496, which are depicted in FIG. 1. In some embodiments, the variant Cas12i2 polypeptide comprises a sequence set forth in any one of SEQ ID NOs: 6-146.

In some embodiments, the compositions described herein comprise one or more individual (e.g., 2, 3, 4, 5, 6, 7, 8, 9, or more) variant Cas12i2 polypeptides. In such embodiments, the individual variant polypeptides may independently comprise one or more of the amino acid substitutions listed in Table 2. In some embodiments, the individual variant Cas12i2 polypeptides comprise a sequence set forth in any one of SEQ ID NOs: 3-5, 495, or 496, which are depicted in FIG. 1. In some embodiments, the individual variant Cas12i2 polypeptides comprise a sequence set forth in any one of SEQ ID NOs: 6-146.

In some embodiments, the variant Cas12i2 polypeptide comprises a mutation or set of mutations as set forth in Table 3, wherein the mutations are relative to the sequence of SEQ ID NO: 2.

TABLE 3
Cas12i2 Variant Substitutions and Sequences.

| SEQ ID NO | Mutations (relative to SEQ ID NO: 2) |
|---|---|
| 3 | D581R D911R I926R V1030G |
| 4 | D581R I926R V1030G |
| 5 | D581R I926R V1030G S1046G |
| 6 | D581R |
| 7 | L589R |
| 8 | E652R |
| 9 | T770R |
| 10 | Q820R |
| 11 | D835R |
| 12 | L836R |
| 13 | K862R |
| 14 | P868R |
| 15 | S879R |
| 16 | S883R |
| 17 | D911R |
| 18 | W912R |
| 19 | N925R |
| 20 | I926R |
| 21 | Q1010G |
| 22 | V1030G |
| 23 | D581R D911R I926R |
| 24 | L836R D911R |
| 25 | D581R L836R D911R I926R |
| 26 | D581R L836R S879R |
| 27 | D581R L836R D911R |
| 28 | D581R S879R |
| 29 | L836R D911R I926R |
| 30 | D581R L836R S879R D911R |
| 31 | L589R L836R S879R D911R |
| 32 | D581 D835G D911R I926R |
| 33 | D581R D911R I926R V1030G |
| 34 | D581R D911R I926R S1046G |
| 35 | D835G L836R D911R |
| 36 | L836R D911R V1030G |
| 37 | L836R D911R S1046G |
| 38 | D581R D835G L836R D911R I926R |
| 39 | D581R L836R D911R I926R V1030G |
| 40 | D581R L836R D911R I926R S1046G |
| 41 | D581R D835G S879R |
| 42 | L836R D911R I926R V1030G |
| 43 | D581R L836R S879R S1046G |
| 44 | D581R L836R S879R D911R S1046G |
| 45 | L836R D911R I926R S1046G |
| 46 | D581R P868R I926R V1030G |
| 47 | D581R P868R I926R V1030G S1046G |
| 48 | D581R P868R D911R I926R V1030G |
| 49 | D581R D911R I926R V1030G S1046G |
| 50 | D581R P868R D911R I926R V1030G S1046G |
| 51 | D581K D911R I926R V1030G |
| 52 | D581N D911R I926R V1030G |
| 53 | D581H D911R I926R V1030G |
| 54 | D581R D911R I926K V1030G |
| 55 | D581R D911R I926N V1030G |
| 56 | D581R D911R I926H V1030G |
| 57 | D581R D911R I926R V1030K |
| 58 | D581R D911R I926R V1030N |
| 59 | D581R D911R I926R V1030H |
| 60 | D581R G587R D911R I926R V1030G |
| 61 | D581R L623R D911R I926R V1030G |
| 62 | D581R G624R D911R I926R V1030G |
| 63 | D581R F626R D911R I926R V1030G |
| 64 | D581R G633R D911R I926R V1030G |
| 55 | D581R L654R D911R I926R V1030G |
| 66 | D581R Q674R D911R I926R V1030G |
| 67 | D581R T676R D911R I926R V1030G |
| 68 | D581R K677R D911R I926R V1030G |
| 69 | D581R N679G D911R I926R V1030G |
| 70 | D581R A689G D911R I926R V1030G |
| 71 | D581R K692G D911R I926R V1030G |
| 72 | D581R Q727G D911R I926R V1030G |
| 73 | D581R Q728G D911R I926R V1030G |
| 74 | D581R Q739G D911R I926R V1030G |
| 75 | D581R L754G D911R I926R V1030G |
| 76 | D581R T756G D911R I926R V1030G |
| 77 | D581R G762R D911R I926R V1030G |
| 78 | D581R K776R D911R I926R V1030G |
| 79 | D581R L801R D911R I926R V1030G |
| 80 | D581R R803G D911R I926R V1030G |
| 81 | D581R S817G D911R I926R V1030G |
| 82 | D581R L823R D911R I926R V1030G |
| 83 | D581R N826R D911R I926R V1030G |
| 84 | D581R G834R D911R I926R V1030G |
| 85 | D581R S837G D911R I926R V1030G |
| 86 | D581R S837R D911R I926R V1030G |
| 87 | D581R T838R D911R I926R V1030G |
| 88 | D581R T839R D911R I926R V1030G |
| 89 | D581R H870R D911R I926R V1030G |
| 90 | D581R G876R D911R I926R V1030G |
| 91 | D581R G878R D911R I926R V1030G |
| 92 | D581R Y881R D911R I926R V1030G |
| 93 | D581R D886R D911R I926R V1030G |
| 94 | D581R P887R D911R I926R V1030G |
| 95 | D581R V889R D911R I926R V1030G |
| 96 | D581R D894R D911R I926R V1030G |
| 97 | D581R K895R D911R I926R V1030G |
| 98 | D581R D911R I926R G929R V1030G |
| 99 | D581R D911R I926R E930R V1030G |
| 100 | D581R D911R I926R L948R V1030G |
| 101 | D581R D911R I926R E949R V1030G |
| 102 | D581R D911R I926R E950R V1030G |
| 103 | D581R D911R I926R P964R V1030G |
| 104 | D581R D911R I926R K979R V1030G |
| 105 | D581R D911R I926R A1018R V1030G |
| 106 | D581R D911R I926R D1019R V1030G |
| 107 | D581R D911R I926R V1021R V1030G |
| 108 | D581R D911R I926R A1022R V1030G |
| 109 | D581R D911R I926R N1025R V1030G |
| 110 | D581R D911R I926R V1030R |
| 111 | D581R D911R I926R V1030G G1032R |
| 112 | D581R D911R I926R V1030G E1035R |
| 113 | D581R D911R I926R V1030G S1037R |
| 114 | D581R G834R D911R N925R I926R E930R V1030G E1035R |
| 115 | D581R G834R D911R N925R I926R V1030G |
| 116 | D581R G834R D911R I926R E930R V1030G |
| 117 | D581R G834R D911R I926R V1030G E1035R |
| 118 | D581R D911R N925R I926R V1030G E1035R |
| 119 | D581R G834R D911R N925R I926R E930R V1030R E1035R |
| 120 | D581R G834R D911R N925R I926R V1030R |
| 121 | D581R G834R D911R I926R E930R V1030R |
| 122 | D581R G834R D911R I926R V1030R E1035R |
| 123 | D581R D911R N925R I926R V1030R E1035R |
| 124 | D581R G624R G834R D911R N925R I926R E930R V1030G E1035R |
| 125 | D581R L623R G624R L654R L801R G834R P868R G876R S883R K895R D911R |
| | N925R I926R E930R L948R K979R A1022R V1030G E1035R |
| 126 | D581R G587R L623R G624R F626R L654R Q674R K677R L801R G834R S837R |
| | T838R P868R G876R Y881R S883R D886R D894R K895R D911R N925R I926R |
| | E930R L948R E949R K979R A1022R V1030G E1035R |
| 127 | D581R G624R G834R N925R I926R E930R V1030G E1035R |
| 128 | D581R L623R G624R L654R L801R G834R P868R G876R S883R K895R N925R |
| | I926R E930R L948R K979R A1022R V1030G E1035R |
| 129 | D581R G587R L623R G624R F626R L654R Q674R K677R L801R G834R S837R |
| | T838R P868R G876R Y881R S883R D886R D894R K895R N925R I926R E930R |
| | L948R E949R K979R A1022R V1030G E1035R |
| 130 | R575H |
| 131 | Q645R |
| 132 | D581R G587R D911R V1030G S1046G |
| 133 | D581R G624R D911R V1030G S1046G |
| 134 | D581R F626R D911R V1030G S1046G |
| 135 | D581R D911R V1030G E1035R S1046G |
| 136 | D581R G587R G624R D911R V1030G S1046G |
| 137 | D581R G587R F626R D911R V1030G S1046G |
| 138 | D581R G587R D911R V1030G E1035R S1046G |
| 139 | D581R G624R F626R D911R V1030G S1046G |
| 140 | D581R G624R D911R V1030G E1035R S1046G |
| 141 | D581R F626R D911R V1030G E1035R S1046G |
| 142 | D581R G587R G624R F626R D911R V1030G S1046G |
| 143 | D581R G587R G624R D911R V1030G E1035R S1046G |
| 144 | D581R G587R F626R D911R V1030G E1035R S1046G |
| 145 | D581R G624R F626R D911R V1030G E1035R S1046G |
| 146 | D581R G587R G624R F626R D911R V1030G E1035R S1046G |
| 495 | D581R G624R F626R I926R V1030G E1035R S1046G |
| 496 | D581R G624R F626R P868T I926R V1030G E1035R S1046G |
| 497 | D581K I926R V1030G S1046G G587R F626R |
| 498 | D581K I926R V1030G S1046G G587R G624R |
| 499 | D581R I926R V1030G S1046G G587R G624R F626R |
| 500 | D581R I926R V1030G S1046G G587R G624R F626R V967K |
| 501 | D581R I926R V1030G S1046G L855Q |
| 502 | D581R I926R V1030G S1046G H870S |
| 503 | D581R I926R V1030G S1046G A867Q |
| 504 | D581R I926R V1030G S1046G V859T |
| 505 | D581R I926R V1030G S1046G P868T |
| 506 | D581R I926R V1030G S1046G R857S |
| 507 | D581R I926R V1030G S1046G N871P |
| 508 | D581R I926R V1030G S1046G R850L |
| 509 | D581R I926R V1030G S1046G Q602L |
| 510 | D581R I926R V1030G S1046G M869L |
| 511 | D581R I926R V1030G S1046G L880W |
| 512 | D581R I926R V1030G S1046G M869W |

In some embodiments, the variant Cas12i2 polypeptide is a polypeptide shown in Table 3. The substitutions in Table 3 are relative to the sequence of SEQ ID NO: 2. In some embodiments, a variant Cas12i2 polypeptide comprises one or more of the amino acid substitutions listed in Table 3.

In some embodiments, the variant Cas12i2 polypeptide of the present invention comprises a polypeptide sequence having 50%, 60%, 65%, 70%, 75%, 80%, 85%, 90%, 91%, 92%, 93%, 94%, 95%, 96%, 97%, 98%, 99%, or 100% identity to any one of SEQ ID NOs: 3-146 and 495-512. In some embodiments, the variant Cas12i2 polypeptide of the present invention comprises a polypeptide sequence having greater than 50%, 60%, 65%, 70%, 75%, 80%, 85%, 90%, 91%, 92%, 93%, 94%, 95%, 96%, 97%, 98%, 99%, or 100% identity to any one of SEQ ID NOs: 3-146 and 495-512.

In some embodiments, one or more amino acids between amino acids 597 to 607 of SEQ ID NO: 2 is altered or mutated. In some embodiments, one or more amino acids between amino acids 597 to 604 of SEQ ID NO: 2 is altered or mutated. In some embodiments, one or more amino acids between amino acids 830 to 833 of SEQ ID NO: 2 is altered or mutated. In some embodiments, one or more amino acids between amino acids 829 to 835 of SEQ ID NO: 2 is altered or mutated. In some embodiments, one or more amino acids between amino acids 882 to 888 of SEQ ID NO: 2 is altered or mutated. In some embodiments, one or more amino acids between amino acids 883 to 889 of SEQ ID NO: 2 is altered or mutated. See Table 2.

In some embodiments, one or more amino acids between amino acids 600 to 607 of SEQ ID NO: 2 is altered or mutated. In some embodiments, one or more amino acids between amino acids 833 to 871 of SEQ ID NO: 2 is altered or mutated. In some embodiments, one or more amino acids between amino acids 877 to 885 of SEQ ID NO: 2 is altered or mutated. See Table 2.

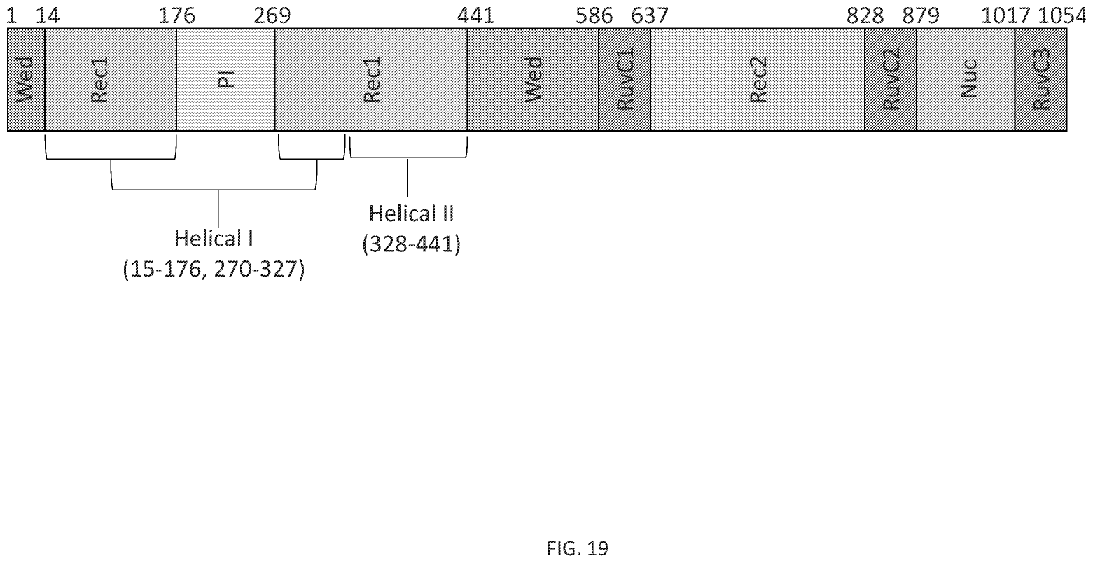

In some embodiments, the variant Cas12i2 polypeptide comprises at least one RuvC domain. In some embodiments, the variant Cas12i2 polypeptide comprises at least one RuvC motif (e.g., one, two or three RuvC motifs). The domains of Cas12i2 polypeptides disclosed herein are depicted in FIG. 19. The Wedge domain comprises residues 1-14 and 442-586 of the Cas12i2 polypeptide. The Rec1 domain comprises residues 15-176 and 270-441 of the Cas12i2 polypeptide. Within the Rec1 domain, the Helical I domain comprises residues 15-176 and 270-327, and the Helical II domain comprises residues 328-441. The PI domain comprises residues 177-269 of the Cas12i2 polypeptide. The Rec2 domain comprises residues 638-828 of the Cas12i2 polypeptide. The Nuc domain comprises residues 880-1017 of the Cas12i2 polypeptide. The RuvC motif comprises residues 587-637 (RuvC1), residues 829-879 (RuvC2), and residues 1018-1054 (RuvC3) of the Cas12i2 polypeptide.

In some embodiments, the variant Cas12i2 polypeptide having a feature as described herein comprises an amino acid sequence having at least about 60%, 65%, 70%, 75%, 80%, 85%, 90%, 91%, 92%, 93%, 94%, 95%, 96%, 97%, 98%, 99%, or 100% identity to any one of SEQ ID NOs: 3-146 and 495-512.

Although the changes described herein may be one or more amino acid changes, changes to the variant Cas12i2 polypeptide may also be of a structural or substantive nature, such as fusion of polypeptides as amino- and/or carboxyl-terminal extensions. For example, variant Cas12i2 polypeptide may contain additional peptides, e.g., one or more peptides. Examples of additional peptides may include epitope peptides for labelling, such as a polyhistidine tag (His-tag), Myc, and FLAG. In some embodiments, the variant Cas12i2 polypeptide described herein can be fused to a detectable moiety such as a fluorescent protein (e.g., green fluorescent protein (GFP) or yellow fluorescent protein (YFP)).

In some embodiments, the variant Cas12i2 polypeptide comprises at least one (e.g., two, three, four, five, six, or more) nuclear localization signal (NLS). In some embodiments, the variant Cas12i2 polypeptide comprises at least one (e.g., two, three, four, five, six, or more) nuclear export signal (NES). In some embodiments, the variant Cas12i2 polypeptide comprises at least one (e.g., two, three, four, five, six, or more) NLS and at least one (e.g., two, three, four, five, six, or more) NES.

In some embodiments, the variant Cas12i2 polypeptide described herein can be self-inactivating. See, Epstein et al., “Engineering a Self-Inactivating CRISPR System for AAV Vectors,” Mol. Ther., 24 (2016): S50, which is incorporated by reference in its entirety.

In some embodiments, the nucleotide sequence encoding the variant Cas12i2 polypeptide described herein can be codon-optimized for use in a particular host cell or organism. For example, the nucleic acid can be codon-optimized for any non-human eukaryote including mice, rats, rabbits, dogs, livestock, or non-human primates. Codon usage tables are readily available, for example, at the “Codon Usage Database” available at www.kazusa.orjp/codon/and these tables can be adapted in a number of ways. See Nakamura et al. Nucl. Acids Res. 28:292 (2000), which is incorporated herein by reference in its entirety. Computer algorithms for codon optimizing a particular sequence for expression in a particular host cell are also available, such as Gene Forge (Aptagen; Jacobus, PA).

### Functionality of Variant Polypeptides

As used herein, a “biologically active portion” is a portion that retains at least one function (e.g. completely, partially, minimally) of the parent polypeptide (e.g., a “minimal” or “core” domain). In some embodiments, the variant Cas12i2 polypeptide retains enzymatic activity at least as active as the parent polypeptide. Accordingly, in some embodiments, a variant Cas12i2 polypeptide has enzymatic activity greater than the parent polypeptide.

Also provided is a variant Cas12i2 polypeptide of the present invention having enzymatic activity, e.g., nuclease or endonuclease activity, and comprising an amino acid sequence which differs from the amino acid sequences of any one of a parent polypeptide and SEQ ID NO: 2 by 50, 40, 35, 30, 25, 20, 19, 18, 17, 16, 15, 14, 13, 12, 11, 10, 9, 8, 7, 6, 5, 4, 3, 2, or 1 amino acid residue(s), when aligned using any of the previously described alignment methods. In some embodiments, the variant Cas12i2 polypeptide having enzymatic activity comprises an amino acid sequence having at least about 60%, 65%, 70%, 75%, 80%, 85%, 90%, 91%, 92%, 93%, 94%, 95%, 96%, 97%, 98%, 99%, or 100% identity to the amino acid sequences of any one of a parent polypeptide and SEQ ID NO: 2, when aligned using any of the previously described alignment methods.

In some aspects, the variant Cas12i2 polypeptide comprises at least one alteration or mutation that enhances enzymatic activity, RNA guide complex (binary complex) formation, RNA guide binding activity, RNA guide affinity, RNA guide binding specificity, protein-RNA interactions, protein-DNA interactions, protein stability, ternary complex formation, on-target binding activity, on-target binding specificity, and/or stability in a ternary complex of the variant Cas12i2 polypeptide. In some embodiments, the variant Cas12i2 polypeptide of the present invention has at least one of enzymatic activity, RNA guide complex (binary complex) formation, RNA guide binding activity, RNA guide affinity, RNA guide binding specificity, protein-RNA interactions, protein-DNA interactions, protein stability, ternary complex formation, on-target binding activity, on-target binding specificity, and/or stability in a ternary complex equivalent to or greater than the parent polypeptide. In some embodiments, the variant Cas12i2 polypeptide comprises at least one alteration or mutation that enhances enzymatic activity, RNA guide complex (binary complex) formation, RNA guide binding activity, RNA guide affinity, RNA guide binding specificity, protein-RNA interactions, protein-DNA interactions, protein stability, ternary complex formation, on-target binding activity, on-target binding specificity, and/or stability in a ternary complex and the variant Cas12i2 polypeptide comprises an amino acid sequence having at least about 60%, 65%, 70%, 75%, 80%, 85%, 90%, 91%, 92%, 93%, 94%, 95%, 96%, 97%, 98%, 99%, or 100% identity to SEQ ID NO: 4.

In some embodiments, the variant Cas12i2 polypeptide comprises enhanced enzymatic activity, RNA guide complex (binary complex) formation, RNA guide binding activity, RNA guide affinity, RNA guide binding specificity, protein-RNA interactions, protein-DNA interactions, protein stability, ternary complex formation, on-target binding activity, on-target binding specificity, and/or stability in a ternary complex and the variant Cas12i2 polypeptide comprises an amino acid sequence of any one of SEQ ID NOs: 3-5, 495, or 496.

In some embodiments, the variant Cas12i2 polypeptide comprises enhanced enzymatic activity, RNA guide complex (binary complex) formation, RNA guide binding activity, RNA guide affinity, RNA guide binding specificity, protein-RNA interactions, protein-DNA interactions, protein stability, ternary complex formation, on-target binding activity, on-target binding specificity, and/or stability in a ternary complex and the variant Cas12i2 polypeptide comprises an amino acid sequence of any one of SEQ ID NOs: 3-146 and 495-512.

In some aspects of the invention, the variant Cas12i2 polypeptide comprises a substitution that alters the ability of the polypeptide to interact (e.g., bind or form a complex) with a nucleic acid (e.g., an RNA guide or DNA). In some aspects of the invention, the variant Cas12i2 polypeptide comprises a substitution that alters the affinity of the polypeptide to a nucleic acid (e.g., an RNA guide or DNA).

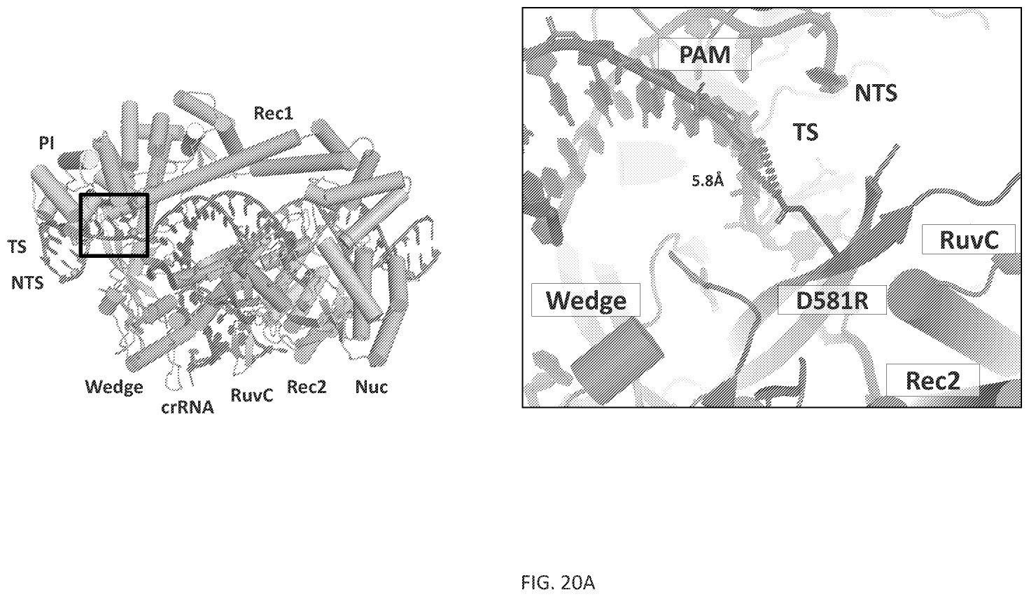

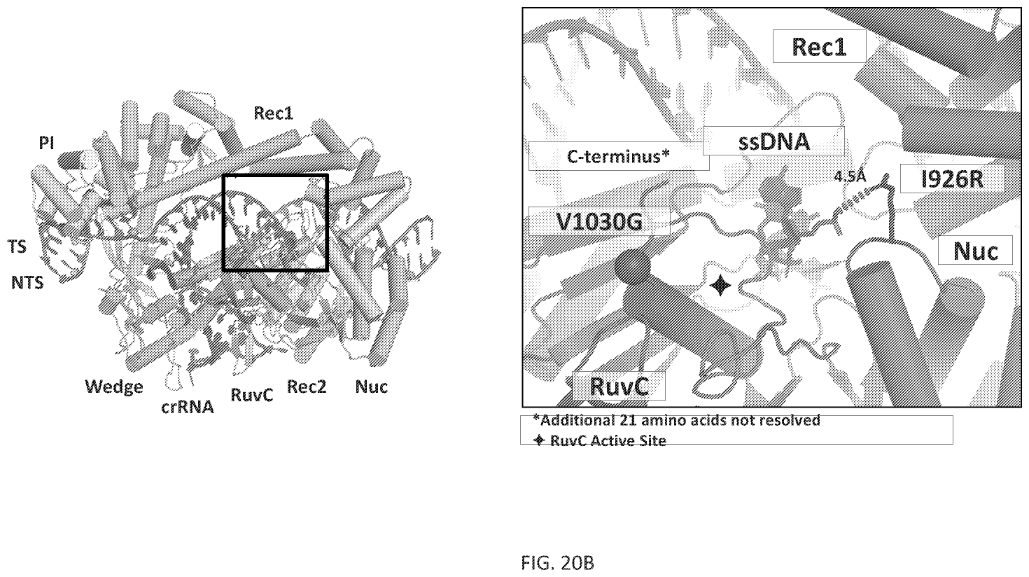

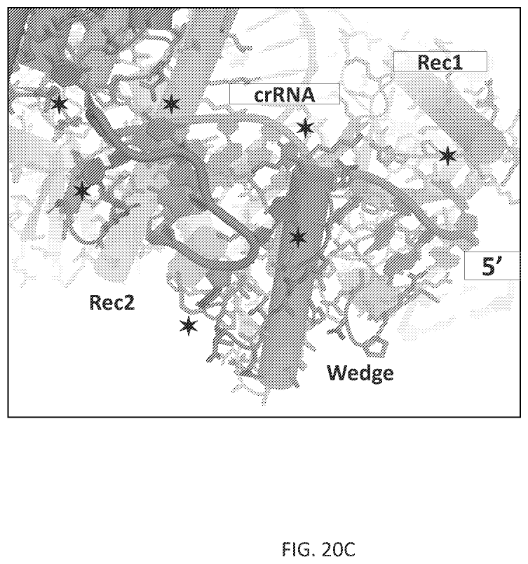

In some embodiments, a variant Cas12i2 polypeptide comprising a D581R substitution exhibits enhanced enzymatic activity. In some embodiments, a variant Cas12i2 polypeptide comprising a D581R substitution interacts with the NTS. In some embodiments, a variant Cas12i2 polypeptide comprising a D581R substitution interacts with the PAM sequence backbone. In some embodiments, a variant Cas12i2 polypeptide comprising a D581R substitution exhibits enhanced electrostatic interactions with DNA at the PAM sequence. In some embodiments, a variant Cas12i2 polypeptide comprising a D581R substitution decreases repulsive interactions with nucleic acids. In some embodiments, a variant Cas12i2 polypeptide comprising a D581R substitution enhances R-loop stability. See FIG. 20A.

In some embodiments, a variant Cas12i2 polypeptide comprising a V1030G substitution exhibits enhanced enzymatic activity. In some embodiments, a variant Cas12i2 polypeptide comprising a V1030G substitution interacts with the NTS. In some embodiments, a variant Cas12i2 polypeptide comprising a V1030G substitution is near the Cas12i2 active site. See FIG. 20B.

In some embodiments, a variant Cas12i2 polypeptide comprising an I926R substitution exhibits enhanced enzymatic activity. In some embodiments, a variant Cas12i2 polypeptide comprising a I926R substitution interacts with the single-stranded DNA near the Cas12i2 active site. In some embodiments, a variant Cas12i2 polypeptide comprising a I926R substitution stabilizes single-stranded DNA. See FIG. 20B.

In some embodiments, a variant Cas12i2 polypeptide comprising a G624R substitution exhibits enhanced enzymatic activity. In some embodiments, a variant Cas12i2 polypeptide comprising a G624R substitution interacts with the NTS. In some embodiments, a variant Cas12i2 polypeptide comprising a G624R substitution enhances R-loop stability.

In some embodiments, a variant Cas12i2 polypeptide comprising a F626R substitution exhibits enhanced enzymatic activity. In some embodiments, a variant Cas12i2 polypeptide comprising a F626R substitution interacts with the NTS. In some embodiments, a variant Cas12i2 polypeptide comprising a F626R substitution enhances R-loop stability.

In some embodiments, the variant Cas12i2 polypeptide of the present invention has at least one of enzymatic activity, RNA guide complex (binary complex) formation, RNA guide binding activity, RNA guide affinity, RNA guide binding specificity, protein-RNA interactions, protein-DNA interactions, protein stability, ternary complex formation, on-target binding activity, on-target binding specificity, and/or stability in a ternary complex at a temperature range from about 20° C. to about 90° C., e.g., about any one of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 50° C., 51° C., 52° C., 53° C., 54° C., 55° C., 56° C., 57° C., 58° C., 59° C., 60° C., 65° C. or greater. In some embodiments, the variant Cas12i2 polypeptide of the present invention has at least one of enzymatic activity, RNA guide complex (binary complex) formation, RNA guide binding activity, RNA guide affinity, RNA guide binding specificity, protein-RNA interactions, protein-DNA interactions, protein stability, ternary complex formation, on-target binding activity, on-target binding specificity, and/or stability in a ternary complex at a temperature of about 20° C. to about 25° C. or at a temperature of about 37° C.

In some embodiments, the variant Cas12i2 polypeptide exhibits enhanced enzymatic activity, RNA guide complex (binary complex) formation, RNA guide binding activity, RNA guide affinity, RNA guide binding specificity, protein-RNA interactions, protein-DNA interactions, protein stability, ternary complex formation, on-target binding activity, on-target binding specificity, and/or stability in a ternary complex, as compared to a parent polypeptide, in a buffer having a pH in a range of about 7.3 to about 8.6 (e.g., 7.3, 7.4, 7.5, 7.6, 7.7, 7.8, 7.9, 8.0, 8.1, 8.2, 8.3, 8.4, 8.5, 8.6, or any value within a range between any combination of these values).

In some embodiments, the variant Cas12i2 polypeptide exhibits at least one of enzymatic activity, RNA guide complex (binary complex) formation, RNA guide binding activity, RNA guide affinity, RNA guide binding specificity, protein-RNA interactions, protein-DNA interactions, protein stability, ternary complex formation, on-target binding activity, on-target binding specificity, and/or stability in a ternary complex, as compared to a parent polypeptide, when the Tₘ value of the variant Cas12i2 polypeptide is at least 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., or 20° C. greater than the Tₘ value of a parent polypeptide. In one embodiment, the variant Cas12i2 polypeptide exhibits enhanced enzymatic activity, RNA guide complex (binary complex) formation, RNA guide binding activity, RNA guide affinity, RNA guide binding specificity, protein-RNA interactions, protein-DNA interactions, protein stability, ternary complex formation, on-target binding activity, on-target binding specificity, and/or stability in a ternary complex when the Tₘ value of the variant Cas12i2 polypeptide is at least 8° C. greater than the Tₘ value of the parent polypeptide.

In some embodiments, the variant Cas12i2 polypeptide of the present invention exhibits increased at least one of enzymatic activity, RNA guide complex (binary complex) formation, RNA guide binding activity, RNA guide affinity, RNA guide binding specificity, protein-RNA interactions, protein-DNA interactions, protein stability, ternary complex formation, on-target binding activity, on-target binding specificity, and/or stability in a ternary complex at about 37° C. over an incubation period of at least about any one of 10 mins, 15 mins, 20 mins, 25 mins, 30 mins, 35 mins, 40 mins, 45 mins, 50 mins, 55 mins, 1 hr, 2 hr, 3 hr, 4 hr, or more hours as compared to a parent polypeptide. In some embodiments, a variant Cas12i2 polypeptide of the present invention exhibits increased at least one of enzymatic activity, RNA guide complex (binary complex) formation, RNA guide binding activity, RNA guide affinity, RNA guide binding specificity, protein-RNA interactions, protein-DNA interactions, protein stability, ternary complex formation, on-target binding activity, on-target binding specificity, and/or stability in a ternary complex over a range of incubation times as compared to a parent polypeptide.

Also provided is a variant Cas12i2 polypeptide of the present invention having decreased dissociation from the RNA guide (dissociation of a binary complex), dissociation from a target nucleic acid (dissociation of a ternary complex), off-target binding to a non-target nucleic acid, and/or activity at a non-target locus of a target nucleic acid and comprising an amino acid sequence which differs from the amino acid sequences of any one of a parent polypeptide and SEQ ID NO: 2 by 50, 40, 35, 30, 25, 20, 19, 18, 17, 16, 15, 14, 13, 12, 11, 10, 9, 8, 7, 6, 5, 4, 3, 2, or 1 amino acid residue(s), when aligned using any of the previously described alignment methods. In some embodiments, the variant Cas12i2 polypeptide having decreased dissociation from the RNA guide (dissociation of a binary complex), dissociation from a target nucleic acid (dissociation of a ternary complex), off-target binding to a non-target nucleic acid, and/or activity at a non-target locus of a target nucleic acid comprises an amino acid sequence having at least about 60%, 65%, 70%, 75%, 80%, 85%, 90%, 91%, 92%, 93%, 94%, 95%, 96%, 97%, 98%, 99%, or 100% identity to the amino acid sequences of any one of a parent polypeptide and SEQ ID NO: 2, when aligned using any of the previously described alignment methods.

In some aspects, the variant Cas12i2 polypeptide comprises at least one alteration or mutation that decreased dissociation from the RNA guide (dissociation of a binary complex), dissociation from a target nucleic acid (dissociation of a ternary complex), off-target binding to a non-target nucleic acid, and/or activity at a non-target locus of a target nucleic acid of the variant Cas12i2 polypeptide. In some embodiments, the variant Cas12i2 polypeptide comprises at least one alteration or mutation that decreased dissociation from the RNA guide (dissociation of a binary complex), dissociation from a target nucleic acid (dissociation of a ternary complex), off-target binding to a non-target nucleic acid, and/or activity at a non-target locus of a target nucleic acid and the variant Cas12i2 polypeptide comprises an amino acid sequence having at least about 60%, 65%, 70%, 75%, 80%, 85%, 90%, 91%, 92%, 93%, 94%, 95%, 96%, 97%, 98%, 99%, or 100% identity to SEQ ID NO: 4.

In some aspects, the variant Cas12i2 polypeptide comprises at least one alteration or mutation that decreases dissociation from the RNA guide (dissociation of a binary complex), dissociation from a target nucleic acid (dissociation of a ternary complex), off-target binding to a non-target nucleic acid, and/or activity at a non-target locus of a target nucleic acid as compared to a parent polypeptide. In some aspects, the variant Cas12i2 polypeptide comprises decreased dissociation from the RNA guide (dissociation of a binary complex), dissociation from a target nucleic acid (dissociation of a ternary complex), off-target binding to a non-target nucleic acid, and/or activity at a non-target locus of a target nucleic acid as compared to a parent polypeptide and the variant Cas12i2 polypeptide comprises an amino acid sequence of any one of SEQ ID NOs: 3-5, 495, or 496.

In some aspects, the variant Cas12i2 polypeptide comprises decreased dissociation from the RNA guide (dissociation of a binary complex), dissociation from a target nucleic acid (dissociation of a ternary complex), off-target binding to a non-target nucleic acid, and/or activity at a non-target locus of a target nucleic acid as compared to a parent polypeptide and the variant Cas12i2 polypeptide comprises an amino acid sequence of any one of SEQ ID NOs: 3-146 and 495-512.

In some embodiments, the variant Cas12i2 polypeptide of the present invention exhibits at least one of dissociation from the RNA guide (dissociation of a binary complex), dissociation from a target nucleic acid (dissociation of a ternary complex), off-target binding to a non-target nucleic acid, and/or activity at a non-target locus of a target nucleic acid at equivalent to or less levels than the parent polypeptide. In some embodiments, the variant Cas12i2 polypeptide of the present invention has equivalent to or decreased dissociation from the RNA guide (dissociation of a binary complex), dissociation from a target nucleic acid (dissociation of a ternary complex), off-target binding to a non-target nucleic acid, and/or activity at a non-target locus of a target nucleic acid at a temperature range from about 20° C. to about 90° C., e.g., about any one of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 50° C., 51° C., 52° C., 53° C., 54° C., 55° C., 56° C., 57° C., 58° C., 59° C., 60° C., 65° C. or greater. In some embodiments, the variant Cas12i2 polypeptide of the present invention performs at least one of dissociation from the RNA guide (dissociation of a binary complex), dissociation from a target nucleic acid (dissociation of a ternary complex), off-target binding to a non-target nucleic acid, and/or activity at a non-target locus of a target nucleic acid at a temperature of about 20° C. to about 25° C. or at a temperature of about 37° C.

In some embodiments, the variant Cas12i2 polypeptide exhibits decreased dissociation from the RNA guide (dissociation of a binary complex), dissociation from a target nucleic acid (dissociation of a ternary complex), off-target binding to a non-target nucleic acid, and/or activity at a non-target locus of a target nucleic acid, as compared to a parent polypeptide, in a buffer having a pH in a range of about 7.3 to about 8.6 (e.g., 7.3, 7.4, 7.5, 7.6, 7.7, 7.8, 7.9, 8.0, 8.1, 8.2, 8.3, 8.4, 8.5, 8.6, or any value within a range between any combination of these values).

In some embodiments, the variant Cas12i2 polypeptide exhibits at least one of dissociation from the RNA guide (dissociation of a binary complex), dissociation from a target nucleic acid (dissociation of a ternary complex), off-target binding to a non-target nucleic acid, and/or activity at a non-target locus of a target nucleic acid, as compared to a parent polypeptide, when the Tₘ value of the variant Cas12i2 polypeptide is at least 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., or 20° C. greater than the Tₘ value of a parent polypeptide. In one embodiment, the variant Cas12i2 polypeptide exhibits decreased dissociation from the RNA guide (dissociation of a binary complex), dissociation from a target nucleic acid (dissociation of a ternary complex), off-target binding to a non-target nucleic acid, and/or activity at a non-target locus of a target nucleic acid when the Tₘ value of the variant Cas12i2 polypeptide is at least 8° C. greater than the Tₘ value of the parent polypeptide.

### Increased RNA Guide Interactions

In some embodiments, the variant Cas12i2 polypeptide comprises an alteration that increases interactions and/or affinity between the variant Cas12i2 polypeptide and the RNA guide, as compared to a parent polypeptide. See FIG. 20C. In some embodiments, the alteration that increases interactions and/or affinity between the variant Cas12i2 polypeptide and the RNA guide is substituting one or more amino acids to an arginine, lysine, glutamine, asparagine, histidine, serine, or tyrosine residue. In some embodiments, the variant Cas12i2 polypeptide comprises a substitution of one or more amino acids in the RNA binding interface to an arginine, lysine, glutamine, asparagine, histidine, serine, or tyrosine residue. In some embodiments, the variant Cas12i2 polypeptide comprises a substitution of one or more amino acids 441-586 or 637-828 to any one of an arginine, lysine, glutamine, asparagine, histidine, serine, or tyrosine residue. In some embodiments, the variant Cas12i2 polypeptide comprises an alteration of one or more amino acids in at least one domain (e.g., the Wedge domain or Rec2 domain) to an arginine, lysine, glutamine, asparagine, histidine, serine, or tyrosine residue. In some embodiments, the RNA binding interface substitution(s) increases RNA guide binding or RNA guide binding affinity by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more or any percentage therebetween, as compared to a parent polypeptide.

In some embodiments, the substitution increases RNA guide complex (binary complex) formation relative to a parent polypeptide. Non-limiting examples of substitutions that can alter the ability of a variant Cas12i2 polypeptide to interact with the direct repeat sequence of an RNA guide are shown in Table 4. In some embodiments, a variant Cas12i2 polypeptide comprising one or more substitutions listed in Table 4 exhibits enhanced RNA guide complex (binary complex) formation relative to a parent polypeptide. In some embodiments, a variant Cas12i2 polypeptide comprising one or more substitutions listed in Table 4 forms a more stable binary complex with an RNA guide, as compared to a binary complex comprising a parent polypeptide.

TABLE 4
Substitutions increasing direct repeat sequence contact.

| Residue | Substitution(s) |
|---|---|
| N459 | R, K, H |
| T513 | N |
| P514 | Q |
| H519 | R, K |
| V536 | N |
| T541 | R, Y |
| E542 | Q, K |
| F646 | Y, R, H, Q |
| Y651 | H, R, K, Q |
| L654 | N, Q |
| L703 | N |
| C741 | N, S |
| L749 | N |
| L801 | R, K |
| T804 | K, R, Q |
| E812 | Q |
| A815 | R |
| N816 | H |
| Q820 | K, R |

In some embodiments, a variant Cas12i2 polypeptide of any one of SEQ ID NOs: 3-146 and 495-512 further comprises one or more substitutions listed in Table 4. In some embodiments, a variant Cas12i2 polypeptide comprises one or more substitutions listed in Table 2 and Table 4.

In some embodiments, a variant Cas12i2 polypeptide exhibiting enhanced RNA guide complex (binary complex) formation comprises two or more substitutions, e.g., L654K Q658A, L520K Q496N, or L520K Q496S. In some embodiments, a variant Cas12i2 polypeptide of any one of SEQ ID NOs: 3-146 and 495-512 further comprises L654K Q658A, L520K Q496N, or L520K Q496S substitutions. In some embodiments, a variant Cas12i2 polypeptide of any one of SEQ ID NOs: 3-146 and 495-512 further comprises L654K Q658A, L520K Q496N, or L520K Q496S substitutions and one or more substitutions listed in Table 4.

In some embodiments, the variant Cas12i2 polypeptide of any one of SEQ ID NOs: 3-146 and 495-512 comprising one or more substitutions listed in Table 4 exhibits increased enzymatic activity. In some embodiments, the variant Cas12i2 polypeptide comprising one or more substitutions listed in Table 4 exhibits increased enzymatic activity. In some embodiments, the variant Cas12i2 polypeptide of any one of SEQ ID NOs: 3-146 and 495-512 that further comprises L654K Q658A, L520K Q496N, or L520K Q496S substitutions and one or more substitutions listed in Table 4 exhibits increased enzymatic activity. In some embodiments, the variant Cas12i2 polypeptide exhibits increased enzymatic activity (e.g., by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more or any percentage therebetween) as compared to a parent polypeptide.

### Increased Double-Stranded DNA Interactions

In some aspects, a variant Cas12i2 polypeptide comprises an alteration that increases interactions with double-stranded DNA relative to a parent polypeptide. In some embodiments, increased interactions with double-stranded DNA are increased electrostatic interactions. In some embodiments, the variant Cas12i2 polypeptide comprises an alteration that increases affinity between the variant Cas12i2 polypeptide and double-stranded DNA relative to a parent polypeptide. In some embodiments, the alteration that increases interactions and/or affinity between the variant Cas12i2 polypeptide and double-stranded DNA increases binding of the variant Cas12i2 polypeptide to a PAM sequence.

In some embodiments, the alteration that increases interactions and/or affinity between the variant Cas12i2 polypeptide and the double-stranded DNA is substituting one or more amino acids to an arginine, lysine, glutamine, asparagine, histidine, or serine residue. In some embodiments, the variant Cas12i2 polypeptide comprises a substitution of one or more amino acids in the double-stranded DNA binding interface to an arginine, lysine, glutamine, asparagine, histidine, or serine residue. In some embodiments, the variant Cas12i2 polypeptide comprises a substitution of one or more amino acids 140-190, 220-300, 440-480, or 560-570 to any one of an arginine, lysine, glutamine, asparagine, histidine, or serine residue. In some embodiments, the variant Cas12i2 polypeptide comprises an alteration of one or more amino acids in at least one domain (e.g., the Rec1 domain, PI domain, or Wedge domain) to an arginine, lysine, glutamine, asparagine, histidine, or serine residue. In some embodiments, the double-stranded DNA binding interface substitution(s) increase double-stranded DNA interactions and/or affinity by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more or any percentage therebetween, as compared to a parent polypeptide. In some embodiments, the double-stranded DNA binding interface substitution(s) increase binding of the variant Cas12i2 polypeptide to a PAM sequence by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more or any percentage therebetween, as compared to a parent polypeptide.

In some embodiments, the substitution that increases double-stranded DNA interactions increases ternary complex formation relative to a parent polypeptide. Non-limiting examples of substitutions that can alter the ability of a variant Cas12i2 polypeptide to interact with double-stranded DNA are shown in Table 5. In some embodiments, a variant Cas12i2 polypeptide comprising one or more substitutions listed in Table 5 exhibits increased double-stranded DNA interactions (ternary complex formation) relative to a parent polypeptide. In some embodiments, a variant Cas12i2 polypeptide comprising one or more substitutions listed in Table 5 forms a more stable ternary complex, as compared to a parent polypeptide.

TABLE 5
Substitutions altering double-stranded interactions.

| Residue | Substitution(s) |
|---|---|
| T147 | R, K |
| T174 | R, K, N, Q |
| E178 | Q, N |
| N229 | R, K |
| E237 | Q, N, S |
| Y287 | K, H |
| D288 | N, S |
| L289 | K, H, N, R |
| T447 | R, K |
| N448 | R, K, H |
| I451 | R |
| L452 | K, H |
| Y472 | R, K |
| T562 | R, K |
| E563 | R, K |
| T567 | N |

In some embodiments, a variant Cas12i2 polypeptide of any one of SEQ ID NOs: 3-146 and 495-512 further comprises one or more substitutions listed in Table 5. In some embodiments, a variant Cas12i2 polypeptide comprises one or more substitutions listed in Table 2 and Table 5.

In some embodiments, a variant Cas12i2 polypeptide exhibiting increased double-stranded DNA interactions comprises two or more substitutions, e.g., T562R E563K, T562R E563K N448K, 1451R L452K, 1451R L452K T562R E563R, 1451R L452K Y472R, N229R Q224N, or N229K Q224N. In some embodiments, a variant Cas12i2 polypeptide exhibiting increased ternary complex formation/stability comprises two or more substitutions, e.g., T562R E563K, T562R E563K N448K, 1451R L452K, 1451R L452K T562R E563R, 1451R L452K Y472R, N229R Q224N, or N229K Q224N. In some embodiments, a variant Cas12i2 polypeptide of any one of SEQ ID NOs: 3-146 and 495-512 further comprises T562R E563K, T562R E563K N448K, 1451R L452K, 1451R L452K T562R E563R, 1451R L452K Y472R, N229R Q224N, or N229K Q224N substitutions. In some embodiments, a variant Cas12i2 polypeptide of any one of SEQ ID NOs: 3-146 and 495-512 further comprises T562R E563K, T562R E563K N448K, 1451R L452K, 1451R L452K T562R E563R, 1451R L452K Y472R, N229R Q224N, or N229K Q224N substitutions and one or more substitutions listed in Table 4 and/or Table 5. In some embodiments, the variant Cas12i2 polypeptide comprises any one or more substitutions in Table 4 and/or Table 5. In some embodiments, the variant Cas12i2 polypeptide with one or more of the substitutions in Table 4 and/or Table 5 exhibits increased double-stranded DNA interactions and/or affinity (e.g., by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more or any percentage therebetween) as compared to a parent polypeptide. In some embodiments, the variant Cas12i2 polypeptide with one or more of the substitutions in Table 4 and/or Table 5 exhibits increased ternary complex formation and/or ternary complex stability (e.g., by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more or any percentage therebetween) as compared to a parent polypeptide.

In some embodiments, the variant Cas12i2 polypeptide of any one of SEQ ID NOs: 3-146 and 495-512 comprising one or more substitutions listed in Table 4 and/or Table 5 exhibits increased enzymatic activity. In some embodiments, the variant Cas12i2 polypeptide comprising one or more substitutions listed in Table 4 and/or Table 5 exhibits increased enzymatic activity. In some embodiments, the variant Cas12i2 polypeptide of any one of SEQ ID NOs: 3-146 and 495-512 that further comprises T562R E563K, T562R E563K N448K, 1451R L452K, 1451R L452K T562R E563R, 1451R L452K Y472R, N229R Q224N, or N229K Q224N substitutions and one or more substitutions listed in Table 4 and/or Table 5 exhibits increased enzymatic activity. In some embodiments, the variant Cas12i2 polypeptide exhibits increased enzymatic activity (e.g., by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more or any percentage therebetween) as compared to a parent polypeptide.

### Increased Single-Stranded DNA Interactions

In some embodiments, a variant Cas12i2 polypeptide comprises an alteration that increases interactions with single-stranded DNA relative to a parent polypeptide. In some embodiments, the variant Cas12i2 polypeptide comprises an alteration that increases affinity between the variant Cas12i2 polypeptide and double-stranded DNA relative to a parent polypeptide. In some embodiments, the single-stranded DNA comprises the non-target strand (NTS). In some embodiments, increased interactions with the single-stranded DNA (e.g., the NTS) are interactions between the PAM sequence and the active site of the variant Cas12i2 polypeptide. In some embodiments, the single-stranded DNA comprises single-stranded DNA that interacts with the variant Cas12i2 polypeptide at or near the active site of the variant Cas12i2 polypeptide. In some embodiments, an alteration that increases interactions and/or affinity between the variant Cas12i2 polypeptide and the single-stranded DNA stabilizes the R-loop. As used herein, the “R-loop” refers to a nucleic acid comprising an RNA guide paired with the target strand (TS) and the single-stranded non-target strand (NTS).

In some embodiments, the alteration that increases interactions and/or affinity between the variant Cas12i2 polypeptide and the single-stranded DNA is substituting one or more amino acids to an arginine, lysine, glutamine, asparagine, histidine, or alanine residue. In some embodiments, the variant Cas12i2 polypeptide comprises a substitution of one or more amino acids in the single-stranded DNA binding interface to an arginine, lysine, glutamine, asparagine, histidine, or alanine residue. In some embodiments, the variant Cas12i2 polypeptide comprises a substitution of one or more amino acids 230-260, 350-400, 580-630, 700-760, 830-900, or 920-1035 to any one of an arginine, lysine, glutamine, asparagine, histidine, or alanine residue. In some embodiments, the variant Cas12i2 polypeptide comprises an alteration of one or more amino acids in at least one domain/motif (e.g., the PI domain, Rec1 domain, Wedge domain, RuvC1 motif, Rec2 domain, RuvC2 motif, Nuc domain, or RuvC3 motif) to an arginine, lysine, glutamine, asparagine, histidine, or alanine residue. In some embodiments, the single-stranded DNA binding interface substitution(s) increase single-stranded DNA interactions and/or affinity by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more or any percentage therebetween, as compared to a parent polypeptide.

In some embodiments, the substitution that increases single-stranded DNA interactions increases ternary complex formation relative to a parent polypeptide. Non-limiting examples of substitutions that can alter the ability of a variant Cas12i2 polypeptide to interact with single-stranded DNA are shown in Table 6. In some embodiments, a variant Cas12i2 polypeptide comprising one or more substitutions listed in Table 6 exhibits increased single-stranded DNA interactions (ternary complex formation) relative to a parent polypeptide. In some embodiments, a variant Cas12i2 polypeptide comprising one or more substitutions listed in Table 6 forms a more stable ternary complex, as compared to a parent polypeptide.

TABLE 6
Substitutions altering single-stranded interactions.

| Residue | Substitution(s) | Contact |
|---|---|---|
| T235 | K | NTS ssDNA |
| D256 | K, N, R | NTS ssDNA |
| G360 | R, K | ssDNA (near active site) |
| D362 | K, R | ssDNA (near active site) |
| V383 | R, K | ssDNA (near active site) |
| L384 | R, K | ssDNA (near active site) |
| N390 | Q | ssDNA (near active site) |
| N391 | R, Q | ssDNA (near active site) |
| G587 | K, R | NTS ssDNA |
| T588 | R, K | NTS ssDNA |
| T603 | K, R, Q | ssDNA (near active site) |
| Y619 | R, K | NTS ssDNA |
| E622 | R, K, A | NTS ssDNA |
| E709 | R, K | ssDNA (near active site) |
| Y712 | R, K | ssDNA (near active site) |
| D716 | R, K | ssDNA (near active site) |
| S752 | R, K | ssDNA (near active site) |
| D835 | K | ssDNA (near active site) |
| N840 | K | ssDNA (near active site) |
| F876 | R, K | NTS ssDNA |
| Y881 | K, R | ssDNA (near active site) |
| H884 | K | ssDNA (near active site) |
| D894 | K, R | NTS ssDNA |
| N925 | K, R | ssDNA (near active site) |
| E930 | R, K | ssDNA (near active site) |
| S957 | R, K | ssDNA (near active site) |
| D958 | K, N, R | ssDNA (near active site) |
| V1030 | R, K | NTS ssDNA |

In some embodiments, a variant Cas12i2 polypeptide of any one of SEQ ID NOs: 3-146 and 495-512 further comprises one or more substitutions listed in Table 6. In some embodiments, a variant Cas12i2 polypeptide comprises one or more substitutions listed in Table 2 and Table 6.

In some embodiments, a variant Cas12i2 polypeptide exhibiting increased single-stranded DNA interactions comprises two or more substitutions, e.g., G587R T588R, G587R T588K, G587R T588K Q590R, or G587R T588R Q590K. In some embodiments, a variant Cas12i2 polypeptide exhibiting increased ternary complex formation/stability comprises two or more substitutions, e.g., G587R T588R, G587R T588K, G587R T588K Q590R, or G587R T588R Q590K. In some embodiments, a variant Cas12i2 polypeptide of any one of SEQ ID NOs: 3-146 and 495-512 further comprises G587R T588R, G587R T588K, G587R T588K Q590R, or G587R T588R Q590K substitutions. In some embodiments, a variant Cas12i2 polypeptide of any one of SEQ ID NOs: 3-146 and 495-512 further comprises G587R T588R, G587R T588K, G587R T588K Q590R, or G587R T588R Q590K substitutions and one or more substitutions listed in Table 4 and/or Table 5 and/or Table 6. In some embodiments, the variant Cas12i2 polypeptide comprises any one or more substitutions in Table 4 and/or Table 5 and/or Table 6. In some embodiments, the variant Cas12i2 polypeptide with one or more of the substitutions in Table 4 and/or Table 5 and/or Table 6 exhibits increased single-stranded DNA interactions and/or affinity (e.g., by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more or any percentage therebetween) as compared to a parent polypeptide. In some embodiments, the variant Cas12i2 polypeptide with one or more of the substitutions in Table 4 and/or Table 5 and/or Table 6 exhibits increased ternary complex formation and/or ternary complex stability (e.g., by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more or any percentage therebetween) as compared to a parent polypeptide.

In some embodiments, a variant Cas12i2 polypeptide comprises a substitution that increases single-stranded DNA stability (e.g., the substitution increases electrostatic interactions between single-stranded DNA and the active site of the variant Cas12i2 polypeptide). In some embodiments, the variant Cas12i2 polypeptide increases single-stranded DNA stability by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more or any percentage therebetween, as compared to a parent polypeptide. Non-limiting examples of substitutions that can alter the ability of a variant Cas12i2 polypeptide to stabilize single-stranded DNA are shown in Table 6. In some embodiments, a variant Cas12i2 polypeptide comprising one or more substitutions listed in Table 6 exhibits increased single-stranded DNA stability relative to a parent polypeptide.

In some embodiments, the variant Cas12i2 polypeptide of any one of SEQ ID NOs: 3-146 and 495-512 comprising one or more substitutions listed in Table 4 and/or Table 5 and/or Table 6 exhibits increased enzymatic activity. In some embodiments, the variant Cas12i2 polypeptide comprising one or more substitutions listed in Table 4 and/or Table 5 and/or Table 6 exhibits increased enzymatic activity. In some embodiments, the variant Cas12i2 polypeptide of any one of SEQ ID NOs: 3-146 and 495-512 that further comprises G587R T588R, G587R T588K, G587R T588K Q590R, or G587R T588R Q590K substitutions and one or more substitutions listed in Table 4 and/or Table 5 and/or Table 6 exhibits increased enzymatic activity. In some embodiments, the variant Cas12i2 polypeptide exhibits increased enzymatic activity (e.g., by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more or any percentage therebetween) as compared to a parent polypeptide.

### Increased Heteroduplex Interactions

In some embodiments, a variant Cas12i2 polypeptide comprises a substitution that increases interactions with a DNA/RNA hybrid molecule relative to a parent polypeptide. In some embodiments, the variant Cas12i2 polypeptide comprises an alteration that increases affinity between the variant Cas12i2 polypeptide and a DNA/RNA hybrid relative to a parent polypeptide. In some embodiments, the DNA/RNA hybrid molecule is a heteroduplex. As used herein, the “heteroduplex” refers to a double helix formed by the spacer of an RNA guide and the target strand (TS). As used herein, the term “seed region” refers to the TS part of the heteroduplex that is immediately downstream of the PAM sequence. The seed region comprises the first bases that pair with the RNA guide in the heteroduplex and are required for RNA-DNA binding and displacement of the TS. In some embodiments, an alteration that increases interactions and/or affinity between the variant Cas12i2 polypeptide and the heteroduplex increase non-specific nucleic acid contacts. In some embodiments, an alteration that increases interactions and/or affinity between the variant Cas12i2 polypeptide and the heteroduplex increases ternary complex formation/stability relative to a parent polypeptide.

In some embodiments, the alteration that increases interactions and/or affinity between the variant Cas12i2 polypeptide and the heteroduplex is substituting one or more amino acids to an arginine, lysine, glutamine, asparagine, histidine, or serine residue. In some embodiments, the variant Cas12i2 polypeptide comprises a substitution of one or more amino acids contacting the heteroduplex to an arginine, lysine, glutamine, asparagine, histidine, or serine residue. In some embodiments, the variant Cas12i2 polypeptide comprises a substitution of one or more amino acids 110-130, 150-170, 250-320, 340-400, 420-450, 670-720, 770-810, or 830-870 to any one of an arginine, lysine, glutamine, asparagine, histidine, or serine residue. In some embodiments, the variant Cas12i2 polypeptide comprises an alteration of one or more amino acids in at least one domain/motif (e.g., the Rec1 domain, PI domain, Rec2 domain, or RuvC2 motif) to an arginine, lysine, glutamine, asparagine, histidine, or serine residue. In some embodiments, the nucleic acid interface substitution(s) increase heteroduplex interactions and/or affinity by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more or any percentage therebetween, as compared to a parent polypeptide.

In some embodiments, the substitution that increases heteroduplex interactions increases ternary complex formation/stability relative to a parent polypeptide. Non-limiting examples of substitutions that can alter the ability of a variant Cas12i2 polypeptide to interact with the heteroduplex are shown in Table 7. In some embodiments, a variant Cas12i2 polypeptide comprising one or more substitutions listed in Table 7 exhibits increased heteroduplex interactions (ternary complex formation) relative to a parent polypeptide. In some embodiments, a variant Cas12i2 polypeptide comprising one or more substitutions listed in Table 7 forms a more stable ternary complex, as compared to a parent polypeptide.

TABLE 7
Substitutions altering heteroduplex interactions.

| Residue | Substitution(s) | Contact |
|---|---|---|
| G116 | K, N, R | Heteroduplex |
| S118 | R, K | Heteroduplex |
| Y119 | K, R | Heteroduplex |
| A156 | K, N | Heteroduplex (seed region) |
| S161 | R, K, Q | Heteroduplex (seed region) |
| N165 | R, K | Heteroduplex (seed region) |
| E294 | Q | Heteroduplex (seed region) |
| N297 | K | Heteroduplex (seed region) |
| T308 | K, R, H, N | Heteroduplex (seed region) |
| N312 | R, H, K | Heteroduplex (seed region) |
| Q316 | R, K | Heteroduplex (seed region) |
| E343 | R, K | Heteroduplex |
| S346 | R, K, Q | Heteroduplex |
| E348 | R, K, Q, H | Heteroduplex |
| Y351 | K, H, N | Heteroduplex |
| V355 | K, R | Heteroduplex |
| H357 | K | Heteroduplex |
| E378 | K, R | Heteroduplex |
| V382 | R | Heteroduplex |
| D386 | R, K | Heteroduplex |
| E395 | K, Q | Heteroduplex |
| N399 | R | Heteroduplex |
| Q424 | R, K | Heteroduplex |
| Q425 | K, R | Heteroduplex |
| D427 | R | Heteroduplex |
| S431 | R, K | Heteroduplex (seed region) |
| V438 | R, K, Q | Heteroduplex (seed region) |
| N441 | R, K | Heteroduplex (seed region) |
| I675 | R, K | Heteroduplex |
| N679 | R, K | Heteroduplex |
| E683 | K | Heteroduplex |
| V685 | K, R, N | Heteroduplex |
| V687 | R, Q, K | Heteroduplex |
| E691 | R, K | Heteroduplex |
| A695 | R, K | Heteroduplex |
| Y712 | R, K | Heteroduplex |
| D716 | R, K | Heteroduplex |
| S775 | R | Heteroduplex |
| S781 | R, K | Heteroduplex |
| D782 | R, N | Heteroduplex |
| E783 | R, K | Heteroduplex |
| Q784 | R | Heteroduplex |
| E797 | R, K | Heteroduplex |
| E800 | N, S, Q | Heteroduplex |
| N841 | R, K | Heteroduplex |
| L855 | K | Heteroduplex (seed region) |
| Q856 | K | Heteroduplex |
| N861 | K, R | Heteroduplex (seed region) |
| Q865 | R, K | Heteroduplex (seed region) |

In some embodiments, a variant Cas12i2 polypeptide of any one of SEQ ID NOs: 3-146 and 495-512 further comprises one or more substitutions listed in Table 7. In some embodiments, a variant Cas12i2 polypeptide comprises one or more substitutions listed in Table 2 and Table 7.

In some embodiments, a variant Cas12i2 polypeptide exhibiting increased heteroduplex interactions comprises two or more substitutions, e.g., E691R A695R, S78K V438G, S78K V438A, S346R E343S, D782R D793N, S78R V438G, S78R V438A, S346K E343S, or D782K D793N. In some embodiments, a variant Cas12i2 polypeptide exhibiting increased ternary complex formation/stability comprises two or more substitutions, e.g., E691R A695R, S78K V438G, S78K V438A, S346R E343S, D782R D793N, S78R V438G, S78R V438A, S346K E343S, or D782K D793N. In some embodiments, a variant Cas12i2 polypeptide of any one of SEQ ID NOs: 3-146 and 495-512 further comprises E691R A695R, S78K V438G, S78K V438A, S346R E343S, D782R D793N, S78R V438G, S78R V438A, S346K E343S, or D782K D793N substitutions. In some embodiments, a variant Cas12i2 polypeptide of any one of SEQ ID NOs: 3-146 and 495-512 further comprises E691R A695R, S78K V438G, S78K V438A, S346R E343S, D782R D793N, S78R V438G, S78R V438A, S346K E343S, or D782K D793N substitutions and one or more substitutions listed in Table 4 and/or Table 5 and/or Table 6 and/or Table 7. In some embodiments, the variant Cas12i2 polypeptide comprises any one or more substitutions in Table 4 and/or Table 5 and/or Table 6 and/or Table 7. In some embodiments, the variant Cas12i2 polypeptide with one or more of the substitutions in Table 4 and/or Table 5 and/or Table 6 and/or Table 7 exhibits increased heteroduplex interactions and/or affinity (e.g., by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more or any percentage therebetween) as compared to a parent polypeptide. In some embodiments, the variant Cas12i2 polypeptide with one or more of the substitutions in Table 4 and/or Table 5 and/or Table 6 and/or Table 7 exhibits increased ternary complex formation and/or ternary complex stability (e.g., by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more or any percentage therebetween) as compared to a parent polypeptide.

In some embodiments, the variant Cas12i2 polypeptide of any one of SEQ ID NOs: 3-146 and 495-512 comprising one or more substitutions listed in Table 4 and/or Table 5 and/or Table 6 and/or Table 7 exhibits increased enzymatic activity. In some embodiments, the variant Cas12i2 polypeptide comprising one or more substitutions listed in Table 4 and/or Table 5 and/or Table 6 and/or Table 7 exhibits increased enzymatic activity. In some embodiments, the variant Cas12i2 polypeptide of any one of SEQ ID NOs: 3-146 and 495-512 that further comprises E691R A695R, S78K V438G, S78K V438A, S346R E343S, D782R D793N, S78R V438G, S78R V438A, S346K E343S, or D782K D793N substitutions and one or more substitutions listed in Table 4 and/or Table 5 and/or Table 6 and/or Table 7 exhibits increased enzymatic activity. In some embodiments, the variant Cas12i2 polypeptide exhibits increased enzymatic activity (e.g., by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more or any percentage therebetween) as compared to a parent polypeptide.

### Increased Double-Stranded DNA Duplex and Heteroduplex Stability

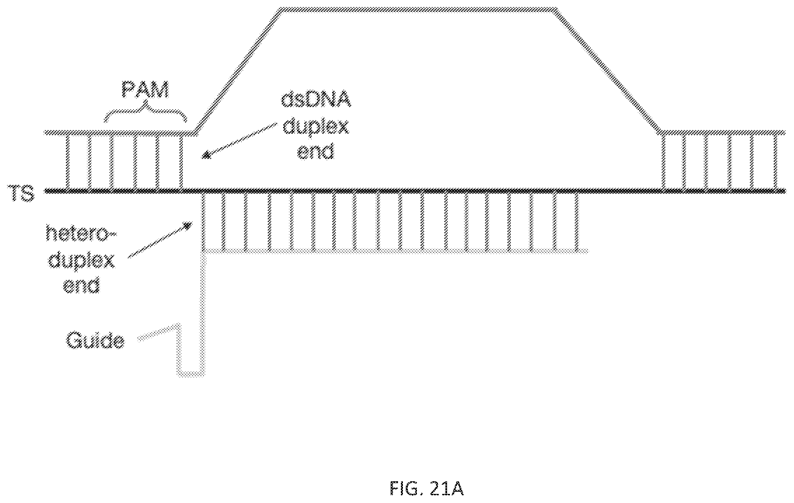

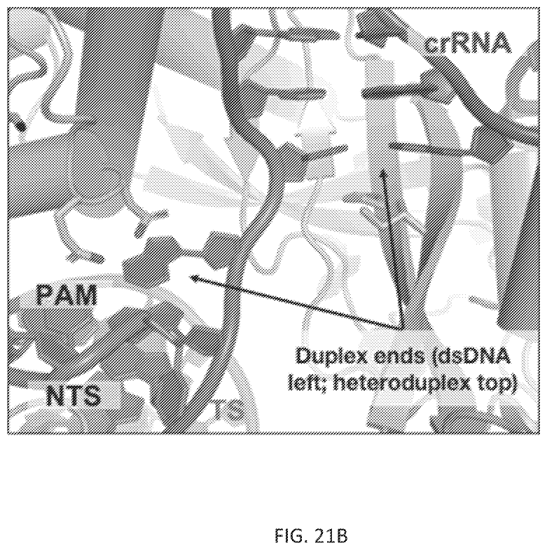

During ternary complex formation, double-stranded DNA downstream of the PAM sequence melts (e.g., unwinds) into a target strand (TS) and a non-target strand (NTS). The spacer of an RNA guide binds to the TS, forming a double helix that is referred to as the heteroduplex. The PAM sequence does not melt and remains as intact double-stranded DNA. This results in partial exposure of these terminal PAM dsDNA base pair to the environment and protein, which may be energetically unfavorable. Similarly, the terminal base pair of the heteroduplex is exposed and may be energetically unfavorable. See FIG. 21. In some embodiments, an alteration that increases aromatic, hydrophobic, Van der Waals, and/or cation-pi interactions between the variant Cas12i2 polypeptide and the exposed terminal PAM bases of the double-stranded DNA duplex or terminal bases of the heteroduplex increases stability of DNA melting during ternary complex formation. In some embodiments, an alteration that increases aromatic, hydrophobic, Van der Waals, and/or cation-pi interactions between the variant Cas12i2 polypeptide and exposed bases of the double-stranded DNA duplex or heteroduplex increases R-loop stability during ternary complex formation. In some embodiments, an alteration that increases aromatic, hydrophobic, Van der Waals, and/or cation-pi interactions between the variant Cas12i2 polypeptide and exposed bases of the double-stranded DNA duplex or heteroduplex increases ternary complex formation. In some embodiments, an alteration that increases aromatic, hydrophobic, Van der Waals, and/or cation-pi interactions between the variant Cas12i2 polypeptide and exposed bases of the double-stranded DNA duplex or heteroduplex increases ternary complex stability. See FIG. 20D.

In some embodiments, the alteration that increases aromatic, hydrophobic, Van der Waals, and/or cation-pi interactions is substituting one or more residues with an arginine, lysine, tryptophan, phenylalanine, tyrosine, methionine, histidine, glutamine, threonine, or valine residue. In some embodiments, the alteration that increases aromatic, hydrophobic, Van der Waals, and/or cation-pi interactions is substituting one or more residues contacting the double-stranded DNA duplex and/or heteroduplex with an arginine, lysine, tryptophan, phenylalanine, tyrosine, methionine, histidine, glutamine, threonine, or valine residue. In some embodiments, the alteration that increases aromatic, hydrophobic, Van der Waals, and/or cation-pi interactions is a substitution listed in Table 8. In some embodiments, a variant Cas12i2 polypeptide comprising a substitution listed in Table 8 exhibits increased aromatic, hydrophobic, Van der Waals, and/or cation-pi interactions between the variant Cas12i2 polypeptide and exposed bases of the double-stranded DNA duplex or heteroduplex as compared to a parent polypeptide. In some embodiments, the alteration includes substituting amino acids adjacent to the terminal duplex base pairs with a positively charged, aromatic, hydrophobic, or branched-chain amino acids (e.g., arginine, lysine, tryptophan, phenylalanine, tyrosine, methionine, histidine, glutamine, threonine, isoleucine or valine) to create energetically more favorable conditions for the double-stranded DNA and heteroduplex.

TABLE 8
Substitutions stabilizing the R-loop.

| Residue | Substitution(s) |
|---|---|
| I5 | K, R, W, F, Y |
| Q163 | K, M, R |
| N164 | H, R, K, Q, M, W, F |
| P577 | T, V |
| M869 | Q |

In some embodiments, a variant Cas12i2 polypeptide of any one of SEQ ID NOs: 3-146 and 495-512 further comprises one or more substitutions listed in Table 8. In some embodiments, a variant Cas12i2 polypeptide comprises one or more substitutions listed in Table 2 and Table 8.

In some embodiments, a variant Cas12i2 polypeptide exhibiting increased ternary complex formation and/or ternary complex stability (e.g., by stabilizing melting of DNA and/or the R-loop) comprises two or more substitutions, e.g., Q163N N164W, Q163M N164W, Q163M N164Q, Q163N N164Q, I5G P577Y, I5G P577F, I5G P577H, or I5M P577L. In some embodiments, a variant Cas12i2 polypeptide of any one of SEQ ID NOs: 3-146 and 495-512 further comprises Q163N N164W, Q163M N164W, Q163M N164Q, Q163N N164Q, 15G P577Y, 15G P577F, 15G P577H, or I5M P577L substitutions. In some embodiments, the variant Cas12i2 polypeptide comprises any one of SEQ ID NOs: 3-146 and 495-512 further comprises Q163N N164W, Q163M N164W, Q163M N164Q, Q163N N164Q, I5G P577Y, I5G P577F, I5G P577H, or I5M P577L substitutions and one or more substitutions from Table 4, Table 5, Table 6, Table 7, and/or Table 8. In some embodiments, the variant Cas12i2 polypeptide comprises any one or more substitutions in Table 4, Table 5, Table 6, Table 7, and/or Table 8. In some embodiments, the variant Cas12i2 polypeptide with one or more of the substitutions in Table 4, Table 5, Table 6, Table 7, and/or Table 8 exhibits increased ternary complex formation and/or ternary complex stability (e.g., by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more or any percentage therebetween) as compared to a parent polypeptide.

In some embodiments, the variant Cas12i2 polypeptide of any one of SEQ ID NOs: 3-146 and 495-512 comprising one or more substitutions listed in Table 4, Table 5, Table 6, Table 7, and/or Table 8 exhibits increased enzymatic activity. In some embodiments, the variant Cas12i2 polypeptide comprising one or more substitutions listed in Table 4, Table 5, Table 6, Table 7, and/or Table 8 exhibits increased enzymatic activity. In some embodiments, the variant Cas12i2 polypeptide of any one of SEQ ID NOs: 3-146 and 495-512 that further comprises Q163N N164W, Q163M N164W, Q163M N164Q, Q163N N164Q, I5G P577Y, I5G P577F, I5G P577H, or I5M P577L substitutions and one or more substitutions listed in Table 4, Table 5, Table 6, Table 7, and/or Table 8 exhibits increased enzymatic activity. In some embodiments, the variant Cas12i2 polypeptide exhibits increased enzymatic activity (e.g., by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more or any percentage therebetween) as compared to a parent polypeptide.

### Increased Conformational Changes

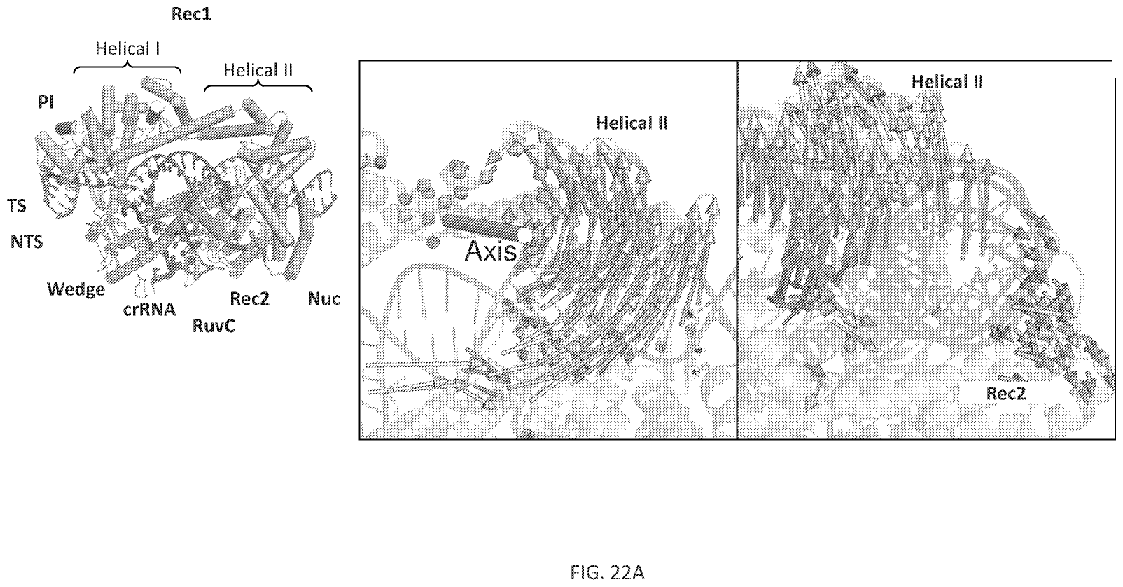

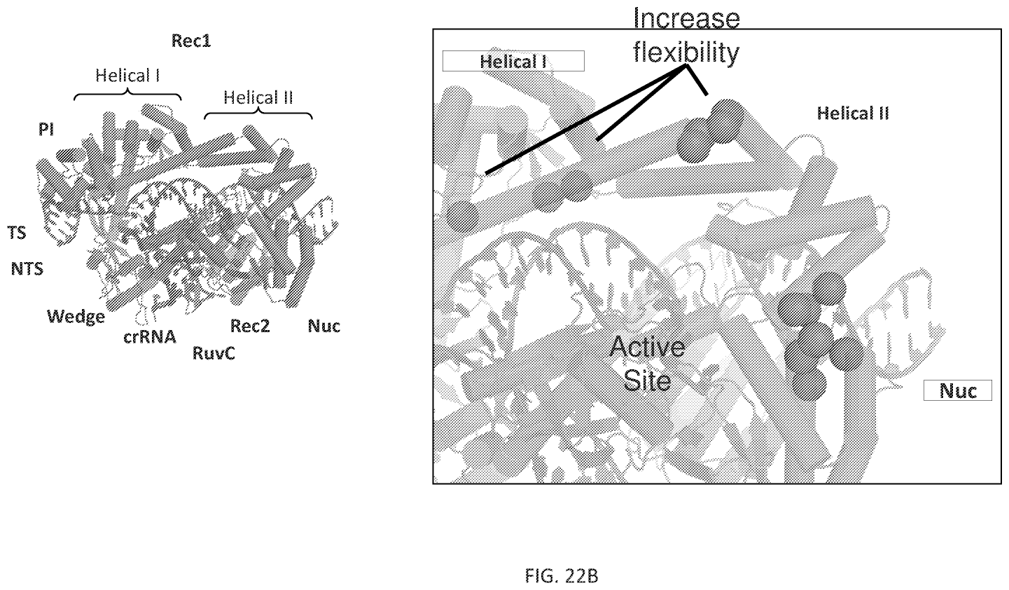

Conformational changes, e.g., upon binding RNA guide or target DNA, impact the function of a variant Cas12i2 polypeptide, e.g., conformational changes may alter kinetics of the variant Cas12i2 polypeptide. The Rec1 (Helical II) domain of Cas12i2 moves and rotates to accommodate DNA binding during ternary complex formation. See FIG. 22A and FIG. 22B. In some embodiments, an alteration that increases movement (e.g., flexibility or conformational changes) of the Helical II domain increases DNA binding/DNA binding affinity. In some embodiments, a substitution to increase flexibility, e.g., a substitution of a bulky amino acid to an amino acid with a small or smaller side chain (alanine, valine, glycine, or serine residue), in the Helical II domain increases ternary complex formation. In some embodiments, an alteration that increases movement (e.g., flexibility or conformational changes) of the Helical II domain increases ternary complex stability. In some embodiments, the alteration that increases conformational changes of the Helical II domain is substituting one or more residues with an alanine, valine, glycine, or serine residue. In some embodiments, the alteration that increases flexibility of the Helical II domain is substituting one or more residues with an alanine, valine, glycine, or serine residue. In some embodiments, a variant Cas12i2 polypeptide comprises an alteration of one or more amino acids near the Helical II domain. In some embodiments, the variant Cas12i2 polypeptide comprises an alteration of one or more amino acids near the Helical II domain to alanine, valine, glycine, or serine. In some embodiments, the variant Cas12i2 polypeptide comprises a substitution of one or more amino acids 327-330 to any one of alanine, valine, glycine, or serine. In some embodiments, a variant Cas12i2 polypeptide comprises a substitution set forth in Table 9.

TABLE 9
Substitutions altering flexibility of the Helical II domain.

| Residue | Substitution(s) |
|---|---|
| H300 | A |
| L327 | V, A |
| N328 | G, S |
| N329 | G, S |
| L330 | A |
| A434 | G |
| P436 | A, G |

In some embodiments, a variant Cas12i2 polypeptide of any one of SEQ ID NOs: 3-146 and 495-512 further comprises one or more substitutions listed in Table 9. In some embodiments, a variant Cas12i2 polypeptide comprises one or more substitutions listed in Table 2 and Table 9.

In some embodiments, the alteration that increases Helical II domain flexibility is a substitution listed in Table 9. In some embodiments, the variant Cas12i2 polypeptide with one or more of the substitutions listed in Table 9 exhibits increased Helical II domain flexibility by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more or any percentage therebetween, as compared to a parent polypeptide. In some embodiments, the alteration that increases DNA binding/DNA affinity is a substitution listed in Table 9. In some embodiments, the variant Cas12i2 polypeptide with one or more of the substitutions listed in Table 9 exhibits increased DNA binding/DNA affinity by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more or any percentage therebetween, as compared to a parent polypeptide. In some embodiments, a variant Cas12i2 polypeptide comprising a substitution listed in Table 9 exhibits increased ternary complex formation and/or ternary complex stability (e.g., by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more or any percentage therebetween) as compared to a parent polypeptide.

In some embodiments, a variant Cas12i2 polypeptide exhibiting increased Helical II domain flexibility comprises two or more substitutions, e.g., L327V N328S N329G L330A, L327A N328S N329G L330A, L327V N328G N329S L330A, or L327V N328G N329G L330A. In some embodiments, a variant Cas12i2 polypeptide exhibiting increased DNA binding/affinity comprises two or more substitutions, e.g., L327V N328S N329G L330A, L327A N328S N329G L330A, L327V N328G N329S L330A, or L327V N328G N329G L330A. In some embodiments, a variant Cas12i2 polypeptide exhibiting increased ternary complex formation/stability comprises two or more substitutions, e.g., L327V N328S N329G L330A, L327A N328S N329G L330A, L327V N328G N329S L330A, or L327V N328G N329G L330A. In some embodiments, a variant Cas12i2 polypeptide of any one of SEQ ID NOs: 3-146 and 495-512 further comprises L327V N328S N329G L330A, L327A N328S N329G L330A, L327V N328G N329S L330A, or L327V N328G N329G L330A substitutions. In some embodiments, a variant Cas12i2 polypeptide of any one of SEQ ID NOs: 3-146 and 495-512 further comprises L327V N328S N329G L330A, L327A N328S N329G L330A, L327V N328G N329S L330A, or L327V N328G N329G L330A substitutions and one or more substitutions listed in Table 4 and/or Table 5 and/or Table 6 and/or Table 7 and/or Table 8 and/or Table 9. In some embodiments, the variant Cas12i2 polypeptide comprises any one or more substitutions in Table 4 and/or Table 5 and/or Table 6 and/or Table 7 and/or Table 8 and/or Table 9. In some embodiments, the variant Cas12i2 polypeptide with one or more of the substitutions in Table 4 and/or Table 5 and/or Table 6 and/or Table 7 and/or Table 8 and/or Table 9 exhibits increased DNA binding/affinity (e.g., by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more or any percentage therebetween) as compared to a parent polypeptide. In some embodiments, the variant Cas12i2 polypeptide with one or more of the substitutions in Table 4 and/or Table 5 and/or Table 6 and/or Table 7 and/or Table 8 and/or Table 9 exhibits increased ternary complex formation and/or ternary complex stability (e.g., by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more or any percentage therebetween) as compared to a parent polypeptide.

In some embodiments, the variant Cas12i2 polypeptide of any one of SEQ ID NOs: 3-146 and 495-512 comprising one or more substitutions listed in Table 4 and/or Table 5 and/or Table 6 and/or Table 7 and/or Table 8 and/or Table 9 exhibits increased enzymatic activity. In some embodiments, the variant Cas12i2 polypeptide comprising one or more substitutions listed in Table 4 and/or Table 5 and/or Table 6 and/or Table 7 and/or Table 8 and/or Table 9 exhibits increased enzymatic activity. In some embodiments, the variant Cas12i2 polypeptide of any one of SEQ ID NOs: 3-146 and 495-512 that further comprises L327V N328S N329G L330A, L327A N328S N329G L330A, L327V N328G N329S L330A, or L327V N328G N329G L330A substitutions and one or more substitutions listed in Table 4 and/or Table 5 and/or Table 6 and/or Table 7 and/or Table 8 and/or Table 9 exhibits increased enzymatic activity. In some embodiments, the variant Cas12i2 polypeptide exhibits increased enzymatic activity (e.g., by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more or any percentage therebetween) as compared to a parent polypeptide.

In some embodiments, an alteration that increases connections between the Nuc and Helical II interface, which forms when target single-stranded DNA is in the active site of a Cas12i2 polypeptide, increases the transition from binary complex to ternary complex. In some embodiments, an alteration that increases connections between the Nuc and Helical II interface increases ternary complex formation. See FIG. 22B. In some embodiments, an alteration that increases connections between the Nuc and Helical II interface increases ternary complex stability. In some embodiments, the alteration that increases connections between the Nuc and Helical II interface is substituting one or more residues with an aspartic acid, glutamic acid, arginine, or lysine residue. In some embodiments, the variant Cas12i2 polypeptide comprises a substitution of one or more amino acids 380-390 or 910-930 to any one of aspartic acid, glutamic acid, arginine, or lysine. In some embodiments, a variant Cas12i2 polypeptide comprises a substitution set forth in Table 10.

TABLE 10
Substitutions increasing connections
at the Nuc and Helical II interface.

| Residue | Substitution(s) |
|---|---|
| V383 | D, E |
| D386 | E |
| D387 | E |
| Q919 | K |
| N925 | D, E, R, K |

In some embodiments, a variant Cas12i2 polypeptide of any one of SEQ ID NOs: 3-146 and 495-512 further comprises one or more substitutions listed in Table 10. In some embodiments, a variant Cas12i2 polypeptide comprises one or more substitutions listed in Table 2 and Table 10.

In some embodiments, a substitution in Table 10 increases connections between the Nuc and Helical II interface. In some embodiments, the variant Cas12i2 polypeptide with one or more of the substitutions in Table 10 increases connections between the Nuc and Helical II interface by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more or any percentage therebetween, as compared to a parent polypeptide. In some embodiments, a variant Cas12i2 polypeptide comprising a substitution listed in Table 10 exhibits increased ternary complex formation and/or ternary complex stability (e.g., by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more or any percentage therebetween) as compared to a parent polypeptide.

In some embodiments, a variant Cas12i2 polypeptide exhibiting increased connections between the Nuc and Helical II interface comprises two or more substitutions, e.g., V383D D387E, V383D D386E, V383E D387E, V383E D386E, Q919K D387E, Q919K D383D, Q919K V383E, D386E D387E, N925D D362R K365R, N925E D362R K365R, N925D D362K K365R, N925E D362K K365R, N925D D362R, N925E D362R, N925D D362K, N925E D362K, N925D K365R, N925E K365R, or D362E N925K. In some embodiments, a variant Cas12i2 polypeptide exhibiting increased ternary complex formation/stability comprises two or more substitutions, e.g., V383D D387E, V383D D386E, V383E D387E, V383E D386E, Q919K D387E, Q919K D383D, Q919K V383E, D386E D387E, N925D D362R K365R, N925E D362R K365R, N925D D362K K365R, N925E D362K K365R, N925D D362R, N925E D362R, N925D D362K, N925E D362K, N925D K365R, N925E K365R, or D362E N925K. In some embodiments, a variant Cas12i2 polypeptide of any one of SEQ ID NOs: 3-146 and 495-512 further comprises V383D D387E, V383D D386E, V383E D387E, V383E D386E, Q919K D387E, Q919K D383D, Q919K V383E, D386E D387E, N925D D362R K365R, N925E D362R K365R, N925D D362K K365R, N925E D362K K365R, N925D D362R, N925E D362R, N925D D362K, N925E D362K, N925D K365R, N925E K365R, or D362E N925K substitutions. In some embodiments, a variant Cas12i2 polypeptide of any one of SEQ ID NOs: 3-146 and 495-512 further comprises V383D D387E, V383D D386E, V383E D387E, V383E D386E, Q919K D387E, Q919K D383D, Q919K V383E, D386E D387E, N925D D362R K365R, N925E D362R K365R, N925D D362K K365R, N925E D362K K365R, N925D D362R, N925E D362R, N925D D362K, N925E D362K, N925D K365R, N925E K365R, or D362E N925K substitutions and one or more substitutions listed in Table 4 and/or Table 5 and/or Table 6 and/or Table 7 and/or Table 8 and/or Table 9 and/or Table 10. In some embodiments, the variant Cas12i2 polypeptide comprises any one or more substitutions in Table 4 and/or Table 5 and/or Table 6 and/or Table 7 and/or Table 8 and/or Table 9 and/or Table 10. In some embodiments, the variant Cas12i2 polypeptide with one or more of the substitutions in Table 4 and/or Table 5 and/or Table 6 and/or Table 7 and/or Table 8 and/or Table 9 and/or Table 10 exhibits increased connections between the Nuc and Helical II interface (e.g., by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more or any percentage therebetween) as compared to a parent polypeptide. In some embodiments, the variant Cas12i2 polypeptide with one or more of the substitutions in Table 4 and/or Table 5 and/or Table 6 and/or Table 7 and/or Table 8 and/or Table 9 and/or Table 10 exhibits increased ternary complex formation and/or ternary complex stability (e.g., by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more or any percentage therebetween) as compared to a parent polypeptide.

In some embodiments, the variant Cas12i2 polypeptide of any one of SEQ ID NOs: 3-146 and 495-512 comprising one or more substitutions listed in Table 4 and/or Table 5 and/or Table 6 and/or Table 7 and/or Table 8 and/or Table 9 and/or Table 10 exhibits increased enzymatic activity. In some embodiments, the variant Cas12i2 polypeptide comprising one or more substitutions listed in Table 4 and/or Table 5 and/or Table 6 and/or Table 7 and/or Table 8 and/or Table 9 and/or Table 10 exhibits increased enzymatic activity. In some embodiments, the variant Cas12i2 polypeptide of any one of SEQ ID NOs: 3-146 and 495-512 that further comprises V383D D387E, V383D D386E, V383E D387E, V383E D386E, Q919K D387E, Q919K D383D, Q919K V383E, D386E D387E, N925D D362R K365R, N925E D362R K365R, N925D D362K K365R, N925E D362K K365R, N925D D362R, N925E D362R, N925D D362K, N925E D362K, N925D K365R, N925E K365R, or D362E N925K substitutions and one or more substitutions listed in Table 4 and/or Table 5 and/or Table 6 and/or Table 7 and/or Table 8 and/or Table 9 and/or Table 10 exhibits increased enzymatic activity. In some embodiments, the variant Cas12i2 polypeptide exhibits increased enzymatic activity (e.g., by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more or any percentage therebetween) as compared to a parent polypeptide.

In some embodiments, an alteration decreases connections between the Nuc and Helical II interface. In some embodiments, an alteration that decreases connections between the Nuc and Helical II interface increases ternary complex formation. In some embodiments, an alteration that decreases connections between the Nuc and Helical II interface is substituting one or more residues with an asparagine or serine residue. In some embodiments, the variant Cas12i2 polypeptide comprises a substitution of one or more amino acids 386, 387, 915, and 956 to asparagine or serine. In some embodiments, the variant Cas12i2 polypeptide comprises D386N R915S, D387N R956S, D386N D387N R915S R956S, or D386N D387N R915S R922S R956S substitutions. In some embodiments, the variant Cas12i2 polypeptide comprises of any one of SEQ ID NOs: 3-146 and 495-512 further comprises D386N R915S, D387N R956S, D386N D387N R915S R956S, or D386N D387N R915S R922S R956S substitutions.

### Increased Fidelity

In some aspects, a variant Cas12i2 polypeptide comprises an alteration that increases on-target specificity relative to a parent polypeptide. In some aspects, a variant Cas12i2 polypeptide comprises an alteration that increases on-target binding relative to a parent polypeptide. In some embodiments, the variant Cas12i2 polypeptide comprises an alteration that increases interactions (e.g., affinity) between the variant Cas12i2 polypeptide and on-target DNA relative to a parent polypeptide.

In some embodiments, the alteration that increases on-target specificity is substituting one or more amino acids to an alanine, serine, valine, glutamine, or asparagine residue. In some aspects, the alteration that increases on-target specificity is truncating a residue that contacts the spacer sequence of an RNA guide (e.g., substituting a residue that contacts the spacer sequence with a residue having a smaller side chain). In some aspects, the alteration that increases on-target specificity is truncating a residue, e.g., substitution to alanine, serine, or valine, that contacts the spacer sequence of an RNA guide. In some embodiments, the variant Cas12i2 polypeptide comprises a substitution of one or more amino acids that contact the spacer sequence of an RNA guide to an alanine, serine, valine, glutamine, or asparagine residue. In some embodiments, the variant Cas12i2 polypeptide comprises a substitution of one or more amino acids 290-320, 340-360, 390-450, 550-580, 710-720, 760-810, or 830-870 to any one of an alanine, serine, valine, glutamine, or asparagine residue. In some embodiments, the variant Cas12i2 polypeptide comprises an alteration of one or more amino acids in at least one domain/motif (e.g., the Wedge domain, Rec1 domain, Rec2 domain, or RuvC2 motif) to an alanine, serine, valine, glutamine, or asparagine residue. In some embodiments, a truncating substitution in the Helical II domain results in a variant Cas12i2 polypeptide exhibiting increased on-target binding specificity. In some aspects, one or more of the following Helical II residues are truncated: E348, E349, E395, 1397, R398, N399, Y351, H356, H357, K394, and R428. In some embodiments, the substitution(s) increase on-target specificity with the variant Cas12i2 polypeptide by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more or any percentage therebetween, as compared to a parent polypeptide. In some embodiments, the substitution(s) increase on-target binding of the variant Cas12i2 polypeptide by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more or any percentage therebetween, as compared to a parent polypeptide. In some embodiments, the substitution(s) increase on-target binding affinity of the variant Cas12i2 polypeptide by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more or any percentage therebetween, as compared to a parent polypeptide.

Non-limiting examples of alterations that can alter the ability of a variant Cas12i2 polypeptide to selectively bind to on-target DNA are substitutions listed in Table 11. In some embodiments, a variant Cas12i2 polypeptide comprising one or more substitutions listed in Table 11 exhibits increased on-target specificity relative to a parent polypeptide. In some embodiments, a variant Cas12i2 polypeptide comprising one or more substitutions listed in Table 11 exhibits increased on-target binding relative to a parent polypeptide. In some embodiments, a variant Cas12i2 polypeptide comprising one or more substitutions listed in Table 11 exhibits increased on-target binding affinity relative to a parent polypeptide. As used herein, the term “TS seed” refers to the seed sequence that forms a duplex with the RNA guide and the term “guide seed” refers to the RNA guide that pairs with the TS seed sequence.

TABLE 11
Substitutions increasing on-target specificity.

| Residue | Substitution(s) | Contact |
|---|---|---|
| K9 | A, N | Heteroduplex guide seed |
| Y119 | A | Heteroduplex guide seed |
| N296 | A | Heteroduplex TS seed |
| T301 | A | Heteroduplex TS seed |
| K304 | A | Heteroduplex TS seed |
| I305 | A | Heteroduplex TS/guide seed |
| K306 | A | Heteroduplex guide seed |
| T308 | A | Heteroduplex TS seed |
| R309 | Q, A | Heteroduplex TS seed |
| N312 | A | Heteroduplex TS seed |
| S346 | A | Heteroduplex guide |
| E348 | A, S | Heteroduplex guide |
| E349 | A, S | Heteroduplex TS |
| Y351 | A, S, H | Heteroduplex TS |
| H356 | A, S | Heteroduplex TS |
| H357 | A, S | Heteroduplex TS |
| K394 | A | Heteroduplex TS |
| E395 | A, S | Heteroduplex TS |
| I397 | V, A | Heteroduplex TS |
| R398 | A, S | Heteroduplex guide |
| N399 | A, S | Heteroduplex guide |
| R402 | A, S | Heteroduplex guide |
| R428 | A, Q | Heteroduplex guide |
| K433 | A, S | Heteroduplex guide seed |
| N435 | A | Heteroduplex guide seed |
| N441 | A | Heteroduplex guide seed |
| Q442 | A | Heteroduplex guide seed |
| T445 | A | Heteroduplex TS seed |
| S565 | A | Heteroduplex TS seed |
| T567 | A | Heteroduplex TS seed |
| R575 | A, Q | Heteroduplex guide seed |
| K678 | A, N | Heteroduplex TS |
| R715 | A, Q | Heteroduplex guide |
| R719 | A, Q | Heteroduplex guide |
| K761 | A, N | Heteroduplex TS |
| Y765 | A | Heteroduplex TS/guide |
| S766 | A | Heteroduplex guide |
| S769 | A | Heteroduplex guide |
| A774 | G | Heteroduplex guide |
| K776 | A | Heteroduplex TS/guide |
| P779 | A | Heteroduplex TS |
| D782 | A | Heteroduplex TS |
| R785 | A, Q | Heteroduplex TS |
| E800 | A | Heteroduplex TS |
| T804 | A | Heteroduplex TS |
| K807 | A | Heteroduplex TS |
| T838 | A | Heteroduplex TS |
| T839 | A | Heteroduplex guide |
| N841 | A | Heteroduplex guide |
| K844 | A, N | Heteroduplex TS |
| K845 | A, N | Heteroduplex TS/guide |
| N848 | A | Heteroduplex guide |
| S849 | G | Heteroduplex guide |
| M852 | A, S | Heteroduplex TS/guide |
| L855 | A | Heteroduplex TS |
| R857 | A, N | Heteroduplex TS |
| N861 | A | Heteroduplex TS |
| K862 | A, N | Heteroduplex guide |
| Q865 | A | Heteroduplex guide seed |
| M866 | A | Heteroduplex guide seed |

In some embodiments, the alteration that increases on-target specificity (e.g., a substitution listed in Table 11) further increases on-target ternary complex formation and/or on-target ternary complex stability (e.g., on-target ternary complex formation/stability). In some embodiments, the alteration that increases on-target specificity increases on-ternary complex formation and/or on-target ternary complex stability by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more or any percentage therebetween as compared to a parent polypeptide.

In some aspects, a variant Cas12i2 polypeptide comprises an alteration that decreases off-target specificity relative to a parent polypeptide. In some aspects, a variant Cas12i2 polypeptide comprises an alteration that decreases off-target binding relative to a parent polypeptide. In some embodiments, the variant Cas12i2 polypeptide comprises an alteration that decreases interactions (e.g., affinity) between the variant Cas12i2 polypeptide and off-target DNA relative to a parent polypeptide.

In some embodiments, the alteration that decreases off-target specificity is substituting one or more amino acids to an alanine, serine, valine, glutamine, or asparagine residue. In some aspects, the alteration that decreases off-target specificity is truncating a residue that contacts the spacer sequence of an RNA guide (e.g., substituting a residue that contacts the spacer sequence with a residue having a smaller side chain). In some aspects, the alteration that decreases off-target specificity is truncating a residue, e.g., substitution to alanine, serine, or valine, that contact the spacer sequence of an RNA guide. In some embodiments, the variant Cas12i2 polypeptide comprises a substitution of one or more amino acids that contact the spacer sequence of an RNA guide to an alanine, serine, valine, glutamine, or asparagine residue. In some embodiments, the variant Cas12i2 polypeptide comprises a substitution of one or more amino acids 290-320, 340-360, 390-450, 550-580, 710-720, 760-810, or 830-870 to any one of an alanine, serine, valine, glutamine, or asparagine residue. In some embodiments, the variant Cas12i2 polypeptide comprises an alteration of one or more amino acids in at least one domain/motif (e.g., the Wedge domain, Rec1 domain, Rec2 domain, or RuvC2 motif) to an alanine, serine, valine, glutamine, or asparagine residue. In some embodiments, a truncating substitution in the Helical II domain results in a variant Cas12i2 polypeptide exhibiting decreased off-target binding specificity. In some aspects, one or more of the following Helical II residues are truncated: E348, E349, E395, 1397, R398, N399, Y351, H356, H357, K394, and R428. In some embodiments, the substitution(s) decrease off-target specificity with the variant Cas12i2 polypeptide by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, as compared to a parent polypeptide. In some embodiments, the substitution(s) decrease off-target binding of the variant Cas12i2 polypeptide by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, as compared to a parent polypeptide. In some embodiments, the substitution(s) decrease off-target binding affinity of the variant Cas12i2 polypeptide by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, as compared to a parent polypeptide.

Non-limiting examples of alterations that can alter the ability of a variant Cas12i2 polypeptide to bind to off-target DNA are substitutions listed in Table 11. In some embodiments, a variant Cas12i2 polypeptide comprising one or more substitutions listed in Table 11 exhibits decreased off-target specificity relative to a parent polypeptide. In some embodiments, a variant Cas12i2 polypeptide comprising one or more substitutions listed in Table 11 exhibits decreased off-target binding relative to a parent polypeptide. In some embodiments, a variant Cas12i2 polypeptide comprising one or more substitutions listed in Table 11 exhibits decreased off-target binding affinity relative to a parent polypeptide.

In some embodiments, the substitution(s) that increase on-target specificity of the variant Cas12i2 polypeptide by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more or any percentage therebetween further decrease off-target specificity of the variant Cas12i2 polypeptide by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, as compared to a parent polypeptide. In some embodiments, the substitution(s) that increase on-target binding of the variant Cas12i2 polypeptide by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more or any percentage therebetween further decrease off-target binding of the variant Cas12i2 polypeptide by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, as compared to a parent polypeptide. In some embodiments, the substitution(s) that increase on-target binding affinity of the variant Cas12i2 polypeptide by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more or any percentage therebetween, further decrease off-target binding affinity of the variant Cas12i2 polypeptide by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, as compared to a parent polypeptide.

In some embodiments, a variant Cas12i2 polypeptide of any one of SEQ ID NOs: 3-146 and 495-512 further comprises one or more substitutions listed in Table 11. In some embodiments, a variant Cas12i2 polypeptide comprises one or more substitutions listed in Table 2 and Table 11.

In some embodiments, the variant Cas12i2 polypeptide of any one of SEQ ID NOs: 3-146 and 495-512 comprising one or more substitutions listed in Table 4 and/or Table 5 and/or Table 6 and/or Table 7 and/or Table 8 and/or Table 9 and/or Table 10 and/or Table 11 exhibits increased on-target enzymatic activity. In some embodiments, the variant Cas12i2 polypeptide comprising one or more substitutions listed in Table 4 and/or Table 5 and/or Table 6 and/or Table 7 and/or Table 8 and/or Table 9 and/or Table 10 and/or Table 11 exhibits increased on-target enzymatic activity. In some embodiments, the variant Cas12i2 polypeptide exhibits increased on-target enzymatic activity (e.g., by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more or any percentage therebetween) as compared to a parent polypeptide.

In some embodiments, the variant Cas12i2 polypeptide of any one of SEQ ID NOs: 3-146 and 495-512 comprising one or more substitutions listed in Table 4 and/or Table 5 and/or Table 6 and/or Table 7 and/or Table 8 and/or Table 9 and/or Table 10 and/or Table 11 exhibits an increased ratio of on-target enzymatic activity to off-target enzymatic activity. In some embodiments, the variant Cas12i2 polypeptide comprising one or more substitutions listed in Table 4 and/or Table 5 and/or Table 6 and/or Table 7 and/or Table 8 and/or Table 9 and/or Table 10 and/or Table 11 exhibits an increased ratio of on-target enzymatic activity to off-target enzymatic activity. In some embodiments, on-target enzymatic activity of the variant Cas12i2 polypeptide (e.g., the variant Cas12i2 polypeptide of any one of SEQ ID NOs: 3-146 and 495-512 comprising one or more substitutions listed in Table 4 and/or Table 5 and/or Table 6 and/or Table 7 and/or Table 8 and/or Table 9 and/or Table 10 and/or Table 11) is at least 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more or any percentage therebetween higher than off-target enzymatic activity of the variant Cas12i2 polypeptide, as compared to a parent polypeptide. In some embodiments, on-target enzymatic activity of the variant Cas12i2 polypeptide (e.g., the variant Cas12i2 polypeptide comprising one or more substitutions listed in Table 4 and/or Table 5 and/or Table 6 and/or Table 7 and/or Table 8 and/or Table 9 and/or Table 10 and/or Table 11) is at least 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more or any percentage therebetween higher than off-target enzymatic activity of the variant Cas12i2 polypeptide, as compared to a parent polypeptide.

In some embodiments, enzymatic activity of the variant Cas12i2 polypeptide (e.g., the variant Cas12i2 polypeptide of any one of SEQ ID NOs: 3-146 and 495-512 at an off-target locus is no more than 10% (e.g., 10%, 9%, 8%, 7%, 6%, 5%, 4%, 3%, 2%, 1%, or 0%) of the enzymatic activity at the on-target locus. In some embodiments, enzymatic activity of the variant Cas12i2 polypeptide (e.g., the variant Cas12i2 polypeptide of any one of SEQ ID NOs: 3-146 and 495-512 comprising one or more substitutions listed in Table 4 and/or Table 5 and/or Table 6 and/or Table 7 and/or Table 8 and/or Table 9 and/or Table 10 and/or Table 11) at an off-target locus is no more than 10% (e.g., 10%, 9%, 8%, 7%, 6%, 5%, 4%, 3%, 2%, 1%, or 0%) of the enzymatic activity at the on-target locus. In some embodiments, enzymatic activity of the variant Cas12i2 polypeptide (e.g., the variant Cas12i2 polypeptide of any one of SEQ ID NOs: 3-146 and 495-512 at an off-target locus is no more than 5% (e.g., 5%, 4%, 3%, 2%, 1%, or 0%) of the enzymatic activity at the on-target locus. In some embodiments, enzymatic activity of the variant Cas12i2 polypeptide (e.g., the variant Cas12i2 polypeptide of any one of SEQ ID NOs: 3-146 and 495-512 comprising one or more substitutions listed in Table 4 and/or Table 5 and/or Table 6 and/or Table 7 and/or Table 8 and/or Table 9 and/or Table 10 and/or Table 11) at an off-target locus is no more than 5% (e.g., 5%, 4%, 3%, 2%, 1%, or 0%) of the enzymatic activity at the on-target locus. By comparison, enzymatic activity of SpCas9 at an off-target locus is up to 95% (e.g., 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, or 0%) of the enzymatic activity at the on-target locus. See Example 16.

In some embodiments, editing efficiency of the variant Cas12i2 polypeptide (e.g., the variant Cas12i2 polypeptide of any one of SEQ ID NOs: 3-146 and 495-512 at an off-target locus is no more than 10% (e.g., 10%, 9%, 8%, 7%, 6%, 5%, 4%, 3%, 2%, 1%, or 0%) of the editing efficiency at the on-target locus. In some embodiments, editing efficiency of the variant Cas12i2 polypeptide (e.g., the variant Cas12i2 polypeptide of any one of SEQ ID NOs: 3-146 and 495-512 comprising one or more substitutions listed in Table 4 and/or Table 5 and/or Table 6 and/or Table 7 and/or Table 8 and/or Table 9 and/or Table 10 and/or Table 11) at an off-target locus is no more than 10% (e.g., 10%, 9%, 8%, 7%, 6%, 5%, 4%, 3%, 2%, 1%, or 0%) of the editing efficiency at the on-target locus. In some embodiments, editing efficiency of the variant Cas12i2 polypeptide (e.g., the variant Cas12i2 polypeptide of any one of SEQ ID NOs: 3-146 and 495-512 at an off-target locus is no more than 5% (e.g., 5%, 4%, 3%, 2%, 1%, or 0%) of the editing efficiency at the on-target locus. In some embodiments, editing efficiency of the variant Cas12i2 polypeptide (e.g., the variant Cas12i2 polypeptide of any one of SEQ ID NOs: 3-146 and 495-512 comprising one or more substitutions listed in Table 4 and/or Table 5 and/or Table 6 and/or Table 7 and/or Table 8 and/or Table 9 and/or Table 10 and/or Table 11) at an off-target locus is no more than 5% (e.g., 5%, 4%, 3%, 2%, 1%, or 0%) of the editing efficiency at the on-target locus. By comparison, editing efficiency of SpCas9 at an off-target locus is up to 95% (e.g., 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, or 0%) of the editing efficiency at the on-target locus. See Example 16.

In some embodiments, editing by the variant Cas12i2 polypeptide (e.g., the variant Cas12i2 polypeptide of any one of SEQ ID NOs: 3-146 and 495-512 at an off-target locus is no more than 10% (e.g., 10%, 9%, 8%, 7%, 6%, 5%, 4%, 3%, 2%, 1%, or 0%) of the editing at the on-target locus. In some embodiments, editing by the variant Cas12i2 polypeptide (e.g., the variant Cas12i2 polypeptide of any one of SEQ ID NOs: 3-146 and 495-512 comprising one or more substitutions listed in Table 4 and/or Table 5 and/or Table 6 and/or Table 7 and/or Table 8 and/or Table 9 and/or Table 10 and/or Table 11) at an off-target locus is no more than 10% (e.g., 10%, 9%, 8%, 7%, 6%, 5%, 4%, 3%, 2%, 1%, or 0%) of the editing at the on-target locus. In some embodiments, editing by the variant Cas12i2 polypeptide (e.g., the variant Cas12i2 polypeptide of any one of SEQ ID NOs: 3-146 and 495-512 at an off-target locus is no more than 5% (e.g., 5%, 4%, 3%, 2%, 1%, or 0%) of the editing at the on-target locus. In some embodiments, editing by the variant Cas12i2 polypeptide (e.g., the variant Cas12i2 polypeptide of any one of SEQ ID NOs: 3-146 and 495-512 comprising one or more substitutions listed in Table 4 and/or Table 5 and/or Table 6 and/or Table 7 and/or Table 8 and/or Table 9 and/or Table 10 and/or Table 11) at an off-target locus is no more than 5% (e.g., 5%, 4%, 3%, 2%, 1%, or 0%) of the editing at the on-target locus. By comparison, editing of SpCas9 at an off-target locus is up to 95% (e.g., 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, or 0%) of the editing at the on-target locus. See Example 16.

In some aspects, a variant Cas12i2 polypeptide comprising an alteration that increases on-target specificity relative to a parent polypeptide is an alteration that decreases the catalysis rate (Kcat) compared to a parent polypeptide. A decreased (e.g., slower) Kcat allows for the variant Cas12i2 polypeptide to discriminate between an on-target sequence and an off-target sequence. In some embodiments, the alteration that decreases the catalysis rate is substituting one or more amino acids to an alanine, serine, threonine, valine, leucine, methionine, asparagine, or isoleucine. In some embodiments, the variant Cas12i2 polypeptide comprises a substitution of one or more amino acids 600-605, 835-840, or 1020-1030 to any one of an alanine, serine, threonine, valine, leucine, methionine, asparagine, or isoleucine. In some embodiments, the variant Cas12i2 polypeptide comprises an alteration of one or more amino acids in at least one RuvC domain/motif (e.g., RuvC1, RuvC2, or RuvC) to an alanine, serine, threonine, valine, leucine, methionine, asparagine, or isoleucine. In some embodiments, the substitution(s) decrease the Kcat of the variant Cas12i2 polypeptide by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, as compared to a parent polypeptide.

In some embodiments, the substitution(s) that decrease the Kcat of the variant Cas12i2 polypeptide further increase on-target specificity by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more or any percentage therebetween, as compared to a parent polypeptide. In some embodiments, the substitution(s) that decrease the Kcat of the variant Cas12i2 polypeptide further increase on-target binding of the variant Cas12i2 polypeptide by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more or any percentage therebetween, as compared to a parent polypeptide. In some embodiments, the substitution(s) that decrease the Kcat of the variant Cas12i2 polypeptide further increase on-target binding affinity of the variant Cas12i2 polypeptide by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more or any percentage therebetween, as compared to a parent polypeptide.

In some embodiments, the substitution(s) that decrease the Kcat of the variant Cas12i2 polypeptide further decrease off-target specificity by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, as compared to a parent polypeptide. In some embodiments, the substitution(s) that decrease the Kcat of the variant Cas12i2 polypeptide further decrease off-target binding of the variant Cas12i2 polypeptide by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, as compared to a parent polypeptide. In some embodiments, the substitution(s) that decrease the Kcat of the variant Cas12i2 polypeptide further decrease off-target binding affinity of the variant Cas12i2 polypeptide by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, as compared to a parent polypeptide.

Non-limiting examples of alterations that can alter the Kcat of a variant Cas12i2 polypeptide are substitutions listed in Table 12. In some embodiments, a variant Cas12i2 polypeptide comprising one or more substitutions listed in Table 12 exhibits increased on-target specificity relative to a parent polypeptide. In some embodiments, a variant Cas12i2 polypeptide comprising one or more substitutions listed in Table 11 exhibits increased on-target binding relative to a parent polypeptide. In some embodiments, a variant Cas12i2 polypeptide comprising one or more substitutions listed in Table 12 exhibits increased on-target binding affinity relative to a parent polypeptide. In some embodiments, a variant Cas12i2 polypeptide comprising one or more substitutions listed in Table 12 exhibits decreased off-target specificity relative to a parent polypeptide. In some embodiments, a variant Cas12i2 polypeptide comprising one or more substitutions listed in Table 11 exhibits decreased off-target binding relative to a parent polypeptide. In some embodiments, a variant Cas12i2 polypeptide comprising one or more substitutions listed in Table 12 exhibits decreased off-target binding affinity relative to a parent polypeptide.

TABLE 12
Substitutions increasing on-target specificity.

| | |
|---|---|
| Q600 | A, S, T, V |
| Q602 | L, A, S, M |
| T603 | S, V, N, A |
| L836 | S, T, V, N, A |
| S837 | A, T, V, L, I |
| A1022 | S |
| I1026 | V, A, N |

In some embodiments, the substitution(s) that decrease the Kcat of the variant Cas12i2 polypeptide by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more or any percentage therebetween further increase on-target specificity by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more or any percentage therebetween and decrease off-target specificity by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, as compared to a parent polypeptide.

In some embodiments, the substitution(s) that decrease the Kcat of the variant Cas12i2 polypeptide by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more or any percentage therebetween further increase on-target binding by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more or any percentage therebetween and decrease off-target binding by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, as compared to a parent polypeptide.

In some embodiments, the substitution(s) that decrease the Kcat of the variant Cas12i2 polypeptide by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more or any percentage therebetween further increase on-target binding affinity by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more or any percentage therebetween and decrease off-target binding affinity by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, as compared to a parent polypeptide.

In some embodiments, the alteration that decreases the Kcat of the variant Cas12i2 polypeptide (e.g., a substitution listed in Table 12) further increases the ratio of on-target ternary complex formation to off-target ternary complex formation. In some embodiments, the alteration that decreases the Kcat increases the ratio of on-target ternary complex formation to off-target ternary complex formation by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more or any percentage therebetween as compared to a parent polypeptide.

In some embodiments, a variant Cas12i2 polypeptide of any one of SEQ ID NOs: 3-146 and 495-512 further comprises one or more substitutions listed in Table 12. In some embodiments, a variant Cas12i2 polypeptide comprises one or more substitutions listed in Table 2 and Table 12.

In some embodiments, the variant Cas12i2 polypeptide of any one of SEQ ID NOs: 3-146 and 495-512 comprising one or more substitutions listed in Table 4 and/or Table 5 and/or Table 6 and/or Table 7 and/or Table 8 and/or Table 9 and/or Table 10 and/or Table 11 and/or Table 12 exhibits increased on-target enzymatic activity. In some embodiments, the variant Cas12i2 polypeptide comprising one or more substitutions listed in Table 4 and/or Table 5 and/or Table 6 and/or Table 7 and/or Table 8 and/or Table 9 and/or Table 10 and/or Table 11 and/or Table 12 exhibits increased on-target enzymatic activity. In some embodiments, the variant Cas12i2 polypeptide exhibits increased on-target enzymatic activity (e.g., by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more or any percentage therebetween) as compared to a parent polypeptide.

In some embodiments, the variant Cas12i2 polypeptide of any one of SEQ ID NOs: 3-146 and 495-512 comprising one or more substitutions listed in Table 4 and/or Table 5 and/or Table 6 and/or Table 7 and/or Table 8 and/or Table 9 and/or Table 10 and/or Table 11 and/or Table 12 exhibits an increased ratio of on-target enzymatic activity to off-target enzymatic activity. In some embodiments, the variant Cas12i2 polypeptide comprising one or more substitutions listed in Table 4 and/or Table 5 and/or Table 6 and/or Table 7 and/or Table 8 and/or Table 9 and/or Table 10 and/or Table 11 and/or Table 12 exhibits an increased ratio of on-target enzymatic activity to off-target enzymatic activity. In some embodiments, on-target enzymatic activity of the variant Cas12i2 polypeptide (e.g., the variant Cas12i2 polypeptide of any one of SEQ ID NOs: 3-146 and 495-512 comprising one or more substitutions listed in Table 4 and/or Table 5 and/or Table 6 and/or Table 7 and/or Table 8 and/or Table 9 and/or Table 10 and/or Table 11 and/or Table 12) is at least 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more or any percentage therebetween higher than off-target enzymatic activity of the variant Cas12i2 polypeptide, as compared to a parent polypeptide. In some embodiments, on-target enzymatic activity of the variant Cas12i2 polypeptide (e.g., the variant Cas12i2 polypeptide comprising one or more substitutions listed in Table 4 and/or Table 5 and/or Table 6 and/or Table 7 and/or Table 8 and/or Table 9 and/or Table 10 and/or Table 11 and/or Table 12) is at least 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more or any percentage therebetween higher than off-target enzymatic activity of the variant Cas12i2 polypeptide, as compared to a parent polypeptide.

In some embodiments, enzymatic activity of the variant Cas12i2 polypeptide (e.g., the variant Cas12i2 polypeptide of any one of SEQ ID NOs: 3-146 and 495-512 comprising one or more substitutions listed in Table 4 and/or Table 5 and/or Table 6 and/or Table 7 and/or Table 8 and/or Table 9 and/or Table 10 and/or Table 11 and/or Table 12) at an off-target locus is no more than 10% (e.g., 10%, 9%, 8%, 7%, 6%, 5%, 4%, 3%, 2%, 1%, or 0%) of the enzymatic activity at the on-target locus. In some embodiments, enzymatic activity of the variant Cas12i2 polypeptide (e.g., the variant Cas12i2 polypeptide of any one of SEQ ID NOs: 3-146 and 495-512 comprising one or more substitutions listed in Table 4 and/or Table 5 and/or Table 6 and/or Table 7 and/or Table 8 and/or Table 9 and/or Table 10 and/or Table 11) at an off-target locus is no more than 5% (e.g., 5%, 4%, 3%, 2%, 1%, or 0%) of the enzymatic activity at the on-target locus. By comparison, enzymatic activity of SpCas9 at an off-target locus is up to 95% (e.g., 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, or 0%) of the enzymatic activity at the on-target locus. See Example 16.

In some embodiments, editing efficiency of the variant Cas12i2 polypeptide (e.g., the variant Cas12i2 polypeptide of any one of SEQ ID NOs: 3-146 and 495-512 comprising one or more substitutions listed in Table 4 and/or Table 5 and/or Table 6 and/or Table 7 and/or Table 8 and/or Table 9 and/or Table 10 and/or Table 11 and/or Table 12) at an off-target locus is no more than 10% (e.g., 10%, 9%, 8%, 7%, 6%, 5%, 4%, 3%, 2%, 1%, or 0%) of the editing efficiency at the on-target locus. In some embodiments, editing efficiency of the variant Cas12i2 polypeptide (e.g., the variant Cas12i2 polypeptide of any one of SEQ ID NOs: 3-146 and 495-512 comprising one or more substitutions listed in Table 4 and/or Table 5 and/or Table 6 and/or Table 7 and/or Table 8 and/or Table 9 and/or Table 10 and/or Table 11) at an off-target locus is no more than 5% (e.g., 5%, 4%, 3%, 2%, 1%, or 0%) of the editing efficiency at the on-target locus. By comparison, editing efficiency of SpCas9 at an off-target locus is up to 95% (e.g., 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, or 0%) of the editing efficiency at the on-target locus. See Example 16.

In some embodiments, editing by the variant Cas12i2 polypeptide (e.g., the variant Cas12i2 polypeptide of any one of SEQ ID NOs: 3-146 and 495-512 comprising one or more substitutions listed in Table 4 and/or Table 5 and/or Table 6 and/or Table 7 and/or Table 8 and/or Table 9 and/or Table 10 and/or Table 11 and/or Table 12) at an off-target locus is no more than 10% (e.g., 10%, 9%, 8%, 7%, 6%, 5%, 4%, 3%, 2%, 1%, or 0%) of the editing at the on-target locus. In some embodiments, editing by the variant Cas12i2 polypeptide (e.g., the variant Cas12i2 polypeptide of any one of SEQ ID NOs: 3-146 and 495-512 comprising one or more substitutions listed in Table 4 and/or Table 5 and/or Table 6 and/or Table 7 and/or Table 8 and/or Table 9 and/or Table 10 and/or Table 11) at an off-target locus is no more than 5% (e.g., 5%, 4%, 3%, 2%, 1%, or 0%) of the editing at the on-target locus. By comparison, editing of SpCas9 at an off-target locus is up to 95% (e.g., 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, or 0%) of the editing at the on-target locus. See Example 16.

### Targeting Moiety

In some embodiments, a composition or complex as described herein comprises a targeting moiety (e.g., an RNA guide, antisense, oligonucleotides, peptide oligonucleotide conjugates) that binds the target nucleic acid and interacts with the variant Cas12i2 polypeptide. The targeting moiety may bind a target nucleic acid (e.g., with specific binding affinity to the target nucleic acid). In some embodiments, a composition described herein comprises two or more targeting moieties, e.g., two or more RNA guides (e.g., 2, 3, 4, 5, 6, 7, 8, 9, or more).

In some embodiments, the targeting moiety comprises, or is, an RNA guide. In some embodiments, the RNA guide directs the variant Cas12i2 polypeptide described herein to a particular nucleic acid sequence. Those skilled in the art reading the below examples of particular kinds of RNA guides will understand that, in some embodiments, an RNA guide is site-specific. That is, in some embodiments, an RNA guide associates specifically with one or more target nucleic acid sequences (e.g., specific DNA or genomic DNA sequences) and not to non-targeted nucleic acid sequences (e.g., non-specific DNA or random sequences). In the case of two or more guides within a composition, the two or more guides may target two or more separate variant Cas12i2 polypeptides (e.g., variant Cas12i2 polypeptides having the same or different sequence) as described herein to two or more (e.g., 2, 3, 4, 5, 6, 7, 8, 9, or more) target nucleic acids or two or more (e.g., 2, 3, 4, 5, 6, 7, 8, 9, or more) target loci of a target nucleic acid.

In some embodiments, the composition as described herein comprises an RNA guide that associates with the variant Cas12i2 polypeptide described herein and directs the variant Cas12i2 polypeptide to a target nucleic acid sequence (e.g., DNA).

The RNA guide may target (e.g., associate with, be directed to, contact, or bind) one or more nucleotides of a target sequence, e.g., a site-specific sequence or a site-specific target. In some embodiments, the variant effector nucleoprotein (e.g., variant Cas12i2 polypeptide plus an RNA guide) is activated upon binding to a target nucleic acid that is complementary to a DNA-targeting sequence in the RNA guide (e.g., a sequence-specific substrate or target nucleic acid).

In some embodiments, an RNA guide comprises a spacer having a length of from about 7 nucleotides to about 100 nucleotides. For example, the DNA-targeting segment can have a length of from about 7 nucleotides to about 80 nucleotides, from about 7 nucleotides to about 50 nucleotides, from about 7 nucleotides to about 40 nucleotides, from about 7 nucleotides to about 30 nucleotides, from about 7 nucleotides to about 25 nucleotides, from about 7 nucleotides to about 20 nucleotides, or from about 7 nucleotides to about 19 nucleotides. For example, the spacer can have a length of from about 7 nucleotides to about 20 nucleotides, from about 7 nucleotides to about 25 nucleotides, from about 7 nucleotides to about 30 nucleotides, from about 7 nucleotides to about 35 nucleotides, from about 7 nucleotides to about 40 nucleotides, from about 7 nucleotides to about 45 nucleotides, from about 7 nucleotides to about 50 nucleotides, from about 7 nucleotides to about 60 nucleotides, from about 7 nucleotides to about 70 nucleotides, from about 7 nucleotides to about 80 nucleotides, from about 7 nucleotides to about 90 nucleotides, from about 7 nucleotides to about 100 nucleotides, from about 10 nucleotides to about 25 nucleotides, from about 10 nucleotides to about 30 nucleotides, from about 10 nucleotides to about 35 nucleotides, from about 10 nucleotides to about 40 nucleotides, from about 10 nucleotides to about 45 nucleotides, from about 10 nucleotides to about 50 nucleotides, from about 10 nucleotides to about 60 nucleotides, from about 10 nucleotides to about 70 nucleotides, from about 10 nucleotides to about 80 nucleotides, from about 10 nucleotides to about 90 nucleotides, or from about 10 nucleotides to about 100 nucleotides.

In some embodiments, the spacer of the RNA guide may be generally designed to have a length of between 7 and 50 nucleotides or between 7 and 30 nucleotides (e.g., 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides) and be complementary to a specific target nucleic acid sequence. In some particular embodiments, the RNA guide may be designed to be complementary to a specific DNA strand, e.g., of a genomic locus. In some embodiments, the DNA targeting sequence is designed to be complementary to a specific DNA strand, e.g., of a genomic locus.

The RNA guide may be substantially identical to a complementary strand of a reference nucleic acid sequence. In some embodiments, the RNA guide comprises a sequence having least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to a complementary strand of a reference nucleic acid sequence, e.g., target nucleic acid. The percent identity between two such nucleic acids can be determined manually by inspection of the two optimally aligned nucleic acid sequences or by using software programs or algorithms (e.g., BLAST, ALIGN, CLUSTAL) using standard parameters.

In some embodiments, the RNA guide has at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to a complementary strand of a target nucleic acid.

In some embodiments, the RNA guide comprises a spacer that is a length of between 7 and 50 nucleotides (e.g., 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides) and at least 80%, at least 90%, at least 95%, at least 96%, at least 97%, at least 98%, at least 99% complementary to a target nucleic acid. In some embodiments, the RNA guide comprises a sequence at least 80%, at least 90%, at least 95%, at least 96%, at least 97%, at least 98%, at least 99% complementary to a target DNA sequence. In some embodiments, the RNA guide comprises a sequence at least 80%, at least 90%, at least 95%, at least 96%, at least 97%, at least 98%, at least 99% complementary to a target genomic sequence. In some embodiments, the RNA guide comprises a sequence, e.g., RNA sequence, that is a length of up to 50 and at least 80%, at least 90%, at least 95%, at least 96%, at least 97%, at least 98%, at least 99% complementary to a target nucleic acid. In some embodiments, the RNA guide comprises a sequence at least 80%, at least 90%, at least 95%, at least 96%, at least 97%, at least 98%, at least 99% complementary to a target DNA sequence. In some embodiments, the RNA guide comprises a sequence at least 80%, at least 90%, at least 95%, at least 96%, at least 97%, at least 98%, at least 99% complementary to a target genomic sequence.

In certain embodiments, the RNA guide includes, consists essentially of, or comprises a direct repeat sequence linked to a DNA targeting sequence. In some embodiments, the RNA guide includes a direct repeat sequence and a DNA targeting sequence or a direct repeat-DNA targeting sequence-direct repeat sequence. In some embodiments, the RNA guide includes a truncated direct repeat sequence and a DNA targeting sequence, which is typical of processed or mature crRNA. In some embodiments, the variant Cas12i2 polypeptide described herein forms a complex with the RNA guide, and the RNA guide directs the complex to associate with site-specific target nucleic acid that is complementary to at least a portion of the RNA guide.

In some embodiments, the direct repeat sequence is at least 90% identical to a sequence of Table 13 or a portion of a sequence of Table 13. In some embodiments, the direct repeat sequence is at least 95% identical to a sequence of Table 13 or a portion of a sequence of Table 13. In some embodiments, the direct repeat sequence is a sequence of Table 13 or a portion of a sequence of Table 13.

In some embodiments, the direct repeat sequence of SEQ ID NO: 492 is a mature (e.g., processed) direct repeat sequence. In some embodiments, a crRNA (e.g., mature crRNA) comprises the direct repeat of SEQ ID NO: 492 and a spacer. In some embodiments, the direct repeat sequences of SEQ ID NOs: 493 and 494 are unprocessed direct repeat sequences. In some embodiments, a crRNA (e.g., pre-crRNA) comprises a first direct repeat of SEQ ID NO: 493, a spacer, and a second direct repeat sequence of SEQ ID NO: 493 (e.g., the two direct repeat sequences flank the spacer). In some embodiments, a crRNA (e.g., pre-crRNA) comprises a first direct repeat of SEQ ID NO: 494, a spacer, and a second direct repeat sequence of SEQ ID NO: 494 (e.g., the two direct repeat sequences flank the spacer).

TABLE 13
Direct repeat sequences.

| Sequence | |
|---|---|
| identifier | Direct Repeat Sequence |
| SEQ ID NO: 493 | GUUGCAAAACCCAAGAAAUCCGUCU |
| | UUCAUUGACGG |
| SEQ ID NO: 494 | GCAACACCUAAGAAAUCCGUCUUUC |
| | AUUGACGGG |
| SEQ ID NO: 492 | AGAAAUCCGUCUUUCAUUGACGG |

In some embodiments, PAMs corresponding to variant Cas12i2 polypeptide of the present invention include 5′-NTTN-3′, wherein N is any nucleotide. In some embodiments, the PAM comprises the 5′-TTH-3′, 5′-TTY-3′, or 5′-TTC-3, wherein N is any nucleotide, H is A, C, or T and Y is C or T. In some embodiments, the PAM comprises 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3, wherein B is any nucleotide except for A, D is any nucleotide except for C, and R is A or G. In some embodiments, the PAM comprises 5′-CTTT-3′, 5′-CTTC-3′, 5′-GTTT-3′, 5′-GTTC-3′, 5′-TTTC-3′, 5′-GTTA-3′, or 5′-GTTG-3′.

In some embodiments, the composition or complex described herein includes one or more (e.g., two, three, four, five, six, seven, eight, or more) RNA guides, e.g., a plurality of RNA guides.

In some embodiments, the RNA guide has an architecture similar to, for example International Publication Nos. WO 2014/093622 and WO 2015/070083, the entire contents of each of which are incorporated herein by reference.

### Modifications

The RNA guide or any of the nucleic acid sequences encoding the variant Cas12i2 polypeptides may include one or more covalent modifications with respect to a reference sequence, in particular the parent polyribonucleotide, which are included within the scope of this invention.

Exemplary modifications can include any modification to the sugar, the nucleobase, the internucleoside linkage (e.g. to a linking phosphate/to a phosphodiester linkage/to the phosphodiester backbone), and any combination thereof. Some of the exemplary modifications provided herein are described in detail below.

The RNA guide or any of the nucleic acid sequences encoding components of the variant Cas12i2 polypeptides may include any useful modification, such as to the sugar, the nucleobase, or the internucleoside linkage (e.g. to a linking phosphate/to a phosphodiester linkage/to the phosphodiester backbone). One or more atoms of a pyrimidine nucleobase may be replaced or substituted with optionally substituted amino, optionally substituted thiol, optionally substituted alkyl (e.g., methyl or ethyl), or halo (e.g., chloro or fluoro). In certain embodiments, modifications (e.g., one or more modifications) are present in each of the sugar and the internucleoside linkage. Modifications may be modifications of ribonucleic acids (RNAs) to deoxyribonucleic acids (DNAs), threose nucleic acids (TNAs), glycol nucleic acids (GNAs), peptide nucleic acids (PNAs), locked nucleic acids (LNAs) or hybrids thereof). Additional modifications are described herein.

In some embodiments, the modification may include a chemical or cellular induced modification. For example, some nonlimiting examples of intracellular RNA modifications are described by Lewis and Pan in “RNA modifications and structures cooperate to guide RNA-protein interactions” from Nat Reviews Mol Cell Biol, 2017, 18:202-210.

Different sugar modifications, nucleotide modifications, and/or internucleoside linkages (e.g., backbone structures) may exist at various positions in the sequence. One of ordinary skill in the art will appreciate that the nucleotide analogs or other modification(s) may be located at any position(s) of the sequence, such that the function of the sequence is not substantially decreased. The sequence may include from about 1% to about 100% modified nucleotides (either in relation to overall nucleotide content, or in relation to one or more types of nucleotide, i.e. any one or more of A, G, U or C) or any intervening percentage (e.g., from 1% to 20%>, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%).

In some embodiments, sugar modifications (e.g., at the 2′ position or 4′ position) or replacement of the sugar at one or more ribonucleotides of the sequence may, as well as backbone modifications, include modification or replacement of the phosphodiester linkages. Specific examples of a sequence include, but are not limited to, sequences including modified backbones or no natural internucleoside linkages such as internucleoside modifications, including modification or replacement of the phosphodiester linkages. Sequences having modified backbones include, among others, those that do not have a phosphorus atom in the backbone. For the purposes of this application, and as sometimes referenced in the art, modified RNAs that do not have a phosphorus atom in their internucleoside backbone can also be considered to be oligonucleosides. In particular embodiments, a sequence will include ribonucleotides with a phosphorus atom in its internucleoside backbone.

Modified sequence backbones may include, for example, phosphorothioates, chiral phosphorothioates, phosphorodithioates, phosphotriesters, aminoalkylphosphotriesters, methyl and other alkyl phosphonates such as 3′-alkylene phosphonates and chiral phosphonates, phosphinates, phosphoramidates such as 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included. In some embodiments, the sequence may be negatively or positively charged.

The modified nucleotides, which may be incorporated into the sequence, can be modified on the internucleoside linkage (e.g., phosphate backbone). Herein, in the context of the polynucleotide backbone, the phrases “phosphate” and “phosphodiester” are used interchangeably. Backbone phosphate groups can be modified by replacing one or more of the oxygen atoms with a different substituent. Further, the modified nucleosides and nucleotides can include the wholesale replacement of an unmodified phosphate moiety with another internucleoside linkage as described herein. Examples of modified phosphate groups include, but are not limited to, phosphorothioate, phosphoroselenates, boranophosphates, boranophosphate esters, hydrogen phosphonates, phosphoramidates, phosphorodiamidates, alkyl or aryl phosphonates, and phosphotriesters. Phosphorodithioates have both non-linking oxygens replaced by sulfur. The phosphate linker can also be modified by the replacement of a linking oxygen with nitrogen (bridged phosphoramidates), sulfur (bridged phosphorothioates), and carbon (bridged methylene-phosphonates).

The α-thio substituted phosphate moiety is provided to confer stability to RNA and DNA polymers through the unnatural phosphorothioate backbone linkages. Phosphorothioate DNA and RNA have increased nuclease resistance and subsequently a longer half-life in a cellular environment.

In specific embodiments, a modified nucleoside includes an alpha-thio-nucleoside (e.g., 5′-O-(1-thiophosphate)-adenosine, 5′-O-(1-thiophosphate)-cytidine (a-thio-cytidine), 5′-O-(1-thiophosphate)-guanosine, 5′-O-(1-thiophosphate)-uridine, or 5′-O-(1-thiophosphate)-pseudouridine).

Other internucleoside linkages that may be employed according to the present invention, including internucleoside linkages which do not contain a phosphorous atom, are described herein.

In some embodiments, the sequence may include one or more cytotoxic nucleosides. For example, cytotoxic nucleosides may be incorporated into sequence, such as bifunctional modification. Cytotoxic nucleoside may include, but are not limited to, adenosine arabinoside, 5-azacytidine, 4′-thio-aracytidine, cyclopentenylcytosine, cladribine, clofarabine, cytarabine, cytosine arabinoside, 1-(2-C-cyano-2-deoxy-beta-D-arabino-pentofuranosyl)-cytosine, decitabine, 5-fluorouracil, fludarabine, floxuridine, gemcitabine, a combination of tegafur and uracil, tegafur ((RS)-5-fluoro-1-(tetrahydrofuran-2-yl)pyrimidine-2,4(1H,3H)-dione), troxacitabine, tezacitabine, 2′-deoxy-2′-methylidenecytidine (DMDC), and 6-mercaptopurine. Additional examples include fludarabine phosphate, N4-behenoyl-1-beta-D-arabinofuranosylcytosine, N4-octadecyl-1-beta-D-arabinofuranosylcytosine, N4-palmitoyl-1-(2-C-cyano-2-deoxy-beta-D-arabino-pentofuranosyl) cytosine, and P-4055 (cytarabine 5′-elaidic acid ester).

In some embodiments, the sequence includes one or more post-transcriptional modifications (e.g., capping, cleavage, polyadenylation, splicing, poly-A sequence, methylation, acylation, phosphorylation, methylation of lysine and arginine residues, acetylation, and nitrosylation of thiol groups and tyrosine residues, etc.). The one or more post-transcriptional modifications can be any post-transcriptional modification, such as any of the more than one hundred different nucleoside modifications that have been identified in RNA (Rozenski, J, Crain, P, and McCloskey, J. (1999). The RNA Modification Database: 1999 update. Nucl Acids Res 27: 196-197) In some embodiments, the first isolated nucleic acid comprises messenger RNA (mRNA). In some embodiments, the mRNA comprises at least one nucleoside selected from the group consisting of pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine. In some embodiments, the mRNA comprises at least one nucleoside selected from the group consisting of 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine. In some embodiments, the mRNA comprises at least one nucleoside selected from the group consisting of 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine. In some embodiments, mRNA comprises at least one nucleoside selected from the group consisting of inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

The sequence may or may not be uniformly modified along the entire length of the molecule. For example, one or more or all types of nucleotide (e.g., naturally-occurring nucleotides, purine or pyrimidine, or any one or more or all of A, G, U, C, I, pU) may or may not be uniformly modified in the sequence, or in a given predetermined sequence region thereof. In some embodiments, the sequence includes a pseudouridine. In some embodiments, the sequence includes an inosine, which may aid in the immune system characterizing the sequence as endogenous versus viral RNAs. The incorporation of inosine may also mediate improved RNA stability/reduced degradation. See for example, Yu, Z. et al. (2015) RNA editing by ADAR1 marks dsRNA as “self”. Cell Res. 25, 1283-1284, which is incorporated by reference in its entirety.

### Target Nucleic Acid

The methods disclosed herein are applicable for a variety of target nucleic acids. In some embodiments, the target nucleic acid is a DNA, such as a DNA locus. In some embodiments, the target nucleic acid is in a nucleus of a cell. In some embodiments, the target nucleic acid is a genomic DNA locus. In some embodiments, the target nucleic acid is single-stranded (e.g., single-stranded DNA). In some embodiments, the target nucleic acid is double-stranded (e.g., double-stranded DNA). In some embodiments, the target nucleic acid comprises both single-stranded and double-stranded regions. In some embodiments, the target nucleic acid is linear. In some embodiments, the target nucleic acid is circular. In some embodiments, the target nucleic acid comprises one or more modified nucleotides, such as methylated nucleotides, damaged nucleotides, or nucleotides analogs. In some embodiments, the target nucleic acid is not modified.

In some embodiments, the composition described herein includes one or more (e.g., two, three, four, five, six, seven, eight, or more) target nucleic acids, e.g., a plurality of target nucleic acids. In some embodiments, the composition described herein includes one or more (e.g., two, three, four, five, six, seven, eight, or more) target loci of a target nucleic acid, e.g., a plurality of target loci.

The target nucleic acid may be of any length, such as about at least any one of 10 bp, 20 bp, 30 bp, 40 bp, 50 bp, 60 bp, 70 bp, 80 bp, 90 bp, 100 bp, 200 bp, 500 bp, 1000 bp, 2000 bp, 5000 bp, 10 kb, 20 kb, 50 kb, 100 kb, 200 kb, 500 kb, 1 Mb, or longer or any length inbetween. The target nucleic acid may also comprise any sequence. In some embodiments, the target nucleic acid is GC-rich, such as having at least about any one of 40%, 45%, 50%, 55%, 60%, 65%, or higher GC content. In some embodiments, the target nucleic acid has a GC content of at least about 70%, 80%, or more. In some embodiments, the target nucleic acid is a GC-rich fragment in a non-GC-rich target nucleic acid. In some embodiments, the target nucleic acid is not GC-rich. In some embodiments, the target nucleic acid has one or more secondary structures or higher-order structures. In some embodiments, the target nucleic acid is not in a condensed state, such as in a chromatin, to render the target nucleic acid inaccessible by the variant Cas12i2 polypeptide/RNA guide complex.

In some embodiments, the target nucleic acid is present in a cell. In some embodiments, the target nucleic acid is present in the nucleus of the cell. In some embodiments, the target nucleic acid is endogenous to the cell. In some embodiments, the target nucleic acid is a genomic DNA. In some embodiments, the target nucleic acid is a chromosomal DNA. In some embodiments, the target nucleic acid is a protein-coding gene or a functional region thereof, such as a coding region, or a regulatory element, such as a promoter, enhancer, a 5′ or 3′ untranslated region, etc. In some embodiments, the target nucleic acid is a non-coding gene, such as transposon, miRNA, tRNA, ribosomal RNA, ribozyme, or lincRNA. In some embodiments, the target nucleic acid is a plasmid.

In some embodiments, the target nucleic acid is exogenous to a cell. In some embodiments, the target nucleic acid is a viral nucleic acid, such as viral DNA or viral RNA. In some embodiments, the target nucleic acid is a horizontally transferred plasmid. In some embodiments, the target nucleic acid is integrated in the genome of the cell. In some embodiments, the target nucleic acid is not integrated in the genome of the cell. In some embodiments, the target nucleic acid is a plasmid in the cell. In some embodiments, the target nucleic acid is present in an extrachromosomal array.

In some embodiments, the target nucleic acid is an isolated nucleic acid, such as an isolated DNA or an isolated RNA. In some embodiments, the target nucleic acid is present in a cell-free environment. In some embodiments, the target nucleic acid is an isolated vector, such as a plasmid. In some embodiments, the target nucleic acid is an ultrapure plasmid.

In some embodiments, a target sample may include a segment of the target nucleic acid that hybridizes to at least a portion of the targeting moiety (e.g., RNA guide). In some embodiments, the target sample has only one copy of the target nucleic acid. In some embodiments, the target sample has more than one copy, such as at least about any one of 2, 3, 4, 5, 10, 100, or more copies of the target nucleic acid. For example, a target sample comprising a repeated sequence (e.g., target nucleic acid sequence) in a genome of a virus or a bacterium and the repeated sequence may be targeted by at least a portion of the targeting moiety.

In some embodiments, the target nucleic acid is present in a readily accessible region of the nucleic acid. In some embodiments, the target nucleic acid is in an exon of a gene. In some embodiments, the target nucleic acid is across an exon-intron junction of a gene. In some embodiments, the target nucleic acid is present in a non-coding region, such as a regulatory region of a gene. In some embodiments, wherein the target nucleic acid is exogenous to a cell, the target nucleic acid comprises a sequence that is not found in the genome of the cell.

Suitable DNA/RNA binding conditions include physiological conditions normally present in a cell. Other suitable DNA/RNA binding conditions (e.g., conditions in a cell-free system) are known in the art; see, e.g., Sambrook, supra. The strand of the target nucleic acid that is complementary to and hybridizes with the targeting moiety (e.g., RNA guide) is referred to as the “complementary strand” and the strand of the target nucleic acid that is complementary to the “complementary strand” (and is therefore not complementary to the RNA guide) is referred to as the “noncomplementary strand” or “non-complementary strand”.

### Binary Complex

In some aspects, a Cas12i2 polypeptide and the targeting moiety form a complex. In some embodiments, a Cas12i2 polypeptide, e.g., a variant Cas12i2 polypeptide, and a targeting moiety, e.g., an RNA guide as described herein, form a complex (e.g., a binary complex, e.g., a ribonucleoprotein or RNP). In some embodiments, a Cas12i2 polypeptide and an RNA guide form a binary complex.

A binary complex described herein, in some embodiments, comprises a variant Cas12i2 polypeptide that associates with at least one RNA guide, wherein each RNA guide targets a target nucleic acid such as DNA. In some embodiments, the variant Cas12i2 polypeptide/RNA guide complex (e.g., variant binary complex) comprises enzymatic activity, such as nuclease activity, that may nick or cleave the target nucleic acid. The variant Cas12i2 polypeptide and the RNA guide, either alone or together, do not naturally occur. Complex formation between the variant Cas12i2 polypeptide and the RNA guide can enhance stability and/or protein-RNA interactions between the two, as compared to a parent polypeptide and RNA guide.

Generally, the variant Cas12i2 polypeptide and the targeting moiety (e.g., RNA guide) bind to each other in a molar ratio of about 1:1 to form the variant binary complex. Binding of the variant Cas12i2 polypeptide and the targeting moiety (e.g., RNA guide) to form the variant binary complex is referred to as loading the RNA guide to the polypeptide.

In some embodiments, the binary complex follows a one-guide rule, i.e., the variant Cas12i2 polypeptide does not dissociate from the bound RNA guide in the complex, or switch RNA guide with a free, unbound RNA. In some embodiments, the ternary complex follows a one-binary complex rule, i.e., the variant binary complex does not dissociate from the bound target nucleic acid (e.g., target DNA substrate) or switch the target nucleic acid with a free, unbound nucleic acid.

### Functionality of Binary Complexes

In some aspects, the variant binary complex comprises a variant Cas12i2 polypeptide with at least one alteration or mutation that enhances at least one of enzymatic activity, target nucleic acid complex formation, target nucleic acid binding activity, target nucleic acid affinity, target nucleic acid binding specificity, protein-nucleic acid interactions, ternary complex formation, on-target binding activity, on-target binding specificity, and/or stability.

In some aspects, the variant binary complex comprises a variant Cas12i2 polypeptide with at least one alteration or mutation and the variant binary complex has decreased at least one of off-target binding to a non-target nucleic acid, activity at a non-target locus of a target nucleic acid, binary complex dissociation, dissociation from the target nucleic acid.

In some aspects, a variant Cas12i2 polypeptide and a targeting moiety (e.g., RNA guide) form a variant binary complex, and the variant binary complex exhibits increased at least one of enzymatic activity, target nucleic acid complex formation, target nucleic acid binding activity, target nucleic acid affinity, target nucleic acid binding specificity, protein-nucleic acid interactions, ternary complex formation, on-target binding activity, on-target binding specificity, and/or stability, as compared to a parent binary complex.

In some embodiments, the variant binary complex comprises a variant Cas12i2 polypeptide with at least one alteration or mutation that increases at least one of enzymatic activity, target nucleic acid complex formation, target nucleic acid binding activity, target nucleic acid affinity, target nucleic acid binding specificity, protein-nucleic acid interactions, ternary complex formation, on-target binding activity, on-target binding specificity, and/or stability of the variant binary complex at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% greater than the enzymatic activity, target nucleic acid complex formation, target nucleic acid binding activity, target nucleic acid affinity, target nucleic acid binding specificity, protein-nucleic acid interactions, ternary complex formation, on-target binding activity, on-target binding specificity, and/or stability of a parent binary complex.

In some embodiments, the variant binary complex exhibits enhanced at least one of enzymatic activity, target nucleic acid complex formation, target nucleic acid binding activity, target nucleic acid affinity, target nucleic acid binding specificity, protein-nucleic acid interactions, ternary complex formation, on-target binding activity, on-target binding specificity, and/or stability, as compared to a parent binary complex, at a temperature in the range of about 20° C. to about 65° C., e.g., about any one of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 50° C., 51° C., 52° C., 53° C., 54° C., 55° C., 56° C., 57° C., 58° C., 59° C., 60° C. or 65° C.

In some embodiments, the variant binary complex exhibits enhanced at least one of enzymatic activity, target nucleic acid complex formation, target nucleic acid binding activity, target nucleic acid affinity, target nucleic acid binding specificity, protein-nucleic acid interactions, ternary complex formation, on-target binding activity, on-target binding specificity, and/or stability, as compared to a parent binary complex, in a buffer having a pH in a range of about 7.3 to about 8.6 (e.g., 7.3, 7.4, 7.5, 7.6, 7.7, 7.8, 7.9, 8.0, 8.1, 8.2, 8.3, 8.4, 8.5, 8.6, or any value within a range between any combination of these values).

In some embodiments, the variant binary complex exhibits enhanced at least one of enzymatic activity, target nucleic acid complex formation, target nucleic acid binding activity, target nucleic acid affinity, target nucleic acid binding specificity, protein-nucleic acid interactions, ternary complex formation, on-target binding activity, on-target binding specificity, and/or stability, as compared to a parent binary complex, when the Tₘ value of the variant binary complex is at least 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., or 20° C. greater than the Tₘ value of a parent binary complex. In one embodiment, the variant binary complex exhibits enhanced at least one of enzymatic activity, target nucleic acid complex formation, target nucleic acid binding activity, target nucleic acid affinity, target nucleic acid binding specificity, protein-nucleic acid interactions, ternary complex formation, on-target binding activity, on-target binding specificity, and/or stability when the Tₘ value of the variant binary complex is at least 8° C. greater than the Tₘ value of the parent binary complex.

In some embodiments, the variant binary complex exhibits increased at least one of enzymatic activity, target nucleic acid complex formation, target nucleic acid binding activity, target nucleic acid affinity, target nucleic acid binding specificity, protein-nucleic acid interactions, ternary complex formation, on-target binding activity, on-target binding specificity, and/or stability at about 37° C. over an incubation period of at least about any one of 10 mins, 15 mins, 20 mins, 25 mins, 30 mins, 35 mins, 40 mins, 45 mins, 50 mins, 55 mins, 1 hr, 2 hr, 3 hr, 4 hr, or more hours as compared to a parent binary complex. In some embodiments, a variant binary complex exhibits increased at least one of enzymatic activity, target nucleic acid complex formation, target nucleic acid binding activity, target nucleic acid affinity, target nucleic acid binding specificity, protein-nucleic acid interactions, ternary complex formation, on-target binding activity, on-target binding specificity, and/or stability over a range of incubation times as compared to a parent binary complex.

In some embodiments, the variant binary complex comprises a variant Cas12i2 polypeptide with at least one alteration or mutation that increases at least one of enzymatic activity, target nucleic acid complex formation, target nucleic acid binding activity, target nucleic acid affinity, target nucleic acid binding specificity, protein-nucleic acid interactions, ternary complex formation, on-target binding activity, on-target binding specificity, and/or stability of the variant binary complex and the variant binary complex comprises a variant Cas12i2 polypeptide comprising an amino acid sequence having at least about 60%, 65%, 70%, 75%, 80%, 85%, 90%, 91%, 92%, 93%, 94%, 95%, 96%, 97%, 98%, 99%, or 100% identity to SEQ ID NO: 4.

In some embodiments, the variant binary complex comprises enhanced at least one of enzymatic activity, target nucleic acid complex formation, target nucleic acid binding activity, target nucleic acid affinity, target nucleic acid binding specificity, protein-nucleic acid interactions, ternary complex formation, on-target binding activity, on-target binding specificity, and/or stability and the variant binary complex comprises a variant Cas12i2 polypeptide comprising an amino acid sequence having at least about 60%, 65%, 70%, 75%, 80%, 85%, 90%, 91%, 92%, 93%, 94%, 95%, 96%, 97%, 98%, 99%, or 100% identity to any one of SEQ ID NOs: 3-5, 495, or 496.

In some embodiments, the variant binary complex comprises enhanced at least one of enzymatic activity, target nucleic acid complex formation, target nucleic acid binding activity, target nucleic acid affinity, target nucleic acid binding specificity, protein-nucleic acid interactions, ternary complex formation, on-target binding activity, on-target binding specificity, and/or stability and the variant binary complex comprises a variant Cas12i2 polypeptide comprising an amino acid sequence having at least about 60%, 65%, 70%, 75%, 80%, 85%, 90%, 91%, 92%, 93%, 94%, 95%, 96%, 97%, 98%, 99%, or 100% identity to any one of SEQ ID NOs: 3-146 and 495-512.

In some aspects, a variant Cas12i2 polypeptide and a targeting moiety (e.g., RNA guide) form a variant binary complex, and the variant binary complex exhibits decreased at least one of off-target binding to a non-target nucleic acid, activity at a non-target locus of a target nucleic acid, binary complex dissociation, and/or dissociation from the target nucleic acid, as compared to a parent binary complex. In some embodiments, the variant binary complex exhibits decreased at least one of off-target binding to a non-target nucleic acid, activity at a non-target locus of a target nucleic acid, binary complex dissociation, and/or dissociation from the target nucleic acid may be at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% less than the off-target binding to a non-target nucleic acid, activity at a non-target locus of a target nucleic acid, binary complex dissociation, and/or dissociation from the target nucleic acid of a parent binary complex.

In some embodiments, the variant binary complex exhibits decreased at least one of off-target binding to a non-target nucleic acid, activity at a non-target locus of a target nucleic acid, binary complex dissociation, and/or dissociation from the target nucleic acid at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% less than the off-target binding to a non-target nucleic acid, activity at a non-target locus of a target nucleic acid, binary complex dissociation, and/or dissociation from the target nucleic acid of a parent binary complex.

In some embodiments, the variant binary complex exhibits decreased at least one of off-target binding to a non-target nucleic acid, activity at a non-target locus of a target nucleic acid, binary complex dissociation, and/or dissociation from the target nucleic acid, as compared to a parent binary complex, at a temperature in the range of about 20° C. to about 65° C., e.g., about any one of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 50° C., 51° C., 52° C., 53° C., 54° C., 55° C., 56° C., 57° C., 58° C., 59° C., 60° C. or 65° C.

In some embodiments, the variant binary complex exhibits decreased at least one of off-target binding to a non-target nucleic acid, activity at a non-target locus of a target nucleic acid, binary complex dissociation, and/or dissociation from the target nucleic acid, as compared to a parent binary complex, in a buffer having a pH in a range of about 7.3 to about 8.6 (e.g., 7.3, 7.4, 7.5, 7.6, 7.7, 7.8, 7.9, 8.0, 8.1, 8.2, 8.3, 8.4, 8.5, 8.6, or any value within a range between any combination of these values).

In some embodiments, the variant binary complex exhibits decreased at least one of off-target binding to a non-target nucleic acid, activity at a non-target locus of a target nucleic acid, binary complex dissociation, and/or dissociation from the target nucleic acid, as compared to a parent binary complex, when the Tₘ value of the variant binary complex is at least 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., or 20° C. greater than the Tₘ value of a parent binary complex. In one embodiment, the variant binary complex exhibits at least one of decreased off-target binding to a non-target nucleic acid, activity at a non-target locus of a target nucleic acid, binary complex dissociation, and/or dissociation from the target nucleic acid when the Tₘ value of the variant binary complex is at least 8° C. greater than the Tₘ value of the parent binary complex.

In some embodiments, the variant binary complex comprises decreased at least one of off-target binding to a non-target nucleic acid, activity at a non-target locus of a target nucleic acid, binary complex dissociation, and/or dissociation from the target nucleic acid and the variant binary complex comprises a variant Cas12i2 polypeptide comprising an amino acid sequence having at least about 60%, 65%, 70%, 75%, 80%, 85%, 90%, 91%, 92%, 93%, 94%, 95%, 96%, 97%, 98%, 99%, or 100% identity to any one of SEQ ID NOs: 4.

In some embodiments, the variant binary complex comprises decreased at least one of off-target binding to a non-target nucleic acid, activity at a non-target locus of a target nucleic acid, binary complex dissociation, and/or dissociation from the target nucleic acid and the variant binary complex comprises a variant Cas12i2 polypeptide comprising an amino acid sequence having at least about 60%, 65%, 70%, 75%, 80%, 85%, 90%, 91%, 92%, 93%, 94%, 95%, 96%, 97%, 98%, 99%, or 100% identity to any one of SEQ ID NOs: 3-5, 495, or 496.

In some embodiments, the variant binary complex comprises decreased at least one of off-target binding to a non-target nucleic acid, activity at a non-target locus of a target nucleic acid, binary complex dissociation, and/or dissociation from the target nucleic acid and the variant binary complex comprises a variant Cas12i2 polypeptide comprising an amino acid sequence having at least about 60%, 65%, 70%, 75%, 80%, 85%, 90%, 91%, 92%, 93%, 94%, 95%, 96%, 97%, 98%, 99%, or 100% identity to any one of SEQ ID NOs: 3-146 and 495-512.

In some embodiments, the variant binary complex exhibits decreased at least one of complex (variant binary complex or variant ternary complex) dissociation and/or dissociation from a target locus at about 37° C. over an incubation period of at least about any one of 10 mins, 15 mins, 20 mins, 25 mins, 30 mins, 35 mins, 40 mins, 45 mins, 50 mins, 55 mins, 1 hr, 2 hr, 3 hr, 4 hr, or more hours as compared to a complex formed by a parent polypeptide and RNA guide. In some embodiments, a variant binary complex exhibits decreased at least one of complex (variant binary complex or variant ternary complex) dissociation and/or dissociation from a target locus over a range of incubation times as compared to a complex formed by a parent polypeptide and RNA guide.

In some embodiments, the variant binary complex exhibits decreased at least one of complex (variant binary complex or variant ternary complex) dissociation and/or dissociation from a target locus at about 37° C. over an incubation period of at least about any one of 10 mins, 15 mins, 20 mins, 25 mins, 30 mins, 35 mins, 40 mins, 45 mins, 50 mins, 55 mins, 1 hr, 2 hr, 3 hr, 4 hr, or more hours as compared to a parent binary complex. In some embodiments, the variant binary complex exhibits decreased at least one of complex (variant binary complex or variant ternary complex) dissociation and/or dissociation from a target locus over a range of incubation times as compared to a parent binary complex.

### Ternary Complex

In some aspects, a Cas12i2 polypeptide, a targeting moiety and a target nucleic acid form a complex. In some embodiments, a Cas12i2 polypeptide, e.g., a variant Cas12i2 polypeptide, a targeting moiety, e.g., an RNA guide as described herein, and a target nucleic acid (e.g., DNA) form a complex (e.g., a ternary complex, e.g., a ribonucleoprotein bound to DNA). In some embodiments, a Cas12i2 polypeptide, an RNA guide and target DNA form a ternary complex.

A ternary complex described herein, in some embodiments, comprises a variant Cas12i2 polypeptide that associates with at least one RNA guide (i.e., forms a variant binary complex), wherein each RNA guide targets and associates with a target nucleic acid such as DNA (i.e., forms a variant ternary complex). In some embodiments, the variant Cas12i2 polypeptide/RNA guide complex (e.g., variant binary complex) comprises enzymatic activity, such as nuclease activity, that may nick or cleave the target nucleic acid of the variant ternary complex. In some embodiments, the variant ternary complex comprises enzymatic activity, such as nuclease activity. The variant Cas12i2 polypeptide, the RNA guide, and the target nucleic acid, either alone or together, do not naturally occur.

Generally, the variant binary complex, e.g., the variant Cas12i2 polypeptide and the targeting moiety, binds to a target nucleic acid in a molar ratio of about 1:1 to form the variant ternary complex. Binding of the variant binary complex to the target nucleic acid, e.g., target DNA substrate, to form the variant ternary complex is referred to as loading the variant binary complex to the target nucleic acid.

Generally, the variant Cas12i2 polypeptide, the targeting moiety (e.g., RNA guide), and the target nucleic acid associate with each other in a molar ratio of about 1:1:1 to form the variant ternary complex.

In some embodiments, a target nucleic acid includes one or more target loci of a variant binary complex or plurality of variant binary complexes. In some embodiments, a target nucleic acid includes one or more non-target loci of a variant binary complex or plurality of variant binary complexes.

### Functionality of Ternary Complexes

In some aspects, the variant ternary complex comprises a variant Cas12i2 polypeptide with at least one alteration or mutation and the variant ternary complex has enhanced at least one of enzymatic activity, target nucleic acid complex formation, target nucleic acid binding activity, target nucleic acid affinity, target nucleic acid binding specificity, protein-nucleic acid interactions, ternary complex formation, on-target binding activity, on-target binding specificity, and/or stability.

In some aspects, the variant ternary complex comprises a variant Cas12i2 polypeptide with at least one alteration or mutation and the variant ternary complex has decreased at least one of dissociation from a target locus, off-target binding to a non-target nucleic acid, activity at a non-target locus of a target nucleic acid, and/or ternary complex dissociation.

In some aspects, a variant binary complex and a target nucleic acid (e.g., DNA) form a variant ternary complex, and the variant ternary complex exhibits increased at least one of enzymatic activity, target nucleic acid complex formation, target nucleic acid binding activity, target nucleic acid affinity, target nucleic acid binding specificity, protein-nucleic acid interactions, ternary complex formation, on-target binding activity, on-target binding specificity, and/or stability, as compared to a parent binary complex.

In some embodiments, the variant ternary complex exhibits at least one of enzymatic activity, target nucleic acid complex formation, target nucleic acid binding activity, target nucleic acid affinity, target nucleic acid binding specificity, protein-nucleic acid interactions, ternary complex formation, on-target binding activity, on-target binding specificity, and/or stability of the variant ternary complex at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% greater than the enzymatic activity, target nucleic acid complex formation, target nucleic acid binding activity, target nucleic acid affinity, target nucleic acid binding specificity, protein-nucleic acid interactions, ternary complex formation, on-target binding activity, on-target binding specificity, and/or stability of a parent binary complex.

In some embodiments, the variant ternary complex exhibits enhanced at least one of enzymatic activity, target nucleic acid complex formation, target nucleic acid binding activity, target nucleic acid affinity, target nucleic acid binding specificity, protein-nucleic acid interactions, ternary complex formation, on-target binding activity, on-target binding specificity, and/or stability, as compared to a parent ternary complex, at a temperature in the range of about 20° C. to about 65° C., e.g., about any one of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 50° C., 51° C., 52° C., 53° C., 54° C., 55° C., 56° C., 57° C., 58° C., 59° C., 60° C. or 65° C.

In some embodiments, the variant ternary complex exhibits enhanced at least one of enzymatic activity, target nucleic acid complex formation, target nucleic acid binding activity, target nucleic acid affinity, target nucleic acid binding specificity, protein-nucleic acid interactions, ternary complex formation, on-target binding activity, on-target binding specificity, and/or stability, as compared to a parent ternary complex, in a buffer having a pH in a range of about 7.3 to about 8.6 (e.g., 7.3, 7.4, 7.5, 7.6, 7.7, 7.8, 7.9, 8.0, 8.1, 8.2, 8.3, 8.4, 8.5, 8.6, or any value within a range between any combination of these values).

In some embodiments, the variant ternary complex exhibits enhanced at least one of enzymatic activity, target nucleic acid complex formation, target nucleic acid binding activity, target nucleic acid affinity, target nucleic acid binding specificity, protein-nucleic acid interactions, ternary complex formation, on-target binding activity, on-target binding specificity, and/or stability, as compared to a parent ternary complex, when the Tₘ value of the variant ternary complex is at least 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., or 20° C. greater than the Tₘ value of a parent ternary complex. In one embodiment, the variant ternary complex exhibits enhanced at least one of enzymatic activity, target nucleic acid complex formation, target nucleic acid binding activity, target nucleic acid affinity, target nucleic acid binding specificity, protein-nucleic acid interactions, ternary complex formation, on-target binding activity, on-target binding specificity, and/or stability when the Tₘ value of the variant ternary complex is at least 8° C. greater than the Tₘ value of the parent ternary complex.

In some embodiments, the variant ternary complex exhibits increased at least one of enzymatic activity, target nucleic acid complex formation, target nucleic acid binding activity, target nucleic acid affinity, target nucleic acid binding specificity, protein-nucleic acid interactions, ternary complex formation, on-target binding activity, on-target binding specificity, and/or stability at about 37° C. over an incubation period of at least about any one of 10 mins, 15 mins, 20 mins, 25 mins, 30 mins, 35 mins, 40 mins, 45 mins, 50 mins, 55 mins, 1 hr, 2 hr, 3 hr, 4 hr, or more hours as compared to a parent ternary complex. In some embodiments, a variant ternary complex exhibits increased at least one of enzymatic activity, target nucleic acid complex formation, target nucleic acid binding activity, target nucleic acid affinity, target nucleic acid binding specificity, protein-nucleic acid interactions, ternary complex formation, on-target binding activity, on-target binding specificity, and/or stability over a range of incubation times as compared to a parent ternary complex.

In some embodiments, the variant ternary complex exhibits enhanced at least one of enzymatic activity, target nucleic acid complex formation, target nucleic acid binding activity, target nucleic acid affinity, target nucleic acid binding specificity, protein-nucleic acid interactions, ternary complex formation, on-target binding activity, on-target binding specificity, and/or stability and the variant ternary complex comprises a variant Cas12i2 polypeptide comprising an amino acid sequence having at least about 60%, 65%, 70%, 75%, 80%, 85%, 90%, 91%, 92%, 93%, 94%, 95%, 96%, 97%, 98%, 99%, or 100% identity to any one of SEQ ID NO: 4.

In some embodiments, the variant ternary complex exhibits enhanced at least one of enzymatic activity, target nucleic acid complex formation, target nucleic acid binding activity, target nucleic acid affinity, target nucleic acid binding specificity, protein-nucleic acid interactions, ternary complex formation, on-target binding activity, on-target binding specificity, and/or stability and the variant ternary complex comprises a variant Cas12i2 polypeptide comprising an amino acid sequence having at least about 60%, 65%, 70%, 75%, 80%, 85%, 90%, 91%, 92%, 93%, 94%, 95%, 96%, 97%, 98%, 99%, or 100% identity to any one of SEQ ID NOs: 3-5, 495, or 496.

In some embodiments, the variant ternary complex exhibits enhanced at least one of enzymatic activity, target nucleic acid complex formation, target nucleic acid binding activity, target nucleic acid affinity, target nucleic acid binding specificity, protein-nucleic acid interactions, ternary complex formation, on-target binding activity, on-target binding specificity, and/or stability and the variant ternary complex comprises a variant Cas12i2 polypeptide comprising an amino acid sequence having at least about 60%, 65%, 70%, 75%, 80%, 85%, 90%, 91%, 92%, 93%, 94%, 95%, 96%, 97%, 98%, 99%, or 100% identity to any one of SEQ ID NOs: 3-146 and 495-512.

In some aspects, a variant binary complex and a target nucleic acid (e.g., DNA) form a variant ternary complex, and the variant ternary complex exhibits decreased at least one of dissociation from a target locus, off-target binding to a non-target nucleic acid, activity at a non-target locus of a target nucleic acid, and/or ternary complex dissociation, as compared to a parent binary complex.

In some embodiments, the variant ternary complex exhibits decreased at least one of dissociation from a target locus, off-target binding to a non-target nucleic acid, activity at a non-target locus of a target nucleic acid, and/or ternary complex dissociation of the variant ternary complex may be at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% less than the dissociation from a target locus, off-target binding to a non-target nucleic acid, activity at a non-target locus of a target nucleic acid, and/or ternary complex dissociation of a parent binary complex.

In some embodiments, the variant ternary complex exhibits decreased at least one of dissociation from a target locus, off-target binding to a non-target nucleic acid, activity at a non-target locus of a target nucleic acid, and/or ternary complex dissociation at a temperature of about any one of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 50° C., 51° C., 52° C., 53° C., 54° C., 55° C., 56° C., 57° C., 58° C., 59° C., 60° C. or 65° C. as compared to a parent ternary complex.

In some embodiments, the variant ternary complex exhibits decreased at least one of dissociation from a target locus, off-target binding to a non-target nucleic acid, activity at a non-target locus of a target nucleic acid, and/or ternary complex dissociation over a range of temperatures, from about 20° C. to about 65° C. as compared to a parent ternary complex. In some embodiments, the variant ternary complex exhibits decreased at least one of dissociation from a target locus, off-target binding to a non-target nucleic acid, activity at a non-target locus of a target nucleic acid, and/or ternary complex dissociation at about 37° C. over an incubation period of at least about any one of 10 mins, 15 mins, 20 mins, 25 mins, 30 mins, 35 mins, 40 mins, 45 mins, 50 mins, 55 mins, 1 hr, 2 hr, 3 hr, 4 hr, or more hours as compared to a parent ternary complex. In some embodiments, the variant ternary complex exhibits decreased at least one of dissociation from a target locus, off-target binding to a non-target nucleic acid, activity at a non-target locus of a target nucleic acid, and/or ternary complex dissociation over a range of incubation times as compared to a parent ternary complex.

In some embodiments, the variant ternary complex exhibits decreased at least one of dissociation from a target locus, off-target binding to a non-target nucleic acid, activity at a non-target locus of a target nucleic acid, and/or ternary complex dissociation in a buffer having a pH in a range of about 7.3 to about 8.6 than a parent ternary complex. In one embodiment, the variant ternary complex exhibits decreased at least one of dissociation from a target locus, off-target binding to a non-target nucleic acid, activity at a non-target locus of a target nucleic acid, and/or ternary complex dissociation in a pH of about 7.3, 7.4, 7.5, 7.6, 7.7, 7.8, 7.9, 8.0, 8.1, 8.2, 8.3, 8.4, 8.5, or 8.6 than a parent ternary complex.

In some embodiments, the variant ternary complex exhibits decreased at least one of dissociation from a target locus, off-target binding to a non-target nucleic acid, activity at a non-target locus of a target nucleic acid, and/or ternary complex dissociation when a Tₘ value of the variant ternary complex is at least 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., or 20° C. greater than the Tₘ value of the reference molecule or Tₘ of a reference value, e.g., of Tₘ of a parent ternary complex. In one embodiment, the variant ternary complex exhibits decreased at least one of dissociation from a target locus, off-target binding to a non-target nucleic acid, activity at a non-target locus of a target nucleic acid, and/or ternary complex dissociation when a Tₘ value of the variant ternary complex is at least 8° C. greater than the Tₘ value of the reference molecule or Tₘ of a reference value, e.g., Tₘ of a parent ternary complex.

In some embodiments, the variant ternary complex exhibits decreased at least one of at least one of dissociation from a target locus, off-target binding to a non-target nucleic acid, activity at a non-target locus of a target nucleic acid, and/or ternary complex dissociation and the variant ternary complex comprises a variant Cas12i2 polypeptide comprising an amino acid sequence having at least about 60%, 65%, 70%, 75%, 80%, 85%, 90%, 91%, 92%, 93%, 94%, 95%, 96%, 97%, 98%, 99%, or 100% identity to any one of SEQ ID NO: 4.

In some embodiments, the variant ternary complex exhibits decreased at least one of at least one of dissociation from a target locus, off-target binding to a non-target nucleic acid, activity at a non-target locus of a target nucleic acid, and/or ternary complex dissociation and the variant ternary complex comprises a variant Cas12i2 polypeptide comprising an amino acid sequence having at least about 60%, 65%, 70%, 75%, 80%, 85%, 90%, 91%, 92%, 93%, 94%, 95%, 96%, 97%, 98%, 99%, or 100% identity to any one of SEQ ID NOs: 3-5, 495, or 496.

In some embodiments, the variant ternary complex exhibits decreased at least one of at least one of dissociation from a target locus, off-target binding to a non-target nucleic acid, activity at a non-target locus of a target nucleic acid, and/or ternary complex dissociation and the variant ternary complex comprises a variant Cas12i2 polypeptide comprising an amino acid sequence having at least about 60%, 65%, 70%, 75%, 80%, 85%, 90%, 91%, 92%, 93%, 94%, 95%, 96%, 97%, 98%, 99%, or 100% identity to any one of SEQ ID NOs: 3-146 and 495-512.

In some embodiments, the variant ternary complex exhibits increased stability at about 37° C. over an incubation period of at least about any one of 10 mins, 15 mins, 20 mins, 25 mins, 30 mins, 35 mins, 40 mins, 45 mins, 50 mins, 55 mins, 1 hr, 2 hr, 3 hr, 4 hr, or more hours as compared to a parent ternary complex. In some embodiments, a variant ternary complex exhibits increased stability over a range of incubation times as compared to a parent ternary complex.

In some aspects, a variant binary complex exhibits decreased activity at a non-target locus of a target nucleic acid as compared to a parent binary complex. In some embodiments, non-target activity is assessed at a PAM-adjacent sequence of a particular Levenshtein distance (e.g., an edit distance of 1, 2, 3, or 4) from an on-target locus sequence. In some embodiments, activity at a non-target locus by a variant binary complex may be at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% less than activity at the non-target locus by a parent binary complex.

### Plurality of Complexes

In some embodiments, two or more (e.g., 2, 3, 4, 5, 6, 7, 8, 9, or more) distinct variant Cas12i2 polypeptides and two or more (e.g., 2, 3, 4, 5, 6, 7, 8, 9, or more) distinct targeting moieties form individual variant binary complexes, and wherein the distinct variant binary complexes exhibit at least one or more of the characteristics of increased binding affinity to the target nucleic acid, increased target binding affinity to a target locus of a target nucleic acid, increased ternary complex formation, and/or increased stability over a range of incubation times. In such embodiments, complex formation may be accomplished simultaneously in a single composition or independently in separate compositions.

In some aspects, a plurality of Cas12i2 polypeptides, e.g., a plurality of variant Cas12i2 polypeptides, and two or more distinct targeting moieties, e.g., two or more distinct RNA guides, individually form binary complexes. For example, a first Cas12i2 polypeptide of SEQ ID NO: AA forms a first binary complex with an RNA guide of SEQ ID NO: BB (e.g., at a molar ratio of about 1:1), and a second Cas12i2 polypeptide of SEQ ID NO: AA forms a second binary complex with an RNA guide of SEQ ID NO: DD (e.g., at a molar ratio of about 1:1). In some embodiments, the two binary complexes specifically bind with distinct target loci of a target nucleic acid. For example, the first binary complex (e.g., Cas12i2 polypeptide of SEQ ID NO: AA/RNA guide of SEQ ID NO: BB) may specifically bind with target locus CC of the target nucleic acid (e.g., at a molar ratio of 1:1) to form a first ternary complex, and the second binary complex (e.g., Cas12i2 polypeptide of SEQ ID NO: AA/RNA guide of SEQ ID NO: YY) may specifically bind with target locus ZZ of the target nucleic acid (e.g., at a molar ratio of 1:1) to form a second ternary complex. As used herein, the term “a binary complex” refers to one or more distinct binary complexes, e.g., binary complexes each having distinct targeting moieties (e.g., distinct RNA guides) targeting distinct target loci (e.g., two or more target loci of a target nucleic acid). As used herein, the term “a ternary complex” refers to one or more distinct ternary complexes, e.g., ternary complexes comprising distinct targeting moieties (e.g., distinct RNA guides) bound to distinct target loci.

In some aspects, a plurality of variant Cas12i2 polypeptides and two or more targeting moieties (e.g., two or more RNA guides) form a plurality of variant binary complexes, and the plurality of variant binary complexes exhibit increased on-target binding to two or more target loci of a target nucleic acid, as compared to a plurality of parent binary complexes. In some embodiments, the on-target binding by the plurality of variant binary complexes may be at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% greater than the on-target binding of a plurality of parent binary complexes to the two or more target loci.

In some aspects, a plurality of variant Cas12i2 polypeptides and two or more targeting moieties (e.g., two or more RNA guides) form a plurality of variant binary complexes, and the plurality of variant binary complexes exhibit increased on-target activity at two or more target loci of a target nucleic acid, as compared to a plurality of parent binary complexes. In some embodiments, the on-target activity by the plurality of variant binary complexes may be at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% greater than the on-target activity of a plurality of parent binary complexes.

In some embodiments, the plurality of variant binary complexes form a plurality of variant ternary complexes with two or more target loci of a target nucleic acid at a temperature of about any one of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 50° C., 51° C., 52° C., 53° C., 54° C., 55° C., 56° C., 57° C., 58° C., 59° C., 60° C. or 65° C. In some embodiments, the plurality of variant binary complexes form a plurality of variant ternary complexes over a range of temperatures, from about 20° C. to about 65° C.

In some embodiments, the plurality of variant binary complexes exhibit increased on-target binding of two or more target loci of a target nucleic acid, increased on-target ternary complex formation with two or more target loci of a target nucleic acid, and/or increased stability at a temperature of about any one of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 50° C., 51° C., 52° C., 53° C., 54° C., 55° C., 56° C., 57° C., 58° C., 59° C., 60° C. or 65° C. as compared to a plurality of parent binary complexes. In some embodiments, the plurality of variant binary complexes exhibit increased on-target binding of two or more target loci of a target nucleic acid, increased on-target ternary complex formation with two or more target loci of a target nucleic acid, and/or increased stability over a range of temperatures, from about 20° C. to about 65° C. as compared to a plurality of parent binary complexes.

In some embodiments, the plurality of variant binary complexes exhibit decreased dissociation from the two or more target loci of a target nucleic acid at a temperature of about any one of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 50° C., 51° C., 52° C., 53° C., 54° C., 55° C., 56° C., 57° C., 58° C., 59° C., 60° C. or 65° C. as compared to a plurality of parent binary complexes. In some embodiments, the plurality of variant binary complexes exhibit decreased dissociation from the two or more target loci of the target nucleic acid over a range of temperatures, from about 20° C. to about 65° C. as compared to a plurality of parent binary complexes.

In some embodiments, the plurality of variant binary complexes exhibit decreased dissociation from the two or more target loci of the target nucleic acid at about 37° C. over an incubation period of at least about any one of 10 mins, 15 mins, 20 mins, 25 mins, 30 mins, 35 mins, 40 mins, 45 mins, 50 mins, 55 mins, 1 hr, 2 hr, 3 hr, 4 hr, or more hours as compared to a plurality of parent binary complexes. In some embodiments, the plurality of variant binary complexes exhibit decreased dissociation from the two or more target loci of the target nucleic acid over a range of incubation times as compared to a plurality of parent binary complexes.

In some embodiments, the plurality of variant binary complexes exhibit decreased dissociation from the two or more target loci of a target nucleic acid in a buffer having a pH in a range of about 7.3 to about 8.6 than a plurality of parent binary complexes. In one embodiment, the plurality of variant binary complexes exhibit decreased dissociation from the two or more target loci of the target nucleic acid in a pH of about 7.3, 7.4, 7.5, 7.6, 7.7, 7.8, 7.9, 8.0, 8.1, 8.2, 8.3, 8.4, 8.5, or 8.6 than a plurality of parent binary complexes.

In some embodiments, the plurality of variant binary complexes exhibit decreased dissociation from the two or more target loci of a target nucleic acid when the Tₘ values of the plurality of variant binary complexes are at least 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., or 20° C. greater than the corresponding Tₘ values of reference complexes or reference Tₘ values, e.g., the Tₘ values of a plurality of parent binary complexes. In one embodiment, the plurality of variant binary complexes exhibit decreased dissociation from the two or more target loci of the target nucleic acid when the Tₘ values of the plurality of variant binary complexes are at least 8° C. greater than the corresponding Tₘ values of reference complexes or reference Tₘ values, e.g., the Tₘ values of a plurality of parent binary complexes.

In some embodiments, the plurality of variant binary complexes exhibit increased on-target binding of two or more target loci of a target nucleic acid, increased on-target ternary complex formation with two or more target loci of a target nucleic acid, and/or increased stability in a buffer having a pH in a range of about 7.3 to about 8.6 than a plurality of parent binary complexes. In one embodiment, the plurality of binary complexes exhibit increased on-target binding of two or more target loci of a target nucleic acid, increased on-target ternary complex formation with two or more target loci of a target nucleic acid, and/or increased stability in a pH of about 7.3, 7.4, 7.5, 7.6, 7.7, 7.8, 7.9, 8.0, 8.1, 8.2, 8.3, 8.4, 8.5, or 8.6 than a plurality of parent binary complexes.

In some embodiments, the plurality of variant binary complexes are stable in a buffer having a pH in a range of about 7.3 to 8.6. In one embodiment, the plurality of variant binary complexes are stable in a pH of about 7.3, 7.4, 7.5, 7.6, 7.7, 7.8, 7.9, 8.0, 8.1, 8.2, 8.3, 8.4, 8.5, or 8.6.

In some embodiments, the plurality of variant binary complexes may be stable if the Tₘ values of the plurality of variant binary complexes are at least 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., or 20° C. greater than the corresponding Tₘ values of the reference complexes or reference Tₘ values, e.g., the Tₘ values of a plurality of parent binary complexes. For example, in certain embodiments, the plurality of variant binary complexes are stable if the Tₘ values of the plurality of variant binary complexes are at least 8° C. greater than the corresponding Tₘ values of the reference complexes or reference Tₘ values, e.g., the Tₘ values of a plurality of parent binary complexes.

In some embodiments, the plurality of variant binary complexes exhibit increased stability when the Tₘ values of the plurality of variant binary complexes are at least 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., or 20° C. greater than the corresponding Tₘ values of the reference complexes or reference Tₘ values, e.g., the Tₘ values of a plurality of parent binary complexes. In one embodiment, the plurality of variant binary complexes exhibit increased stability when the Tₘ values of the plurality of variant binary complexes are at least 8° C. greater than the corresponding Tₘ values of the reference complexes or reference Tₘ values, e.g., the Tₘ values of a plurality of parent binary complexes.

In some embodiments, a plurality of variant binary complexes (e.g., binary complexes formed from a plurality of variant Cas12i2 polypeptides and a plurality of distinct targeting moieties) specifically bind to a plurality of target loci of a target nucleic acid. In some embodiments, the plurality of variant binary complexes exhibit increased on-target binding with two or more (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 162, 164, 164, 165, 166, 167, 168, 169, 170, 171, 172, 173, 174, 175, 176, 177, 178, 179, 180, 181, 182, 183, 184, 185, 186, 187, 188, 189, 190, 191, 193, 194, 195, 196, 197, 198, 199, 200, or more) target loci, as compared to a plurality of parent binary complexes.

In some embodiments, a plurality of variant binary complexes (e.g., binary complexes formed from a plurality of variant Cas12i2 polypeptides and a plurality of RNA guides) specifically bind to a plurality of target loci of a target nucleic acid. In some embodiments, the plurality of variant binary complexes exhibit increased on-target binding with two or more (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 162, 164, 164, 165, 166, 167, 168, 169, 170, 171, 172, 173, 174, 175, 176, 177, 178, 179, 180, 181, 182, 183, 184, 185, 186, 187, 188, 189, 190, 191, 193, 194, 195, 196, 197, 198, 199, 200, or more) target loci, as compared to a plurality of parent binary complexes.

In some embodiments, a plurality of variant binary complexes (e.g., binary complexes formed from a plurality of variant Cas12i2 polypeptides and a plurality of distinct targeting moieties) exhibit ternary complex formation with a plurality of target loci of a target nucleic acid. In some embodiments, the plurality of variant binary complexes exhibit increased ternary complex formation with two or more (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 162, 164, 164, 165, 166, 167, 168, 169, 170, 171, 172, 173, 174, 175, 176, 177, 178, 179, 180, 181, 182, 183, 184, 185, 186, 187, 188, 189, 190, 191, 193, 194, 195, 196, 197, 198, 199, 200, or more) target loci, as compared to a plurality of parent binary complexes.

In some embodiments, a plurality of variant binary complexes (e.g., binary complexes formed from a plurality of variant Cas12i2 polypeptides and a plurality of RNA guides) exhibit ternary complex formation with a plurality of target loci of a target nucleic acid. In some embodiments, the plurality of variant binary complexes exhibit increased ternary complex formation with two or more (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 162, 164, 164, 165, 166, 167, 168, 169, 170, 171, 172, 173, 174, 175, 176, 177, 178, 179, 180, 181, 182, 183, 184, 185, 186, 187, 188, 189, 190, 191, 193, 194, 195, 196, 197, 198, 199, 200, or more) target loci, as compared to a plurality of parent binary complexes.

In some embodiments, the plurality of variant ternary complexes exhibit increased stability at a temperature of about any one of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 50° C., 51° C., 52° C., 53° C., 54° C., 55° C., 56° C., 57° C., 58° C., 59° C., 60° C. or 65° C. as compared to a plurality of parent ternary complexes. In some embodiments, the plurality of variant ternary complexes exhibit increased ternary complex formation at a target locus and/or increased stability over a range of temperatures, from about 20° C. to about 65° C. as compared to a plurality of parent ternary complexes.

In some embodiments, the plurality of variant ternary complexes exhibit increased stability at about 37° C. over an incubation period of at least about any one of 10 mins, 15 mins, 20 mins, 25 mins, 30 mins, 35 mins, 40 mins, 45 mins, 50 mins, 55 mins, 1 hr, 2 hr, 3 hr, 4 hr, or more hours as compared to a plurality of parent ternary complexes. In some embodiments, the plurality of variant ternary complexes exhibit increased stability over a range of incubation times as compared to a plurality of parent ternary complexes.

In some embodiments, the plurality of variant ternary complexes exhibit increased stability in a buffer having a pH in a range of about 7.3 to 8.6. In one embodiment, the plurality of variant ternary complexes exhibit increased stability in a pH of about 7.3, 7.4, 7.5, 7.6, 7.7, 7.8, 7.9, 8.0, 8.1, 8.2, 8.3, 8.4, 8.5, or 8.6. In some embodiments, the plurality of variant ternary complexes exhibit increased stability in a buffer having a pH in a range of about 7.3 to 8.6 as compared to a plurality of parent ternary complexes. In one embodiment, the plurality of variant ternary complexes exhibit increased stability in a pH of about 7.3, 7.4, 7.5, 7.6, 7.7, 7.8, 7.9, 8.0, 8.1, 8.2, 8.3, 8.4, 8.5, or 8.6 as compared to a plurality of parent ternary complexes.

In some embodiments, the plurality of variant ternary complexes exhibit increased stability if the Tₘ values of the plurality of variant ternary complexes is at least 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., or 20° C. greater than the Tₘ values of reference complexes or Tₘ reference values, e.g., the Tₘ values of a plurality of parent ternary complexes. For example, in certain embodiments, the plurality of variant ternary complexes exhibit increased stability if the Tₘ values of the plurality of variant ternary complexes are at least 8° C. greater than the Tₘ values of reference complexes or Tₘ reference values, e.g., the Tₘ values of a plurality of parent ternary complexes.

In some embodiments, the plurality of variant ternary complexes exhibit decreased dissociation of two or more target loci at a range of temperatures, incubation times, pH values, and Tₘ values.

In some embodiments, the plurality of variant ternary complexes exhibit decreased dissociation of their two or more target loci at a temperature of about any one of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 50° C., 51° C., 52° C., 53° C., 54° C., 55° C., 56° C., 57° C., 58° C., 59° C., 60° C. or 65° C. as compared to a plurality of parent ternary complexes. In some embodiments, the plurality of variant ternary complexes exhibit decreased dissociation of their two or more target loci over a range of temperatures, from about 20° C. to about 65° C. as compared to a plurality of parent ternary complexes.

In some embodiments, the plurality of variant ternary complexes exhibit decreased dissociation of their two or more target loci at about 37° C. over an incubation period of at least about any one of 10 mins, 15 mins, 20 mins, 25 mins, 30 mins, 35 mins, 40 mins, 45 mins, 50 mins, 55 mins, 1 hr, 2 hr, 3 hr, 4 hr, or more hours as compared to a plurality of parent ternary complexes. In some embodiments, the plurality of variant ternary complexes exhibit decreased dissociation of their two or more target loci over a range of incubation times as compared to a plurality of parent ternary complexes.

In some embodiments, the plurality of variant ternary complexes exhibit decreased dissociation of their two or more target loci in a buffer having a pH in a range of about 7.3 to about 8.6 than a plurality of parent ternary complexes. In some embodiments, the plurality of variant ternary complexes exhibit decreased dissociation of their two or more target loci in a pH of about 7.3, 7.4, 7.5, 7.6, 7.7, 7.8, 7.9, 8.0, 8.1, 8.2, 8.3, 8.4, 8.5, or 8.6 than a plurality of parent ternary complexes.

In some embodiments, the plurality of variant ternary complexes exhibit decreased dissociation of their two or more target loci when a Tₘ values of the plurality of variant ternary complexes are at least 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., or 20° C. greater than the Tₘ values of reference complexes or Tₘ reference values, e.g., of Tₘ values of a plurality of parent ternary complexes. In some embodiments, the plurality of variant ternary complexes exhibit decreased dissociation of their two or more target loci when the Tₘ values of the variant ternary complexes are at least 8° C. greater than the Tₘ values of reference complexes or Tₘ reference values, e.g., the Tₘ values of a plurality of parent ternary complexes.

In some aspects, a plurality of variant Cas12i2 polypeptides and two or more targeting moieties (e.g., two or more RNA guides) form a plurality of variant binary complexes, and the plurality of variant binary complexes exhibit decreased off-target binding to two or more non-target loci of a target nucleic acid, as compared to a plurality of parent binary complexes. In some embodiments, the off-target binding by the plurality of variant binary complexes may be at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% less than the off-target binding of a parent binary complex to the two or more non-target loci.

In some aspects, a plurality of variant Cas12i2 polypeptides and two or more targeting moieties (e.g., two or more RNA guides) form a plurality of variant binary complexes, and the plurality of variant binary complexes exhibit decreased off-target activity at two or more non-target loci of a target nucleic acid, as compared to a parent binary complex. In some embodiments, the off-target activity by the plurality of variant binary complexes may be at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% less than the off-target activity of a plurality of parent binary complexes.

Unless otherwise noted, all compositions and complexes and polypeptides provided herein are made in reference to the active level of that composition or complex or polypeptide, and are exclusive of impurities, for example, residual solvents or by-products, which may be present in commercially available sources. Enzymatic component weights are based on total active protein. All percentages and ratios are calculated by weight unless otherwise indicated. All percentages and ratios are calculated based on the total composition unless otherwise indicated. In the exemplified composition, the enzymatic levels are expressed by pure enzyme by weight of the total composition and unless otherwise specified, the ingredients are expressed by weight of the total compositions.

### Activity

Described in the invention herein are variant Cas12i2 polypeptides and variant binary complexes having higher on-target specificity and lowered off-target effects, such as nicking or cleaving an off-target locus or multiple off-target loci. Off-target effects, even with low frequency of occurrence, can lead to genetic instability and disruption of gene function.

In some embodiments, compositions described herein comprise a complex, e.g., a binary complex comprising a variant Cas12i2 polypeptide (e.g., a variant binary complex) having decreased off-target interactions and/or decreased activity at a non-target locus or non-target loci, e.g., decreased interactions and/or activity with a non-target nucleic acid. Indeed, in the examples, it is shown that the invention involves variant Cas12i2 polypeptides that form variant binary complexes having increased on-target activity and decreased off-target activity.

In some embodiments, an on-target locus and a non-target locus are on the same nucleic acid (e.g., the same chromosome). In some embodiments, an on-target locus and a non-target locus are on different nucleic acids (e.g., different chromosomes).

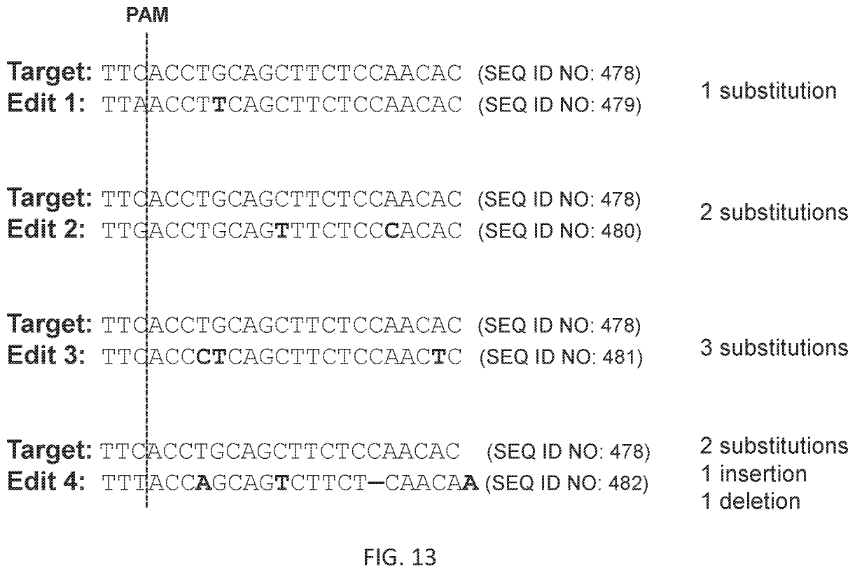

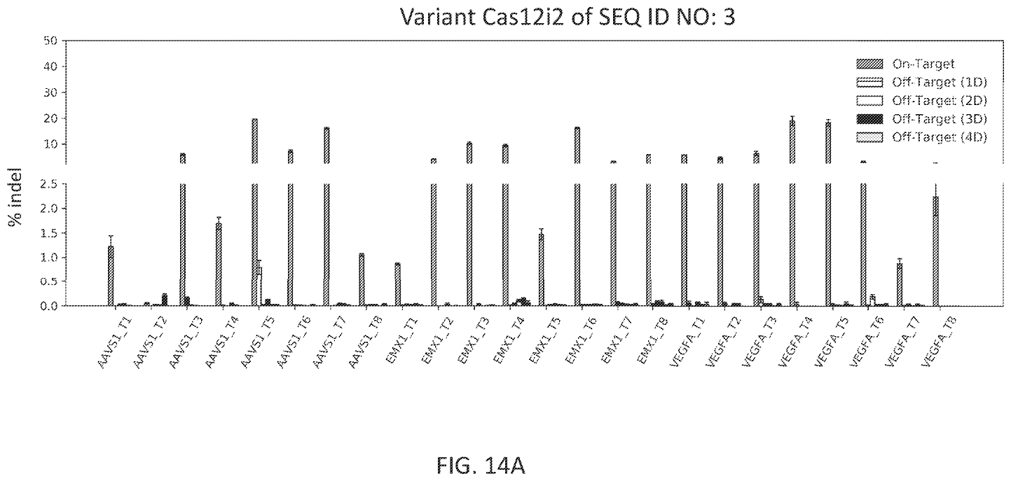

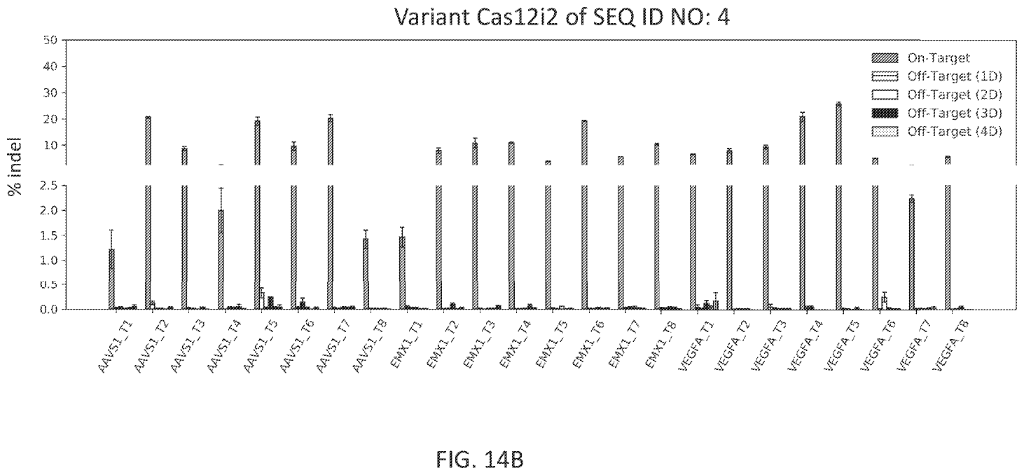

In some embodiments, on-target activity and off-target activity are measured by first identifying non-target loci sequences of a particular Levenshtein distance (also referred to as an edit distance) from an on-target locus sequence and measuring indels at both the on-target locus and the non-target locus, as shown in Example 6. Edit distance refers to the minimum number of edits (e.g., insertions, deletions, or substitutions) required to change a first sequence (e.g., an off-target sequence) to a second sequence (e.g., an on-target sequence). For example, two edits (e.g., two insertions, two deletions, two substitutions, one insertion and one deletion, one insertion and one substitution, or one deletion and one substitution) are required to change the sequence of an off-target sequence with an edit distance of 2 to an on-target sequence. Five exemplary sequences are shown in FIG. 13: an on-target sequence, an off-target sequence with an edit distance of 1, an off-target sequence with an edit distance of 2, an off-target sequence with an edit distance of 3, and an off-target sequence with an edit distance of 4.

In some embodiments, a variant binary complex is formed with a variant Cas12i2 polypeptide and a targeting moiety (e.g., an RNA guide) targeting a target locus, and activity (e.g., nuclease activity) of the variant binary complex is measured at the target locus and a non-target locus to determine both on-target activity and off-target activity. In some embodiments, a sequence having an edit distance of 1, 2, 3, 4, 5, 6, 7, 8, 9, 10 or greater is selected to measure off-target activity, as described in Example 14. In some embodiments, the variant binary complex exhibits decreased activity or lack of edits at a non-target locus.

Accordingly, in the context of the present invention, the degree of decreased off-target activity (e.g., off-target nuclease activity) and/or increased on-target activity (e.g., on-target nuclease activity) between a variant binary complex and a parent binary complex may differ by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or more.

In some embodiments, off-target activity by a variant binary complex is decreased by at least about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% as compared to off-target activity by a parent binary complex.

In some embodiments, on-target activity by a variant binary complex is increased by at least about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more as compared to on-target activity by a parent binary complex.

In some embodiments, increased activity of a variant binary complex at a target locus (on-target activity), as compared to a reference, is associated with decreased activity of the variant binary complex at a non-target locus (e.g., off-target activity), as compared to a reference. In some embodiments, activity of a variant binary complex at a target locus (on-target activity) is inversely associated with activity of the variant binary complex at a non-target locus (e.g., off-target activity). In some embodiments, increased activity of a variant binary complex at a target locus by at least about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more, as compared to a reference (e.g., on-target activity by a parent binary complex), is associated with decreased activity of the variant binary complex at a non-target locus by at least about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more, as compared to a reference (e.g., off-target activity by a parent binary complex). In some embodiments, increased activity of a variant binary complex at a target locus (on-target activity), as compared to a reference, results in decreased activity of the variant binary complex at a non-target locus (e.g., off-target activity), as compared to a reference.

In some embodiments, increased activity of a plurality of variant binary complexes at two or more target loci (on-target activity), as compared to a reference, is associated with decreased activity of the plurality of variant binary complexes at two or more non-target loci (e.g., off-target activity), as compared to a reference. In some embodiments, activity of a plurality of variant binary complexes at two or more target loci (e.g., on-target activity) is inversely associated with activity of the plurality of variant binary complexes at two or more non-target loci (e.g., off-target activity). In some embodiments, increased activity of a plurality of variant binary complexes at two or more target loci (on-target activity) by at least about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more, as compared to a reference (e.g., on-target activity by a parent binary complex), is associated with decreased activity of the plurality of variant binary complexes at two or more non-target loci (e.g., off-target activity) by at least about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more as compared to a reference (e.g., off-target activity by a parent binary complex). In some embodiments, increased activity of a plurality of variant binary complexes at two or more target loci (e.g., on-target activity), as compared to a reference, results in decreased activity of the plurality of variant binary complexes at two or more non-target loci (e.g., off-target activity), as compared to a reference.

### Binding Activity

As used herein, the term “binding affinity” generally refers to an overall binding property of a first agent (e.g., a variant Cas12i2 polypeptide and/or a binary complex) interacting with a second agent (e.g., a targeting moiety and/or on-target locus of a target nucleic acid). In some embodiments, the binding affinity may be assessed or measured under a specific condition, and the overall binding property may be dependent on one or more intrinsic characteristics of the first agent and/or second agent including, but not limited to, surface composition of the first agent and/or the second agent (e.g., but not limited to, concentration of first agent in the present of the second agent), intermolecular-bond affinity (e.g., ionic bond formation), avidity, as well as the surrounding/ambient condition for the binding interaction, e.g., but not limited to, concentration of the first agent and/or the second agent, and/or the presence of a third agent (e.g., a target nucleic acid, a non-target nucleic acid, and/or an interfering agent) during the binding interaction between the first and the second agents.

Different measures of determining binding affinity of an agent are known in the art. In some embodiments, the binding affinity of a variant Cas12i2 polypeptide to a targeting moiety can be indicated by a dissociation constant (KD) for binding of the variant Cas12i2 polypeptide to the targeting moiety. In some embodiments, the binding affinity of binary complex to a target nucleic acid can be indicated by a dissociation constant (KD) for binding of the binary complex to the target nucleic acid. The dissociation constant (KD) is an equilibrium constant that generally measures the propensity of a binary or ternary complex to separate (dissociate) reversibly into separate agents. In these embodiments, a higher dissociation constant indicates a lower binding affinity. Alternatively, the binding affinity of a first agent (e.g., variant Cas12i2 polypeptide or binary complex) to a second agent (e.g., targeting moiety or target nucleic acid) can be indicated by an association constant (KA) for binding of the first agent to the second agent.

In some embodiments, the binding affinity is indicated by a dissociation constant (KD) for (non-specific) binding of a variant Cas12i2 polypeptide or binary complex to a non-targeting moiety or non-target nucleic acid. In some embodiments, the binding affinity of a variant Cas12i2 polypeptide to a non-target nucleic acid can be indicated by a dissociation constant (KD) for binding of the variant Cas12i2 polypeptide to the non-targeting moiety. In some embodiments, the binding affinity of a binary complex to a non-target nucleic acid can be indicated by a dissociation constant (KD) for binding of the binary complex to the non-target nucleic acid. The dissociation constant (KD) is an equilibrium constant that generally measures the propensity of a binary or ternary complex to separate (dissociate) reversibly into separate agents. In these embodiments, a higher dissociation constant indicates a lower binding affinity. Alternatively, the binding affinity of a first agent (e.g., variant Cas12i2 polypeptide or binary complex) to a second agent (e.g., non-targeting moiety or non-target nucleic acid) can be indicated by an association constant (KA) for binding of the first agent to the second agent.

In some embodiments, detection methods known in the art can be performed to measure binding affinity, e.g., using a “sandwich” method such as ELISA or any other detection methods.

In some embodiments, the KD value of the present invention may be below a KD value said to be characteristic for a non-specific binding of a first agent to a second agent. As will be acknowledged by the ones skilled in the art, the KD value of a binary or ternary complex comprising a variant Cas12i2 polypeptide may be within a certain range as compared to a binary or ternary complex comprising a parent polypeptide. The above-mentioned KD of the binary or ternary complex comprising a parent polypeptide may be an upper limit for the KD value of the binary or ternary complex comprising a variant Cas12i2 polypeptide. It is within the present invention that the KD values of individual variant Cas12i2 polypeptides in respective binary or ternary complexes may exhibit different KD values.

In the context of the present invention, the degree of decreased binding affinity to a non-target locus and/or increased on-target binding affinity between a variant binary complex and a parent binary complex may differ by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or more.

In some embodiments, binding affinity of a variant binary complex to a non-target locus (e.g., off-target binding affinity) is decreased by at least about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% as compared to binding affinity of a parent binary complex to the non-target locus.

In some embodiments, binding affinity of a plurality of variant binary complexes to two or more non-target loci (e.g., off-target binding affinity) is decreased by at least about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% as compared to binding affinity of a plurality of parent binary complexes to the two or more non-target loci.

In some embodiments, binding affinity of a variant binary complex to a target locus (e.g., on-target binding affinity) is increased by at least about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200% or more as compared to binding affinity of a parent binary complex to the target locus.

In some embodiments, binding affinity of a plurality of variant binary complexes to two or more target loci (e.g., on-target binding affinity) is increased by at least about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200% or more as compared to binding affinity of a plurality of parent binary complexes to the two or more target loci.

In the context of the present invention, the degree of decreased off-target binding and/or increased on-target binding between a variant binary complex and a parent binary complex may differ by about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or more.

In some embodiments, off-target binding of a variant binary complex to a non-target locus is decreased by at least about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% as compared to off-target binding of a parent binary complex to the non-target locus.

In some embodiments, off-target binding of a plurality of variant binary complexes to two or more non-target loci is decreased by at least about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% as compared to off-target binding of a plurality of parent binary complexes to the two or more non-target loci.

In some embodiments, on-target binding of a variant binary complex to a target locus is increased by at least about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200% or more as compared to on-target binding of a parent binary complex to the target locus.

In some embodiments, on-target binding of a plurality of variant binary complexes to two or more target loci is increased by at least about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200% or more as compared to on-target binding of a plurality of parent binary complexes to the two or more target loci.

In some embodiments, increased binding affinity of a variant binary complex to a target locus (e.g., on-target binding affinity), as compared to a reference, is associated with decreased binding affinity of the variant binary complex to a non-target locus (e.g., off-target binding affinity), as compared to a reference. In some embodiments, binding affinity of a variant binary complex at a target locus (e.g., on-target binding affinity) is inversely associated with binding affinity of the variant binary complex at a non-target locus (e.g., off-target binding affinity). In some embodiments, increased binding affinity of a variant binary complex at a target locus by at least about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more, as compared to a reference (e.g., on-target binding affinity by a parent binary complex), is associated with decreased binding affinity of the variant binary complex at a non-target locus by at least about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more, as compared to a reference (e.g., off-target binding affinity by a parent binary complex). In some embodiments, increased binding affinity of a variant binary complex to a target locus (e.g., on-target binding affinity), as compared to a reference, results in increased binding affinity of the variant binary complex at the target locus (e.g., on-target binding affinity), as compared to a reference.

In some embodiments, increased binding affinity of a plurality of variant binary complexes to two or more target loci (e.g., on-target binding affinity), as compared to a reference, is associated with decreased binding affinity of the plurality of variant binary complexes to two or more non-target loci (e.g., off-target binding affinity), as compared to a reference. In some embodiments, binding affinity of a plurality of variant binary complexes at two or more target loci (e.g., on-target binding affinity) is inversely associated with binding affinity of the plurality of variant binary complexes at two or more non-target loci (e.g., off-target binding affinity). In some embodiments, increased binding affinity of a plurality of variant binary complexes at two or more target loci (e.g., on-target binding affinity) by at least about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more, as compared to a reference (e.g., on-target binding affinity by a parent binary complex), is associated with decreased binding affinity of the plurality of variant binary complexes at two or more non-target loci (e.g., off-target binding affinity) by at least about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more as compared to a reference (e.g., off-target binding affinity by a parent binary complex). In some embodiments, increased binding affinity of a plurality of variant binary complexes to two or more target loci (e.g., on-target binding affinity), as compared to a reference, results in decreased binding affinity of the plurality of variant binary complexes to two or more non-target loci (e.g., off-target binding affinity), as compared to a reference.

In some embodiments, increased binding of a variant binary complex to a target locus (e.g., on-target binding), as compared to a reference, is associated with decreased binding of the variant binary complex to a non-target locus (e.g., off-target binding), as compared to a reference. In some embodiments, binding of a variant binary complex at a target locus (e.g., on-target binding) is inversely associated with binding of the variant binary complex at a non-target locus (e.g., off-target binding). In some embodiments, increased binding of a variant binary complex at a target locus by at least about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more, as compared to a reference (e.g., on-target binding by a parent binary complex), is associated with decreased binding of the variant binary complex at a non-target locus by at least about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more, as compared to a reference (e.g., off-target binding by a parent binary complex). In some embodiments, increased binding of a variant binary complex to a target locus (e.g., on-target binding), as compared to a reference, results in decreased binding of the variant binary complex to a non-target locus (e.g., off-target binding), as compared to a reference.

In some embodiments, increased binding of a plurality of variant binary complexes to two or more target loci (e.g., on-target binding), as compared to a reference, is associated with decreased binding of the plurality of variant binary complexes to two or more non-target loci (e.g., off-target binding), as compared to a reference. In some embodiments, binding of a plurality of variant binary complexes at two or more target loci (e.g., on-target binding) is inversely associated with binding of the plurality of variant binary complexes at two or more non-target loci (e.g., off-target binding). In some embodiments, increased binding of a plurality of variant binary complexes at two or more target loci (on-target binding) by at least about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more, as compared to a reference (e.g., on-target binding by a parent binary complex), is associated with decreased binding of the plurality of variant binary complexes at two or more non-target loci (e.g., off-target binding) by at least about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, or more, as compared to a reference (e.g., off-target binding by a parent binary complex). In some embodiments, increased binding of a plurality of variant binary complexes to two or more target loci (e.g., on-target binding), as compared to a reference, results in decreased binding of the plurality of variant binary complexes to two or more non-target loci (e.g., off-target binding), as compared to a reference.

In some embodiments, increased binding affinity of a variant binary complex to a target locus is associated with increased activity of the variant binary complex at the target locus (e.g., on-target activity). In some embodiments, increased binding affinity of a variant binary complex to a target locus results in increased activity of the variant binary complex at the target locus (e.g., on-target activity).

In some embodiments, increased binding affinity of a plurality of variant binary complexes to two or more target loci is associated with increased activity of the plurality of variant binary complexes at the two or more target loci (e.g., on-target activity). In some embodiments, increased binding affinity of a plurality of variant binary complexes to two or more target loci results in increased activity of the plurality of variant binary complexes at the two or more target loci (e.g., on-target activity).

In some embodiments, increased binding of a variant binary complex to a target locus is associated with increased activity of the variant binary complex at the target locus (e.g., on-target activity). In some embodiments, increased binding of a variant binary complex to a target locus results in increased activity of the variant binary complex at the target locus (e.g., on-target activity).

In some embodiments, increased binding of a plurality of variant binary complexes to two or more target loci is associated with increased activity of the plurality of variant binary complexes at the two or more target loci (e.g., on-target activity). In some embodiments, increased binding of a plurality of variant binary complexes to two or more target loci results in increased activity of the plurality of variant binary complexes at the two or more target loci (e.g., on-target activity).

In some embodiments, decreased binding affinity of a variant binary complex to a non-target locus is associated with decreased activity of the variant binary complex at the non-target locus (e.g., off-target activity). In some embodiments, decreased binding affinity of a variant binary complex to a non-target locus results in decreased activity of the variant binary complex at the non-target locus (e.g., off-target activity).

In some embodiments, decreased binding affinity of a plurality of variant binary complexes to two or more non-target loci is associated with decreased activity of the plurality of variant binary complexes at the two or more non-target loci (e.g., off-target activity). In some embodiments, decreased binding affinity of a plurality of variant binary complexes to two or more non-target loci results in decreased activity of the plurality of variant binary complexes at the two or more non-target loci (e.g., off-target activity).

In some embodiments, decreased binding of a variant binary complex to a non-target locus is associated with decreased activity of the variant binary complex at the non-target locus (e.g., off-target activity). In some embodiments, decreased binding of a variant binary complex to a non-target locus results in decreased activity of the variant binary complex at the non-target locus (e.g., off-target activity).

In some embodiments, decreased binding of a plurality of variant binary complexes to two or more non-target loci is associated with decreased activity of the plurality of variant binary complexes at the two or more non-target loci (e.g., off-target activity). In some embodiments, decreased binding of a plurality of variant binary complexes to two or more non-target loci results in decreased activity of the plurality of variant binary complexes at the two or more non-target loci (e.g., off-target activity).

In some embodiments, decreased binding affinity of a variant binary complex to a non-target locus is associated with increased activity of the variant binary complex at a target locus (e.g., on-target activity). In some embodiments, decreased binding affinity of a variant binary complex to a non-target locus results in increased activity of the variant binary complex at a non-target locus (e.g., on-target activity).

In some embodiments, decreased binding affinity of a plurality of variant binary complexes to two or more non-target loci is associated with increased activity of the plurality of variant binary complexes at two or more on-target loci (e.g., on-target activity). In some embodiments, decreased binding affinity of a plurality of variant binary complexes to two or more non-target loci results in increased activity of the plurality of variant binary complexes at two or more on-target loci (e.g., on-target activity).

In some embodiments, decreased binding of a variant binary complex to a non-target locus is associated with increased activity of the variant binary complex at an on-target locus (e.g., on-target activity). In some embodiments, decreased binding of a variant binary complex to a non-target locus results in increased activity of the variant binary complex at an on-target locus (e.g., on-target activity).

In some embodiments, decreased binding of a plurality of variant binary complexes to two or more non-target loci is associated with increased activity of the plurality of variant binary complexes at two or more on-target loci (e.g., on-target activity). In some embodiments, decreased binding of a plurality of variant binary complexes to two or more non-target loci results in increased activity of the plurality of variant binary complexes at two or more non-target loci (e.g., on-target activity).

In some embodiments, binding affinity of a variant binary complex to a target locus is associated with a ratio of on-target activity (e.g., at the target locus) to off-target activity (e.g., at a non-target locus) of the variant binary complex, as compared to a parent binary complex. In some embodiments, increased binding affinity of a variant binary complex to a target locus is associated with and/or results in an increased ratio of on-target activity (e.g., at the target locus) to off-target activity (e.g., at a non-target locus) of the variant binary complex, as compared to a parent binary complex.

In some embodiments, binding affinity of a plurality of variant binary complexes to two or more target loci is associated with a ratio of on-target activity (e.g., at the two or more target loci) to off-target activity (e.g., at two or more non-target loci) of the plurality of variant binary complexes, as compared to a plurality of parent binary complexes. In some embodiments, increased binding affinity of a plurality of variant binary complexes to two or more target loci is associated with and/or results in an increased ratio of on-target activity (e.g., at the two or more target loci) to off-target activity (e.g., at two or more non-target loci) of the plurality of variant binary complexes, as compared to a plurality of parent binary complexes.

In some embodiments, binding of a variant binary complex to a target locus is associated with a ratio of on-target activity (e.g., at the target locus) to off-target activity (e.g., at a non-target locus) of the variant binary complex, as compared to a parent binary complex. In some embodiments, increased binding of a variant binary complex to a target locus is associated with and/or results in an increased ratio of on-target activity (e.g., at the target locus) to off-target activity (e.g., at a non-target locus) of the variant binary complex, as compared to a parent binary complex.

In some embodiments, binding of a plurality of variant binary complexes to two or more target loci is associated with a ratio of on-target activity (e.g., at the two or more target loci) to off-target activity (e.g., at two or more non-target loci) of the plurality of variant binary complexes, as compared to a plurality of parent binary complexes. In some embodiments, increased binding of a plurality of variant binary complexes to two or more target loci is associated with and/or results in an increased ratio of on-target activity (e.g., at the two or more target loci) to off-target activity (e.g., at two or more non-target loci) of the plurality of variant binary complexes, as compared to a plurality of parent binary complexes.

In some embodiments, a ratio of on-target binding affinity to off-target binding affinity for a variant binary complex is associated with on-target activity (e.g., at a target locus), as compared to a parent binary complex. In some embodiments, an increased ratio of on-target binding affinity to off-target binding affinity for a variant binary complex is associated with and/or results in increased on-target activity (e.g., at a target locus), as compared to a parent binary complex.

In some embodiments, a ratio of on-target binding affinity to off-target binding affinity for a plurality of variant binary complexes is associated with on-target activity (e.g., at two or more target loci), as compared to a plurality of parent binary complexes. In some embodiments, an increased ratio of on-target binding affinity to off-target binding affinity for a plurality of variant binary complexes is associated with and/or results in increased on-target activity (e.g., at two or more target loci), as compared to a plurality of parent binary complexes.

In some embodiments, a ratio of on-target binding to off-target binding for a variant binary complex is associated with on-target activity (e.g., at a target locus), as compared to a parent binary complex. In some embodiments, an increased ratio of on-target binding to off-target binding for a variant binary complex is associated with and/or results in increased on-target activity (e.g., at a target locus), as compared to a parent binary complex.

In some embodiments, a ratio of on-target binding to off-target binding for a plurality of variant binary complexes is associated with on-target activity (e.g., at two or more target loci), as compared to a plurality of parent binary complexes. In some embodiments, an increased ratio of on-target binding to off-target binding for a plurality of variant binary complexes is associated with and/or results in increased on-target activity (e.g., at two or more target loci), as compared to a plurality of parent binary complexes.

### Indel Activity

In some embodiments, the variant binary complex, variant ternary complex formation, or on-target binding of a variant complex induces a DNA break that results in a deletion (e.g., a nucleotide deletion or DNA deletion) adjacent to a 5′-NTTN-3′ sequence, wherein N is any nucleotide. In some embodiments, the on-target binding of a variant complex results in a deletion adjacent to a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence, wherein Y is C or T, B is any nucleotide except for A, D is any nucleotide except for C, and R is A or G. In some embodiments, the on-target binding of a variant complex results in a deletion adjacent to a 5′-CTTT-3′, 5′-CTTC-3′, 5′-GTTT-3′, 5′-GTTC-3′, 5′-TTTC-3′, 5′-GTTA-3′, or 5′-GTTG-3′ sequence. In some embodiments, the variant binary complex, variant ternary complex formation, or on-target binding of a variant complex results in a deletion adjacent to a T/C-rich sequence.

In some embodiments, the variant binary complex, variant ternary complex formation, or on-target binding of a variant complex results in a deletion downstream of a 5′-NTTN-3′ sequence. In some embodiments, the variant binary complex, variant ternary complex formation, or on-target binding of a variant complex results in a deletion downstream of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the variant binary complex, variant ternary complex formation, or on-target binding of a variant complex results in a deletion downstream of a 5′-CTTT-3′, 5′-CTTC-3′, 5′-GTTT-3′, 5′-GTTC-3′, 5′-TTTC-3′, 5′-GTTA-3′, or 5′-GTTG-3′ sequence. In some embodiments, the variant binary complex, variant ternary complex formation, or on-target binding of a variant complex results in a deletion downstream of a T/C-rich sequence.

In some embodiments, the deletion starts within about 5 to about 15 nucleotides (e.g., about 3, 4, 5, 6, 7, 8, 9, 10, 11, 12, 13, 14, 15, 16, or 17 nucleotides) of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) of a 5′-CTTT-3′, 5′-CTTC-3′, 5′-GTTT-3′, 5′-GTTC-3′, 5′-TTTC-3′, 5′-GTTA-3′, or 5′-GTTG-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) of a T/C-rich sequence.

In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a 5′-CTTT-3′, 5′-CTTC-3′, 5′-GTTT-3′, 5′-GTTC-3′, 5′-TTTC-3′, 5′-GTTA-3′, or 5′-GTTG-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a T/C-rich sequence.

In some embodiments, the deletion starts within about 5 to about 10 nucleotides (e.g., about 3, 4, 5, 6, 7, 8, 9, 10, 11, or 12 nucleotides) of the 5′-NTTN-3′ sequence. In some embodiments, the deletion starts within about 5 to about 10 nucleotides (e.g., about 3, 4, 5, 6, 7, 8, 9, 10, 11, or 12 nucleotides) of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the deletion starts within about 5 to about 10 nucleotides (e.g., about 3, 4, 5, 6, 7, 8, 9, 10, 11, or 12 nucleotides) of a 5′-CTTT-3′, 5′-CTTC-3′, 5′-GTTT-3′, 5′-GTTC-3′, 5′-TTTC-3′, 5′-GTTA-3′, or 5′-GTTG-3′ sequence. In some embodiments, the deletion starts within about 5 to about 10 nucleotides (e.g., about 3, 4, 5, 6, 7, 8, 9, 10, 11, or 12 nucleotides) of a T/C-rich sequence.

In some embodiments, the deletion starts within about 5 to about 10 nucleotides (e.g., about 3, 4, 5, 6, 7, 8, 9, 10, 11, or 12 nucleotides) downstream of the 5′-NTTN-3′ sequence. In some embodiments, the deletion starts within about 5 to about 10 nucleotides (e.g., about 3, 4, 5, 6, 7, 8, 9, 10, 11, or 12 nucleotides) downstream of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the deletion starts within about 5 to about 10 nucleotides (e.g., about 3, 4, 5, 6, 7, 8, 9, 10, 11, or 12 nucleotides) downstream of a 5′-CTTT-3′, 5′-CTTC-3′, 5′-GTTT-3′, 5′-GTTC-3′, 5′-TTTC-3′, 5′-GTTA-3′, or 5′-GTTG-3′ sequence. In some embodiments, the deletion starts within about 5 to about 10 nucleotides (e.g., about 3, 4, 5, 6, 7, 8, 9, 10, 11, or 12 nucleotides) downstream of a T/C-rich sequence.

In some embodiments, the deletion starts within about 10 to about 15 nucleotides (e.g., about 8, 9, 10, 11, 12, 13, 14, 15, 16, or 17 nucleotides) of the 5′-NTTN-3′ sequence. In some embodiments, the deletion starts within about 10 to about 15 nucleotides (e.g., about 8, 9, 10, 11, 12, 13, 14, 15, 16, or 17 nucleotides) of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the deletion starts within about 10 to about 15 nucleotides (e.g., about 8, 9, 10, 11, 12, 13, 14, 15, 16, or 17 nucleotides) of a 5′-CTTT-3′, 5′-CTTC-3′, 5′-GTTT-3′, 5′-GTTC-3′, 5′-TTTC-3′, 5′-GTTA-3′, or 5′-GTTG-3′ sequence. In some embodiments, the deletion starts within about 10 to about 15 nucleotides (e.g., about 8, 9, 10, 11, 12, 13, 14, 15, 16, or 17 nucleotides) of a T/C-rich sequence.

In some embodiments, the deletion starts within about 10 to about 15 nucleotides (e.g., about 8, 9, 10, 11, 12, 13, 14, 15, 16, or 17 nucleotides) downstream of the 5′-NTTN-3′ sequence. In some embodiments, the deletion starts within about 10 to about 15 nucleotides (e.g., about 8, 9, 10, 11, 12, 13, 14, 15, 16, or 17 nucleotides) downstream of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the deletion starts within about 10 to about 15 nucleotides (e.g., about 8, 9, 10, 11, 12, 13, 14, 15, 16, or 17 nucleotides) downstream of a 5′-CTTT-3′, 5′-CTTC-3′, 5′-GTTT-3′, 5′-GTTC-3′, 5′-TTTC-3′, 5′-GTTA-3′, or 5′-GTTG-3′ sequence. In some embodiments, the deletion starts within about 10 to about 15 nucleotides (e.g., about 8, 9, 10, 11, 12, 13, 14, 15, 16, or 17 nucleotides) downstream of a T/C-rich sequence.

In some embodiments, the deletion ends within about 20 to about 30 nucleotides (e.g., about 17, 18, 19, 20, 21, 22, 23, 24, 25, 26, 27, 28, 29, 30, 31, 32, or 33 nucleotides) of the 5′-NTTN-3′ sequence. In some embodiments, the deletion ends within about 20 to about 30 nucleotides (e.g., about 17, 18, 19, 20, 21, 22, 23, 24, 25, 26, 27, 28, 29, 30, 31, 32, or 33 nucleotides) of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the deletion ends within about 20 to about 30 nucleotides (e.g., about 17, 18, 19, 20, 21, 22, 23, 24, 25, 26, 27, 28, 29, 30, 31, 32, or 33 nucleotides) of a 5′-CTTT-3′, 5′-CTTC-3′, 5′-GTTT-3′, 5′-GTTC-3′, 5′-TTTC-3′, 5′-GTTA-3′, or 5′-GTTG-3′ sequence. In some embodiments, the deletion ends within about 20 to about 30 nucleotides (e.g., about 17, 18, 19, 20, 21, 22, 23, 24, 25, 26, 27, 28, 29, 30, 31, 32, or 33 nucleotides) of a T/C-rich sequence.

In some embodiments, the deletion ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-NTTN-3′ sequence. In some embodiments, the deletion ends within about 20 to about 30 nucleotides (e.g., about 17, 18, 19, 20, 21, 22, 23, 24, 25, 26, 27, 28, 29, 30, 31, 32, or 33 nucleotides) downstream of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the deletion ends within about 20 to about 30 nucleotides (e.g., about 17, 18, 19, 20, 21, 22, 23, 24, 25, 26, 27, 28, 29, 30, 31, 32, or 33 nucleotides) downstream of a 5′-CTTT-3′, 5′-CTTC-3′, 5′-GTTT-3′, 5′-GTTC-3′, 5′-TTTC-3′, 5′-GTTA-3′, or 5′-GTTG-3′ sequence. In some embodiments, the deletion ends within about 20 to about 30 nucleotides (e.g., about 17, 18, 19, 20, 21, 22, 23, 24, 25, 26, 27, 28, 29, 30, 31, 32, or 33 nucleotides) downstream of a T/C-rich sequence.

In some embodiments, the deletion ends within about 20 to about 25 nucleotides (e.g., about 17, 18, 19, 20, 21, 22, 23, 24, 25, 26, 27, or 28 nucleotides) of the 5′-NTTN-3′ sequence. In some embodiments, the deletion ends within about 20 to about 25 nucleotides (e.g., about 17, 18, 19, 20, 21, 22, 23, 24, 25, 26, 27, or 28 nucleotides) of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the deletion ends within about 20 to about 25 nucleotides (e.g., about 17, 18, 19, 20, 21, 22, 23, 24, 25, 26, 27, or 28 nucleotides) of a 5′-CTTT-3′, 5′-CTTC-3′, 5′-GTTT-3′, 5′-GTTC-3′, 5′-TTTC-3′, 5′-GTTA-3′, or 5′-GTTG-3′ sequence. In some embodiments, the deletion ends within about 20 to about 25 nucleotides (e.g., about 17, 18, 19, 20, 21, 22, 23, 24, 25, 26, 27, or 28 nucleotides) of a T/C-rich sequence.

In some embodiments, the deletion ends within about 20 to about 25 nucleotides (e.g., about 17, 18, 19, 20, 21, 22, 23, 24, 25, 26, 27, or 28 nucleotides) downstream of the 5′-NTTN-3′ sequence. In some embodiments, the deletion ends within about 20 to about 25 nucleotides (e.g., about 17, 18, 19, 20, 21, 22, 23, 24, 25, 26, 27, or 28 nucleotides) downstream of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the deletion ends within about 20 to about 25 nucleotides (e.g., about 17, 18, 19, 20, 21, 22, 23, 24, 25, 26, 27, or 28 nucleotides) downstream of a 5′-CTTT-3′, 5′-CTTC-3′, 5′-GTTT-3′, 5′-GTTC-3′, 5′-TTTC-3′, 5′-GTTA-3′, or 5′-GTTG-3′ sequence. In some embodiments, the deletion ends within about 20 to about 25 nucleotides (e.g., about 17, 18, 19, 20, 21, 22, 23, 24, 25, 26, 27, or 28 nucleotides) downstream of a T/C-rich sequence.

In some embodiments, the deletion ends within about 25 to about 30 nucleotides (e.g., about 22, 23, 24, 25, 26, 27, 28, 29, 30, 31, 32, or 33 nucleotides) of the 5′-NTTN-3′ sequence. In some embodiments, the deletion ends within about 25 to about 30 nucleotides (e.g., about 22, 23, 24, 25, 26, 27, 28, 29, 30, 31, 32, or 33 nucleotides) of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the deletion ends within about 25 to about 30 nucleotides (e.g., about 22, 23, 24, 25, 26, 27, 28, 29, 30, 31, 32, or 33 nucleotides) of a 5′-CTTT-3′, 5′-CTTC-3′, 5′-GTTT-3′, 5′-GTTC-3′, 5′-TTTC-3′, 5′-GTTA-3′, or 5′-GTTG-3′ sequence. In some embodiments, the deletion ends within about 25 to about 30 nucleotides (e.g., about 22, 23, 24, 25, 26, 27, 28, 29, 30, 31, 32, or 33 nucleotides) of a T/C-rich sequence.

In some embodiments, the deletion ends within about 25 to about 30 nucleotides (e.g., about 22, 23, 24, 25, 26, 27, 28, 29, 30, 31, 32, or 33 nucleotides) downstream of the 5′-NTTN-3′ sequence. In some embodiments, the deletion ends within about 25 to about 30 nucleotides (e.g., about 22, 23, 24, 25, 26, 27, 28, 29, 30, 31, 32, or 33 nucleotides) downstream of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the deletion ends within about 25 to about 30 nucleotides (e.g., about 22, 23, 24, 25, 26, 27, 28, 29, 30, 31, 32, or 33 nucleotides) downstream of a 5′-CTTT-3′, 5′-CTTC-3′, 5′-GTTT-3′, 5′-GTTC-3′, 5′-TTTC-3′, 5′-GTTA-3′, or 5′-GTTG-3′ sequence. In some embodiments, the deletion ends within about 25 to about 30 nucleotides (e.g., about 22, 23, 24, 25, 26, 27, 28, 29, 30, 31, 32, or 33 nucleotides) downstream of a T/C-rich sequence.

In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of a 5′-CTTT-3′, 5′-CTTC-3′, 5′-GTTT-3′, 5′-GTTC-3′, 5′-TTTC-3′, 5′-GTTA-3′, or 5′-GTTG-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of a T/C-rich sequence.

In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of the 5′-NTTN-3′ sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a 5′-CTTT-3′, 5′-CTTC-3′, 5′-GTTT-3′, 5′-GTTC-3′, 5′-TTTC-3′, 5′-GTTA-3′, or 5′-GTTG-3′ sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-CTTT-3′, 5′-CTTC-3′, 5′-GTTT-3′, 5′-GTTC-3′, 5′-TTTC-3′, 5′-GTTA-3′, or 5′-GTTG-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a T/C-rich sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the T/C-rich sequence.

In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 20 to about 25 nucleotides (e.g., about 17, 18, 19, 20, 21, 22, 23, 24, 25, 26, 27, or 28 nucleotides) of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 20 to about 25 nucleotides (e.g., about 17, 18, 19, 20, 21, 22, 23, 24, 25, 26, 27, or 28 nucleotides) of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 20 to about 25 nucleotides (e.g., about 17, 18, 19, 20, 21, 22, 23, 24, 25, 26, 27, or 28 nucleotides) of a 5′-CTTT-3′, 5′-CTTC-3′, 5′-GTTT-3′, 5′-GTTC-3′, 5′-TTTC-3′, 5′-GTTA-3′, or 5′-GTTG-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 20 to about 25 nucleotides (e.g., about 17, 18, 19, 20, 21, 22, 23, 24, 25, 26, 27, or 28 nucleotides) of a T/C-rich sequence.

In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of the 5′-NTTN-3′ sequence and ends within about 20 to about 25 nucleotides (e.g., about 17, 18, 19, 20, 21, 22, 23, 24, 25, 26, 27, or 28 nucleotides) downstream of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence and ends within about 20 to about 25 nucleotides (e.g., about 17, 18, 19, 20, 21, 22, 23, 24, 25, 26, 27, or 28 nucleotides) downstream of the 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a 5′-CTTT-3′, 5′-CTTC-3′, 5′-GTTT-3′, 5′-GTTC-3′, 5′-TTTC-3′, 5′-GTTA-3′, or 5′-GTTG-3′ sequence and ends within about 20 to about 25 nucleotides (e.g., about 17, 18, 19, 20, 21, 22, 23, 24, 25, 26, 27, or 28 nucleotides) downstream of the 5′-CTTT-3′, 5′-CTTC-3′, 5′-GTTT-3′, 5′-GTTC-3′, 5′-TTTC-3′, 5′-GTTA-3′, or 5′-GTTG-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a T/C-rich sequence and ends within about 20 to about 25 nucleotides (e.g., about 17, 18, 19, 20, 21, 22, 23, 24, 25, 26, 27, or 28 nucleotides) downstream of the T/C-rich sequence.

In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 25 to about 30 nucleotides (e.g., about 22, 23, 24, 25, 26, 27, 28, 29, 30, 31, 32, or 33 nucleotides) of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 25 to about 30 nucleotides (e.g., about 22, 23, 24, 25, 26, 27, 28, 29, 30, 31, 32, or 33 nucleotides) of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 25 to about 30 nucleotides (e.g., about 22, 23, 24, 25, 26, 27, 28, 29, 30, 31, 32, or 33 nucleotides) of a 5′-CTTT-3′, 5′-CTTC-3′, 5′-GTTT-3′, 5′-GTTC-3′, 5′-TTTC-3′, 5′-GTTA-3′, or 5′-GTTG-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 25 to about 30 nucleotides (e.g., about 22, 23, 24, 25, 26, 27, 28, 29, 30, 31, 32, or 33 nucleotides) of a T/C-rich sequence.

In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of the 5′-NTTN-3′ sequence and ends within about 25 to about 30 nucleotides (e.g., about 22, 23, 24, 25, 26, 27, 28, 29, 30, 31, 32, or 33 nucleotides) downstream of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence and ends within about 25 to about 30 nucleotides (e.g., about 22, 23, 24, 25, 26, 27, 28, 29, 30, 31, 32, or 33 nucleotides) downstream of the 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a 5′-CTTT-3′, 5′-CTTC-3′, 5′-GTTT-3′, 5′-GTTC-3′, 5′-TTTC-3′, 5′-GTTA-3′, or 5′-GTTG-3′ sequence and ends within about 25 to about 30 nucleotides (e.g., about 22, 23, 24, 25, 26, 27, 28, 29, 30, 31, 32, or 33 nucleotides) downstream of the 5′-CTTT-3′, 5′-CTTC-3′, 5′-GTTT-3′, 5′-GTTC-3′, 5′-TTTC-3′, 5′-GTTA-3′, or 5′-GTTG-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a T/C-rich sequence and ends within about 25 to about 30 nucleotides (e.g., about 22, 23, 24, 25, 26, 27, 28, 29, 30, 31, 32, or 33 nucleotides) downstream of the T/C-rich sequence.

In some embodiments, the deletion starts within about 5 to about 10 nucleotides (e.g., about 3, 4, 5, 6, 7, 8, 9, 10, 11, or 12 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of the 5′-NTTN-3′ sequence. In some embodiments, the deletion starts within about 5 to about 10 nucleotides (e.g., about 3, 4, 5, 6, 7, 8, 9, 10, 11, or 12 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the deletion starts within about 5 to about 10 nucleotides (e.g., about 3, 4, 5, 6, 7, 8, 9, 10, 11, or 12 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of a 5′-CTTT-3′, 5′-CTTC-3′, 5′-GTTT-3′, 5′-GTTC-3′, 5′-TTTC-3′, 5′-GTTA-3′, or 5′-GTTG-3′ sequence. In some embodiments, the deletion starts within about 5 to about 10 nucleotides (e.g., about 3, 4, 5, 6, 7, 8, 9, 10, 11, or 12 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of a T/C-rich sequence.

In some embodiments, the deletion starts within about 5 to about 10 nucleotides (e.g., about 3, 4, 5, 6, 7, 8, 9, 10, 11, or 12 nucleotides) downstream of the 5′-NTTN-3′ sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-NTTN-3′ sequence. In some embodiments, the deletion starts within about 5 to about 10 nucleotides (e.g., about 3, 4, 5, 6, 7, 8, 9, 10, 11, or 12 nucleotides) downstream of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the deletion starts within about 5 to about 10 nucleotides (e.g., about 3, 4, 5, 6, 7, 8, 9, 10, 11, or 12 nucleotides) downstream of a 5′-CTTT-3′, 5′-CTTC-3′, 5′-GTTT-3′, 5′-GTTC-3′, 5′-TTTC-3′, 5′-GTTA-3′, or 5′-GTTG-3′ sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-CTTT-3′, 5′-CTTC-3′, 5′-GTTT-3′, 5′-GTTC-3′, 5′-TTTC-3′, 5′-GTTA-3′, or 5′-GTTG-3′ sequence. In some embodiments, the deletion starts within about 5 to about 10 nucleotides (e.g., about 3, 4, 5, 6, 7, 8, 9, 10, 11, or 12 nucleotides) downstream of a T/C-rich sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the T/C-rich sequence.

In some embodiments, the deletion starts within about 5 to about 10 nucleotides (e.g., about 3, 4, 5, 6, 7, 8, 9, 10, 11, or 12 nucleotides) and ends within about 20 to about 25 nucleotides (e.g., about 17, 18, 19, 20, 21, 22, 23, 24, 25, 26, 27, or 28 nucleotides) of the 5′-NTTN-3′ sequence. In some embodiments, the deletion starts within about 5 to about 10 nucleotides (e.g., about 3, 4, 5, 6, 7, 8, 9, 10, 11, or 12 nucleotides) and ends within about 20 to about 25 nucleotides (e.g., about 17, 18, 19, 20, 21, 22, 23, 24, 25, 26, 27, or 28 nucleotides) of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the deletion starts within about 5 to about 10 nucleotides (e.g., about 3, 4, 5, 6, 7, 8, 9, 10, 11, or 12 nucleotides) and ends within about 20 to about 25 nucleotides (e.g., about 17, 18, 19, 20, 21, 22, 23, 24, 25, 26, 27, or 28 nucleotides) of a 5′-CTTT-3′, 5′-CTTC-3′, 5′-GTTT-3′, 5′-GTTC-3′, 5′-TTTC-3′, 5′-GTTA-3′, or 5′-GTTG-3′ sequence. In some embodiments, the deletion starts within about 5 to about 10 nucleotides and ends within about 20 to about 25 nucleotides (e.g., about 17, 18, 19, 20, 21, 22, 23, 24, 25, 26, 27, or 28 nucleotides) (e.g., about 3, 4, 5, 6, 7, 8, 9, 10, 11, or 12 nucleotides) of a T/C-rich sequence.

In some embodiments, the deletion starts within about 5 to about 10 nucleotides (e.g., about 3, 4, 5, 6, 7, 8, 9, 10, 11, or 12 nucleotides) downstream of the 5′-NTTN-3′ sequence and ends within about 20 to about 25 nucleotides (e.g., about 17, 18, 19, 20, 21, 22, 23, 24, 25, 26, 27, or 28 nucleotides) downstream of the 5′-NTTN-3′ sequence. In some embodiments, the deletion starts within about 5 to about 10 nucleotides (e.g., about 3, 4, 5, 6, 7, 8, 9, 10, 11, or 12 nucleotides) downstream of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence and ends within about 20 to about 25 nucleotides (e.g., about 17, 18, 19, 20, 21, 22, 23, 24, 25, 26, 27, or 28 nucleotides) downstream of the 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the deletion starts within about 5 to about 10 nucleotides (e.g., about 3, 4, 5, 6, 7, 8, 9, 10, 11, or 12 nucleotides) downstream of a 5′-CTTT-3′, 5′-CTTC-3′, 5′-GTTT-3′, 5′-GTTC-3′, 5′-TTTC-3′, 5′-GTTA-3′, or 5′-GTTG-3′ sequence and ends within about 20 to about 25 nucleotides (e.g., about 17, 18, 19, 20, 21, 22, 23, 24, 25, 26, 27, or 28 nucleotides) downstream of the 5′-CTTT-3′, 5′-CTTC-3′, 5′-GTTT-3′, 5′-GTTC-3′, 5′-TTTC-3′, 5′-GTTA-3′, or 5′-GTTG-3′ sequence. In some embodiments, the deletion starts within about 5 to about 10 nucleotides (e.g., about 3, 4, 5, 6, 7, 8, 9, 10, 11, or 12 nucleotides) downstream of a T/C-rich sequence and ends within about 20 to about 25 nucleotides (e.g., about 17, 18, 19, 20, 21, 22, 23, 24, 25, 26, 27, or 28 nucleotides) downstream of the T/C-rich sequence.

In some embodiments, the deletion starts within about 5 to about 10 nucleotides (e.g., about 3, 4, 5, 6, 7, 8, 9, 10, 11, or 12 nucleotides) and ends within about 25 to about 30 nucleotides (e.g., about 22, 23, 24, 25, 26, 27, 28, 29, 30, 31, 32, or 33 nucleotides) of the 5′-NTTN-3′ sequence. In some embodiments, the deletion starts within about 5 to about 10 nucleotides (e.g., about 3, 4, 5, 6, 7, 8, 9, 10, 11, or 12 nucleotides) and ends within about 25 to about 30 nucleotides (e.g., about 22, 23, 24, 25, 26, 27, 28, 29, 30, 31, 32, or 33 nucleotides) of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the deletion starts within about 5 to about 10 nucleotides (e.g., about 3, 4, 5, 6, 7, 8, 9, 10, 11, or 12 nucleotides) and ends within about 25 to about 30 nucleotides (e.g., about 22, 23, 24, 25, 26, 27, 28, 29, 30, 31, 32, or 33 nucleotides) of a 5′-CTTT-3′, 5′-CTTC-3′, 5′-GTTT-3′, 5′-GTTC-3′, 5′-TTTC-3′, 5′-GTTA-3′, or 5′-GTTG-3′ sequence. In some embodiments, the deletion starts within about 5 to about 10 nucleotides (e.g., about 3, 4, 5, 6, 7, 8, 9, 10, 11, or 12 nucleotides) and ends within about 25 to about 30 nucleotides (e.g., about 22, 23, 24, 25, 26, 27, 28, 29, 30, 31, 32, or 33 nucleotides) of a T/C-rich sequence.

In some embodiments, the deletion starts within about 5 to about 10 nucleotides (e.g., about 3, 4, 5, 6, 7, 8, 9, 10, 11, or 12 nucleotides) downstream of the 5′-NTTN-3′ sequence and ends within about 25 to about 30 nucleotides (e.g., about 22, 23, 24, 25, 26, 27, 28, 29, 30, 31, 32, or 33 nucleotides) downstream of the 5′-NTTN-3′ sequence. In some embodiments, the deletion starts within about 5 to about 10 nucleotides (e.g., about 3, 4, 5, 6, 7, 8, 9, 10, 11, or 12 nucleotides) downstream of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence and ends within about 25 to about 30 nucleotides (e.g., about 22, 23, 24, 25, 26, 27, 28, 29, 30, 31, 32, or 33 nucleotides) downstream of the 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the deletion starts within about 5 to about 10 nucleotides (e.g., about 3, 4, 5, 6, 7, 8, 9, 10, 11, or 12 nucleotides) downstream of a 5′-CTTT-3′, 5′-CTTC-3′, 5′-GTTT-3′, 5′-GTTC-3′, 5′-TTTC-3′, 5′-GTTA-3′, or 5′-GTTG-3′ sequence and ends within about 25 to about 30 nucleotides (e.g., about 22, 23, 24, 25, 26, 27, 28, 29, 30, 31, 32, or 33 nucleotides) downstream of the 5′-CTTT-3′, 5′-CTTC-3′, 5′-GTTT-3′, 5′-GTTC-3′, 5′-TTTC-3′, 5′-GTTA-3′, or 5′-GTTG-3′ sequence. In some embodiments, the deletion starts within about 5 to about 10 nucleotides (e.g., about 3, 4, 5, 6, 7, 8, 9, 10, 11, or 12 nucleotides) downstream of a T/C-rich sequence and ends within about 25 to about 30 nucleotides (e.g., about 22, 23, 24, 25, 26, 27, 28, 29, 30, 31, 32, or 33 nucleotides) downstream of the T/C-rich sequence.

In some embodiments, the deletion starts within about 10 to about 15 nucleotides (e.g., about 8, 9, 10, 11, 12, 13, 14, 15, 16, or 17 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of the 5′-NTTN-3′ sequence. In some embodiments, the deletion starts within about 10 to about 15 nucleotides (e.g., about 8, 9, 10, 11, 12, 13, 14, 15, 16, or 17 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the deletion starts within about 10 to about 15 nucleotides (e.g., about 8, 9, 10, 11, 12, 13, 14, 15, 16, or 17 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of a 5′-CTTT-3′, 5′-CTTC-3′, 5′-GTTT-3′, 5′-GTTC-3′, 5′-TTTC-3′, 5′-GTTA-3′, or 5′-GTTG-3′ sequence. In some embodiments, the deletion starts within about 10 to about 15 nucleotides (e.g., about 8, 9, 10, 11, 12, 13, 14, 15, 16, or 17 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of a T/C-rich sequence.

In some embodiments, the deletion starts within about 10 to about 15 nucleotides (e.g., about 8, 9, 10, 11, 12, 13, 14, 15, 16, or 17 nucleotides) downstream of the 5′-NTTN-3′ sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-NTTN-3′ sequence. In some embodiments, the deletion starts within about 10 to about 15 nucleotides (e.g., about 8, 9, 10, 11, 12, 13, 14, 15, 16, or 17 nucleotides) downstream of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the deletion starts within about 10 to about 15 nucleotides (e.g., about 8, 9, 10, 11, 12, 13, 14, 15, 16, or 17 nucleotides) downstream of a 5′-CTTT-3′, 5′-CTTC-3′, 5′-GTTT-3′, 5′-GTTC-3′, 5′-TTTC-3′, 5′-GTTA-3′, or 5′-GTTG-3′ sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-CTTT-3′, 5′-CTTC-3′, 5′-GTTT-3′, 5′-GTTC-3′, 5′-TTTC-3′, 5′-GTTA-3′, or 5′-GTTG-3′ sequence. In some embodiments, the deletion starts within about 10 to about 15 nucleotides (e.g., about 8, 9, 10, 11, 12, 13, 14, 15, 16, or 17 nucleotides) downstream of a T/C-rich sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the T/C-rich sequence.

In some embodiments, the deletion starts within about 10 to about 15 nucleotides (e.g., about 8, 9, 10, 11, 12, 13, 14, 15, 16, or 17 nucleotides) and ends within about 20 to about 25 nucleotides (e.g., about 17, 18, 19, 20, 21, 22, 23, 24, 25, 26, 27, or 28 nucleotides) of the 5′-NTTN-3′ sequence. In some embodiments, the deletion starts within about 10 to about 15 nucleotides (e.g., about 8, 9, 10, 11, 12, 13, 14, 15, 16, or 17 nucleotides) and ends within about 20 to about 25 nucleotides (e.g., about 17, 18, 19, 20, 21, 22, 23, 24, 25, 26, 27, or 28 nucleotides) of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the deletion starts within about 10 to about 15 nucleotides (e.g., about 8, 9, 10, 11, 12, 13, 14, 15, 16, or 17 nucleotides) and ends within about 20 to about 25 nucleotides (e.g., about 17, 18, 19, 20, 21, 22, 23, 24, 25, 26, 27, or 28 nucleotides) of a 5′-CTTT-3′, 5′-CTTC-3′, 5′-GTTT-3′, 5′-GTTC-3′, 5′-TTTC-3′, 5′-GTTA-3′, or 5′-GTTG-3′ sequence. In some embodiments, the deletion starts within about 10 to about 15 nucleotides (e.g., about 8, 9, 10, 11, 12, 13, 14, 15, 16, or 17 nucleotides) and ends within about 20 to about 25 nucleotides (e.g., about 17, 18, 19, 20, 21, 22, 23, 24, 25, 26, 27, or 28 nucleotides) of a T/C-rich sequence.

In some embodiments, the deletion starts within about 10 to about 15 nucleotides (e.g., about 8, 9, 10, 11, 12, 13, 14, 15, 16, or 17 nucleotides) downstream of the 5′-NTTN-3′ sequence and ends within about 20 to about 25 nucleotides (e.g., about 17, 18, 19, 20, 21, 22, 23, 24, 25, 26, 27, or 28 nucleotides) downstream of the 5′-NTTN-3′ sequence. In some embodiments, the deletion starts within about 10 to about 15 nucleotides (e.g., about 8, 9, 10, 11, 12, 13, 14, 15, 16, or 17 nucleotides) downstream of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence and ends within about 20 to about 25 nucleotides (e.g., about 17, 18, 19, 20, 21, 22, 23, 24, 25, 26, 27, or 28 nucleotides) downstream of the 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the deletion starts within about 10 to about 15 nucleotides (e.g., about 8, 9, 10, 11, 12, 13, 14, 15, 16, or 17 nucleotides) downstream of a 5′-CTTT-3′, 5′-CTTC-3′, 5′-GTTT-3′, 5′-GTTC-3′, 5′-TTTC-3′, 5′-GTTA-3′, or 5′-GTTG-3′ sequence and ends within about 20 to about 25 nucleotides (e.g., about 17, 18, 19, 20, 21, 22, 23, 24, 25, 26, 27, or 28 nucleotides) downstream of the 5′-CTTT-3′, 5′-CTTC-3′, 5′-GTTT-3′, 5′-GTTC-3′, 5′-TTTC-3′, 5′-GTTA-3′, or 5′-GTTG-3′ sequence. In some embodiments, the deletion starts within about 10 to about 15 nucleotides (e.g., about 8, 9, 10, 11, 12, 13, 14, 15, 16, or 17 nucleotides) downstream of a T/C-rich sequence and ends within about 20 to about 25 nucleotides (e.g., about 17, 18, 19, 20, 21, 22, 23, 24, 25, 26, 27, or 28 nucleotides) downstream of the T/C-rich sequence.

In some embodiments, the deletion starts within about 10 to about 15 nucleotides (e.g., about 8, 9, 10, 11, 12, 13, 14, 15, 16, or 17 nucleotides) and ends within about 25 to about 30 nucleotides (e.g., about 22, 23, 24, 25, 26, 27, 28, 29, 30, 31, 32, or 33 nucleotides) of the 5′-NTTN-3′ sequence. In some embodiments, the deletion starts within about 10 to about 15 nucleotides (e.g., about 8, 9, 10, 11, 12, 13, 14, 15, 16, or 17 nucleotides) and ends within about 25 to about 30 nucleotides (e.g., about 22, 23, 24, 25, 26, 27, 28, 29, 30, 31, 32, or 33 nucleotides) of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the deletion starts within about 10 to about 15 nucleotides (e.g., about 8, 9, 10, 11, 12, 13, 14, 15, 16, or 17 nucleotides) and ends within about 25 to about 30 nucleotides (e.g., about 22, 23, 24, 25, 26, 27, 28, 29, 30, 31, 32, or 33 nucleotides) of a 5′-CTTT-3′, 5′-CTTC-3′, 5′-GTTT-3′, 5′-GTTC-3′, 5′-TTTC-3′, 5′-GTTA-3′, or 5′-GTTG-3′ sequence. In some embodiments, the deletion starts within about 10 to about 15 nucleotides (e.g., about 8, 9, 10, 11, 12, 13, 14, 15, 16, or 17 nucleotides) and ends within about 25 to about 30 nucleotides (e.g., about 22, 23, 24, 25, 26, 27, 28, 29, 30, 31, 32, or 33 nucleotides) of a T/C-rich sequence.

In some embodiments, the deletion starts within about 10 to about 15 nucleotides (e.g., about 8, 9, 10, 11, 12, 13, 14, 15, 16, or 17 nucleotides) downstream of the 5′-NTTN-3′ sequence and ends within about 25 to about 30 nucleotides (e.g., about 22, 23, 24, 25, 26, 27, 28, 29, 30, 31, 32, or 33 nucleotides) downstream of the 5′-NTTN-3′ sequence. In some embodiments, the deletion starts within about 10 to about 15 nucleotides (e.g., about 8, 9, 10, 11, 12, 13, 14, 15, 16, or 17 nucleotides) downstream of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence and ends within about 25 to about 30 nucleotides (e.g., about 22, 23, 24, 25, 26, 27, 28, 29, 30, 31, 32, or 33 nucleotides) downstream of the 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the deletion starts within about 10 to about 15 nucleotides (e.g., about 8, 9, 10, 11, 12, 13, 14, 15, 16, or 17 nucleotides) downstream of a 5′-CTTT-3′, 5′-CTTC-3′, 5′-GTTT-3′, 5′-GTTC-3′, 5′-TTTC-3′, 5′-GTTA-3′, or 5′-GTTG-3′ sequence and ends within about 25 to about 30 nucleotides (e.g., about 22, 23, 24, 25, 26, 27, 28, 29, 30, 31, 32, or 33 nucleotides) downstream of the 5′-CTTT-3′, 5′-CTTC-3′, 5′-GTTT-3′, 5′-GTTC-3′, 5′-TTTC-3′, 5′-GTTA-3′, or 5′-GTTG-3′ sequence. In some embodiments, the deletion starts within about 10 to about 15 nucleotides (e.g., about 8, 9, 10, 11, 12, 13, 14, 15, 16, or 17 nucleotides) downstream of a T/C-rich sequence and ends within about 25 to about 30 nucleotides (e.g., about 22, 23, 24, 25, 26, 27, 28, 29, 30, 31, 32, or 33 nucleotides) downstream of the T/C-rich sequence.

In some embodiments, the deletion is up to about 40 nucleotides in length (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 nucleotides). In some embodiments, the deletion is between about 4 nucleotides and about 40 nucleotides in length (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 nucleotides). In some embodiments, the deletion is between about 4 nucleotides and about 25 nucleotides in length (e.g., about 3, 4, 5, 6, 7, 8, 9, 10, 11, 12, 13, 14, 15, 16, 17, 18, 19, 20, 21, 22, 23, 24, 25, 26, 27, or 28 nucleotides). In some embodiments, the deletion is between about 10 nucleotides and about 25 nucleotides in length (e.g., about 7, 8, 9, 10, 11, 12, 13, 14, 15, 16, 17, 18, 19, 20, 21, 22, 23, 24, 25, 26, 27, or 28 nucleotides). In some embodiments, the deletion is between about 10 nucleotides and about 15 nucleotides in length (e.g., about 7, 8, 9, 10, 11, 12, 13, 14, 15, 16, or 17 nucleotides).

In some embodiments, the gene having a deletion is present in the nucleus of a cell. In some embodiments, the gene having a deletion is endogenous to the cell. In some embodiments, the gene having a deletion is a genomic DNA. In some embodiments, the gene having a deletion is a chromosomal DNA. In some embodiments, the gene having a deletion is a protein-coding gene (e.g., a gene encoding B2M, TRAC, or PDCD1) or a functional region thereof, such as a coding region, or a regulatory element, such as a promoter, enhancer, a 5′ or 3′ untranslated region, etc. In some embodiments, the deletion is in an exon (e.g., an exon of B2M, TRAC, or PDCD1) or an intron. In some embodiments, the gene having the deletion is a non-coding gene, such as transposon, miRNA, tRNA, ribosomal RNA, ribozyme, or lncRNA.

In some embodiments, the deletion alters expression of the gene. In some embodiments, the deletion alters function of the gene. In some embodiments, the deletion inactivates the gene. In some embodiments, the deletion is a frameshifting deletion. In some embodiments, the deletion is a non-frameshifting deletion. In some embodiments, the deletion leads to cell toxicity or cell death (e.g., apoptosis).

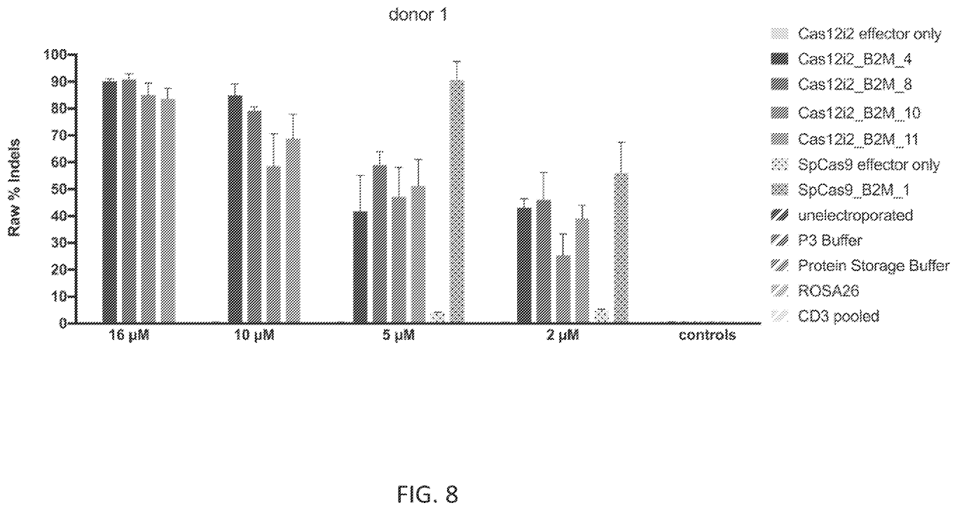

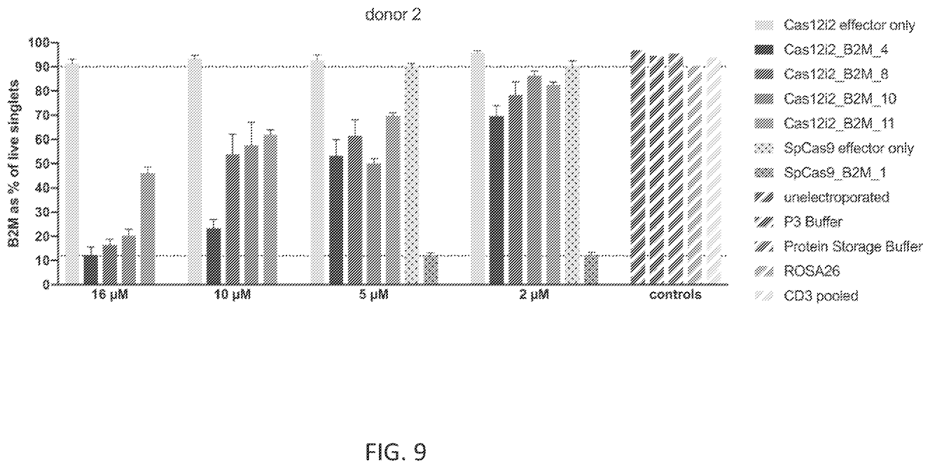

In some embodiments, a variant binary complex (e.g., a variant binary complex exhibiting increased complex formation with a targeting moiety as compared to a parent polypeptide) induces a DNA break that results in a deletion described herein in B2M. In some embodiments, the variant binary complex comprises a crRNA of Table 22. Indel activity in B2M is shown in FIG. 8, and reduction of B2M expression is shown in FIG. 9. See Example 13.

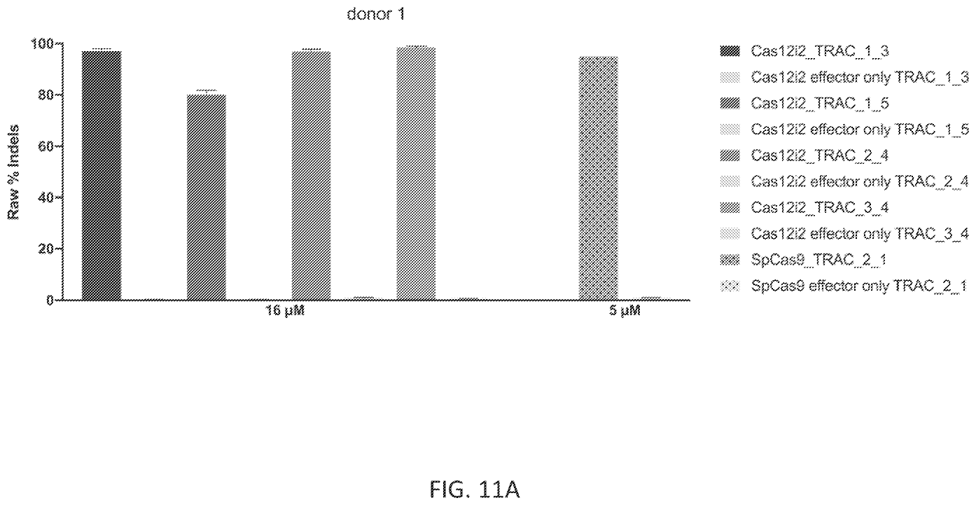

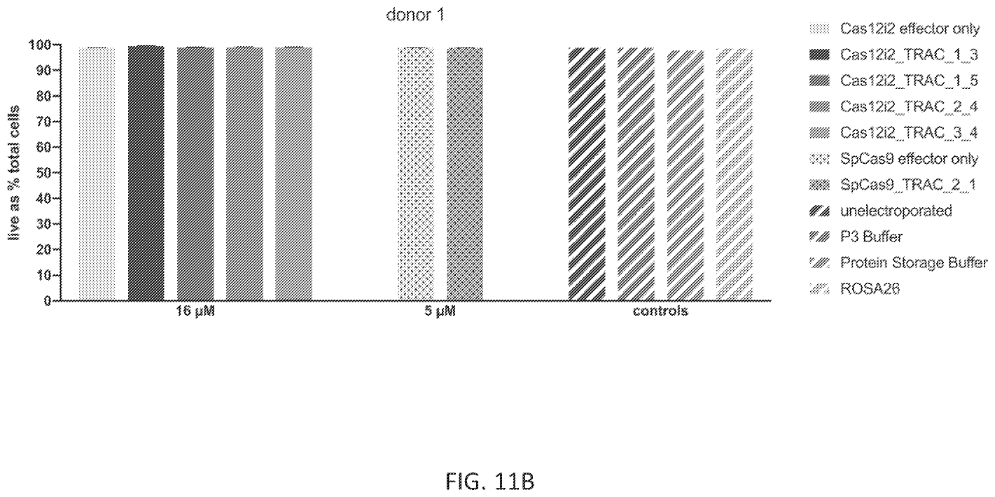

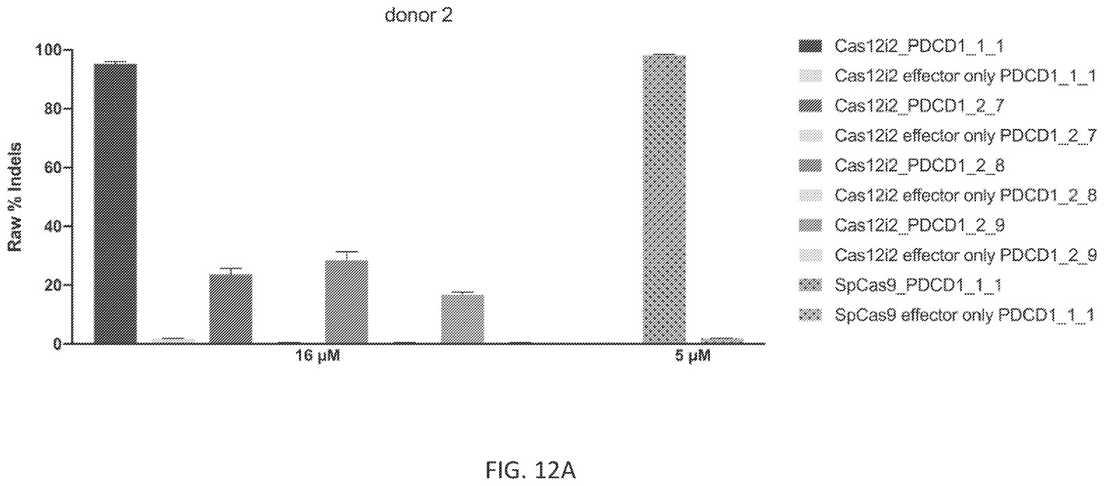

In some embodiments, a variant binary complex (e.g., a variant binary complex exhibiting increased complex formation with a targeting moiety as compared to a parent polypeptide) induces a DNA break that results in a deletion described herein in TRAC. In some embodiments, the variant binary complex comprises a crRNA of Table 23. Indel activity in TRAC is shown in FIG. 11A. See Example 13.

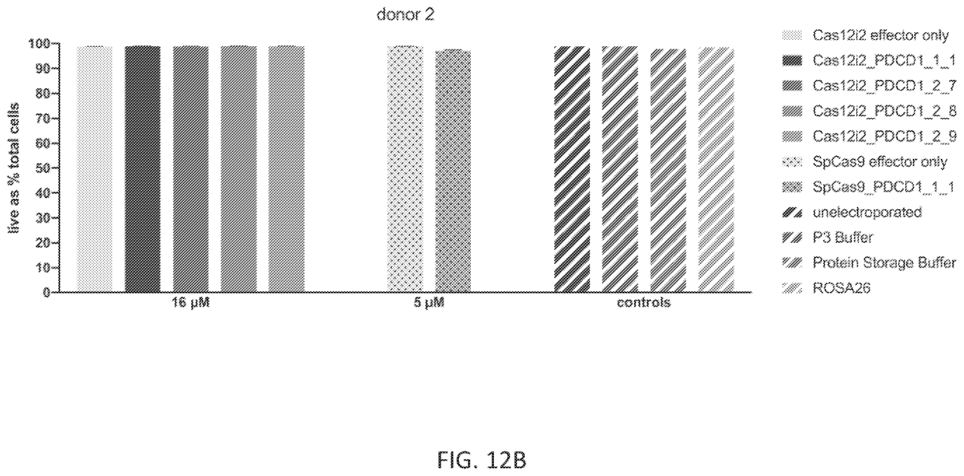

In some embodiments, a variant binary complex (e.g., a variant binary complex exhibiting increased complex formation with a targeting moiety as compared to a parent polypeptide) induces a DNA break that results in a deletion described herein in PDCD1. In some embodiments, the variant binary complex comprises a crRNA of Table 23. Indel activity in PDCD1 is shown in FIG. 12A. See Example 13.

In some embodiments, the deletion overlaps with a mutation in the gene. In some embodiments, the deletion overlaps with an insertion within the gene. For example, in some embodiments, the deletion removes a repeat expansion of the gene. In some embodiments, the deletion disrupts one allele of the gene. In some embodiments, the deletion disrupts both alleles of the gene.

In some embodiments, the deletion is induced in a eukaryotic cell or a prokaryotic cell. In some embodiments, the deletion is induced in an animal cell, a plant cell, or a fungal cell or the cell is derived from an animal cell, a plant cell, or a fungal cell. In some embodiments, the deletion is induced in a mammalian cell or derived from a mammalian cell. In some embodiments, the deletion is induced in a human cell or derived from a human cell. In some embodiments, the deletion is induced in a primary cell. In some embodiments, the deletion is induced in a cell line. In some embodiments, the deletion is induced in a T cell. In some embodiments, the deletion is induced in a stem cell (e.g., a totipotent/omnipotent stem cell, a pluripotent stem cell, a multipotent stem cell, an oligopotent stem cell, or an unipotent stem cell), a differentiated cell, or a terminally differentiated cell. In some embodiments, the variant binary complex selectively induces a DNA break that results in a deletion described herein in a cell (e.g., the variant binary complex does not form a non-target variant ternary complex, or the variant binary complex induces a DNA break that results in a higher ratio of on-target deletions to off-target deletions as compared to a parent binary complex. In some embodiments, a deletion described herein is induced in a cell that lacks additional deletions induced by a variant binary complex, as compared to an unmodified cell. For example, in some embodiments, a deletion described herein is induced in a cell that lacks additional variant Cas12i2 polypeptide-induced deletions greater to or less than about 10 nucleotides in length (e.g., additional deletions of about 40, 30, 25, 20, 15, 10, 9, 8, 7, 6, 5, 4, or 3 nucleotides in length), as compared to an unmodified cell. In some embodiments, the deletion is induced in a cell that lacks Cas12i2 polypeptide-induced insertions greater to or less than about 10 nucleotides in length (e.g., insertions of about 10, 9, 8, 7, 6, 5, 4, 3, 2, or 1 nucleotides in length), as compared to an unmodified cell. In some embodiments, 2 or more (e.g., multiplexed targeted deletions) deletions are induced.

In some aspects, the invention described herein includes a method of obtaining a deletion in a cell, wherein the method comprises contacting a variant Cas12i2 polypeptide as described herein with DNA in the cell.

In some aspects, the invention described herein includes a composition or formulation comprising the variant Cas12i2 polypeptide as described herein, a targeting moiety, and a cell.

### Preparation

In some embodiments, the variant Cas12i2 polypeptide of the present invention can be prepared by (a) culturing bacteria which produce the variant Cas12i2 polypeptide of the present invention, isolating the variant Cas12i2 polypeptide, optionally, purifying the variant Cas12i2 polypeptide, and complexing the variant Cas12i2 polypeptide with RNA guide. The variant Cas12i2 polypeptide can be also prepared by (b) a known genetic engineering technique, specifically, by isolating a gene encoding the variant Cas12i2 polypeptide of the present invention from bacteria, constructing a recombinant expression vector, and then transferring the vector into an appropriate host cell that expresses the RNA guide for expression of a recombinant protein that complexes with the RNA guide in the host cell. Alternatively, the variant Cas12i2 polypeptide can be prepared by (c) an in vitro coupled transcription-translation system and then complexes with RNA guide. Bacteria that can be used for preparation of the variant Cas12i2 polypeptide of the present invention are not particularly limited as long as they can produce the variant Cas12i2 polypeptide of the present invention. Some nonlimiting examples of the bacteria include E. coli cells described herein.

### Vectors

The present invention provides a vector for expressing the variant Cas12i2 polypeptide described herein or nucleic acids encoding the variant described herein may be incorporated into a vector. In some embodiments, a vector of the invention includes a nucleotide sequence encoding variant Cas12i2 polypeptide. In some embodiments, a vector of the invention includes a nucleotide sequence encoding the variant Cas12i2 polypeptide.

The present invention also provides a vector that may be used for preparation of the variant Cas12i2 polypeptide or compositions comprising the variant Cas12i2 polypeptide as described herein. In some embodiments, the invention includes the composition or vector described herein in a cell. In some embodiments, the invention includes a method of expressing the composition comprising the variant Cas12i2 polypeptide, or vector or nucleic acid encoding the variant Cas12i2 polypeptide, in a cell. The method may comprise the steps of providing the composition, e.g., vector or nucleic acid, and delivering the composition to the cell.

Expression of natural or synthetic polynucleotides is typically achieved by operably linking a polynucleotide encoding the gene of interest, e.g., nucleotide sequence encoding the variant Cas12i2 polypeptide, to a promoter and incorporating the construct into an expression vector. The expression vector is not particularly limited as long as it includes a polynucleotide encoding the variant Cas12i2 polypeptide of the present invention and can be suitable for replication and integration in eukaryotic cells.

Typical expression vectors include transcription and translation terminators, initiation sequences, and promoters useful for expression of the desired polynucleotide. For example, plasmid vectors carrying a recognition sequence for RNA polymerase (pSP64, pBluescript, etc.). may be used. Vectors including those derived from retroviruses such as lentivirus are suitable tools to achieve long-term gene transfer since they allow long-term, stable integration of a transgene and its propagation in daughter cells. Examples of vectors include expression vectors, replication vectors, probe generation vectors, and sequencing vectors. The expression vector may be provided to a cell in the form of a viral vector.

Viral vector technology is well known in the art and described in a variety of virology and molecular biology manuals. Viruses which are useful as vectors include, but are not limited to phage viruses, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers.

The kind of the vector is not particularly limited, and a vector that can be expressed in host cells can be appropriately selected. To be more specific, depending on the kind of the host cell, a promoter sequence to ensure the expression of the variant Cas12i2 polypeptide from the polynucleotide is appropriately selected, and this promoter sequence and the polynucleotide are inserted into any of various plasmids etc. for preparation of the expression vector.

Additional promoter elements, e.g., enhancing sequences, regulate the frequency of transcriptional initiation. Typically, these are located in the region 30-110 bp upstream of the start site, although a number of promoters have recently been shown to contain functional elements downstream of the start site as well. Depending on the promoter, it appears that individual elements can function either cooperatively or independently to activate transcription.

Further, the disclosure should not be limited to the use of constitutive promoters. Inducible promoters are also contemplated as part of the disclosure. The use of an inducible promoter provides a molecular switch capable of turning on expression of the polynucleotide sequence which it is operatively linked when such expression is desired or turning off the expression when expression is not desired. Examples of inducible promoters include, but are not limited to a metallothionine promoter, a glucocorticoid promoter, a progesterone promoter, and a tetracycline promoter.

The expression vector to be introduced can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate transcriptional control sequences to enable expression in the host cells. Examples of such a marker include a dihydrofolate reductase gene and a neomycin resistance gene for eukaryotic cell culture; and a tetracycline resistance gene and an ampicillin resistance gene for culture of E. coli and other bacteria. By use of such a selection marker, it can be confirmed whether the polynucleotide encoding the variant Cas12i2 polypeptide of the present invention has been transferred into the host cells and then expressed without fail.

The preparation method for recombinant expression vectors is not particularly limited, and examples thereof include methods using a plasmid, a phage or a cosmid.

### Methods of Expression

The present invention includes a method for protein expression, comprising translating the variant Cas12i2 polypeptide described herein.

In some embodiments, a host cell described herein is used to express the variant Cas12i2 polypeptide. The host cell is not particularly limited, and various known cells can be preferably used. Specific examples of the host cell include bacteria such as E. coli, yeasts (budding yeast, Saccharomyces cerevisiae, and fission yeast, Schizosaccharomyces pombe), nematodes (Caenorhabditis elegans), Xenopus laevis oocytes, and animal cells (for example, CHO cells, COS cells and HEK293 cells). The method for transferring the expression vector described above into host cells, i.e., the transformation method, is not particularly limited, and known methods such as electroporation, the calcium phosphate method, the liposome method and the DEAE dextran method can be used.

After a host is transformed with the expression vector, the host cells may be cultured, cultivated or bred, for production of the variant Cas12i2 polypeptide. After expression of the variant Cas12i2 polypeptide, the host cells can be collected and variant Cas12i2 polypeptide purified from the cultures etc. according to conventional methods (for example, filtration, centrifugation, cell disruption, gel filtration chromatography, ion exchange chromatography, etc.).

In some embodiments, the methods for variant Cas12i2 polypeptide expression comprises translation of at least 5 amino acids, at least 10 amino acids, at least 15 amino acids, at least 20 amino acids, at least 50 amino acids, at least 100 amino acids, at least 150 amino acids, at least 200 amino acids, at least 250 amino acids, at least 300 amino acids, at least 400 amino acids, at least 500 amino acids, at least 600 amino acids, at least 700 amino acids, at least 800 amino acids, at least 900 amino acids, or at least 1000 amino acids of the variant Cas12i2 polypeptide. In some embodiments, the methods for protein expression comprises translation of about 5 amino acids, about 10 amino acids, about 15 amino acids, about 20 amino acids, about 50 amino acids, about 100 amino acids, about 150 amino acids, about 200 amino acids, about 250 amino acids, about 300 amino acids, about 400 amino acids, about 500 amino acids, about 600 amino acids, about 700 amino acids, about 800 amino acids, about 900 amino acids, about 1000 amino acids or more of the variant Cas12i2 polypeptide.

A variety of methods can be used to determine the level of production of a mature variant Cas12i2 polypeptide in a host cell. Such methods include, but are not limited to, for example, methods that utilize either polyclonal or monoclonal antibodies specific for the variant Cas12i2 polypeptide or a labeling tag as described elsewhere herein. Exemplary methods include, but are not limited to, enzyme-linked immunosorbent assays (ELISA), radioimmunoassays (MA), fluorescent immunoassays (FIA), and fluorescent activated cell sorting (FACS). These and other assays are well known in the art (See, e.g., Maddox et al., J. Exp. Med. 158:1211 [1983]).

The present disclosure provides methods of in vivo expression of the variant Cas12i2 polypeptide in a cell, comprising providing a polyribonucleotide encoding the variant Cas12i2 polypeptide to a host cell wherein the polyribonucleotide encodes the variant Cas12i2 polypeptide, expressing the variant Cas12i2 polypeptide in the cell, and obtaining the variant Cas12i2 polypeptide from the cell.

### Introduction of Alteration or Mutation

Nucleic acid sequences encoding variant polypeptides or variant polypeptides may be generated by synthetic methods known in the art. Using the nucleic acid sequence encoding the parent polypeptide itself as a framework, alternations or mutations can be inserted one or more at a time to alter the nucleic acid sequence encoding the parent polypeptide. Along the same lines, the parent polypeptide may be altered or mutated by introducing the changes into the polypeptide sequence as it is synthetically synthesized. This may be accomplished by methods well known in the art.

The production and introduction of alteration or mutation into a parent polypeptide sequence can be accomplished using any methods known by those of skill in the art. In particular, in some embodiments, oligonucleotide primers for PCR may be used for the rapid synthesis of a DNA template including the one or more alterations or mutations in the nucleic acid sequence encoding for the variant polypeptide. Site-specific mutagenesis may also be used as a technique useful in the preparation of individual peptides, or biologically functional equivalent proteins or peptides, through specific mutagenesis of the underlying DNA. The technique further provides a ready ability to prepare and test variants, incorporating one or more of the foregoing considerations, by introducing one or more nucleotide sequence changes into the DNA. Site-specific mutagenesis allows the production of variants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 25 nucleotides in length is preferred, with about 5 to 10 residues on both sides of the junction of the sequence being altered.

Introduction of structural variations, such as fusion of polypeptides as amino- and/or carboxyl-terminal extensions can be accomplished in a similar fashion as introduction of alterations or mutations into the parent polypeptide. The additional peptides may be added to the parent polypeptide or variant polypeptide by including the appropriate nucleic acid sequence encoding the additional peptides to the nucleic acid sequence encoding the parent polypeptide or variant polypeptide. Optionally, the additional peptides may be appended directly to the variant polypeptide through synthetic polypeptide production.

In an aspect, the invention also provides methods for introducing an alteration or mutation into the parent polypeptide sequence to produce a variant Cas12i2 polypeptide that has increased on-target binding with two or more loci (e.g., 2, 3, 4, 5, 6, 7, 8, 9, or more) of a target nucleic acid, as compared to a parent polypeptide.

In an aspect, the invention also provides methods for introducing an alteration or mutation into the parent polypeptide sequence to produce a plurality of variant Cas12i2 polypeptides (e.g., separate variant Cas12i2 polypeptides having the same amino acid sequence), that when individually complexed with a plurality of distinct RNA guides, have increased on-target binding with two or more loci of a target nucleic acid, as compared to a plurality of parent polypeptides and RNA guides.

In an aspect, the invention also provides methods for introducing an alteration or mutation into the parent polypeptide sequence to produce a variant Cas12i2 polypeptide that has increased on-target ternary complex formation with two or more target loci of a target nucleic acid, as compared to a parent polypeptide.

In an aspect, the invention also provides methods for introducing an alteration or mutation into the parent polypeptide sequence to produce a plurality of variant Cas12i2 polypeptides (e.g., separate variant Cas12i2 polypeptides having the same amino acid sequence), that when individually complexed with a plurality of distinct RNA guides, have increased ternary complex formation with two or more loci of a target nucleic acid, as compared to a plurality of parent polypeptides and RNA guides.

In an aspect, the invention also provides methods for introducing an alteration or mutation into the parent polypeptide sequence to produce a Cas12i2 polypeptides exhibit targeting of an increased number of target nucleic acids or target loci, as compared to a parent polypeptide.

In an aspect, the invention also provides methods for introducing an alteration or mutation into the parent polypeptide sequence to produce a plurality of variant Cas12i2 polypeptides (e.g., separate variant Cas12i2 polypeptides having the same amino acid sequence), that when individually complexed with a plurality of distinct RNA guides, exhibit targeting of an increased number of target nucleic acids or target loci, as compared to a plurality of parent polypeptides and RNA guides.

In an aspect, the invention also provides methods for introducing an alteration or mutation into the parent polypeptide sequence to enhance stability of the Cas12i2 polypeptide. Stability of the Cas12i2 polypeptide can be determined by or may include a technique not limited to thermal denaturation assays, thermal shift assays, differential scanning calorimetry (DSC), differential scanning fluorimetry (DSF), isothermal titration calorimetry (ITC), pulse-chase methods, bleach-chase methods, cycloheximide-chase methods, circular dichroism (CD) spectroscopy, crystallization, and fluorescence-based activity assays.

### Variant Selection for Functionality

In an aspect, the invention provides methods for introducing an alteration or mutation into the parent polypeptide sequence to enhance binary complex formation, RNA guide binding activity, and/or RNA guide binding specificity.

In an aspect, the invention also provides methods for introducing an alteration or mutation into the parent polypeptide sequence to enhance ternary complex formation, on-target binding affinity, on-target binding activity, on-target binding, and/or on-target binding specificity. In an aspect, the invention also provides methods for introducing an alteration or mutation into the parent polypeptide sequence to enhance on-target binding affinity (e.g., affinity or time it takes to interact with target), on-target binding activity (e.g., effector activity when interacting with target), on-target binding (e.g., strength of interaction with target), and/or on-target binding specificity (e.g., preference for specific target) of a binary complex (e.g., ribonucleoprotein).

In some embodiments, an alteration or mutation is introduced to the parent polypeptide sequence to produce a variant Cas12i2 polypeptide that has increased on-target binding and/or activity. Also, in such embodiments, off-target binding and/or activity can be decreased in the variant Cas12i2 polypeptide, as compared to the parent polypeptide. Moreover, there can be increased or decreased specificity as to on-target binding vs. off-target binding.

In some embodiments, an alteration or mutation is introduced to the parent polypeptide sequence to produce a variant Cas12i2 polypeptide, that when complexed with an RNA guide, has increased on-target binding. Also, in such embodiments, off-target binding can be decreased in the complex comprising the variant Cas12i2 polypeptide and RNA guide. Moreover, there can be increased or decreased specificity as to on-target binding/activity vs. off-target binding/activity.

In certain embodiments, an alteration or mutation is introduced to the parent polypeptide sequence to produce a variant Cas12i2 polypeptide that enhances stability and/or protein-RNA interactions. In certain embodiments, variant Cas12i2 polypeptide includes at least one alteration that promotes stability and/or RNA interactions as well as enzymatic activity of the variant Cas12i2 polypeptide, as compared to a parent polypeptide.

In certain embodiments, an alteration or mutation is introduced to the parent polypeptide sequence to produce a variant Cas12i2 polypeptide that (a) lacks enzymatic activity, yet (b) retains enhanced stability and/or protein-RNA interactions. In certain embodiments, variant Cas12i2 polypeptide includes at least one alteration that promotes stability and/or RNA interactions, but not enzymatic activity of the variant Cas12i2 polypeptide, as compared to a parent polypeptide.

In certain embodiments, an alteration or mutation is introduced to the parent polypeptide sequence to produce a variant Cas12i2 polypeptide that (a) enhances enzymatic activity, and (b) enhances binary complex formation, RNA guide binding activity, and/or RNA guide binding specificity. In certain embodiments, variant Cas12i2 polypeptide includes at least one alteration that promotes RNA guide complex formation, RNA guide binding activity, and/or RNA guide binding specificity as well as enzymatic activity of the variant Cas12i2 polypeptide, as compared to a parent polypeptide.

In certain embodiments, an alteration or mutation is introduced to the parent polypeptide sequence to produce a variant Cas12i2 polypeptide that (a) lacks enzymatic activity, yet (b) retains enhanced binary complex formation, RNA guide binding activity, and/or RNA guide binding specificity. In certain embodiments, variant Cas12i2 polypeptide includes at least one alteration that promotes binary complex formation, RNA guide binding activity, and/or RNA guide binding specificity, but not enzymatic activity of the variant Cas12i2 polypeptide, as compared to a parent polypeptide.

In certain embodiments, an alteration or mutation is introduced to the parent polypeptide sequence to produce a variant Cas12i2 polypeptide that (a) enhances enzymatic activity, and (b) enhances on-target ternary complex formation, on-target binding affinity, on-target binding activity, and/or on-target binding specificity. In certain embodiments, variant Cas12i2 polypeptide includes at least one alteration that promotes on-target ternary complex formation, on-target binding affinity, on-target binding activity, and/or on-target binding specificity as well as enzymatic activity of the variant Cas12i2 polypeptide, as compared to a parent polypeptide.

In certain embodiments, an alteration or mutation is introduced to the parent polypeptide sequence to produce a variant Cas12i2 polypeptide that (a) lacks enzymatic activity, yet (b) retains enhanced on-target ternary complex formation, on-target binding affinity, on-target binding activity, and/or on-target binding specificity. In certain embodiments, variant Cas12i2 polypeptide includes at least one alteration that promotes on-target ternary complex formation, on-target binding affinity, on-target binding activity, and/or on-target binding specificity, but not enzymatic activity of the variant Cas12i2 polypeptide, as compared to a parent polypeptide.

In some embodiments, at least one alteration is introduced into the parent polypeptide of SEQ ID NO: 2 to produce a variant Cas12i2 polypeptide that exhibits (a) decreased enzymatic activity and (b) enhanced RNA affinity relative to the parent polypeptide of SEQ ID NO: 2. In some embodiments, at least one alteration is introduced into the parent polypeptide of SEQ ID NO: 2 to produce a variant Cas12i2 polypeptide that exhibits (a) increased enzymatic activity and (b) enhanced RNA affinity, relative to the parent polypeptide of SEQ ID NO: 2. In some embodiments, at least one alteration is introduced into the parent polypeptide of SEQ ID NO: 2 to produce a variant Cas12i2 polypeptide that exhibits (a) retained enzymatic activity and (b) enhanced RNA affinity, relative to the parent polypeptide of SEQ ID NO: 2. In some embodiments, the variant Cas12i2 polypeptide having a feature as described herein comprises an amino acid sequence having at least about 60%, 65%, 70%, 75%, 80%, 85%, 90%, 91%, 92%, 93%, 94%, 95%, 96%, 97%, 98%, 99%, or 100% identity to any one of SEQ ID NOs: 3-146 and 495-512.

In some embodiments, at least one alteration is introduced into the parent polypeptide of SEQ ID NO: 2 to produce a variant Cas12i2 polypeptide that exhibits (a) decreased enzymatic activity and (b) enhanced binary complex formation relative to the parent polypeptide of SEQ ID NO: 2. In some embodiments, at least one alteration is introduced into the parent polypeptide of SEQ ID NO: 2 to produce a variant Cas12i2 polypeptide that exhibits (a) increased enzymatic activity and (b) enhanced binary complex formation, relative to the parent polypeptide of SEQ ID NO: 2. In some embodiments, at least one alteration is introduced into the parent polypeptide of SEQ ID NO: 2 to produce a variant Cas12i2 polypeptide that exhibits (a) retained enzymatic activity and (b) enhanced binary complex formation, relative to the parent polypeptide of SEQ ID NO: 2. In some embodiments, the variant Cas12i2 polypeptide having a feature as described herein comprises an amino acid sequence having at least about 60%, 65%, 70%, 75%, 80%, 85%, 90%, 91%, 92%, 93%, 94%, 95%, 96%, 97%, 98%, 99%, or 100% identity to any one of SEQ ID NOs: 3-146 and 495-512.

In some embodiments, at least one alteration is introduced into the parent polypeptide of SEQ ID NO: 2 to produce a variant Cas12i2 polypeptide that exhibits (a) decreased enzymatic activity and (b) enhanced RNA guide binding activity relative to the parent polypeptide of SEQ ID NO: 2. In some embodiments, at least one alteration is introduced into the parent polypeptide of SEQ ID NO: 2 to produce a variant Cas12i2 polypeptide that exhibits (a) increased enzymatic activity and (b) enhanced RNA guide binding activity, relative to the parent polypeptide of SEQ ID NO: 2. In some embodiments, at least one alteration is introduced into the parent polypeptide of SEQ ID NO: 2 to produce a variant Cas12i2 polypeptide that exhibits (a) retained enzymatic activity and (b) enhanced RNA guide binding activity, relative to the parent polypeptide of SEQ ID NO: 2. In some embodiments, the variant Cas12i2 polypeptide having a feature as described herein comprises an amino acid sequence having at least about 60%, 65%, 70%, 75%, 80%, 85%, 90%, 91%, 92%, 93%, 94%, 95%, 96%, 97%, 98%, 99%, or 100% identity to any one of SEQ ID NOs: 3-146 and 495-512.

In some embodiments, at least one alteration is introduced into the parent polypeptide of SEQ ID NO: 2 to produce a variant Cas12i2 polypeptide that exhibits (a) decreased enzymatic activity and (b) enhanced RNA guide binding specificity relative to the parent polypeptide of SEQ ID NO: 2. In some embodiments, at least one alteration is introduced into the parent polypeptide of SEQ ID NO: 2 to produce a variant Cas12i2 polypeptide that exhibits (a) increased enzymatic activity and (b) enhanced RNA guide binding specificity, relative to the parent polypeptide of SEQ ID NO: 2. In some embodiments, at least one alteration is introduced into the parent polypeptide of SEQ ID NO: 2 to produce a variant Cas12i2 polypeptide that exhibits (a) retained enzymatic activity and (b) enhanced RNA guide binding specificity, relative to the parent polypeptide of SEQ ID NO: 2. In some embodiments, the variant Cas12i2 polypeptide having a feature as described herein comprises an amino acid sequence having at least about 60%, 65%, 70%, 75%, 80%, 85%, 90%, 91%, 92%, 93%, 94%, 95%, 96%, 97%, 98%, 99%, or 100% identity to any one of SEQ ID NOs: 3-146 and 495-512.

In some embodiments, at least one alteration is introduced into the parent polypeptide of SEQ ID NO: 2 to produce a variant Cas12i2 polypeptide that exhibits (a) decreased enzymatic activity and (b) enhanced protein-RNA interactions relative to the parent polypeptide of SEQ ID NO: 2. In some embodiments, at least one alteration is introduced into the parent polypeptide of SEQ ID NO: 2 to produce a variant Cas12i2 polypeptide that exhibits (a) increased enzymatic activity and (b) enhanced protein-RNA interactions, relative to the parent polypeptide of SEQ ID NO: 2. In some embodiments, at least one alteration is introduced into the parent polypeptide of SEQ ID NO: 2 to produce a variant Cas12i2 polypeptide that exhibits (a) retained enzymatic activity and (b) enhanced protein-RNA interactions, relative to the parent polypeptide of SEQ ID NO: 2. In some embodiments, the variant Cas12i2 polypeptide having a feature as described herein comprises an amino acid sequence having at least about 60%, 65%, 70%, 75%, 80%, 85%, 90%, 91%, 92%, 93%, 94%, 95%, 96%, 97%, 98%, 99%, or 100% identity to any one of SEQ ID NOs: 3-146 and 495-512.

In some embodiments, at least one alteration is introduced into the parent polypeptide of SEQ ID NO: 2 to produce a variant Cas12i2 polypeptide that exhibits (a) decreased enzymatic activity and (b) enhanced protein stability relative to the parent polypeptide of SEQ ID NO: 2. In some embodiments, at least one alteration is introduced into the parent polypeptide of SEQ ID NO: 2 to produce a variant Cas12i2 polypeptide that exhibits (a) increased enzymatic activity and (b) enhanced protein stability, relative to the parent polypeptide of SEQ ID NO: 2. In some embodiments, at least one alteration is introduced into the parent polypeptide of SEQ ID NO: 2 to produce a variant Cas12i2 polypeptide that exhibits (a) retained enzymatic activity and (b) enhanced protein stability, relative to the parent polypeptide of SEQ ID NO: 2. In some embodiments, the variant Cas12i2 polypeptide having a feature as described herein comprises an amino acid sequence having at least about 60%, 65%, 70%, 75%, 80%, 85%, 90%, 91%, 92%, 93%, 94%, 95%, 96%, 97%, 98%, 99%, or 100% identity to any one of SEQ ID NOs: 3-146 and 495-512.

In some embodiments, at least one alteration is introduced into the parent polypeptide of SEQ ID NO: 2 to produce a variant Cas12i2 polypeptide that exhibits (a) decreased enzymatic activity and (b) decreased dissociation from an RNA guide relative to the parent polypeptide of SEQ ID NO: 2. In some embodiments, at least one alteration is introduced into the parent polypeptide of SEQ ID NO: 2 to produce a variant Cas12i2 polypeptide that exhibits (a) increased enzymatic activity and (b) decreased dissociation from an RNA guide, relative to the parent polypeptide of SEQ ID NO: 2. In some embodiments, at least one alteration is introduced into the parent polypeptide of SEQ ID NO: 2 to produce a variant Cas12i2 polypeptide that exhibits (a) retained enzymatic activity and (b) decreased dissociation from an RNA guide, relative to the parent polypeptide of SEQ ID NO: 2. In some embodiments, the variant Cas12i2 polypeptide having a feature as described herein comprises an amino acid sequence having at least about 60%, 65%, 70%, 75%, 80%, 85%, 90%, 91%, 92%, 93%, 94%, 95%, 96%, 97%, 98%, 99%, or 100% identity to any one of SEQ ID NOs: 3-146 and 495-512.

In some embodiments, at least one alteration is introduced into the parent polypeptide of SEQ ID NO: 2 to produce a variant Cas12i2 polypeptide that exhibits (a) decreased enzymatic activity and (b) enhanced ternary complex formation relative to the parent polypeptide of SEQ ID NO: 2. In some embodiments, at least one alteration is introduced into the parent polypeptide of SEQ ID NO: 2 to produce a variant Cas12i2 polypeptide that exhibits (a) increased enzymatic activity and (b) enhanced ternary complex formation, relative to the parent polypeptide of SEQ ID NO: 2. In some embodiments, at least one alteration is introduced into the parent polypeptide of SEQ ID NO: 2 to produce a variant Cas12i2 polypeptide that exhibits (a) retained enzymatic activity and (b) enhanced ternary complex formation, relative to the parent polypeptide of SEQ ID NO: 2. In some embodiments, the variant Cas12i2 polypeptide having a feature as described herein comprises an amino acid sequence having at least about 60%, 65%, 70%, 75%, 80%, 85%, 90%, 91%, 92%, 93%, 94%, 95%, 96%, 97%, 98%, 99%, or 100% identity to any one of SEQ ID NOs: 3-146 and 495-512.

In some embodiments, at least one alteration is introduced into the parent polypeptide of SEQ ID NO: 2 to produce a variant Cas12i2 polypeptide that forms a variant binary complex exhibiting (a) decreased enzymatic activity and (b) enhanced binding affinity to a target nucleic acid, relative to a parent binary complex comprising the polypeptide of SEQ ID NO: 2. In some embodiments, at least one alteration is introduced into the parent polypeptide of SEQ ID NO: 2 to produce a variant Cas12i2 polypeptide that forms a variant binary complex exhibiting (a) increased enzymatic activity and (b) enhanced binding affinity to a target nucleic acid, relative to a parent binary complex comprising the polypeptide of SEQ ID NO: 2. In some embodiments, at least one alteration is introduced into the parent polypeptide of SEQ ID NO: 2 to produce a variant Cas12i2 polypeptide that forms a variant binary complex that exhibits (a) retained enzymatic activity and (b) enhanced binding affinity to a target nucleic acid, relative to a parent binary complex comprising the polypeptide of SEQ ID NO: 2. In some embodiments, the variant Cas12i2 polypeptide having a feature as described herein comprises an amino acid sequence having at least about 60%, 65%, 70%, 75%, 80%, 85%, 90%, 91%, 92%, 93%, 94%, 95%, 96%, 97%, 98%, 99%, or 100% identity to any one of SEQ ID NOs: 3-146 and 495-512.

In some embodiments, at least one alteration is introduced into the parent polypeptide of SEQ ID NO: 2 to produce a variant Cas12i2 polypeptide that forms a variant binary complex exhibiting (a) decreased enzymatic activity and (b) enhanced on-target binding activity, relative to a parent binary complex comprising the polypeptide of SEQ ID NO: 2. In some embodiments, at least one alteration is introduced into the parent polypeptide of SEQ ID NO: 2 to produce a variant Cas12i2 polypeptide that forms a variant binary complex exhibiting (a) increased enzymatic activity and (b) enhanced on-target binding activity, relative to a parent binary complex comprising the polypeptide of SEQ ID NO: 2. In some embodiments, at least one alteration is introduced into the parent polypeptide of SEQ ID NO: 2 to produce a variant Cas12i2 polypeptide that forms a variant binary complex that exhibits (a) retained enzymatic activity and (b) enhanced on-target binding activity, relative to a parent binary complex comprising the polypeptide of SEQ ID NO: 2. In some embodiments, the variant Cas12i2 polypeptide having a feature as described herein comprises an amino acid sequence having at least about 60%, 65%, 70%, 75%, 80%, 85%, 90%, 91%, 92%, 93%, 94%, 95%, 96%, 97%, 98%, 99%, or 100% identity to any one of SEQ ID NOs: 3-146 and 495-512.

In some embodiments, at least one alteration is introduced into the parent polypeptide of SEQ ID NO: 2 to produce a variant Cas12i2 polypeptide that forms a variant binary complex exhibiting (a) decreased enzymatic activity and (b) enhanced on-target binding specificity, relative to a parent binary complex comprising the polypeptide of SEQ ID NO: 2. In some embodiments, at least one alteration is introduced into the parent polypeptide of SEQ ID NO: 2 to produce a variant Cas12i2 polypeptide that forms a variant binary complex exhibiting (a) increased enzymatic activity and (b) enhanced on-target binding specificity, relative to a parent binary complex comprising the polypeptide of SEQ ID NO: 2. In some embodiments, at least one alteration is introduced into the parent polypeptide of SEQ ID NO: 2 to produce a variant Cas12i2 polypeptide that forms a variant binary complex that exhibits (a) retained enzymatic activity and (b) enhanced on-target binding specificity, relative to a parent binary complex comprising the polypeptide of SEQ ID NO: 2. In some embodiments, the variant Cas12i2 polypeptide having a feature as described herein comprises an amino acid sequence having at least about 60%, 65%, 70%, 75%, 80%, 85%, 90%, 91%, 92%, 93%, 94%, 95%, 96%, 97%, 98%, 99%, or 100% identity to any one of SEQ ID NOs: 3-146 and 495-512.

In some embodiments, at least one alteration is introduced into the parent polypeptide of SEQ ID NO: 2 to produce a variant Cas12i2 polypeptide that forms a variant binary complex exhibiting (a) decreased enzymatic activity and (b) decreased off-target binding to a non-target nucleic acid, relative to a parent binary complex comprising the polypeptide of SEQ ID NO: 2. In some embodiments, at least one alteration is introduced into the parent polypeptide of SEQ ID NO: 2 to produce a variant Cas12i2 polypeptide that forms a variant binary complex exhibiting (a) increased enzymatic activity and (b) decreased off-target binding to a non-target nucleic acid, relative to a parent binary complex comprising the polypeptide of SEQ ID NO: 2. In some embodiments, at least one alteration is introduced into the parent polypeptide of SEQ ID NO: 2 to produce a variant Cas12i2 polypeptide that forms a variant binary complex exhibiting (a) retained enzymatic activity and (b) decreased off-target binding to a non-target nucleic acid, relative to a parent binary complex comprising the polypeptide of SEQ ID NO: 2. In some embodiments, the variant Cas12i2 polypeptide having a feature as described herein comprises an amino acid sequence having at least about 60%, 65%, 70%, 75%, 80%, 85%, 90%, 91%, 92%, 93%, 94%, 95%, 96%, 97%, 98%, 99%, or 100% identity to any one of SEQ ID NOs: 3-146 and 495-512.

In some embodiments, at least one alteration is introduced into the parent polypeptide of SEQ ID NO: 2 to produce a variant Cas12i2 polypeptide that forms a variant binary complex exhibiting (a) decreased enzymatic activity and (b) decreased dissociation from the target nucleic acid, relative to a parent binary complex comprising the polypeptide of SEQ ID NO: 2. In some embodiments, at least one alteration is introduced into the parent polypeptide of SEQ ID NO: 2 to produce a variant Cas12i2 polypeptide that forms a variant binary complex exhibiting (a) increased enzymatic activity and (b) decreased dissociation from the target nucleic acid, relative to a parent binary complex comprising the polypeptide of SEQ ID NO: 2. In some embodiments, at least one alteration is introduced into the parent polypeptide of SEQ ID NO: 2 to produce a variant Cas12i2 polypeptide that forms a variant binary complex exhibiting (a) retained enzymatic activity and (b) decreased dissociation from the target nucleic acid, relative to a parent binary complex comprising the polypeptide of SEQ ID NO: 2. In some embodiments, the variant Cas12i2 polypeptide having a feature as described herein comprises an amino acid sequence having at least about 60%, 65%, 70%, 75%, 80%, 85%, 90%, 91%, 92%, 93%, 94%, 95%, 96%, 97%, 98%, 99%, or 100% identity to any one of SEQ ID NOs: 3-146 and 495-512.

### Variant Binary Complexing

Generally, the variant Cas12i2 polypeptide and the RNA guide bind to each other in a molar ratio of about 1:1 to form the variant binary complex. The variant Cas12i2 polypeptide and the RNA guide, either alone or together, do not naturally occur.

In some embodiments, the variant Cas12i2 polypeptide can be overexpressed in a host cell and purified as described herein, then complexed with the RNA guide (e.g., in a test tube) to form a variant effector RNP (e.g., variant binary complex).

In some embodiments, the variant binary complex exhibits increased binding affinity to a target nucleic acid, increased on-target binding activity, increased on-target binding specificity, increased ternary complex formation with a target nucleic acid, and/or increased stability over a range of incubation times. In some embodiments, the variant binary complex exhibits decreased off-target binding to a non-target nucleic acid and/or decreased dissociation from a target nucleic acid over a range of incubation times. In some embodiments, the variant binary complex exhibits increased target nucleic acid complex formation, target nucleic acid activity, and/or target nucleic acid specificity over a range of incubation times.

In some embodiments, complexation of a binary complex may occur at a temperature in the range of about 20° C. to about 55° C., e.g., about any one of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 50° C., or 55° C. In some embodiments, the variant Cas12i2 polypeptide does not dissociate from the RNA guide or bind to a free RNA at about 37° C. over an incubation period of at least about any one of 10 mins, 15 mins, 20 mins, 25 mins, 30 mins, 35 mins, 40 mins, 45 mins, 50 mins, 55 mins, 1 hr, 2 hr, 3 hr, 4 hr, or more hours. In some embodiments, after binary complex formation, the variant effector ribonucleoprotein complex does not exchange the RNA guide with a different RNA.

In some embodiments, the variant Cas12i2 polypeptide and RNA guide are complexed in a binary complexation buffer. In some embodiments, the variant Cas12i2 polypeptide is stored in a buffer that is replaced with a binary complexation buffer to form a complex with the RNA guide. In some embodiments, the variant Cas12i2 polypeptide is stored in a binary complexation buffer.

In some embodiments, the binary complexation buffer has a pH in a range of about 7.3 to 8.6. In one embodiment, the pH of the binary complexation buffer is about 7.3. In one embodiment, the pH of the binary complexation buffer is about 7.4. In one embodiment, the pH of the binary complexation buffer is about 7.5. In one embodiment, the pH of the binary complexation buffer is about 7.6. In one embodiment, the pH of the binary complexation buffer is about 7.7. In one embodiment, the pH of the binary complexation buffer is about 7.8. In one embodiment, the pH of the binary complexation buffer is about 7.9. In one embodiment, the pH of the binary complexation buffer is about 8.0. In one embodiment, the pH of the binary complexation buffer is about 8.1. In one embodiment, the pH of the binary complexation buffer is about 8.2. In one embodiment, the pH of the binary complexation buffer is about 8.3. In one embodiment, the pH of the binary complexation buffer is about 8.4. In one embodiment, the pH of the binary complexation buffer is about 8.5. In one embodiment, the pH of the binary complexation buffer is about 8.6.

The thermostability of the variant Cas12i2 polypeptide can increase under favorable conditions such as the addition of an RNA guide, e.g., binding an RNA guide.

In some embodiments, the variant Cas12i2 polypeptide can be overexpressed and complexed with the RNA guide in a host cell prior to purification as described herein. In some embodiments, mRNA or DNA encoding the variant Cas12i2 polypeptide is introduced into a cell so that the variant Cas12i2 polypeptide is expressed in the cell. The RNA guide, which guides the variant Cas12i2 polypeptide to the desired target nucleic acid is also introduced into the cell, whether simultaneously, separately or sequentially from a single mRNA or DNA construct, such that the necessary ribonucleoprotein complex is formed in the cell.

### Assessing Variant Binary Complex Stability and Functionality

Provided herein in certain embodiments are methods for identifying an optimal variant Cas12i2 polypeptide/RNA guide complex (referred to herein as the variant binary complex) including (a) combining a variant Cas12i2 polypeptide and an RNA guide in a sample to form the variant binary complex; (b) measuring a value of the variant binary complex; and (c) determining the variant binary complex is optimal over the reference molecule, if the value of the variant binary complex is greater than a value of a reference molecule. In some embodiments, the value may include, but is not limited to, a stability measurement (e.g., Tₘ value, thermostability), a rate of binary complex formation, RNA guide binding specificity, and/or complex activity.

In some embodiments, an optimal variant Cas12i2 polypeptide/RNA guide complex (i.e., a variant binary complex) is identified by the steps of: (a) combining a variant Cas12i2 polypeptide and an RNA guide in a sample to form the variant binary complex; (b) detecting a Tₘ value of the variant binary complex; and (c) determining the variant binary complex is stable if the Tₘ value of the variant binary complex is greater than a Tₘ value of a reference molecule or a Tₘ reference value by at least 8° C.

The methods involving a step of measuring the thermostability of a variant Cas12i2 polypeptide/RNA guide complex (i.e., a variant binary complex) may include, without limitation, methods of determining the stability of a variant binary complex, methods of determining a condition that promotes a stable variant binary complex, methods of screening for a stable variant binary complex, and methods for identifying an optimal gRNA to form a stable variant binary complex. In certain embodiments, a thermostability value of a variant binary complex may be measured.

Additionally, in certain embodiments, a thermostability value of a reference molecule may also be measured. In certain embodiments, a variant binary complex may be determined to be stable if the measured thermostability value of the variant binary complex is greater than the measured thermostability value of the reference molecule or a thermostability reference value, measured under the same experimental conditions, as described herein. In certain embodiments, the reference molecule may be the variant Cas12i2 polypeptide absent an RNA guide.

In certain embodiments, the thermostability value that is measured may be a denaturation temperature value. In these embodiments, the thermostability reference value is a denaturation temperature reference value. In certain embodiments, the thermostability value that is measured may be a Tₘ value. In these embodiments, the thermostability reference value may be a Tₘ reference value. In certain embodiments, the thermostability value may be measured using a thermal shift assay. In certain embodiments, an assay used to measure thermostability may involve a technique described herein including, but not limited to, thermal denaturation assays, thermal shift assays, differential scanning calorimetry (DSC), differential scanning fluorimetry (DSF), isothermal titration calorimetry (ITC), pulse-chase methods, bleach-chase methods, cycloheximide-chase methods, circular dichroism (CD) spectroscopy, crystallization, and fluorescence-based activity assays.

In certain embodiments, a variant binary complex may be identified if the rate of variant Cas12i2 polypeptide/RNA guide complex formation, RNA guide binding specificity, and/or complex activity of the variant binary complex is greater than a value of the reference molecule or the reference value (e.g., a value of a parent polypeptide/RNA guide complex, referred to herein as a parent binary complex). For example, in certain embodiments, the variant binary complex may be identified if the value of a rate of variant Cas12i2 polypeptide/RNA guide complex formation, RNA guide binding specificity, and/or complex activity of the variant binary complex is at least X % greater than a value of the reference molecule or the reference value (e.g., a value of a parent binary complex). In certain embodiments, the methods described herein may further comprise steps that include measuring the activity of the variant binary complex as described herein.

### Variant Ternary Complexing

In some embodiments, the variant Cas12i2 polypeptide, RNA guide, and target nucleic acid, as described herein, form a variant ternary complex (e.g., in a test tube or cell). Generally, the variant Cas12i2 polypeptide, the RNA guide, and the target nucleic acid associate with each other in a molar ratio of about 1:1:1 to form the variant ternary complex. The variant Cas12i2 polypeptide, the RNA guide, and the target nucleic acid, either alone or together, do not naturally occur.

In some embodiments, the variant binary complex (e.g., complex of variant Cas12i2 polypeptide and RNA guide) as described herein, is further complexed with the target nucleic acid (e.g., in a test tube or cell) to form a variant ternary complex.

In some embodiments, complexation of the ternary complex occurs at a temperature in the range of about 20° C. to about 55° C., e.g., about any one of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 50° C., or 55° C. In some embodiments, the variant binary complex does not dissociate from the target nucleic acid or bind to a free nucleic acid (e.g., free DNA) at about 37° C. over an incubation period of at least about any one of 10 mins, 15 mins, 20 mins, 25 mins, 30 mins, 35 mins, 40 mins, 45 mins, 50 mins, 55 mins, 1 hr, 2 hr, 3 hr, 4 hr, or more hours. In some embodiments, after ternary complex formation, a variant binary complex does not exchange the target nucleic acid with a different nucleic acid.

In some embodiments, the variant Cas12i2 polypeptide, RNA guide, and target nucleic acid are complexed in a ternary complexation buffer. In some embodiments, the variant Cas12i2 polypeptide is stored in a buffer that is replaced with a ternary complexation buffer to form a complex with the RNA guide and target nucleic acid. In some embodiments, the variant Cas12i2 polypeptide is stored in a ternary complexation buffer.

In some embodiments, the variant binary complex and target nucleic acid are complexed in a ternary complexation buffer. In some embodiments, the variant binary complex is stored in a buffer that is replaced with a ternary complexation buffer to form a complex with the target nucleic acid. In some embodiments, the variant binary complex is stored in a ternary complexation buffer.

In some embodiments, the ternary complexation buffer has a pH in a range of about 7.3 to 8.6. In one embodiment, the pH of the ternary complexation buffer is about 7.3. In one embodiment, the pH of the ternary complexation buffer is about 7.4. In one embodiment, the pH of the ternary complexation buffer is about 7.5. In one embodiment, the pH of the ternary complexation buffer is about 7.6. In one embodiment, the pH of the ternary complexation buffer is about 7.7. In one embodiment, the pH of the ternary complexation buffer is about 7.8. In one embodiment, the pH of the ternary complexation buffer is about 7.9. In one embodiment, the pH of the ternary complexation buffer is about 8.0. In one embodiment, the pH of the ternary complexation buffer is about 8.1. In one embodiment, the pH of the ternary complexation buffer is about 8.2. In one embodiment, the pH of the ternary complexation buffer is about 8.3. In one embodiment, the pH of the ternary complexation buffer is about 8.4. In one embodiment, the pH of the ternary complexation buffer is about 8.5. In one embodiment, the pH of the ternary complexation buffer is about 8.6.

The thermostability of a variant Cas12i2 polypeptide can increase under favorable conditions such as the addition of an RNA guide and target nucleic acid.

### Assessing Variant Ternary Complex Stability and Functionality

Provided herein in certain embodiments are methods for identifying an optimal variant ternary complex including (a) combining a variant Cas12i2 polypeptide, an RNA guide, and a target nucleic acid in a sample to form the variant ternary complex; (b) measuring a value of the variant ternary complex; and (c) determining the variant ternary complex is optimal over the reference molecule, if the value of the variant ternary complex is greater than a value of a reference molecule. In some embodiments, the value may include, but is not limited to, a stability measurement (e.g., Tₘ value, thermostability), a rate of ternary complex formation, a DNA binding affinity measurement, a DNA binding specificity measurement, and/or a complex activity measurement (e.g., nuclease activity measurement).

In some embodiments, an optimal variant ternary complex is identified by the steps of: (a) combining a variant Cas12i2 polypeptide, an RNA guide, and a target nucleic acid in a sample to form the variant ternary complex; (b) detecting a Tₘ value of the variant ternary complex; and (c) determining the variant ternary complex is stable if the Tₘ value of the variant ternary complex is greater than a Tₘ value of a reference molecule or a Tₘ reference value by at least 8° C.

The methods involving a step of measuring the thermostability of a variant ternary complex may include, without limitation, methods of determining the stability of a variant ternary complex, methods of determining a condition that promotes a stable variant ternary complex, methods of screening for a stable variant ternary complex, and methods for identifying an optimal binary complex to form a stable variant ternary complex. In certain embodiments, a thermostability value of a variant ternary complex may be measured.

Additionally, in certain embodiments, a thermostability value of a reference molecule may also be measured. In certain embodiments, a variant ternary complex may be determined to be stable if the measured thermostability value of the variant ternary complex is greater than the measured thermostability value of the reference molecule or a thermostability reference value, measured under the same experimental conditions, as described herein. In certain embodiments, the reference molecule may be the variant Cas12i2 polypeptide absent an RNA guide and/or target nucleic acid.

In certain embodiments, the thermostability value that is measured may be a denaturation temperature value. In these embodiments, the thermostability reference value is a denaturation temperature reference value. In certain embodiments, the thermostability value that is measured may be a Tₘ value. In these embodiments, the thermostability reference value may be a Tₘ reference value. In certain embodiments, the thermostability value may be measured using a thermal shift assay. In certain embodiments, an assay used to measure thermostability may involve a technique described herein including, but not limited to, differential scanning fluorimetry (DSF), differential scanning calorimetry (DSC), or isothermal titration calorimetry (ITC).

In certain embodiments, a variant ternary complex may be identified if the rate of ternary complex formation, DNA binding affinity, DNA binding specificity, and/or complex activity (e.g., nuclease activity) of the variant ternary complex is greater than a value of the reference molecule or the reference value (e.g., a value of a parent ternary complex). For example, in certain embodiments, the variant ternary complex may be identified if the value of a rate of ternary complex formation, DNA binding affinity, DNA binding specificity, and/or complex activity of the variant ternary complex is at least X % greater than a value of the reference molecule or the reference value (e.g., a value of a parent ternary complex). In certain embodiments, the methods described herein may further comprise steps that include measuring the activity of the variant ternary complex as described herein.

### Delivery

Compositions or complexes described herein may be formulated, for example, including a carrier, such as a carrier and/or a polymeric carrier, e.g., a liposome, and delivered by known methods to a cell (e.g., a prokaryotic, eukaryotic, plant, mammalian, etc.). Such methods include, but not limited to, transfection (e.g., lipid-mediated, cationic polymers, calcium phosphate, dendrimers); electroporation or other methods of membrane disruption (e.g., nucleofection), viral delivery (e.g., lentivirus, retrovirus, adenovirus, AAV), microinjection, microprojectile bombardment (“gene gun”), fugene, direct sonic loading, cell squeezing, optical transfection, protoplast fusion, impalefection, magnetofection, exosome-mediated transfer, lipid nanoparticle-mediated transfer, and any combination thereof.

In some embodiments, the method comprises delivering one or more nucleic acids (e.g., nucleic acids encoding the variant Cas12i2 polypeptide, RNA guide, donor DNA, etc.), one or more transcripts thereof, and/or a pre-formed variant Cas12i2 polypeptide/RNA guide complex (i.e., variant binary complex) to a cell. Exemplary intracellular delivery methods, include, but are not limited to: viruses or virus-like agents; chemical-based transfection methods, such as those using calcium phosphate, dendrimers, liposomes, or cationic polymers (e.g., DEAE-dextran or polyethylenimine); non-chemical methods, such as microinjection, electroporation, cell squeezing, sonoporation, optical transfection, impalefection, protoplast fusion, bacterial conjugation, delivery of plasmids or transposons; particle-based methods, such as using a gene gun, magnetofection or magnet assisted transfection, particle bombardment; and hybrid methods, such as nucleofection. In some embodiments, the present application further provides cells produced by such methods, and organisms (such as animals, plants, or fungi) comprising or produced from such cells.

### Cells

Compositions or complexes described herein may be delivered to a variety of cells. In some embodiments, the cell is an isolated cell. In some embodiments the cell is in cell culture. In some embodiments, the cell is ex vivo. In some embodiments, the cell is obtained from a living organism, and maintained in a cell culture. In some embodiments, the cell is a single-cellular organism.

In some embodiments, the cell is a prokaryotic cell. In some embodiments, the cell is a bacterial cell or derived from a bacterial cell. In some embodiments, the bacterial cell is not related to the bacterial species from which the parent polypeptide is derived. In some embodiments, the cell is an archaeal cell or derived from an archaeal cell. In some embodiments, the cell is a eukaryotic cell. In some embodiments, the cell is a plant cell or derived from a plant cell. In some embodiments, the cell is a fungal cell or derived from a fungal cell. In some embodiments, the cell is an animal cell or derived from an animal cell. In some embodiments, the cell is an invertebrate cell or derived from an invertebrate cell. In some embodiments, the cell is a vertebrate cell or derived from a vertebrate cell. In some embodiments, the cell is a mammalian cell or derived from a mammalian cell. In some embodiments, the cell is a human cell. In some embodiments, the cell is a zebra fish cell. In some embodiments, the cell is a rodent cell. In some embodiments, the cell is synthetically made, sometimes termed an artificial cell.

In some embodiments, the cell is derived from a cell line. A wide variety of cell lines for tissue culture are known in the art. Examples of cell lines include, but are not limited to, 293T, MF7, K562, HeLa, and transgenic varieties thereof. Cell lines are available from a variety of sources known to those with skill in the art (see, e.g., the American Type Culture Collection (ATCC) (Manassas, Va.)). In some embodiments, a cell transfected with one or more nucleic acids (such as Ago-coding vector and gDNA) or Ago-gDNA complex described herein is used to establish a new cell line comprising one or more vector-derived sequences to establish a new cell line comprising modification to the target nucleic acid. In some embodiments, cells transiently or non-transiently transfected with one or more nucleic acids (such as variant Cas12i2 polypeptide-encoding vector and RNA guide) or variant Cas12i2 polypeptide/RNA guide complex (i.e., variant binary complex) described herein, or cell lines derived from such cells are used in assessing one or more test compounds.

In some embodiments, the cell is a primary cell. For example, cultures of primary cells can be passaged 0 times, 1 time, 2 times, 4 times, 5 times, 10 times, 15 times or more. In some embodiments, the primary cells are harvest from an individual by any known method. For example, leukocytes may be harvested by apheresis, leukocytapheresis, density gradient separation, etc. Cells from tissues such as skin, muscle, bone marrow, spleen, liver, pancreas, lung, intestine, stomach, etc. can be harvested by biopsy. An appropriate solution may be used for dispersion or suspension of the harvested cells. Such solution can generally be a balanced salt solution, (e.g. normal saline, phosphate-buffered saline (PBS), Hank's balanced salt solution, etc.), conveniently supplemented with fetal calf serum or other naturally occurring factors, in conjunction with an acceptable buffer at low concentration. Buffers can include HEPES, phosphate buffers, lactate buffers, etc. Cells may be used immediately, or they may be stored (e.g., by freezing). Frozen cells can be thawed and can be capable of being reused. Cells can be frozen in a DMSO, serum, medium buffer (e.g., 10% DMSO, 50% serum, 40% buffered medium), and/or some other such common solution used to preserve cells at freezing temperatures.

In some embodiments, the variant Cas12i2 polypeptide has nuclease activity that induces double-stranded breaks or single-stranded breaks in a target nucleic acid, (e.g. genomic DNA). The double-stranded break can stimulate cellular endogenous DNA-repair pathways, including Homology Directed Recombination (HDR), Non-Homologous End Joining (NHEJ), or Alternative Non-Homologues End-Joining (A-NHEJ). NHEJ can repair cleaved target nucleic acid without the need for a homologous template. This can result in deletion or insertion of one or more nucleotides into the target nucleic acid. HDR can occur with a homologous template, such as the donor DNA. The homologous template can comprise sequences that are homologous to sequences flanking the target nucleic acid cleavage site. In some cases, HDR can insert an exogenous polynucleotide sequence into the cleaved target nucleic acid. The modifications of the target DNA due to NHEJ and/or HDR can lead to, for example, mutations, deletions, alterations, integrations, gene correction, gene replacement, gene tagging, transgene knock-in, gene disruption, and/or gene knock-outs.

In some embodiments, the cell culture is synchronized to enhance the efficiency of the methods. In some embodiments, cells in S and G2 phases are used for HDR-mediated gene editing. In some embodiments, the cell can be subjected to the method at any cell cycle. In some embodiments, cell over-plating significantly reduces the efficacy of the method. In some embodiments, the method is applied to a cell culture at no more than about any one of 40%, 45%, 50%, 55%, 60%, 65%, or 70% confluency.

In some embodiments, binding of the variant Cas12i2 polypeptide/RNA guide complex (i.e., variant binary complex) to the target nucleic acid in the cell recruits one or more endogenous cellular molecules or pathways other than DNA repair pathways to modify the target nucleic acid. In some embodiments, binding of the variant binary complex blocks access of one or more endogenous cellular molecules or pathways to the target nucleic acid, thereby modifying the target nucleic acid. For example, binding of the variant binary complex may block endogenous transcription or translation machinery to decrease the expression of the target nucleic acid.

In some embodiments, delivery of a variant Cas12i2 polypeptide does not substantially affect viability of the cell. In some embodiments, a cell remains viable following delivery of a variant Cas12i2 polypeptide. In some embodiments, a cell remains viable at least 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 36 hours, 48 hours, 60 hours, 72 hours, or more following delivery of a variant Cas12i2 polypeptide. In some embodiments, at least 70% (e.g., 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) of a plurality of cells remain viable following delivery of a variant Cas12i2 polypeptide. In some embodiments, at least 70% (e.g., 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) of a plurality of cells remain viable at least 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 36 hours, 48 hours, 60 hours, 72 hours, or more following delivery of a variant Cas12i2 polypeptide. In some embodiments, at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) of a plurality of cells remain viable following delivery of a variant Cas12i2 polypeptide. In some embodiments, at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) of a plurality of cells remain viable at least 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 36 hours, 48 hours, 60 hours, 72 hours, or more following delivery of a variant Cas12i2 polypeptide. In some embodiments, at least 90% (e.g., 90%, 91%, 92%, 93%, 94%, 95%, 96%, 97%, 98%, 99%, or 100%) of a plurality of cells remain viable following delivery of a variant Cas12i2 polypeptide. In some embodiments, at least 90% (e.g., 90%, 91%, 92%, 93%, 94%, 95%, 96%, 97%, 98%, 99%, or 100%) of a plurality of cells remain viable at least 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 36 hours, 48 hours, 60 hours, 72 hours, or more following delivery of a variant Cas12i2 polypeptide.

In some embodiments, delivery of a variant Cas12i2 binary complex (e.g., RNP) does not substantially affect viability of the cell. In some embodiments, a cell remains viable following delivery of a variant Cas12i2 binary complex (e.g., RNP). In some embodiments, a cell remains viable at least 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 36 hours, 48 hours, 60 hours, 72 hours, or more following delivery of a variant Cas12i2 binary complex (e.g., RNP). In some embodiments, at least 70% (e.g., 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) of a plurality of cells remain viable following delivery of a variant Cas12i2 binary complex (e.g., RNP). In some embodiments, at least 70% (e.g., 71%, 72%, 73% 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) of a plurality of cells remain viable at least 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 36 hours, 48 hours, 60 hours, 72 hours, or more following delivery of a variant Cas12i2 binary complex (e.g., RNP). In some embodiments, at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) of a plurality of cells remain viable following delivery of a variant Cas12i2 binary complex (e.g., RNP). In some embodiments, at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) of a plurality of cells remain viable at least 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 36 hours, 48 hours, 60 hours, 72 hours, or more following delivery of a variant Cas12i2 binary complex (e.g., RNP). In some embodiments, at least 90% (e.g., 90%, 91%, 92%, 93%, 94%, 95%, 96%, 97%, 98%, 99%, or 100%) of a plurality of cells remain viable following delivery of a variant Cas12i2 binary complex (e.g., RNP). In some embodiments, at least 90% (e.g., 90%, 91%, 92%, 93%, 94%, 95%, 96%, 97%, 98%, 99%, or 100%) of a plurality of cells remain viable at least 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 36 hours, 48 hours, 60 hours, 72 hours, or more following delivery of a variant Cas12i2 binary complex (e.g., RNP).

### Kits

The invention also provides kits that can be used, for example, to carry out a method described herein. In some embodiments, the kits include a variant Cas12i2 polypeptide of the invention, e.g., a variant comprising a substitution of Table 2 or a variant polypeptide of Table 3. In some embodiments, the kits include a polynucleotide that encodes such a variant Cas12i2 polypeptide, and optionally the polynucleotide is comprised within a vector, e.g., as described herein. The kits also can optionally include an RNA guide, e.g., as described herein. The RNA guide of the kits of the invention can be designed to target a sequence of interest, as is known in the art. The effector variant and the RNA guide can be packaged within the same vial or other vessel within a kit or can be packaged in separate vials or other vessels, the contents of which can be mixed prior to use. The kits can additionally include, optionally, a buffer and/or instructions for use of the effector variant and/or RNA guide.

All references and publications cited herein are hereby incorporated by reference.

### EXAMPLES

The following examples are provided to further illustrate some embodiments of the present invention but are not intended to limit the scope of the invention; it will be understood by their exemplary nature that other procedures, methodologies, or techniques known to those skilled in the art may alternatively be used.

### Example 1—Engineering of Cas12i2 Variant Constructs

In this Example, Cas12i2 variant constructs were generated.

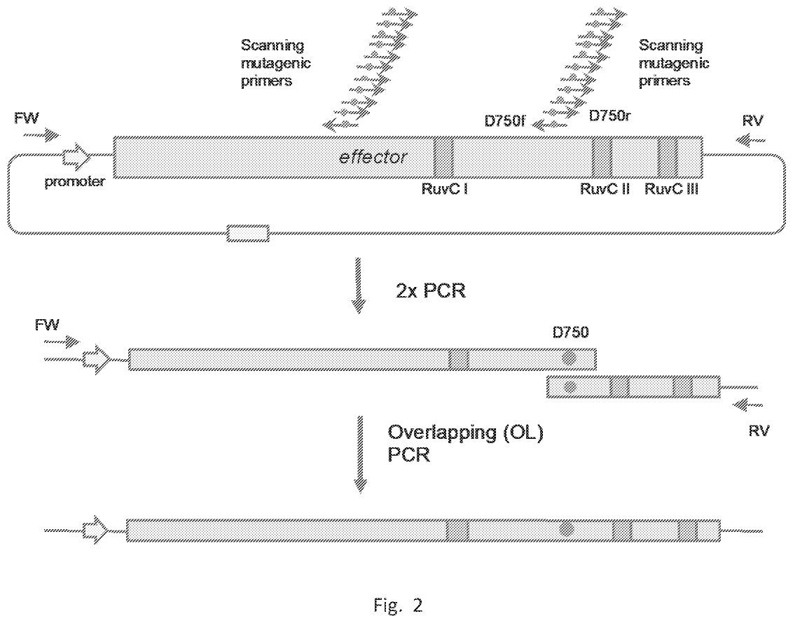

DNA templates comprising single mutations were constructed via two PCR steps (FIG. 2) using mutagenic forward and mutagenic reverse primers ordered from IDT. In the first step, two sets of PCR reactions were conducted in 384 plates to generate two fragments. The overlapping regions of two PCR fragments contained the desired single mutations and allowed the assembly of the entire DNA template via a second PCR. In the second step, the purified fragments from the first step were used as the template for the overlapping PCR (OL PCR) and the Fw and Rv oligos annealing to the vector backbone as the OL PCR primers. The resulting linear DNA templates contained a T7 promoter, a T7 terminator, and the open-reading frame for the Cas12i2 effector.

These linear DNA templates were used directly in a cell-free transcription and translation system to express the Cas12i2 effector variants containing the single mutations. The variant constructs were further individually transferred into transient transfection vectors. Additionally, DNA templates comprising combinatorial mutations were prepared by PCR and subsequently transferred into transient transfection vectors.

### Example 2—Florescence Polarization Assay for Variant Cas12i2 Binary Complex Detection

In this Example, the ability of a Cas12i2 polypeptide (wild-type or variant Cas12i2) and an RNA guide to form a binary complex is assessed through a fluorescence polarization assay.

Linear ssDNA fragments comprising the reverse complement of the T7 RNA polymerase promoter sequence upstream of the mature Cas12i2 direct repeat sequence and desired 20 bp RNA guide target are synthesized by IDT. Linear dsDNA in vitro transcription (IVT) templates are then generated by annealing a universal T7 forward oligo (95-4° C. at 5° C./minute) to the reverse complement ssDNA and filled in with Klenow fragment (New England Biolabs) for 15 minutes at 25° C. The resulting IVT template is then transcribed into an RNA guide using the HiScribe T7 High Yield RNA Synthesis Kit (New England Biolabs) at 37° C. for 4 hours. Following transcription, each RNA guide is purified using an RNA Clean and Concentrator Kit (Zymo) and stored at −20° C. until use.

The RNA guide is then labeled with 6-carboxyfluorescein (6-FAM) (IDT). 25 nM Cas12i2 polypeptide (wild-type or variant Cas12i2) in 1× assay buffer (20 mM Tris-HCl (pH 7.5), 150 mM KCl, 5 mM MgCl₂, 1 mM DTT) is titrated with increasing concentrations of labeled RNA guide (7.5-250 nM). Complexes are incubated at 37° C. for 30 minutes before taking fluorescence polarization measurements using a microplate reader (Infinite 200 Pro, Tecan).

Binary complex formation at different temperatures is also investigated. Further binding experiments as described above are performed isothermally at 25, 50, 60, and 70° C.

Formation of a binary complex upon titration of a Cas12i2 polypeptide (wild-type or variant Cas12i2) with increasing concentrations of RNA guide (or formation of a binary complex upon titration of RNA guide with increasing concentrations of a Cas12i2 polypeptide) results in changes in fluorescence polarization signal, in millipolarization (mP) units. A binding curve is generated by plotting changes in fluorescence polarization signal over a range of RNA guide concentrations.

This Example indicates how binding affinities of Cas12i2 polypeptides (wild-type or variant Cas12i2) to RNA guides can be determined and compared.

### Example 3—RNA Electrophoretic Mobility Shift Assay for Variant Cas12i2 Binary Complex Detection

This Example describes use of an RNA EMSA to determine the ability of a Cas12i2 polypeptide (wild-type or variant) to bind to an RNA guide.

Synthetic RNA guides from IDT are labeled with a 5′ IR800 dye using 5′ EndTag Labeling Kit (Vector Labs) and IRDye 800CW Maleimide (LI-COR Biosciences), as previously detailed in Yan et al., 2018. After labeling, the RNA guides are cleaned and concentrated via phenol chloroform extraction. Concentrations are quantified by Nanodrop.

For RNA binding assays, Cas12i2 polypeptides (wild-type Cas12i2 or a variant Cas12i2) are diluted to 2.5 μM in 1× binding buffer (50 mM NaCl, 10 mM Tris-HCl, 10 mM MgCl₂, 1 mM DTT, pH 7.9. Polypeptides are then serially diluted from 2.5 μM to 37.5 μM in 1× binding buffer. The polypeptides are again diluted 1:10 in 1× binding buffer plus 50 nM IR800 labeled RNA guide and mixed thoroughly. These reactions can further include 0.5-5 μg tRNA, which serves as a competitive inhibitor to decrease nonspecific binding of polypeptide to RNA and thereby facilitate accurate specific binding determinations. Reactions are incubated at 37° C. for 1 hour. 1 μL 100× bromophenol blue is added to the reactions for dye front visualization, then the entire reaction is loaded onto a 6% DNA Retardation Gel (Thermofisher), which runs for 90 minutes at 80V. The gel is imaged on the Licor Odyssey CLx.

This assay relies on the principle that the rate at which RNA migrates through the gel is determined by its size. An RNA only sample is able to migrate a particular distance. However, if the RNA binds to a polypeptide, a band that represents a larger, less mobile RNA complex appears, which is “upshifted” on the gel.

Therefore, the intensities of two bands are measured: 1) an RNA only band and 2) a polypeptide-bound “upshifted” RNA band. If all RNA is bound to a polypeptide, only an upshifted band is observed. As the concentration of polypeptide decreases, the intensity of the upshifted band decreases, while the intensity of the RNA only band increases. In comparing RNA binding affinities for Cas12i2 polypeptides (wild-type or variant Cas12i2), a higher polypeptide/RNA affinity is characterized by more specific binding at lower concentrations of polypeptide.

This Example indicates how binding affinities of wild-type Cas12i2 to RNA guides and binding affinities of Cas12i2 variants to RNA guides can be determined and compared.

### Example 4—In Vitro Cleavage Assay for Variant Cas12i2 Binary Complexes

This Example describes methods for preparing Cas12i2 RNPs and for determining in vitro biochemical activity of Cas12i2 (wild-type or variant Cas12i2) RNPs.

Cas12i2 vectors are transformed into E. coli BL21 (DE3) (New England BioLabs) and expressed under a T7 promoter. Transformed cells are initially grown overnight in 5 mL Luria Broth (Teknova)+50 μg/mL kanamycin, followed by inoculation into 1 L Terrific Broth media (Teknova)+50 μg/mL kanamycin. Cells are grown at 37° C. until an OD₆₀₀ of 0.6-0.8, then protein expression is induced with 0.5 mM IPTG. Cultures are then grown at 18° C. for an additional 14-18 hours. Cultures are harvested and pelleted via centrifugation, then resuspended in 1 mL extraction buffer per 5 g cell pellet (50 mM HEPES, pH 7.5, 500 mM NaCl, 5% glycerol, 0.5 mM TCEP). Cells are lysed via cell disruptor (Constant System Limited), then centrifuged at 20,000×g for 20 minutes at 4° C. in order to clarify the lysate. 0.2% polyethylenimine (PEI) is added to the clarified lysate and incubated at 4° C. with constant end-over-end rotation for 20 minutes. The lysate is then centrifuged again at 20,000×g for 10 minutes. The lysate is purified via ion exchange chromatography. After purification, fractions are run on SDS-PAGE gels, and fractions containing protein of the appropriate size are pooled and concentrated using 30 kD Amicon Ultra15 Centrifugal Units. Proteins are buffer exchanged into 12.5 mM HEPES pH 7.0, 120 mM NaCl, 0.5 mM TCEP, and 50% glycerol. Concentrations are then measured using the Nanodrop (Thermofisher), and proteins are stored at −20° C.

RNPs are prepared using a 2:1 ratio of synthetic crRNA (Integrated DNA Technologies) to protein. The RNPs are complexed for 30 minutes at 37° C. in 1× NEB2 buffer (50 mM NaCl, 10 mM Tris-HCl, 10 mM MgCl₂, 1 mM DTT, pH 7.9). After complexing, the RNPs are diluted using 1× NEB2 as a dilution buffer. Apo reactions (protein without RNA guide) are prepared in the same manner, making up the volume of crRNA with H₂O.

A target dsDNA substrate (Integrated DNA Technologies) is added at 20 nM to the RNP and apo samples. Reactions are mixed thoroughly then incubated at 37° C. for 1 hour, then quenched with 1 μL 20 mg/mL Proteinase K (Thermofisher). Reactions are incubated for another 15 minutes at 50° C., then the entire reaction is run on a 2% agarose E-gel (Thermofisher). Gels are visualized by ethidium bromide on a Gel Doc EZ Gel Imager (BioRad).

The intensities of two types of bands are measured: 1) a full-length (uncleaved) DNA band and 2) one or more downshifted cleaved DNA bands. An inactive RNP is characterized by a full-length DNA band. An active RNP yields one or more downshifted cleaved DNA bands. As the concentration of an active RNP decreases, the intensity of the full-length band increases, and the intensity of the cleaved band(s) decreases. In comparing activity of multiple RNPs, an RNP having higher activity than another is characterized by more intense cleaved bands at lower RNP concentrations.

The method of this Example allows for the comparison of in vitro cleavage activity of wild-type or variant Cas12i2 RNPs (binary complexes) on target DNA.

### Example 5—In Vitro Stability Assays of Variant Cas12i2 Polypeptides and Variant Cas12i2 Binary Complexes

In this Example, the stability of a Cas12i2 (wild-type or variant Cas12i2) RNP is assessed.

For the accelerated stability study, RNPs (5 μM) are generated in the same manner as described in Example 4, and the samples are subsequently stored at 25° C. for 48 hours.

In vitro cleavage assays (as described in Example 4) are performed on the RNP samples. These results are compared with those of Example 4 to determine the extent to which Cas12i2 RNPs (wild-type or variant Cas12i2 RNPs) stored at 25° C. for 48 hours retain biochemical activity.

Apo polypeptide (without RNA guide) is also incubated at 25° C. for 48 hours. RNA EMSA assays are performed on the apo samples using the method described in Example 3. These results are compared with those of Example 3 to determine the extent to which Cas12i2 (wild-type or variant Cas12i2) is able to form a binary complex with an RNA guide.

Apo samples incubated at 25° C. for 48 hours are also complexed with RNA guides to form RNPs, using the method described in Example 4. In vitro cleavage assays are then performed according to the methods of Example 4. The assay results are compared with those of Example 4 to assess activity levels of Cas12i2 RNPs formed with protein incubated at 25° C.

The methods of this Example allow for comparison of the stability of wild-type and variant Cas12i2 polypeptides and wild-type and variant Cas12i2 RNPs (binary complexes). A Cas12i2 polypeptide demonstrating greater specific binding to an RNA guide than another Cas12i2 polypeptide to the RNA guide is indicative of a more stable polypeptide. A Cas12i2 RNP demonstrating more robust in vitro cleavage of a target DNA than cleavage by another Cas12i2 polypeptide is indicative of a more stable binary complex.

### Example 6—Florescence Polarization Assay for Variant Cas12i2 Ternary Complex Detection

In this Example, the ability of an RNA guide, Cas12i2 polypeptide (wild-type or variant Cas12i2), and target DNA molecule to form a ternary complex is assessed through a fluorescence polarization assay.

Linear ssDNA fragments comprising the reverse complement of the T7 RNA polymerase promoter sequence upstream of the mature Cas12i2 direct repeat sequence and desired 20 bp RNA guide target are synthesized by IDT. Linear dsDNA in vitro transcription (IVT) templates are then generated by annealing a universal T7 forward oligo (95-4° C. at 5° C./minute) to the reverse complement ssDNA and filled in with Klenow fragment (New England Biolabs) for 15 minutes at 25° C. The resulting IVT template is then transcribed into an RNA guide using the HiScribe T7 High Yield RNA Synthesis Kit (New England Biolabs) at 37° C. for 4 hours. Following transcription, each RNA guide is purified using an RNA Clean and Concentrator Kit (Zymo) and stored at −20° C. until use.

To generate an RNP, a Cas12i2 polypeptide (wild-type or variant Cas12i2) and an RNA guide are combined in a 1:1.2 molar ratio for a final molar concentration of 10 μM RNP in 1× assay buffer (20 mM Tris-HCl (pH 7.5), 150 mM KCl, 5 mM MgCl₂, 1 mM DTT). The components are incubated at 37° C. for 30 minutes to allow for RNP formation.

Cas12i2 RNPs (wild-type or variant Cas12i2 RNPs) are diluted to a working concentration of 1 μM in assay buffer. A 7-point 2-fold dilution is generated from 1 μM to 15.6 nM. A Cas12i2 RNP is incubated with 6-FAM labeled DNA target (IDT) at 37° C. for 30 minutes. After incubation, fluorescence polarization values are measured using a microplate reader (Infinite 200 Pro, Tecan).

To assess ternary complex formation at different temperatures, the above protocols are repeated at a constant temperature of 25, 50, 60, and 70° C.

Binding of a Cas12i2 RNP to target DNA is characterized by changes in fluorescence polarization values. Formation of a ternary complex is observed as changing (e.g., increasing or decreasing) mP units over time and/or with increasing concentrations of Cas12i2.

This Example indicates how binding affinities of wild-type Cas12i2 RNPs to target DNAs and binding affinities of Cas12i2 variant RNPs to target DNAs can be determined and compared.

### Example 7—DNA Electrophoretic Mobility Shift Assay for Variant Cas12i2 Ternary Complex Detection

This Example describes use of a DNA EMSA to determine the ability of an RNA guide, a Cas12i2 polypeptide (wild-type or variant Cas12i2), and a target DNA substrate to form a ternary complex.

Cas12i2 vectors were transformed into E. coli BL21 (DE3) (New England BioLabs) and expressed under a T7 promoter. Transformed cells were initially grown overnight in 5 mL Luria Broth (Teknova)+50 μg/mL kanamycin, followed by inoculation into 1 L Terrific Broth media (Teknova)+50 μg/mL kanamycin. Cells were grown at 37° C. until an OD₆₀₀ of 0.6-0.8, then protein expression was induced with 0.5 mM IPTG. Cultures were then grown at 18° C. for an additional 14-18 hours. Cultures were harvested and pelleted via centrifugation, then resuspended in 1 mL extraction buffer per 5 g cell pellet (50 mM HEPES, pH 7.5, 500 mM NaCl, 5% glycerol, 0.5 mM TCEP). Cells were lysed via cell disruptor (Constant System Limited), then centrifuged at 20,000×g for 20 minutes at 4° C. in order to clarify the lysate. 0.2% polyethylenimine (PEI) was added to the clarified lysate and incubated at 4° C. with constant end-over-end rotation for 20 minutes. The lysate was then centrifuged again at 20,000×g for 10 minutes. The lysate was purified via ion exchange chromatography. After purification, fractions were run on SDS-PAGE gels, and fractions containing protein of the appropriate size were pooled and concentrated using 30 kD Amicon Ultra15 Centrifugal Units. Proteins were buffer exchanged into 12.5 mM HEPES pH 7.0, 120 mM NaCl, 0.5 mM TCEP, and 50% glycerol. Concentrations were then measured using the Nanodrop (Thermofisher) and proteins were stored at −20° C.

RNPs were prepared using a 2:1 ratio of synthetic RNA guide (Integrated DNA Technologies) to polypeptide. The RNA guide sequences are shown in Table 14. crRNA 1 (SEQ ID NO: 147) corresponded to Target 1 (SEQ ID NO: 150), crRNA 2 (SEQ ID NO: 148) corresponded to Target 2 (SEQ ID NO: 151), and crRNA 3 (SEQ ID NO: 149) corresponded to Target 3 (SEQ ID NO: 152). The RNPs were complexed for 30 minutes at 37° C. in 1× NEB2 buffer (50 mM NaCl, 10 mM Tris-HCl, 10 mM MgCl₂, 1 mM DTT, pH 7.9). After complexing, a 5 point 1:2 serial dilution from 5 μM to 37.5 μM was performed, using 1× NEB2 as a dilution buffer. Apo reactions (polypeptide without RNA guide) were prepared in the same manner, making up the volume of RNA guide with H₂O.

TABLE 14
DNA EMSA RNA guide sequences.

| RNA Guide | Sequence |
|---|---|
| crRNA 1 | AGAAATCCGTCTTTCATTGACGGGGTGAGGGAGGAGA |
| (AAVS1) | GATGCC (SEQ ID NO: 147) |
| crRNA 2 | AGAAATCCGTCTTTCATTGACGGTGTGCCCGTGGGGA |
| (VEGFA) | CCCCCG (SEQ ID NO: 148) |
| crRNA 3 | AGAAATCCGTCTTTCATTGACGGTGGATAAAACTTCT |
| (EMX1) | CGGAGG (SEQ ID NO: 149) |

dsDNA target substrates of the sequences in Table 15 were generated by PCR from an oligo (Integrated DNA Technologies) using the primers in Table 16. Before PCR, the 5′ end of the forward primer was labeled an IR800 dye, as described in Yan et al., 2018. Using Amplitaq Gold (Thermofisher), the dsDNA substrate was then amplified with the IR800 labeled forward primer and unlabeled reverse primer. The resulting dsDNA was purified with a DNA Clean and Concentrator Kit (Zymo) and quantified by Nanodrop (Thermofisher).

TABLE 15
DNA EMSA Target Substrates.

| Target | |
|---|---|
| Substrate | Sequence |
| Target 1 | TTTCTGTCTGCAGCTTGTGGCCTGGGTCACCTCTACGGCTGGCCCAGATCCTT |
| (AAVS1) | CCCTGCCGCCTCCTTCAGGTTCCGTCTTCCTCCACTCCCTCTTCCCCTTGCTC |
| | TCTGCTGTGTTGCTGCCCAAGGATGCTCTTTCCGGAGCACTTCCTTCTCGGCG |
| | CTGCACCACGTGATGTCCTCTGAGCGGATCCTCCCCGTGTCTGGGTCCTCTCC |
| | GGGCATCTCTCCTCCCTCACCCAACCCCATGCCGTCTTCACTCGCTGGGTTCC |
| | CTTTTCCTTCTCCTTCTGGGGCCTGTGCCATCTCTCGTTTCTTAGGATGGCCT |
| | TCTCCGACGGATGTCTCCCTTGCGTCCCGCCTCCCCTTCTTGTAGGCCTGCAT |
| | CATCACCGTTTTTCTGGACAACCCCAAAGTACCCCGTCTCCCTGGCTTTAGCC |
| | ACCTCTCCATCCTCTTGCTTTCTTTGCCTGGACACCCCGTTCTCCTGTGGATT |
| | CGGGTCACCTCTCACTCCTTTCATTTGGGCAGCTCCCCTACCCCCCTTACCTC |
| | TCTAGTCTGTGCTAGCTCTTCCAGCCCCCTGTCATGGCATCTTCCAGGGGTCC |
| | GAGAGCTCAGCTAGTCTTCTTCCTCCAACCCGGGCCCCTATGTCCACTTCAGG |
| | ACAGCATGTTTG (SEQ ID NO: 150) |
| Target 2 | GACCTTAATACTTACCATGGCTTTGGACCAGGGAACTAGGGGGATAGTGAGAG |
| (VEGFA) | CAGGGAGAGGGAAGTGTGGGGAAGGTACAGGGGACCTCGACAGTGAAGCATTC |
| | TGGGGTTTTCCTCCTGCATTTCGAGCTCCCCAGCCCCCAACATCTGGTTAGTC |
| | TTTAACTTCCTCGGGTTCATAACCATAGCAGTCCAGGAGTGGTGGGCATATTC |
| | TGTGCCCGTGGGGACCCCCGGTTGTGTCCTGTTCGACTCAGAAGACTTGGAGA |
| | AGCCAGAGGCTGTTGGTGGGAGGGAAGTGAGGAGGGAGGAGGGGCTGGGTGGC |
| | TGGGCCTGTGCACCCCAGCCCCTGCCCATGCCCATGCCTTGCTCTCTTTCTGT |
| | CCTCAGTGGTCCCAGGCTGCACCCATGGCAGAAGGAGGAGGGCAGAATCATCA |
| | CGAAGGTGAGTCCCCCTGGCTGTTGGATGGGGTTCCCTGTCCTCTCAGGGGAT |
| | GGGTGGATGGCCTAATTCCTTTTTCTTCAGAACTGTGGGGAGGAAG (SEQ ID |
| | NO: 151) |
| Target 3 | AACAGGCCTGAGCATCTTAGCTCTGGGAGGCAGAGGCCAAGCCTGTCTGTTTC |
| (EMX1) | TTACAGGACCCAGCTCAGTGCCCGGGATGGAGTACATGCTCAATAAACATGTA |
| | TTGAATTAATGAGCACATTTCTCTTTGCCCATACAAATACACACTAACTTTAT |
| | CAGTCATTCCCCTTGCTCTCTGCTGTCATTGCTCCCTCCCTGTCCCTCTCCTT |
| | CTATCTTTCCCTTGTACTTTCACAGCTGATTGTTGATTTAGATTATGCATATA |
| | CCAGTTTGTGGATAAAACTTCTCGGAGGGTTACTCAGATCAGTGTGTGAATGA |
| | GCTCTTAATCCAGATCTCAGAAGTCTGTGCACTCCCCAAGCTTTAGCCGGGTG |
| | CTAGGAGGIGGGCAACCTGGGTGACTCTGTGTGTTTAGTGGGAGTGGGGTATT |
| | CGTGCTGGGATGGCCAGTGCCTCAATCTAGGAGATGAGGGAAGAGCCCTGGGC |
| | AAGGGCTAGTTCTCCCTTCAGGTTCTAATGACTTGTTCCTCACTGCTTGGGTG |
| | CCGCCCTGGAGTATGACCAGGAAGGTACCAGTCTAAGCTTCAGTCCTG (SEQ |
| | ID NO: 152) |

| | Forward Primer | Reverse Primer |
|---|---|---|
| Target Substrate | Sequence | Sequence |
| Target 1 (AAVS1) | TTTCTGTCTGCAGCTT | GCAAACATGCTGTCCT |
| (SEQ ID NO: 150) | GTGGCCTGG | GAAGTGGACATAGGG |
| | (SEQ ID NO: 153) | (SEQ ID NO: 154) |
| Target 2 (VEGFA) | GACCTTAATACTTACC | CTTCCTCCCCACAGTT |
| (SEQ ID NO: 151) | ATGGCTTTGGACCAGGG | CTGAAGAAAAAGG |
| | (SEQ ID NO: 155) | (SEQ ID NO: 156) |
| Target 3 (EMX1) | AACAGGCCTGAGCATC | CAGGACTGAAGCTTAG |
| (SEQ ID NO: 152) | TTAGCTCTGG | ACTGGTACCTTCC |
| | (SEQ ID NO: 157) | (SEQ ID NO: 158) |

RNP samples and Apo (control) samples were diluted 1:10 into 1× binding buffer (50 mM NaCl, 10 mM Tris-HCl, 1 mM TCEP, 10% glycerol, 2 mM EDTA, pH 8.0) plus 20 nM IR800 labeled target DNA substrate and mixed thoroughly. Reactions were incubated at 37° C. for 1 hour. Bromophenol blue was added to the reactions for dye front visualization, then the entire reaction was loaded onto a 6% DNA Retardation Gel (Thermofisher), which ran for 90 minutes at 80V. The gel was imaged on the Licor Odyssey CLx.

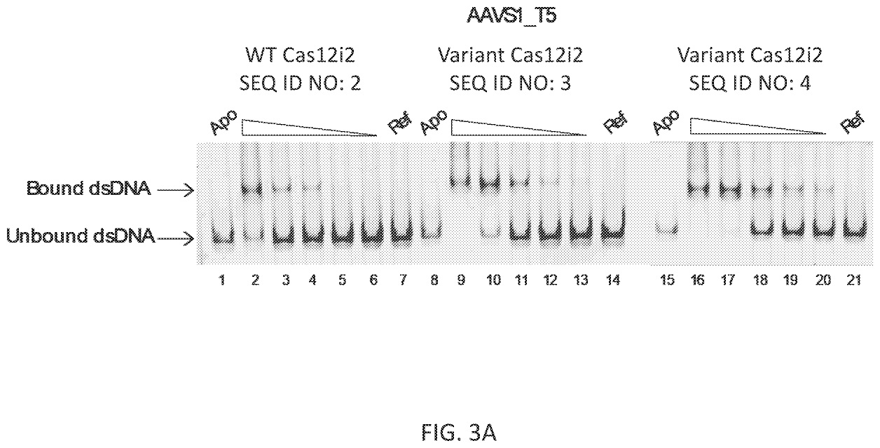

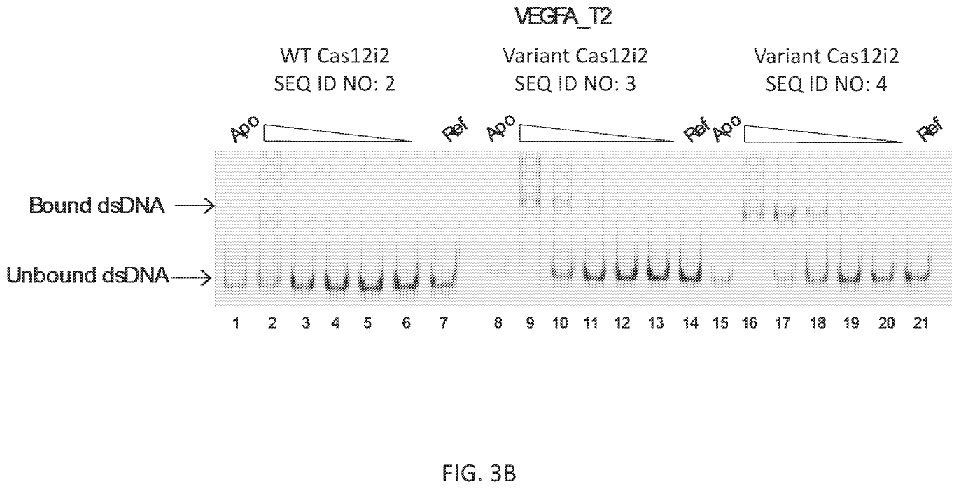

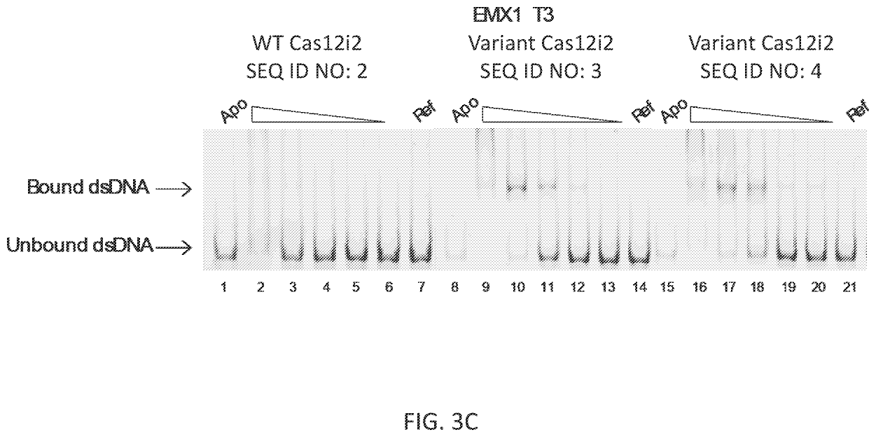

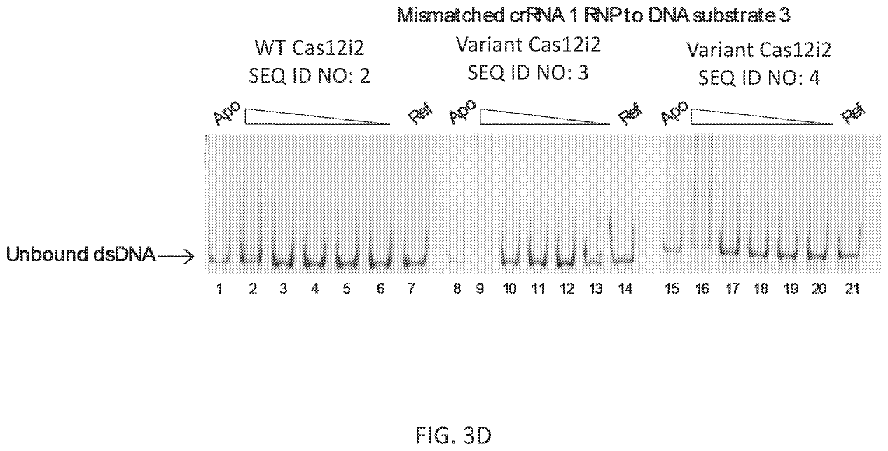

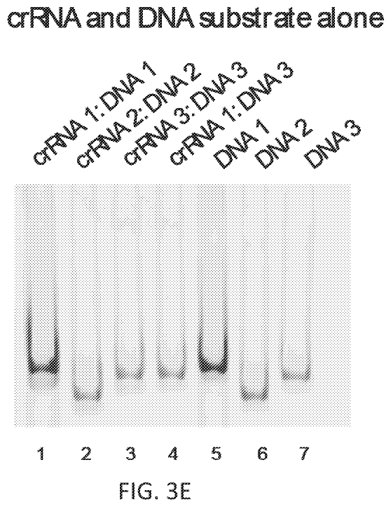

FIG. 3A, FIG. 31B, and FIG. 3C show EMSA gels for Target 1 (AAVS1), Target 2 (VEGFA), and Target 3 (EMX1), respectively. In each gel, the “Apo” lanes (lanes 1, 9, and 15) included target DNA plus wild-type Cas12i2 (lane 1), Cas12i2 variant of SEQ ID NO:3 (lane 9), or Cas12i2 variant of SEQ ID NO: 4 (lane 15). The “Ref” lanes included target DNA alone (SEQ ID NO: 150, SEQ ID NO: 151, or SEQ ID NO: 152). Lanes 2-6 in FIG. 3A, FIG. 31B, and FIG. 3C corresponded to decreasing concentrations of RNPs comprising wild-type Cas12i2 (SEQ ID NO: 2), from 1 μM to 37 nM. Lanes 9-13 in FIG. 3A, FIG. 31B, and FIG. 3C corresponded to decreasing concentrations of RNPs comprising the Cas12i2 variant of SEQ ID NO: 3, from 1 μM to 37 nM. Lanes 16-20 in FIG. 3A, FIG. 31B, and FIG. 3C corresponded to decreasing concentrations of RNPs comprising the Cas12i2 variant of SEQ ID NO: 4, from 1 μM to 37 nM.

The gels of FIG. 3A, FIG. 31B, and FIG. 3C show bands of DNA that migrated different distances. In this assay, the rate at which DNA migrates through the gel is determined by its size. A DNA only sample is able to migrate a particular distance. However, if an RNP binds to the DNA, a band that represents a larger, less mobile DNA complex appears, which is “upshifted” on the gel. Therefore, the arrows in FIG. 3A, FIG. 3B, and FIG. 3C point to “unbound dsDNA” and the “bound dsDNA,” wherein the “bound dsDNA” migrated less than the “unbound dsDNA.”

FIG. 3A shows that for the highest concentration of wild-type Cas12i2 RNP (lane 2), a faint bound dsDNA band was observed, indicating that a small amount of wild-type Cas12i2 RNP bound to the AAVS1 target DNA. However, the bound dsDNA bands were more intense with variant Cas12i2 (SEQ ID NO: 3 and SEQ ID NO: 4) RNPs. Additionally, bound dsDNA bands appeared across multiple concentrations of RNPs prepared with variant Cas12i2 polypeptides (variant Cas12i2 of SEQ ID NO: 3 in lanes 9-11 of FIG. 3A and variant Cas12i2 of SEQ ID NO: 4 in lanes 16-20 of FIG. 3A). This indicated that RNPs prepared with variant Cas12i2 polypeptides of SEQ ID NO: 3 and SEQ ID NO: 4 had a higher affinity for AAVS1 target DNA.

FIG. 3B shows that even at the highest concentrations of wild-type Cas12i2 RNP (lane 2), only unbound dsDNA bands were present, indicating that wild-type Cas12i2 RNPs did not form a ternary complex with VEGFA target DNA. However, bound dsDNA bands were observed with RNPs prepared with variant Cas12i2 polypeptides (variant Cas12i2 of SEQ ID NO: 3 in lanes 9-11 of FIG. 3B and variant Cas12i2 of SEQ ID NO: 4 in lanes 16-20 of FIG. 3B). Therefore, RNPs prepared with variant Cas12i2 polypeptides of SEQ ID NO: 3 and SEQ ID NO: 4 had a higher affinity for VEGFA target DNA than wild-type Cas12i2.

Likewise, FIG. 3C shows that at even the highest concentrations of wild-type Cas12i2 RNP (lane 2), only unbound dsDNA bands were present, indicating that wild-type Cas12i2 RNPs did not form a ternary complex with EMX1 target DNA. However, bound dsDNA bands were observed with RNPs prepared with variant Cas12i2 polypeptides (variant Cas12i2 of SEQ ID NO: 3 in lanes 9-12 of FIG. 3C and variant Cas12i2 of SEQ ID NO: 4 in lanes 16-20). Therefore, RNPs prepared with variant Cas12i2 polypeptides of SEQ ID NO: 3 and SEQ ID NO: 4 had a higher affinity for EMX1 target DNA than wild-type Cas12i2.

Based upon the data in FIG. 3A, FIG. 3B, and FIG. 3C, RNPs prepared with variant Cas12i2 polypeptides of SEQ ID NO: 3 and SEQ ID NO: 4 had a higher affinity for multiple dsDNA targets, compared to the affinity of wild-type Cas12i2 RNPs for dsDNA targets.

In order to show that upshifting of substrate DNA was sequence dependent, RNPs were incubated with mis-matching target substrates. These reactions were carried out in the same manner, making up any volumes of polypeptide with 1× NEB2 buffer. Reactions comprising Cas12i2 polypeptide (wild-type or variant), crRNA 1 (SEQ ID NO: 147), and DNA Target 3 (SEQ ID NO: 152) are shown in FIG. 3D.

In the gel in FIG. 3D, the “Apo” lanes (lanes 1, 9, and 15) included Target 3 DNA (SEQ ID NO: 152) plus wild-type Cas12i2 (lane 1), variant Cas12i2 of SEQ ID NO: 3 (lane 9), or variant Cas12i2 of SEQ ID NO: 4 (lane 15). The “Ref” lanes included Target 3 DNA alone. Lanes 2-6 in FIG. 3D corresponded to decreasing concentrations of wild-type Cas12i2 RNPs prepared with crRNA 1 (SEQ ID NO: 147), from 1 μM to 37 nM. Lanes 9-13 in FIG. 3D corresponded to decreasing concentrations of RNPs prepared with variant Cas12i2 of SEQ ID NO: 3 and crRNA 1 (SEQ ID NO: 147), from 1 UM to 37 nM. Lanes 16-20 in FIG. 3D corresponded to decreasing concentrations of RNPs prepared with variant Cas12i2 of SEQ ID NO: 4 and crRNA 1 (SEQ ID NO: 147), from 1 μM to 37 nM.

As shown in FIG. 3D, only one weakly bound dsDNA complex was observed at the highest concentration of variant Cas12i2 of SEQ ID NO: 4. All other dsDNAs remained unbound by RNP, indicating that the majority of the RNPs were unable to form a ternary complex. Therefore, the ability of an RNP to bind to a target DNA substrate, as shown in FIG. 3A, FIG. 3B, and FIG. 3C, was dependent upon the sequences of the RNA guide and the target DNA substrate.

Furthermore, in order to show that upshifting of substrate DNA was not due to base pairing of the DNA target and RNA guide, reactions comprising only RNA guide and its corresponding target DNA were analyzed. No upshifts were observed (FIG. 3E). As shown in FIG. 3E, a sample comprising crRNA 1 (SEQ ID NO: 147) and Target 1 substrate DNA (SEQ ID NO: 150) (lane 1) migrated the same distance as Target 1 substrate DNA (lane 5). A sample comprising crRNA 2 (SEQ ID NO: 148) and Target 2 substrate DNA (SEQ ID NO: 151) (lane 2) migrated the same distance as Target 2 substrate DNA (lane 6). A sample comprising crRNA 3 (SEQ ID NO: 149) and Target 3 substrate DNA (SEQ ID NO: 152) (lane 3) migrated the same distance as Target 3 substrate DNA (lane 7). Similarly, a sample comprising crRNA 1 (SEQ ID NO: 147) and Target 3 substrate DNA (SEQ ID NO: 152) (lane 4) migrated the same distance as Target 3 substrate DNA (lane 7). Therefore, the bound dsDNA bands observed in FIG. 3A, FIG. 3B, and FIG. 3C were due to RNP binding to the target DNA rather than due to base pairing between the RNA guide and target DNA substrate.

Overall, this Example shows that RNPs (binary complexes) prepared with variant Cas12i2 polypeptides had higher affinity to multiple DNA targets (to produce a ternary complex) than the affinity of wild-type Cas12i2 RNPs to the DNA targets.

### Example 8—In Vitro Cleavage Assay for Determination of Variant Cas12i2 Ternary Complex Formation

This Example describes methods for assessing in vitro biochemical activity of Cas12i2 (wild-type or variant Cas12i2) RNPs on a target DNA substrate as a means for determining ternary complex formation.

The RNA guides and dsDNA substrates in this Example are identical to those in Table 14 and Table 15, respectively. dsDNA substrates in this assay remain unlabeled. RNP and apo samples are generated and incubated in the same manner as described in Example 7, then serially diluted from 5 μM to 37.5 nM in 1× NEB2. RNP and apo samples are then further diluted 1:10 into 1× NEB2, and a target dsDNA substrate is added at 20 nM. Reactions are mixed thoroughly then incubated at 37° C. for 1 hour, then quenched with 1 μL 20 mg/mL Proteinase K (Thermofisher). Reactions are incubated for another 15 minutes at 50° C., then the entire reaction is run on a 2% agarose E-gel (Thermofisher). Gels are visualized by ethidium bromide on a Gel Doc EZ Gel Imager (BioRad).

The intensities of two types of bands are measured: 1) a full-length (uncleaved) DNA band and 2) one or more downshifted cleaved DNA bands. An inactive RNP is characterized by a full-length DNA band (e.g., the RNP was unable to form a ternary complex with the DNA substrate). An active RNP yields one or more downshifted cleaved DNA bands (e.g., the RNP was able to form a ternary complex with the DNA substrate). As the concentration of an active RNP decreases, the intensity of the full-length band increases, and the intensity of the cleaved band(s) decreases. In comparing activity of multiple RNPs, an RNP having higher activity than another is characterized by more intense cleaved bands at lower RNP concentrations.

The method of this Example allows for the comparison of in vitro cleavage activity of wild-type or variant Cas12i2 RNPs (binary complexes) on target DNA. Cleavage activity is indicative of an RNP (binary complex) forming a ternary complex with target DNA.

### Example 9—In Vitro Stability Assays for Variant Cas12i2 Binary and Ternary Complexes

In this Example, the stability of a Cas12i2 (wild-type or variant Cas12i2) RNP is assessed, wherein a sTable 8as12i2 RNP (binary complex) is able to form a ternary complex with a target DNA substrate.

For the accelerated stability study, RNPs (5 μM) are generated in the same manner as described in Example 7, and the samples are subsequently stored at 25° C. for 48 hours.

DNA EMSA assays (as described in Example 7) and in vitro cleavage assays (as described in Example 8) are performed on the RNP samples. These results are compared with those of Example 7 and Example 8 to determine the extent to which Cas12i2 RNPs (wild-type or variant Cas12i2 RNPs) stored at 25° C. for 48 hours retain DNA binding and cleavage activity. DNA binding is indicative of formation of a ternary complex, and cleavage activity is indicative of an active ternary complex.

Apo polypeptide (without RNA guide) is also incubated at 25° C. for 48 hours. The apo samples are then complexed with RNA guides to form RNPs, using the method described in Example 7. DNA EMSAs and in vitro cleavage assays are then performed, according to the methods of Example 7 and Example 8, respectively. The DNA EMSA results are compared with those of Example 7 to determine the extent to which Cas12i2 RNPs (formed after incubation of pre-complexed Cas12i2 polypeptides at 25° C.) are able to form a ternary complex with target DNA. Further, the in vitro cleavage assay results are compared with those of Example 8 to determine the extent to which Cas12i2 RNPs (formed after incubation of pre-complexed Cas12i2 polypeptides at 25° C.) are active (e.g., form a functional ternary complex with target DNA.)

The methods of this Example allow for comparison of the stability of wild-type and variant Cas12i2 polypeptides and wild-type and variant Cas12i2 RNPs (binary complexes). In this Example, a Cas12i2 RNP that retains in vitro cleavage activity on a target DNA is indicative of a more stable binary complex that is able to form a ternary complex, as compared to a Cas12i2 RNP demonstrating decreased in vitro cleavage activity on a target DNA.

### Example 10-In Vitro Targeting of GFP by Cas12i2 Variants

This Example describes use of a fluorescence depletion assay (FDA) to measure activity of wild-type Cas12i2 and Cas12i2 variants.

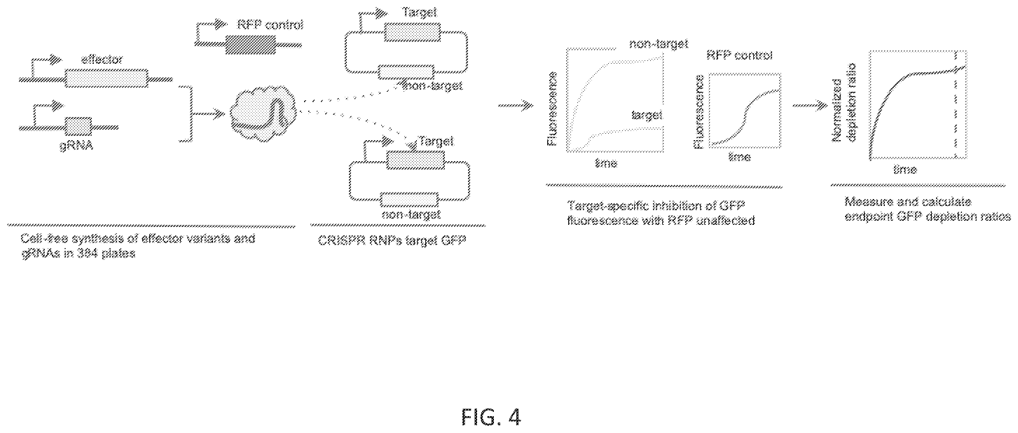

In this assay, an active CRISPR system designed to target GFP binds and cleaves the double-stranded DNA region encoding GFP, resulting in depletion of GFP fluorescence. The FDA assay involves in vitro transcription and translation, allowing production of an RNP from a DNA template encoding a Cas12i2 polypeptide and a DNA template containing a pre-crRNA sequence under a T7 promoter with direct repeat (DR)-spacer-direct repeat (DR); the spacer targeted GFP. In the same one-pot reaction, GFP and RFP were also produced as both the target and the fluorescence reporter (FIG. 4). The target GFP plasmid sequence is set forth in SEQ ID NO: 159, and the fluorescence reporter RFP plasmid sequence is set forth in SEQ ID NO: 160. GFP and RFP fluorescence values were measured every 20 min at 37° C. for 12 hr, using a TECAN Infinite F Plex plate reader. Since RFP was not targeted, its fluorescence was not affected and was therefore used as an internal signal control.

A total of 20 GFP targets (plus 1 non-target) were designed for screening the activities of wild-type Cas12i2 and Cas12i2 variants. Pre-crRNA sequences, target sequences, and non-target control sequences used for the FDA assay are listed in Table 17.

TABLE 17
pre-CRNA and Target Sequences for FDA Assay.

| Target | pre-crRNA Sequence | Target Sequence |
|---|---|---|
| top01 | gaaattaatacgactcactatagGTTGCAAAACCCAAG | TAAAGGTTGTTTTACGACAG |
| | AAATCCGTCTTTCATTGACGGTAAAGGTTGTTTTACGA | ACGATAACAGG (SEQ ID |
| | CAGACGATAACAGGGTTGCAAAACCCAAGAAATCCGTC | NO: 162) |
| | TTTCATTGACGGctaacccctctctaaacggaggggtt | |
| | t (SEQ ID NO: 161) | |
| top02 | gaaattaatacgactcactatagGTTGCAAAACCCAAG | AGTGTTTCGCCAAGTACCCG |
| | AAATCCGTCTTTCATTGACGGAGTGTTTCGCCAAGTAC | AGCCACATCAA (SEQ ID |
| | CCGAGCCACATCAAGTTGCAAAACCCAAGAAATCCGTC | NO: 164) |
| | TTTCATTGACGGctaacccctctctaaacggaggggtt | |
| | t (SEQ ID NO: 163) | |
| top03 | gaaattaatacgactcactatagGTTGCAAAACCCAAG | GCCAAGTACCCGAGCCACAT |
| | AAATCCGTCTTTCATTGACGGGCCAAGTACCCGAGCCA | CAAGGATTTCT (SEQ ID |
| | CATCAAGGATTTCTGTTGCAAAACCCAAGAAATCCGTC | NO: 166) |
| | TTTCATTGACGGctaacccctctctaaacggaggggtt | |
| | t (SEQ ID NO: 165) | |
| top04 | gaaattaatacgactcactatagGTTGCAAAACCCAAG | TTTAAGAGCGCCATGCCGGA |
| | AAATCCGTCTTTCATTGACGGTTTAAGAGCGCCATGCC | AGGTTATACCC (SEQ ID |
| | GGAAGGTTATACCCGTTGCAAAACCCAAGAAATCCGTC | NO: 168) |
| | TTTCATTGACGGctaacccctctctaaacggaggggtt | |
| | t (SEQ ID NO: 167) | |
| top05 | gaaattaatacgactcactatagGTTGCAAAACCCAAG | GAAGGCGACGGCGTGTACAA |
| | AAATCCGTCTTTCATTGACGGGAAGGCGACGGCGTGTA | GACGCGTGCTA (SEQ ID |
| | CAAGACGCGTGCTAGTTGCAAAACCCAAGAAATCCGTC | NO: 170) |
| | TTTCATTGACGGctaacccctctctaaacggaggggtt | |
| | t (SEQ ID NO: 169) | |
| top06 | gaaattaatacgactcactatagGTTGCAAAACCCAAG | TATCTACAATCGTGTCACGC |
| | AAATCCGTCTTTCATTGACGGTATCTACAATCGTGTCA | TGACTGGTGAG (SEQ ID |
| | CGCTGACTGGTGAGGTTGCAAAACCCAAGAAATCCGTC | NO: 172) |
| | TTTCATTGACGGctaacccctctctaaacggaggggtt | |
| | t (SEQ ID NO: 171) | |
| top07 | gaaattaatacgactcactatagGTTGCAAAACCCAAG | TGCGTAAGAACGTTGCATTC |
| | AAATCCGTCTTTCATTGACGGTGCGTAAGAACGTTGCA | CAATGCCCGCC (SEQ ID |
| | TTCCAATGCCCGCCGTTGCAAAACCCAAGAAATCCGTC | NO: 174) |
| | TTTCATTGACGGctaacccctctctaaacggaggggtt | |
| | t (SEQ ID NO: 173) | |
| top08 | gaaattaatacgactcactatagGTTGCAAAACCCAAG | CAATGCCCGCCAAGCATTCT |
| | AAATCCGTCTTTCATTGACGGCAATGCCCGCCAAGCAT | GTATATTCTGC (SEQ ID |
| | TCTGTATATTCTGCGTTGCAAAACCCAAGAAATCCGTC | NO: 176) |
| | TTTCATTGACGGctaacccctctctaaacggaggggtt | |
| | t (SEQ ID NO: 175) | |
| top09 | gaaattaatacgactcactatagGTTGCAAAACCCAAG | TGCCTGACACCGTTAACAAT |
| | AAATCCGTCTTTCATTGACGGTGCCTGACACCGTTAAC | GGCATCCGCGT (SEQ ID |
| | AATGGCATCCGCGTGTTGCAAAACCCAAGAAATCCGTC | NO: 178) |
| | TTTCATTGACGGctaacccctctctaaacggaggggtt | |
| | t (SEQ ID NO: 177) | |
| top10 | gaaattaatacgactcactatagGTTGCAAAACCCAAG | AACCAGGCGTACGATATTGA |
| | AAATCCGTCTTTCATTGACGGAACCAGGCGTACGATAT | AGGTGTGACCG (SEQ ID |
| | TGAAGGTGTGACCGGTTGCAAAACCCAAGAAATCCGTC | NO: 180) |
| | TTTCATTGACGGctaacccctctctaaacggaggggtt | |
| | t (SEQ ID NO: 179) | |
| bot01 | gaaattaatacgactcactatagGTTGCAAAACCCAAG | AACCCTGTTATCGTCTGTCG |
| | AAATCCGTCTTTCATTGACGGAACCCTGTTATCGTCTG | TAAAACAACCT (SEQ ID |
| | TCGTAAAACAACCTGTTGCAAAACCCAAGAAATCCGTC | NO: 182) |
| | TTTCATTGACGGctaacccctctctaaacggaggggtt | |
| | t (SEQ ID NO: 181) | |
| bot02 | gaaattaatacgactcactatagGTTGCAAAACCCAAG | TCAAACAGTTTTGCACCTTC |
| | AAATCCGTCTTTCATTGACGGTCAAACAGTTTTGCACC | CGTCAATGCCG (SEQ ID |
| | TTCCGTCAATGCCGGTTGCAAAACCCAAGAAATCCGTC | NO: 184) |
| | TTTCATTGACGGctaacccctctctaaacggaggggtt | |
| | t (SEQ ID NO: 183) | |
| bot03 | gaaattaatacgactcactatagGTTGCAAAACCCAAG | ATACCTTCGACGTCGCCTTC |
| | AAATCCGTCTTTCATTGACGGATACCTTCGACGTCGCC | CAGTTCGGTGA (SEQ ID |
| | TTCCAGTTCGGTGAGTTGCAAAACCCAAGAAATCCGTC | NO: 186) |
| | TTTCATTGACGGctaacccctctctaaacggaggggtt | |
| | t (SEQ ID NO: 185) | |
| bot04 | gaaattaatacgactcactatagGTTGCAAAACCCAAG | GCTTTAATGGTACCCGTGGT |
| | AAATCCGTCTTTCATTGACGGGCTTTAATGGTACCCGT | CGCGTCACCGG (SEQ ID |
| | GGTCGCGTCACCGGGTTGCAAAACCCAAGAAATCCGTC | NO: 188) |
| | TTTCATTGACGGctaacccctctctaaacggaggggtt | |
| | t (SEQ ID NO: 187) | |
| bot05 | gaaattaatacgactcactatagGTTGCAAAACCCAAG | GAAGCTGATGGTACGCTCTT |
| | AAATCCGTCTTTCATTGACGGGAAGCTGATGGTACGCT | GGGTATAACCT (SEQ ID |
| | CTTGGGTATAACCTGTTGCAAAACCCAAGAAATCCGTC | NO: 190) |
| | TTTCATTGACGGctaacccctctctaaacggaggggtt | |
| | t (SEQ ID NO: 189) | |
| bot06 | gaaattaatacgactcactatagGTTGCAAAACCCAAG | GTAGGTAACCATAGCACGCG |
| | AAATCCGTCTTTCATTGACGGGTAGGTAACCATAGCAC | TCTTGTACACG (SEQ ID |
| | GCGTCTTGTACACGGTTGCAAAACCCAAGAAATCCGTC | NO: 192) |
| | TTTCATTGACGGctaacccctctctaaacggaggggtt | |
| | t (SEQ ID NO: 191) | |
| bot07 | gaaattaatacgactcactatagGTTGCAAAACCCAAG | TCACCAGTCAGCGTGACACG |
| | AAATCCGTCTTTCATTGACGGTCACCAGTCAGCGTGAC | ATTGTAGATAG (SEQ ID |
| | ACGATTGTAGATAGGTTGCAAAACCCAAGAAATCCGTC | NO: 194) |
| | TTTCATTGACGGctaacccctctctaaacggaggggtt | |
| | t (SEQ ID NO: 193) | |
| bot08 | gaaattaatacgactcactatagGTTGCAAAACCCAAG | TTAAAGTTCTCACCAGTCAG |
| | AAATCCGTCTTTCATTGACGGTTAAAGTTCTCACCAGT | CGTGACACGAT (SEQ ID |
| | CAGCGTGACACGATGTTGCAAAACCCAAGAAATCCGTC | NO: 196) |
| | TTTCATTGACGGctaacccctctctaaacggaggggtt | |
| | t (SEQ ID NO: 195) | |
| bot09 | gaaattaatacgactcactatagGTTGCAAAACCCAAG | TTACGCAGAATGTGACCGTC |
| | AAATCCGTCTTTCATTGACGGTTACGCAGAATGTGACC | TTTCTTAAAGT (SEQ ID |
| | GTCTTTCTTAAAGTGTTGCAAAACCCAAGAAATCCGTC | NO: 198) |
| | TTTCATTGACGGctaacccctctctaaacggaggggtt | |
| | t (SEQ ID NO: 197) | |
| bot010 | gaaattaatacgactcactatagGTTGCAAAACCCAAG | GGTCACACCTTCAATATCGT |
| | AAATCCGTCTTTCATTGACGGGGTCACACCTTCAATAT | ACGCCTGGTTG (SEQ ID |
| | CGTACGCCTGGTTGGTTGCAAAACCCAAGAAATCCGTC | NO: 200) |
| | TTTCATTGACGGctaacccctctctaaacggaggggtt | |
| | t (SEQ ID NO: 199) | |
| non- | gaaattaatacgactcactatagGTTGCAAAACCCAAG | |
| target_ | AAATCCGTCTTTCATTGACGGGTGATTGCGCCTGAGCG | |
| top12 | AGACGAAATACGCGGTTGCAAAACCCAAGAAATCCGTC | |
| | TTTCATTGACGGctaacccctctctaaacggaggggtt | |
| | t (SEQ ID NO: 201) | |

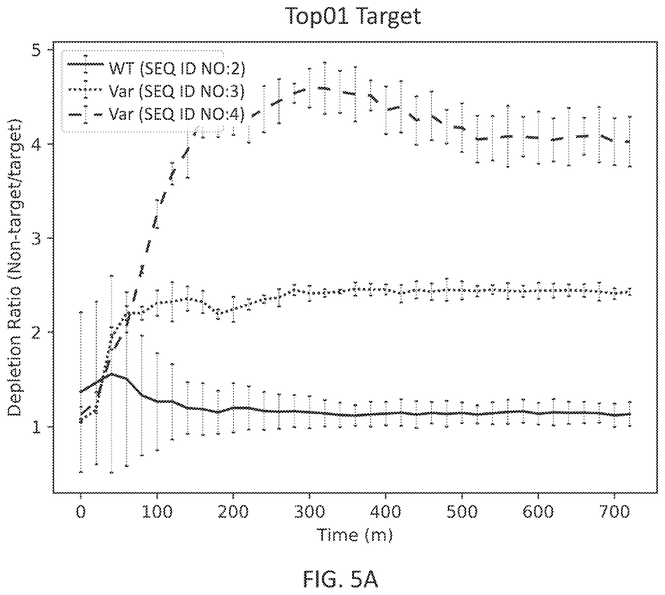

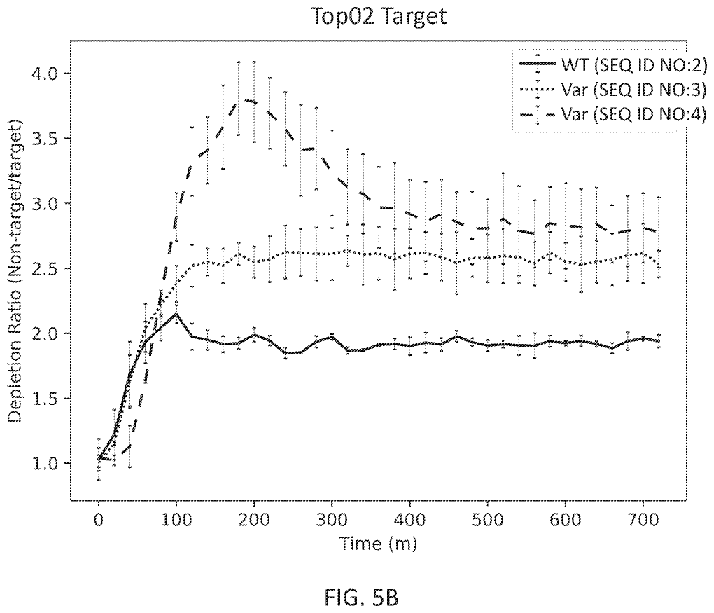

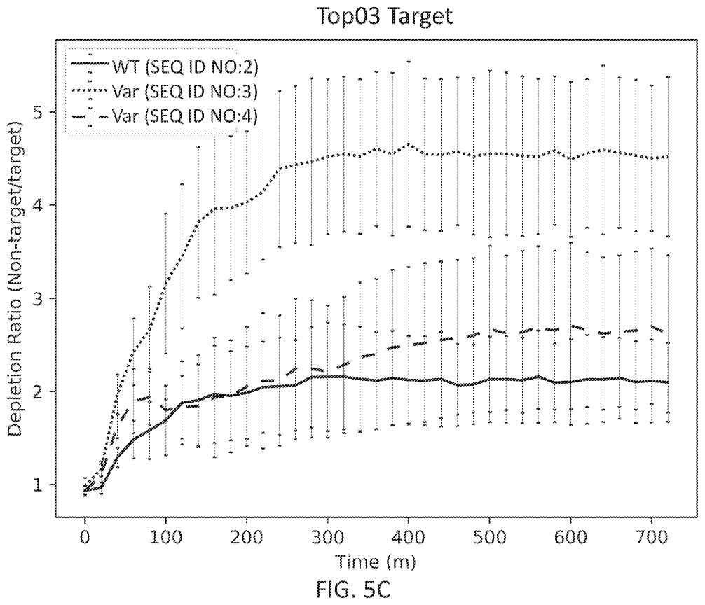

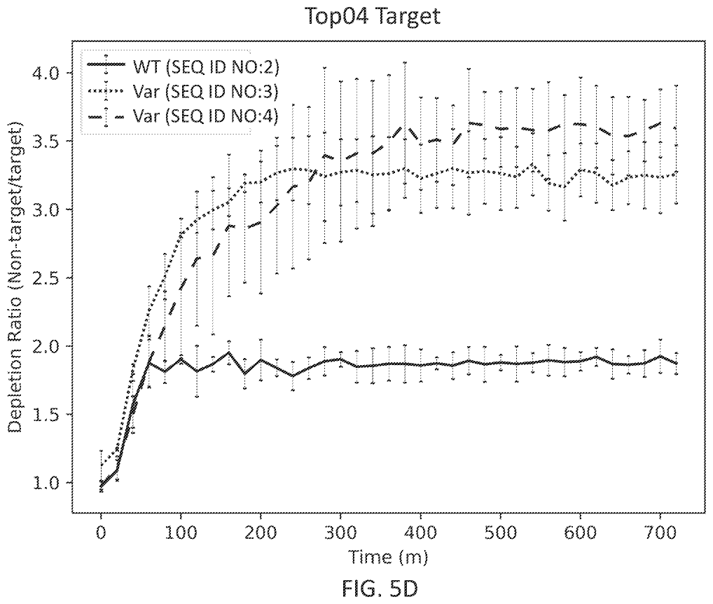

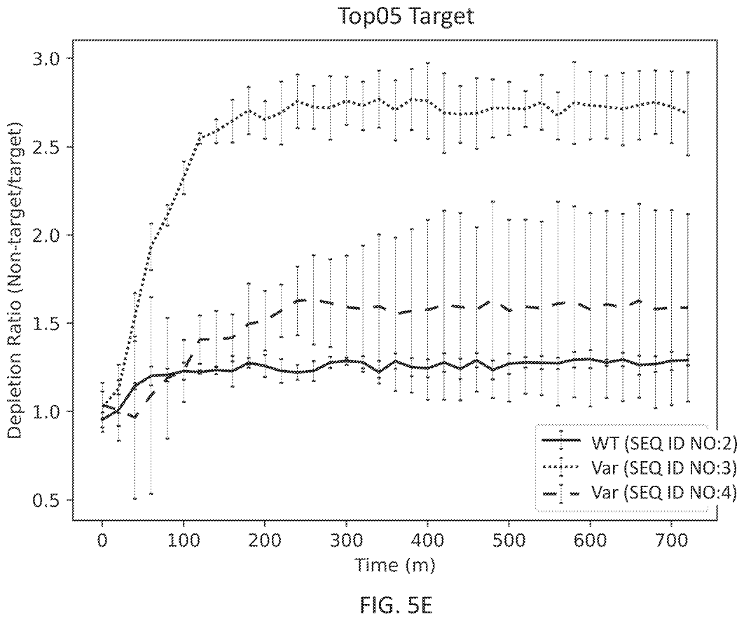

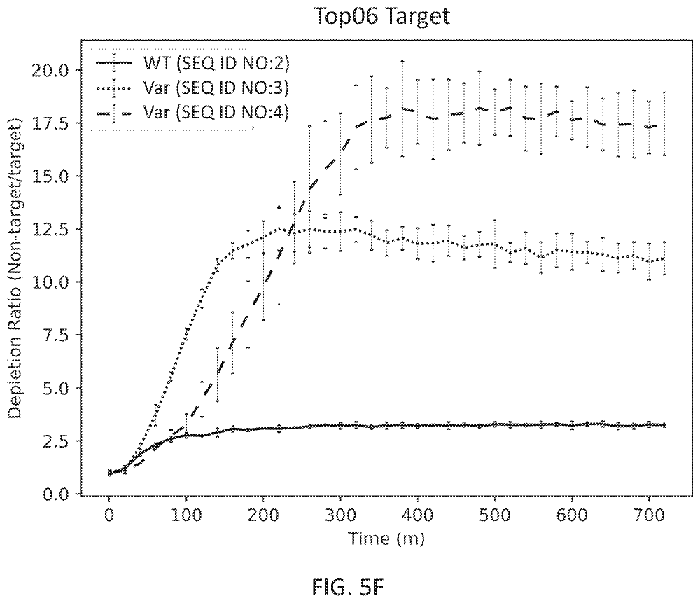

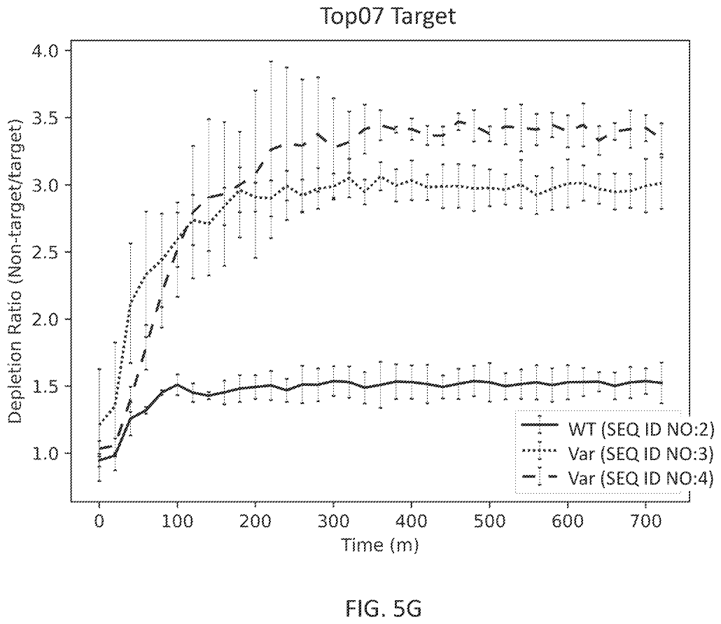

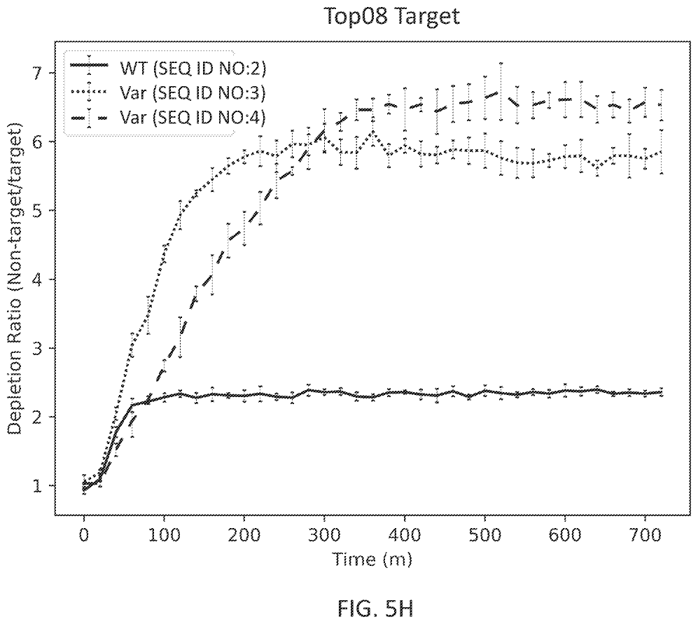

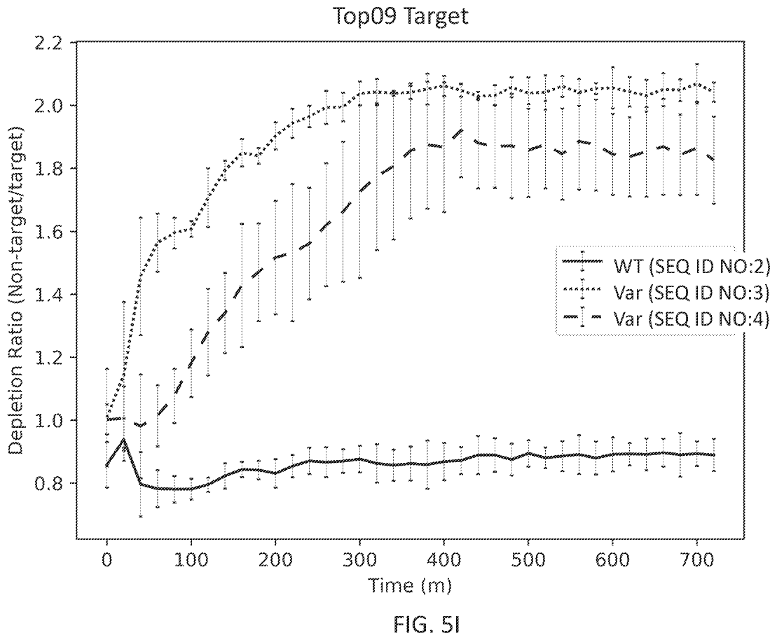

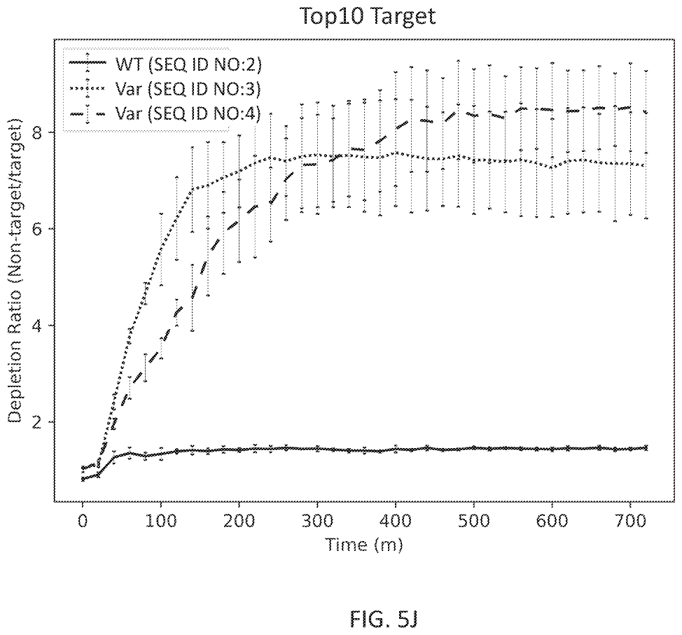

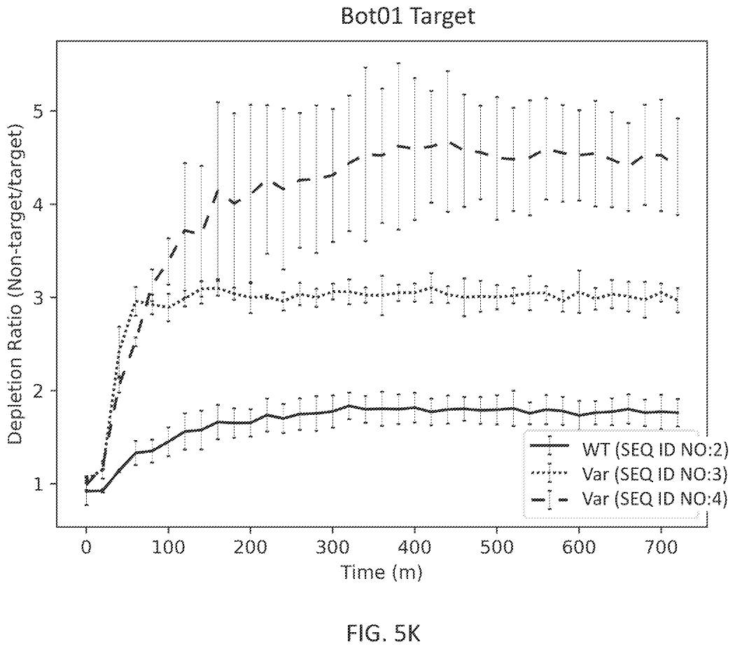

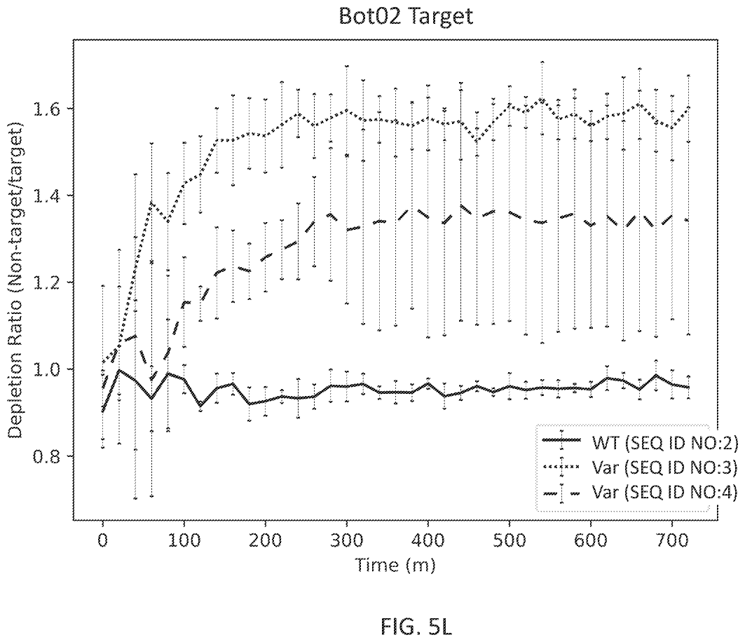

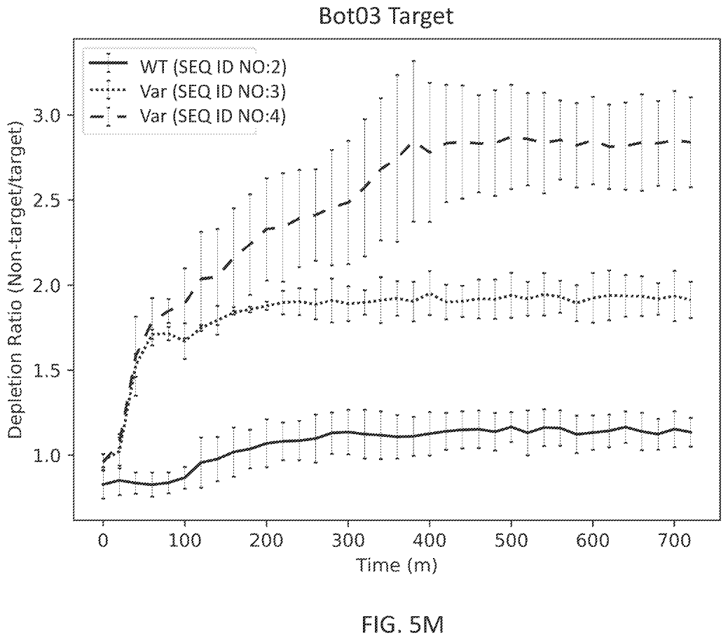

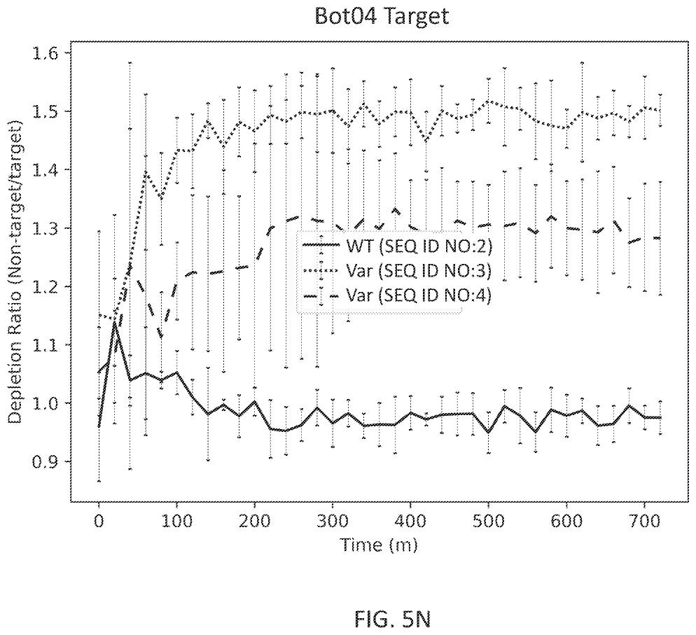

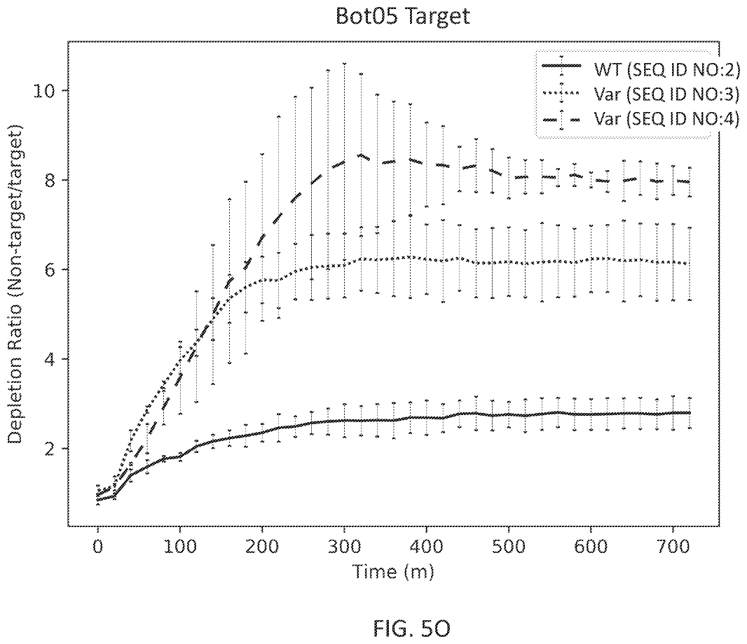

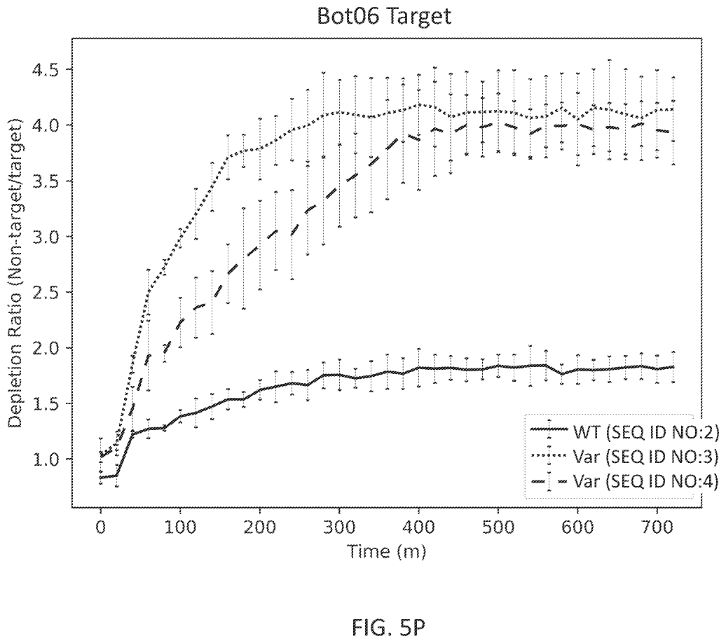

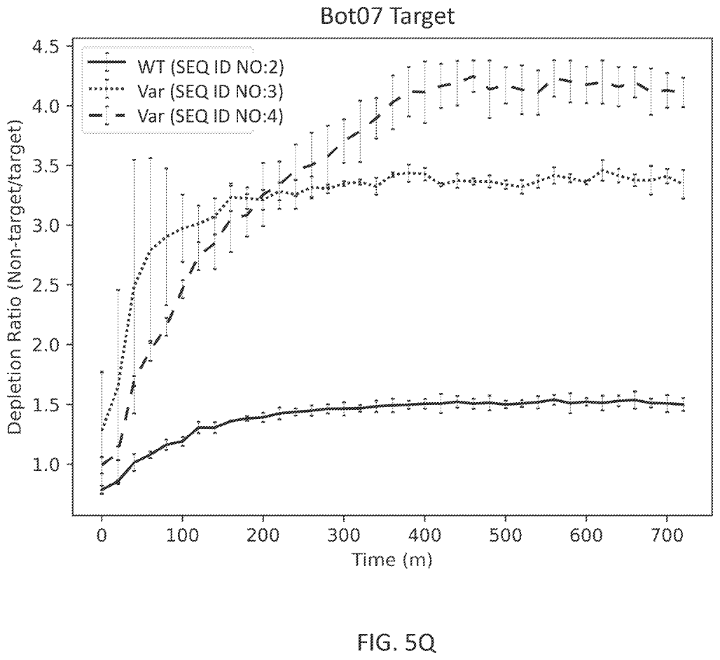

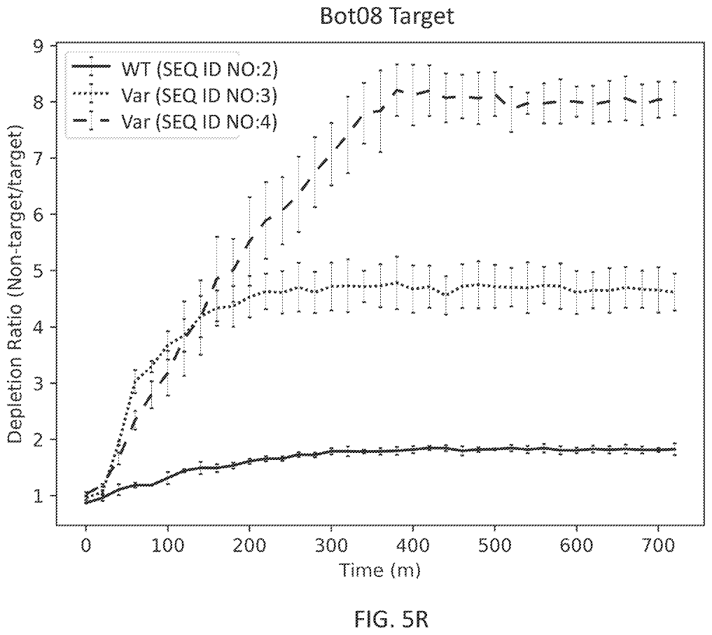

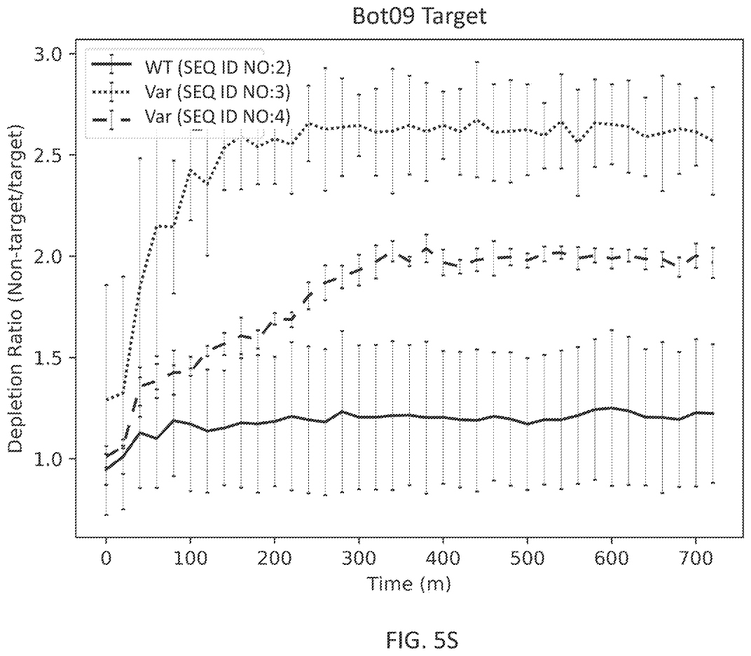

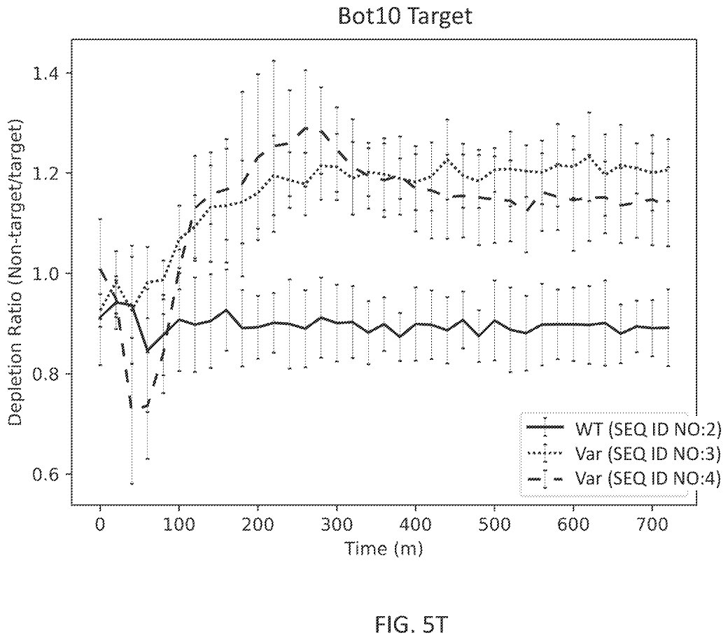

GFP signal was normalized to RFP signal, then the average fluorescence of three technical replicates was taken at each time point. GFP fluorescence depletion was then calculated by dividing the GFP signal of an effector incubated with a non-GFP targeting pre-crRNA (which instead targets a kanamycin resistance gene) by the GFP signal of an effector incubated with a GFP targeting pre-crRNA. The resulting value is referred to as “Depletion Ratio (Non-target/target)” in FIGS. 5A-T.

A Depletion Ratio (Non-target/target) of one or approximately one indicated that there was little to no difference in GFP depletion with respect to a non-GFP targeting pre-crRNA and a GFP targeting pre-crRNA (e.g., 10 RFU/10 RFU=1). A Depletion Ratio (Non-target/target) of greater than one indicated that there was a difference in GFP depletion with respect to a non-GFP targeting pre-crRNA and a GFP targeting pre-crRNA (e.g., 10 RFU/5 RFU=2). Depletion of the GFP signal indicated that the effector formed a functional RNP and interfered with the production of GFP by introducing double-stranded DNA cleavage within the GFP coding region. The extent of the GFP depletion was largely correlated to the specific activity of a Cas12i2 CRISPR system (wild-type Cas12i2 or variant Cas12i2 system).

FIGS. 5A-T show graphs of Depletion Ratios (Non-target/target) for RNPs formed by wild-type Cas12i2 (SEQ ID NO: 2), variant Cas12i2 of SEQ ID NO: 3, and variant Cas12i2 of SEQ ID NO: 4 measured every 20 minutes for each of the 20 GFP targets (top1, top2, top3, top4, top5, top6, top7, top8, top9, top10, bot1, bot2, bot3, bot4, bot5, bot6, bot7, bot8, bot9, bot10.) Wild-type Cas12i2 is depicted with a solid line, variant Cas12i2 of SEQ ID NO: 3 is depicted by a dotted line, and variant Cas12i2 of SEQ ID NO: 4 is depicted by a dashed line.

At each target, the Depletion Ratios for RNPs formed with variant Cas12i2 of SEQ ID NO: 3 or variant Cas12i2 of SEQ ID NO: 4 were greater than the Depletion Ratios for RNPs formed with wild-type Cas12i2. This indicated that the specific activity of the Cas12i2 variants was higher than the specific activity of wild-type Cas12i2 at each target site.

Furthermore, at several targets (e.g., top01, top05, top09, top10, bot02, bot03, bot04, bot09, bot10), the Depletion Ratios for wild-type Cas12i2 were one or about one, indicating that wild-type Cas12i2 was not active at these target sites (i.e., any measured GFP depletion induced by a non-GFP targeting pre-crRNA was essentially equal to GFP depletion induced by a GFP targeting pre-crRNA). See FIG. 5A, FIG. 5D, FIG. 5I, FIG. 5J, FIG. 5L, FIG. 5M, FIG. 5N, FIG. 5S, and FIG. 5T. However, at each of these targets, the Depletion Ratios for RNPs formed with variant Cas12i2 of SEQ ID NO: 3 or variant Cas12i2 of SEQ ID NO: 4 were greater than one, indicating that the Cas12i2 variants were active in depleting the GFP signal at each of sites. Therefore, the Cas12i2 variants were able to target sites on GFP that were not able to be targeted by wild-type Cas12i2.

This Example shows that Cas12i2 variant binary complexes demonstrated increased ternary complex formation with GFP target sites, as compared to wild-type Cas12i2 binary complexes. This Example further shows that the Cas12i2 variants expanded the targeting space by cleaving double-stranded DNA at GFP target sites unable to be cleaved by wild-type Cas12i2. Furthermore, Cas12i2 variants more efficiently cleaved double-stranded DNA at multiple GFP target sites, compared to wild-type Cas12i2.

### Example 11—Targeting of Mammalian Genes by Cas12i2 Variants

This Example describes indel assessment on multiple targets using wild-type Cas12i2 and Cas12i2 variants introduced into mammalian cells by transient transfection.

Wild-type Cas12i2, variant Cas12i2 of SEQ ID NO: 3, and variant Cas12i2 of SEQ ID NO: 4 were cloned into a pcda3.1 backbone (Invitrogen). The plasmids were then maxi-prepped and diluted to 1 μg/μL. For RNA guide preparation, a dsDNA fragment encoding a crRNA was derived by ultramers containing the target sequence scaffold, and the U6 promoter. Ultramers were resuspended in 10 mM Tris·HCl at a pH of 7.5 to a final stock concentration of 100 μM. Working stocks were subsequently diluted to 10 μM, again using 10 mM Tris·HCl to serve as the template for the PCR reaction. The amplification of the crRNA was done in 50 μL reactions with the following components: 0.02 μl of aforementioned template, 2.5 μl forward primer, 2.5 μl reverse primer, 25 μL NEB HiFi Polymerase, and 20 μl water. Cycling conditions were: 1×(30s at 98° C.), 30×(10s at 98° C., 15s at 67° C.), 1×(2 min at 72° C.). PCR products were cleaned up with a 1.8× SPRI treatment and normalized to 25 ng/μL. The prepared crRNA sequences and their corresponding target sequences are shown in Table 18.

TABLE 18
crRNA and Target Sequences for Transient Transfection.

| Target | crRNA sequence | Target Sequence |
|---|---|---|
| 1 (AAVS1) | AGAAATCCGTCTTTCATTGACGGTGTCCCCCCAAGT | TGTCCCCCCAAGTTTTGGA |
| | TTTGGAC | C |
| | (SEQ ID NO: 202) | (SEQ ID NO: 203) |
| 2 (AAVS1) | AGAAATCCGTCTTTCATTGACGGGGAGAGGTGAGGG | GGAGAGGTGAGGGACTTGG |
| | ACTTGGG | G |
| | (SEQ ID NO: 204) | (SEQ ID NO: 205) |
| 3 (AAVS) | AGAAATCCGTCTTTCATTGACGGGTGAGAATGGTGC | GTGAGAATGGTGCGTCCTA |
| | GTCCTAG | G |
| | (SEQ ID NO: 206) | (SEQ ID NO: 207) |
| 4 (AAVS1) | AGAAATCCGTCTTTCATTGACGGGGGGTTGTCCAGA | GGGGTTGTCCAGAAAAACG |
| | AAAACGG | G |
| | (SEQ ID NO: 208) | (SEQ ID NO: 209) |
| 5 (AAVS1) | AGAAATCCGTCTTTCATTGACGGAACTGGCCCTGGC | AACTGGCCCTGGCTTTGGC |
| | TTTGGCA | A |
| | (SEQ ID NO: 210) | (SEQ ID NO: 211) |
| 6 (EMX1) | AGAAATCCGTCTTTCATTGACGGGGGCGCAGGGCCA | GGGCGCAGGGCCACCTGGA |
| | CCTGGAC | C |
| | (SEQ ID NO: 212) | (SEQ ID NO: 213) |
| 7 (EMX1) | AGAAATCCGTCTTTCATTGACGGGGATGGCGACTTC | GGATGGCGACTTCAGGCAC |
| | AGGCACA | A |
| | (SEQ ID NO: 214) | (SEQ ID NO: 215) |
| 8 (EMX1) | AGAAATCCGTCTTTCATTGACGGATGTGATTGATGC | ATGTGATTGATGCCCAAAG |
| | CCAAAGG | G |
| | (SEQ ID NO: 216) | (SEQ ID NO: 217) |
| 9 (EMX1) | AGAAATCCGTCTTTCATTGACGGGGGGAGGCCTGGA | GGGGAGGCCTGGAGTCATG |
| | GTCATGG | G |
| | (SEQ ID NO: 218) | (SEQ ID NO: 219) |
| 10 (EMX1) | AGAAATCCGTCTTTCATTGACGGTATTATTCCCATA | TATTATTCCCATAGGGAAG |
| | GGGAAGG | G |
| | (SEQ ID NO: 220) | (SEQ ID NO: 221) |
| 11 (VEGFA) | AGAAATCCGTCTTTCATTGACGGTGGGGGTGACCGC | TGGGGGTGACCGCCGGAGC |
| | CGGAGCG | G |
| | (SEQ ID NO: 222) | (SEQ ID NO: 223) |
| 12 (VEGFA) | AGAAATCCGTCTTTCATTGACGGAATCCTCCACCAG | AATCCTCCACCAGTCATGG |
| | TCATGGT | T |
| | (SEQ ID NO: 224) | (SEQ ID NO: 225) |
| 13 (VEGFA) | AGAAATCCGTCTTTCATTGACGGGTTGACATTGTCC | GTTGACATTGTCCACACCT |
| | ACACCTG | G |
| | (SEQ ID NO: 226) | (SEQ ID NO: 227) |
| 14 (VEGFA) | AGAAATCCGTCTTTCATTGACGGGGAAATCTATTGA | GGAAATCTATTGAGGCTCT |
| | GGCTCTG | G |
| | (SEQ ID NO: 228) | (SEQ ID NO: 229) |
| 15 (VEGFA) | AGAAATCCGTCTTTCATTGACGGTTAAACTCTCCAT | TTAAACTCTCCATGGACCA |
| | GGACCAG | G |
| | (SEQ ID NO: 230) | (SEQ ID NO: 231) |

Approximately 16 hours prior to transfection, 100 μl of 25,000 HEK293T cells in DMEM/10% FBS+Pen/Strep were plated into each well of a 96-well plate. On the day of transfection, the cells were 70-90% confluent. For each well to be transfected, a mixture of 0.5 μl of Lipofectamine 2000 and 9.5 μl of Opti-MEM was prepared and then incubated at room temperature for 5-20 minutes (Solution 1). After incubation, the lipofectamine:OptiMEM mixture was added to a separate mixture containing 182 ng of effector plasmid and 14 ng of crRNA and water up to 10 μL (Solution 2). The solution 1 and solution 2 mixtures were mixed by pipetting up and down and then incubated at room temperature for 25 minutes. Following incubation, 20 μL of the Solution 1 and Solution 2 mixture were added dropwise to each well of a 96 well plate containing the cells. 72 hours post transfection, cells are trypsinized by adding 10 μL of TrypLE to the center of each well and incubated for approximately 5 minutes. 100 μL of D10 media was then added to each well and mixed to resuspend cells. The cells were then spun down at 500 g for 10 minutes, and the supernatant was discarded. QuickExtract buffer was added to ⅕ the amount of the original cell suspension volume. Cells were incubated at 65° C. for 15 minutes, 68° C. for 15 minutes, and 98° C. for 10 minutes.

Samples for Next Generation Sequencing were prepared by two rounds of PCR. The first round (PCR1) was used to amplify specific genomic regions depending on the target. PCR1 products were purified by column purification. Round 2 PCR (PCR2) was done to add Illumina adapters and indexes. Reactions were then pooled and purified by column purification. Sequencing runs were done with a 150 cycle NextSeq v2.5 mid or high output kit.

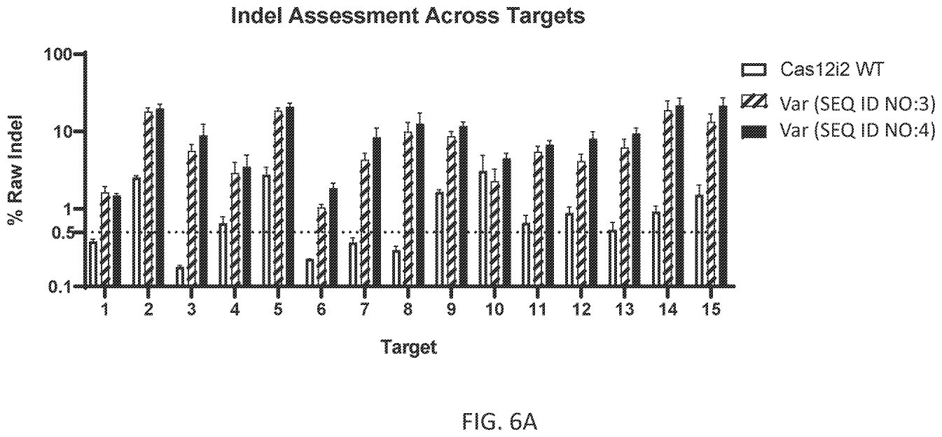

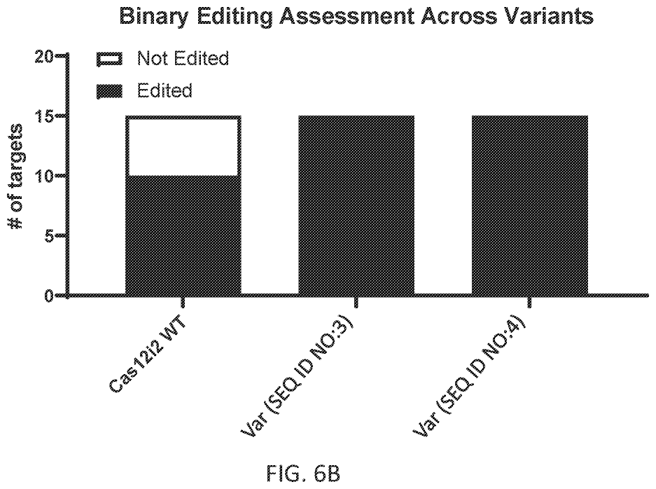

FIG. 6A shows targeting of the fifteen genetic regions in Table 18 by wild-type Cas12i2, variant Cas12i2 of SEQ ID NO: 3, and variant Cas12i2 of SEQ ID NO: 4. Edited targets were defined as targets that showed indel levels above background (>0.5% in this assay, marked by the dotted line). Across the same target set, wild-type Cas12i2 edited 10 out of the 15 targets above background but failed to edit 5 out of the 15 targets above background (FIG. 6A and FIG. 6B). Variant Cas12i2 of SEQ ID NO: 3 and variant Cas12i2 of SEQ ID NO: 4, however, edited each of the 15 targets above background (FIG. 6A and FIG. 6B).

This Example shows that Cas12i2 variant binary complexes demonstrated increased ternary complex formation with target mammalian genes, as compared to wild-type Cas12i2 binary complexes. The Example further shows that the Cas12i2 variants expanded the targeting space by introducing indels in targets unable to be edited by wild-type Cas12i2.

### Example 12—Increased Indel Rates by Cas12i2 Variants

This Example describes increased enzymatic (e.g., nuclease) activity of Cas12i2 variants, as compared to wild-type Cas12i2, over multiple targets.

Cas12i2 plasmids and RNA guides were prepared as described in Example 11. The prepared crRNA sequences and their corresponding target sequences are shown in Table 19. Furthermore, cells were transfected and harvested, and samples were prepared for Next Generation sequencing as described in Example 11.

TABLE 19
crRNA and Target Sequences for
Transient Transfection.

| Target | crRNA sequence | Target Sequence |
|---|---|---|
| 1 | AGAAATCCGTCTTTCATTGAC | GGTGAGGGAGGAGAGATG |
| (AAVS1) | GGGGTGAGGGAGGAGAGATGC | CC (SEQ ID NO: 232) |
| | C (SEQ ID NO: 147) | |
| 2 | AGAAATCCGTCTTTCATTGAC | TCTTGAGATGGGCTCGGG |
| (EMX1) | GGTCTTGAGATGGGCTCGGGC | CT (SEQ ID NO: 234) |
| | T (SEQ ID NO: 233) | |
| 3 | AGAAATCCGTCTTTCATTGAC | TCCGCTCTGAGCAAGGCC |
| (VEGFA) | GGTCCGCTCTGAGCAAGGCCC | CA (SEQ ID NO: 236) |
| | A (SEQ ID NO: 235) | |

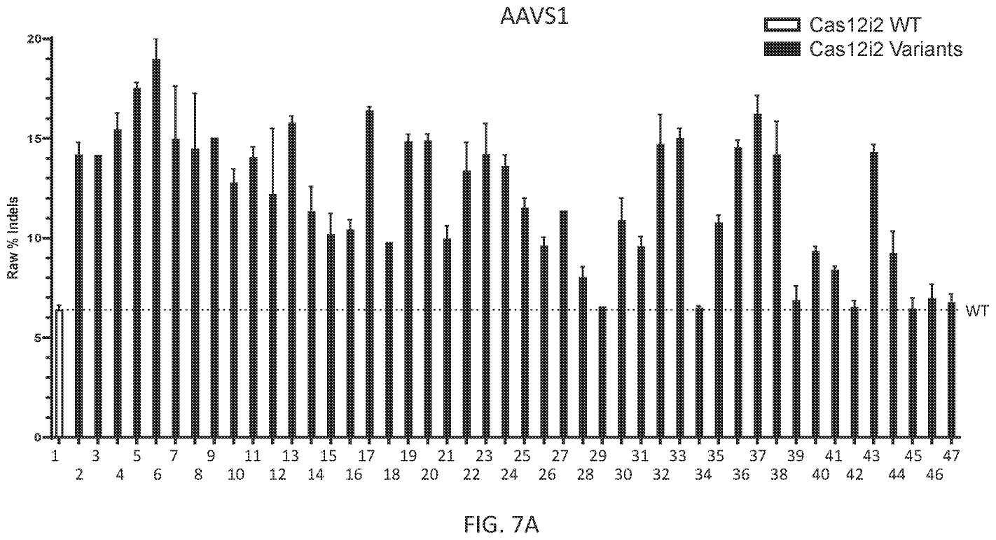

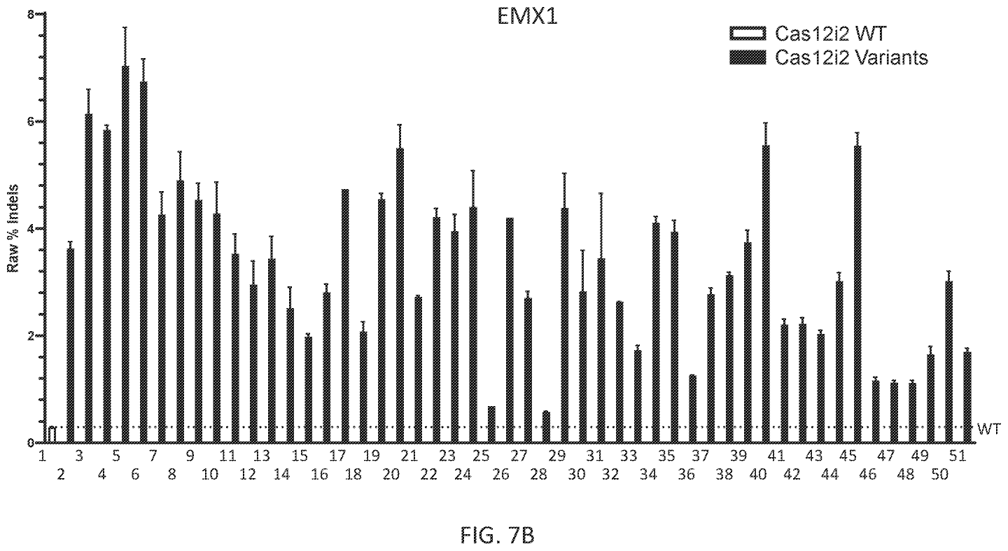

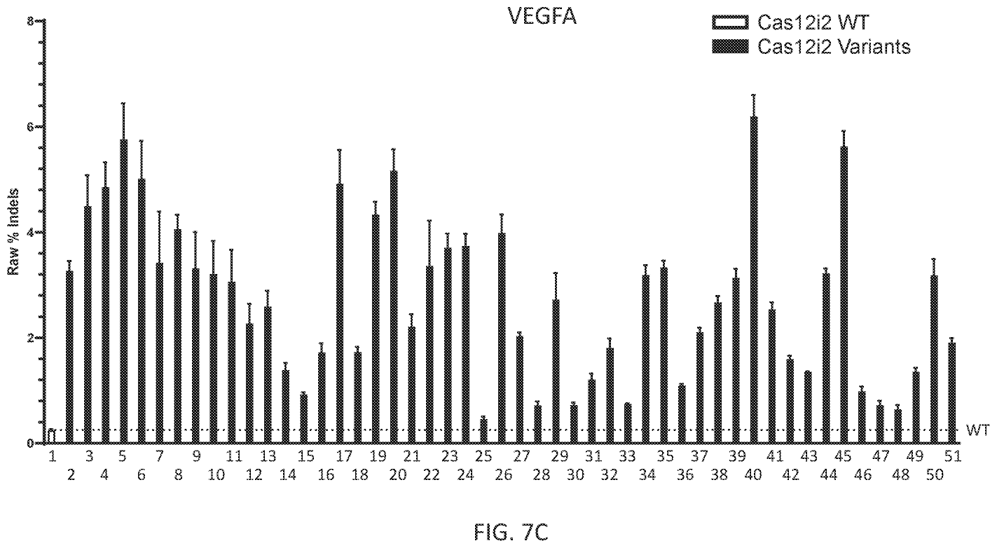

Wild-type Cas12i2 and variant Cas12i2 performance was assessed by comparing indels at AAVS1, EMX1, and VEGFA. As shown in FIG. 7A, FIG. 71B, and FIG. 7C, Cas12i2 variants increased indel rates as compared to wild-type Cas12i2 at an AAVS1 target, EMX1 target, and VEGFA target, respectively. The dotted lines demark Cas12i2 indel rates at AAVS1, EMX1, and VEGFA. Sequence identifiers of the Cas12i2 variants corresponding to each bar in the bar graphs of FIG. 7A are indicated in Table 20, and sequence identifiers of the Cas12i2 variants corresponding to each bar in the bar graphs of FIG. 71B and FIG. 7C are indicated in Table 21.

TABLE 20
Cas12i2 Variant Sequences used in
Indel Assay for AAVS1 (FIG. 7A).

| Bar | Cas12i2 SEQ ID NO |
|---|---|
| 1 | 2 (WT) |
| 2 | 3 |
| 3 | 4 |
| 4 | 46 |
| 5 | 5 |
| 6 | 47 |
| 7 | 50 |
| 8 | 51 |
| 9 | 52 |
| 10 | 53 |
| 11 | 54 |
| 12 | 55 |
| 13 | 56 |
| 14 | 57 |
| 15 | 58 |
| 16 | 59 |
| 17 | 60 |
| 18 | 61 |
| 19 | 62 |
| 20 | 63 |
| 21 | 65 |
| 22 | 66 |
| 23 | 67 |
| 24 | 68 |
| 25 | 79 |
| 26 | 84 |
| 27 | 87 |
| 28 | 88 |
| 29 | 89 |
| 30 | 90 |
| 31 | 95 |
| 32 | 96 |
| 33 | 97 |
| 34 | 99 |
| 35 | 101 |
| 36 | 103 |
| 37 | 104 |
| 38 | 112 |
| 39 | 114 |
| 40 | 115 |
| 41 | 116 |
| 42 | 117 |
| 43 | 118 |
| 44 | 123 |
| 45 | 130 |
| 46 | 131 |
| 47 | 130 |

TABLE 21
Cas12i2 Variant Sequences used in Indel Assays
for EMX1 (FIG. 7B) and VEGFA (FIG. 7C)

| Bar | Cas12i2 SEQ ID NO |
|---|---|
| 1 | 2 (WT) |
| 2 | 3 |
| 3 | 4 |
| 4 | 46 |
| 5 | 5 |
| 6 | 47 |
| 7 | 50 |
| 8 | 51 |
| 9 | 52 |
| 10 | 53 |
| 11 | 54 |
| 12 | 55 |
| 13 | 56 |
| 14 | 57 |
| 15 | 58 |
| 16 | 59 |
| 17 | 60 |
| 18 | 61 |
| 19 | 62 |
| 20 | 63 |
| 21 | 65 |
| 22 | 66 |
| 23 | 67 |
| 24 | 68 |
| 25 | 76 |
| 26 | 79 |
| 27 | 84 |
| 28 | 86 |
| 29 | 87 |
| 30 | 88 |
| 31 | 89 |
| 32 | 90 |
| 33 | 95 |
| 34 | 96 |
| 35 | 97 |
| 36 | 99 |
| 37 | 101 |
| 38 | 103 |
| 39 | 104 |
| 40 | 112 |
| 41 | 114 |
| 42 | 115 |
| 43 | 116 |
| 44 | 117 |
| 45 | 118 |
| 46 | 119 |
| 47 | 120 |
| 48 | 121 |
| 49 | 122 |
| 50 | 123 |
| 51 | 124 |

This Example shows that Cas12i2 variant binary complexes demonstrated increased ternary complex formation with target mammalian genes, as compared to wild-type Cas12i2 binary complexes. Furthermore, not only did Cas12i2 variants introduce indels at target loci not capable of being targeted by wild-type Cas12i2 (Example 11), Cas12i2 variants had increased enzymatic activity at multiple target loci, compared to wild-type Cas12i2.

### Example 13—Efficient Immune Cell Editing with a Cas12i2 Variant

This Example describes RNP transfection followed by FACS staining and indel assessment on multiple targets using an engineered variant Cas12i2 nuclease (SEQ ID NO: 4) and SpCas9 control in primary T cells.

CRISPR RNA-guided nucleases have gained considerable interest for their role in revolutionizing existing ex vivo approaches to engineered cell therapies. Here we present a novel engineered Type V CRISPR-Cas variant, Cas12i, as an alternative to the widely used Cas9 and Cpf1 CRISPR nuclease systems. Although effectors of subtypes V-A (Cas12a, also known as Cpf1) and V-B (Cas12b) have been studied in detail, the distinct domain architectures and diverged RuvC sequences of uncharacterized Cas12 proteins suggest unexplored functional diversity (Yan et al., Science 363(6422):88-91, 2019). In an effort to uncover such functional diversity, rigorous characterization of Cas12i revealed therapeutically differentiating attributes over classical gene modification nucleases, including compact size (1054 amino acid protein, 43 nucleotide tracr-less guide RNA), the validation of an optimal T-rich PAM in mammalian cells, distinct editing outcomes, and potential for multiplexing. We engaged in a high-throughput evaluation of engineered variants of Cas12i resulting in a Cas12i variant that yielded dramatically increased (˜40-fold) activity over the original Cas12i and has enhanced specificity as compared to SpCas9. RNP-mediated delivery of the Cas12i variant to T cells targeting therapeutically relevant loci revealed robust editing (>90% indel across multiple targets and donors). Additionally, specificity studies using in silico prediction coupled with targeted NGS sequencing demonstrated the robust activity and specificity of the Cas12i variant. Taken together, these data show that this variant of Cas12i is uniquely differentiated to enable robust and precise engineered cell therapies. Details of these studies are as follows.

CD3+ T cells from three individual donors were revived and counted using an automated cell counter. A sample from each donor was collected and stained for CD3E and DAPI for flow cytometry analysis of surface expression and viability, respectively. Cell density was adjusted to 1e6 cells/mL and cells were stimulated for 3 days with a cocktail of anti-CD3:CD28 antibodies.

Variant Cas12i2 RNP complexation reactions were made by mixing purified variant Cas12i2 (400 μM; SEQ ID NO: 4) with crRNA (1 mM in 250 mM NaCl; see sequences in Tables 13 and 14) at a 1:1 (effector:crRNA) volume ratio (2.5:1 crRNA:effector molar ratio). SpCas9 RNP complexation reactions were made by mixing purified SpCas9 (Aldevron; 62 μM) with sgRNA (1 mM in water; see sequences in Table 22) at a 6.45:1 (effector:sgRNA) volume ratio (2.5:1 sgRNA:effector molar ratio). For “effector only” controls, variant Cas12i2 or SpCas9 were mixed with Protein Storage Buffer (25 mM Tris, pH 7.5, 250 m NaCl, 1 mM TCEP, 50% glycerol) at the same volume ratio as the crRNA or sgRNA, respectively. Additional controls were included: SpCas9 (Aldevron) with either Lethal #1 (transfection control guide), pooled CD3, or ROSA26 sgRNAs and SpCas9 (Horizon) with either Lethal #1, pooled CD3, or ROSA26 sgRNAs. Complexations were incubated at 37° C. for 30-60 min. Following incubation, RNPs were diluted to 20 μM, 50 μM, 100 μM, or 160 μM effector concentration for variant Cas12i2 and 20 μM or 50 μM for SpCas9.

TABLE 22
crRNA and sgRNA sequences for RNP transfection
(SEQ ID NOs: 237-241).

| | | | | Target | |
|---|---|---|---|---|---|
| Guide Name | Gene | Effector | PAM | Strand | crRNA/sgRNA |
| Cas1212_B2M | B2M | Cas12i2 | CTTC | TS | rArGrArArArUrCrCrGrUrCrUrUrUrCrArU |
| exon2_target4 | | | | | rUrGrArCrGrGrArArUrGrUrCrGrGrArUr |
| | | | | | GrGrArUrGrArArArCrC |
| Cas12i2_B2M | B2M | Cas12i2 | CTTT | BS | rArGrArArArUrCrCrGrUrCrUrUrUrCrArU |
| exon2_target8 | | | | | rUrGrArCrGrGrCrUrArUrCrUrCrUrUrGrU |
| | | | | | rArCrUrArCrArCrUrG |
| Cas12i2_B2M | B2M | Cas12i2 | GTTC | TS | rArGrArArArUrCrCrGrUrCrUrUrUrCrArU |
| exon2_target10 | | | | | rUrGrArCrGrGrArCrArCrGrGrCrArGrGrC |
| | | | | | rArUrArCrUrCrArUrC |
| Cas12i2_B2M | B2M | Cas12i2 | CTTT | BS | rArGrArArArUrCrCrGrUrCrUrUrUrCrArU |
| exon2_target11 | | | | | rUrGrArCrGrGrGrUrCrArCrArGrCrCrCrA |
| | | | | | rArGrArUr ArGrUrUrA |
| SpCas9_B2M | B2M | SpCas9 | TGG | BS | mG*mG*mC*rCrGrArGrArUrGrUrCrUrCr |
| exon1_target1 | | | | | GrCrUrCrCrGrGrUrUrUrUrArGrArGrCrUr |
| | | | | | ArGrArAr ArUrArGrCrArArGrUrUrArArA |
| | | | | | rArUrArArGrGrCrUrArGrUrCrCrGrUrUrA |
| | | | | | rUrCrArArCrUrUrGrArArArArArGrUrGr |
| | | | | | GrCrArCrCrGrArGrUrCrGrGrUrGrCmU* |
| | | | | | mU*mU*rU |

TABLE 23
crRNA and sgRNA sequences for RNP transfection (SEQ ID NOs: 242-251).

| | | | | Target | |
|---|---|---|---|---|---|
| Guide Name | Gene | Effector | PAM | Strand | crRNA/sgRNA |
| Cas12i2_TRAC_ | TRAC | Cas12i2 | CTTC | TS | rArGrArArArUrCrCrGrUrCrUrUrUrCrAr |
| exon1_target3 | | | | | UrUrGrArCrGrGrArArGrArGrCrArArCr |
| | | | | | ArGrUrGrCrUrGrUrGrGrC |
| Cas1212_TRAC | TRAC | Cas12i2 | CTTC | BS | rArGrArArArUrCrCrGrUrCrUrUrUrCrAr |
| exon1_target5 | | | | | UrUrGrArCrGrGrArArCrArArCrArGrCr |
| | | | | | ArUrUrArUrUrCrCrArGrA |
| Cas12i2_TRAC_ | TRAC | Cas12i2 | CTTT | TS | rArGrArArArUrCrCrGrUrCrUrUrUrCrAr |
| exon2_target4 | | | | | UrUrGrArCrGrGrGrArArArCrArGrGrUr |
| | | | | | ArArGrArCrArGrGrGrGrU |
| Cas12i2_TRAC_ | TRAC | Cas12i2 | CTTT | BS | rArGrArArArUrCrCrGrUrCrUrUrUrCrAr |
| exon3_target4 | | | | | UrUrGrArCrGrGrCrArGrGrArGrGrArGr |
| | | | | | GrArUrUrCrGrGrArArCrC |
| SpCas9_TRAC_ | TRAC | SpCas9 | TGG | BS | mA*mA*mG*rUrUrCrCrUrGrUrGrArUr |
| exon2_target1 | | | | | GrUrCrArArGrCrGrUrUrUrUrArGrArGr |
| | | | | | CrUrArGrArArArUrArGrCrArArGrUrUr |
| | | | | | ArArArArUrArArGrGrCrUrArGrUrCrCr |
| | | | | | GrUrUrArUrCrArArCrUrUrGrArArArAr |
| | | | | | ArGrUrGrGrCrArCrCrGrArGrUrCrGrGr |
| | | | | | UrGrCmU*mU*mU*rU |
| Cas1212_PDCD1_ | PDCD1 | Cas12i2 | GTTC | BS | rArGrArArArUrCrCrGrUrCrUrUrUrCrAr |
| exon1_target1 | | | | | UrUrGrArCrGrGrUrUrArGrGrUrArGrGr |
| | | | | | UrGrGrGrGrUrCrGrGrCrG |
| Cas1212_PDCD1_ | PDCD1 | Cas12i2 | CTTC | BS | rArGrArArArUrCrCrGrUrCrUrUrUrCrAr |
| exon2_target7 | | | | | UrUrGrArCrGrGrCrCrCrGrArGrGrArCr |
| | | | | | CrGrCrArGrCrCrArGrCrC |
| Cas12i2_PDCD1_ | PDCD1 | Cas12i2 | CTTC | BS | rArGrArArArUrCrCrGrUrCrUrUrUrCrAr |
| exon2_target8 | | | | | UrUrGrArCrGrGrCrGrUrGrUrCrArCrAr |
| | | | | | CrArArCrUrGrCrCrCrArA |
| Cas12i2_PDCD1_ | PDCD1 | Cas12i2 | CTTC | BS | rArGrArArArUrCrCrGrUrCrUrUrUrCrAr |
| exon2_target9 | | | | | UrUrGrArCrGrGrCrArCrArUrGrArGrCr |
| | | | | | GrUrGrGrUrCrArGrGrGrC |
| SpCas9_PDCD1_ | PDCD1 | SpCas9 | AGG | BS | mU*mC*mC*rArGrGrCrArUrGrCrArGr |
| exon1_target1 | | | | | ArUrCrCrCrArCrGrUrUrUrUrArGrArGr |
| | | | | | CrUrArGrArArArUrArGrCrArArGrUrUr |
| | | | | | ArArArArUrArArGrGrCrUrArGrUrCrCr |
| | | | | | GrUrUrArUrCrArArCrUrUrGrArArArAr |
| | | | | | ArGrUrGrGrCrArCrCrGrArGrUrCrGrGr |
| | | | | | UrGrCmU*mU*mU*rU |

Diluted complexed reactions were dispensed at 2 μL per well into a 384-well electroporation plate. Cell suspensions were collected and counted using an automated cell counter. Cell density was adjusted to 1.1e7 cells/mL in P3 buffer and was dispensed at 2e5 cells/reaction (18 μL). Final concentration of variant Cas12i2 RNPs was 2 μM, 5 μM, 10 μM, or 16 μM. Final concentration of SpCas9 RNPs was 2 or 5 μM. The following controls were set up: unelectroporated cells only, cells in P3 primary cell buffer (Lonza #VXP-3032) only, cells in Protein Storage Buffer only. The plate was electroporated using an electroporation device (program EO-115-AA, Lonza H T), excluding the unelectroporated conditions. Each well was split into four 96-well editing plates (containing 200 μL total volume) using robotics (StarLab Hamilton). Editing plates were incubated for 7 days at 37° C. with 100 μL media replacement at day 4. After 7 days, plates were spun down and the supernatant was removed. Pellets were resuspended in 200 μL of PBS. 100 μL of sample was collected and stained with either the antibody panel (anti-B2M, anti-TRAC, anti-PD1) or anti-CD3E antibody (lethal #1, pooled CD3E, ROSA26, Protein Storage Buffer and unelectroporated for Cas9 controls). All cells were stained with DAPI to assess viability. Remaining cell suspension was transferred to a 96-well PCR plate and pelleted at 500×g for 5 min. Supernatants were removed and pellets were frozen at −80° C.

For gDNA extraction, pellets were thawed to room temperature and resuspended in appropriate volume of DNA extraction buffer (QuickExtract) to give final concentration of 1000 cells/μL. Samples were then cycled in PCR machine at 65° C. for 15 min, 68° C. for 15 min, 98° C. for 10 min. Samples were then frozen at −20° C.

Samples for Next Generation Sequencing (NGS) were prepared by rounds of PCR. The first round (PCR I) was used to amplify the genomic regions flanking the target site and add NGS adapters. The second round (PCR II) was used to add NGS indexes. Reactions were then pooled, purified by column purification, and quantified on a fluorometer (Qubit). Sequencing runs were done using a 150 cycle NGS instrument (NextSeq v2.5) mid or high output kit and run on an NGS instrument (NextSeq 550).

For NGS analysis, the indel mapping function used a sample's fastq file, the amplicon reference sequence, and the forward primer sequence. For each read, a kmer-scanning algorithm was used to calculate the edit operations (match, mismatch, insertion, deletion) between the read and the reference sequence. In order to remove small amounts of primer dimer present in some samples, the first 30 nt of each read was required to match the reference and reads where over half of the mapping nucleotides were mismatches were filtered out as well. Up to 50,000 reads passing those filters were used for analysis, and reads were counted as an indel read if they contained an insertion or deletion. The indel % was calculated as the number of indel-containing reads divided by the number of reads analyzed (reads passing filters up to 50,000). The QC standard for the minimum number of reads passing filters was 10,000.

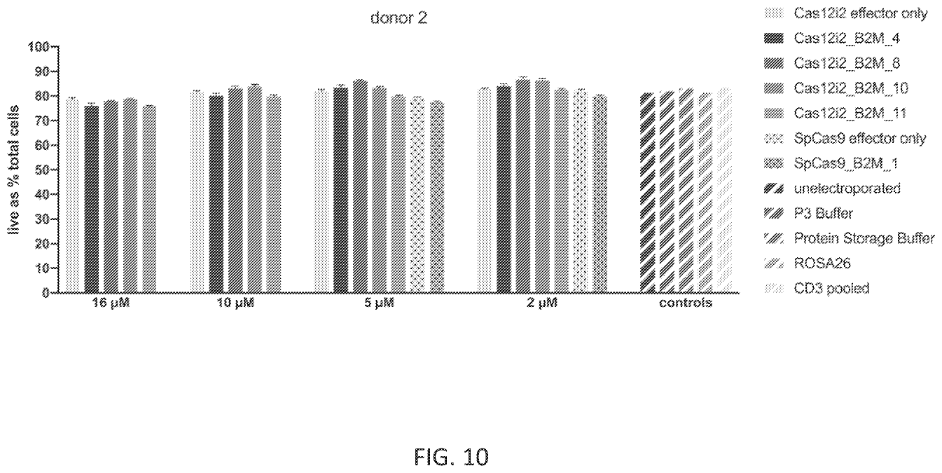

These results demonstrated robust indel activity by variant Cas12i2 RNP targeting multiple B2M targets in primary T cells (FIG. 8), with activity peaking at 16 μM. Flow cytometry staining showed significant reduction of B2M protein expression in T cells following variant Cas12i2 RNP (FIG. 9). The results additionally show indels in TRAC and PDCD1 induced by variant Cas12i2 RNP targeting in primary T cells (FIG. 11A and FIG. 12A, respectively). Cell viability remained high for all conditions seven days post electroporation of the Cas12i2 RNPs targeting B2M (FIG. 10), the Cas12i2 RNPs targeting TRAC (FIG. 11B), and the Cas12i2 RNPs targeting PDCD1 (FIG. 12B).

This Example shows that Cas12i2 variant binary complexes (e.g., variant Cas12i2 RNPs) formed stable ternary complexes with multiple targets and have activity in primary T cells. This Example further shows that variant Cas12i2 formed a stable binary complex (e.g., RNP) that had increased (on target) binding affinity to a target nucleic acid and had activity in primary T cells.

### Example 14—Determination of Off-Target Enzymatic Activity by Cas12i2 Variants

This Example describes methods for assessing nuclease activity of a Cas12i2 binary complex at on-target and non-target loci.

The target sequences in Table 24 were selected for this assay. For each target sequence, potential non-target sites were identified by searching for other genomic sequences adjacent to a PAM sequence and calculating the Levenshtein distance between the target sequence and the PAM-adjacent sequences. The Levenshtein distance (e.g., edit distance) corresponds to the minimum number of edits (e.g., insertions, deletions, or substitutions) required to change one sequence into another (e.g., to change the sequence of a potential non-target locus into the sequence of the on-target locus), as shown in FIG. 13. Potential non-target sequences with an edit distance of 1, 2, 3, or 4 were chosen and are shown in Table 25.

TABLE 24
On-target sequences and corresponding crRNA sequences.

| Target | | |
|---|---|---|
| Locus | Target | crRNA |
| AAVS1_ | TGTCCCCCCAAGTTTTGGAC | AGAAATCCGTCTTTCATTGACGGTGTCCCCCCAAGT |
| T1 | (SEQ ID NO: 252) | TTTGGAC (SEQ ID NO: 253) |
| AAVS1_ | GGAGAGGTGAGGGACTTGGG | AGAAATCCGTCTTTCATTGACGGGGAGAGGTGAGGG |
| T2 | (SEQ ID NO: 254) | ACTTGGG (SEQ ID NO: 255) |
| AAVS1_ | GTGAGAATGGTGCGTCCTAG | AGAAATCCGTCTTTCATTGACGGGTGAGAATGGTGC |
| T3 | (SEQ ID NO: 256) | GTCCTAG (SEQ ID NO: 257) |
| AAVS1_ | GGGGTTGTCCAGAAAAACGG | AGAAATCCGTCTTTCATTGACGGGGGGTTGTCCAGA |
| T4 | (SEQ ID NO: 258) | AAAACGG (SEQ ID NO: 259) |
| AAVS1_ | AACTGGCCCTGGCTTTGGCA | AGAAATCCGTCTTTCATTGACGGAACTGGCCCTGGC |
| T5 | (SEQ ID NO: 260) | TTTGGCA (SEQ ID NO: 261) |
| AAVS1_ | GTAGCCTCTCCCGCTCTGGT | AGAAATCCGTCTTTCATTGACGGGTAGCCTCTCCCG |
| T6 | (SEQ ID NO: 262) | CTCTGGT (SEQ ID NO: 263) |
| AAVS1_ | GGGAAGTGGTTGGTCAGCAT | AGAAATCCGTCTTTCATTGACGGGGGAAGTGGTTGG |
| T7 | (SEQ ID NO: 264) | TCAGCAT (SEQ ID NO: 265) |
| AAVS1_ | AGCGGGTATGGGAAGGGCTT | AGAAATCCGTCTTTCATTGACGGAGCGGGTATGGGA |
| T8 | (SEQ ID NO: 266) | AGGGCTT (SEQ ID NO: 267) |
| EMX1_T1 | GGGCGCAGGGCCACCTGGAC | AGAAATCCGTCTTTCATTGACGGGGGCGCAGGGCCA |
| | (SEQ ID NO: 268) | CCTGGAC (SEQ ID NO: 269) |
| EMX1_T2 | GGATGGCGACTTCAGGCACA | AGAAATCCGTCTTTCATTGACGGGGATGGCGACTTC |
| | (SEQ ID NO: 270) | AGGCACA (SEQ ID NO: 271) |
| EMX1_T3 | ATGTGATTGATGCCCAAAGG | AGAAATCCGTCTTTCATTGACGGATGTGATTGATGC |
| | (SEQ ID NO: 272) | CCAAAGG (SEQ ID NO: 273) |
| EMX1_T4 | GGGGAGGCCTGGAGTCATGG | AGAAATCCGTCTTTCATTGACGGGGGGAGGCCTGGA |
| | (SEQ ID NO: 274) | GTCATGG (SEQ ID NO: 275) |
| EMX1_T5 | TATTATTCCCATAGGGAAGG | AGAAATCCGTCTTTCATTGACGGTATTATTCCCATA |
| | (SEQ ID NO: 276) | GGGAAGG (SEQ ID NO: 277) |
| EMX1_T6 | GAGCCAGTGTTGCTAGTCAA | AGAAATCCGTCTTTCATTGACGGGAGCCAGTGTTGC |
| | (SEQ ID NO: 278) | TAGTCAA (SEQ ID NO: 279) |
| EMX1_T7 | AGCAAGGGACTATTCAGGGA | AGAAATCCGTCTTTCATTGACGGAGCAAGGGACTAT |
| | (SEQ ID NO: 280) | TCAGGGA (SEQ ID NO: 281) |
| EMX1_T8 | AAAATTGAGCAATCTACCCT | AGAAATCCGTCTTTCATTGACGGAAAATTGAGCAAT |
| | (SEQ ID NO: 282) | CTACCCT (SEQ ID NO: 283) |
| VEGFA_ | TGGGGGTGACCGCCGGAGCG | AGAAATCCGTCTTTCATTGACGGTGGGGGTGACCGC |
| T1 | (SEQ ID NO: 284) | CGGAGCG (SEQ ID NO: 285) |
| VEGFA_ | AATCCTCCACCAGTCATGGT | AGAAATCCGTCTTTCATTGACGGAATCCTCCACCAG |
| T2 | (SEQ ID NO: 286) | TCATGGT (SEQ ID NO: 287) |
| VEGFA_ | GTTGACATTGTCCACACCTG | AGAAATCCGTCTTTCATTGACGGGTTGACATTGTCC |
| T3 | (SEQ ID NO: 288) | ACACCTG (SEQ ID NO: 289) |
| VEGFA_ | GGAAATCTATTGAGGCTCTG | AGAAATCCGTCTTTCATTGACGGGGAAATCTATTGA |
| T4 | (SEQ ID NO: 290) | GGCTCTG (SEQ ID NO: 291) |
| VEGFA_ | TTAAACTCTCCATGGACCAG | AGAAATCCGTCTTTCATTGACGGTTAAACTCTCCAT |
| T5 | (SEQ ID NO: 292) | GGACCAG (SEQ ID NO: 293) |
| VEGFA_ | GCCCATACTGGGGACCAAGG | AGAAATCCGTCTTTCATTGACGGGCCCATACTGGGG |
| T6 | (SEQ ID NO: 294) | ACCAAGG (SEQ ID NO: 295) |
| VEGFA_ | GCCGTAACCCTTCGTGGGTA | AGAAATCCGTCTTTCATTGACGGGCCGTAACCCTTC |
| T7 | (SEQ ID NO: 296) | GIGGGTA (SEQ ID NO: 297) |
| VEGFA_ | AGCTCTCGTGGGGGIGGGTC | AGAAATCCGTCTTTCATTGACGGAGCTCTCGTGGGG |
| T8 | (SEQ ID NO: 298) | GTGGGTC (SEQ ID NO: 299) |

TABLE 25
Non-target loci sequences and edit distances.

| Target | Non-target | Edit | Non-target |
|---|---|---|---|
| Locus | Reference | Distance | Locus Sequence |
| AAVS1_T1 | OT-0 | 2 | TGTCCCCCCAAGATTTGGAT |
| | | | (SEQ ID NO: 300) |
| AAVS1_T1 | OT-1 | 2 | TGTCCCCCAAACTTTTGGAC |
| | | | (SEQ ID NO: 301) |
| AAVS1_T1 | OT-2 | 3 | TGTCTCCGCCAAGTTTTGGA |
| | | | (SEQ ID NO: 302) |
| AAVS1_T1 | OT-3 | 3 | GTCCCCCAAAGCTTTTGGAC |
| | | | (SEQ ID NO: 303) |
| AAVS1_T1 | OT-4 | 3 | TGTCCCCAGAAGTTTTGGAA |
| | | | (SEQ ID NO: 304) |
| AAVS1_T1 | OT-5 | 3 | AGTCCCCCCAAGTCCTGGAC |
| | | | (SEQ ID NO: 305) |
| AAVS1_T1 | OT-6 | 3 | TGTCTCCACCAAGTTTTGGC |
| | | | (SEQ ID NO: 306) |
| AAVS1_T1 | OT-7 | 3 | TGTCCCCCCAAGTGTTGAAA |
| | | | (SEQ ID NO: 307) |
| AAVS1_T2 | OT-0 | 2 | GGAGGGGTGAGGGAGTTGGG |
| | | | (SEQ ID NO: 308) |
| AAVS1_T2 | OT-1 | 2 | GGAGTGGTGAGGGACTTGAG |
| | | | (SEQ ID NO: 309) |
| AAVS1_T2 | OT-2 | 2 | GGAGAGGATGGGGACTTGGG |
| | | | (SEQ ID NO: 310) |
| AAVS1_T2 | OT-3 | 2 | GAGAGGTGAGGGACTTGGGG |
| | | | (SEQ ID NO: 311) |
| AAVS1_T2 | OT-4 | 3 | GGAGAGGTGGGGGTGTTGGG |
| | | | (SEQ ID NO: 312) |
| AAVS1_T2 | OT-5 | 3 | GGGAGGCTGAGGCACTTGGG |
| | | | (SEQ ID NO: 313) |
| AAVS1_T2 | OT-6 | 3 | GGAGAGGGGAAGGATTTGGG |
| | | | (SEQ ID NO: 314) |
| AAVS1_T2 | OT-7 | 3 | GGAGAGGAGAGGGTACTTGG |
| | | | (SEQ ID NO: 315) |
| AAVS1_T3 | OT-0 | 2 | TGAGAATGGTGCGTCCTAGG |
| | | | (SEQ ID NO: 316) |
| AAVS1_T3 | OT-1 | 3 | GTGAGAATGGTGTGTCTTAC |
| | | | (SEQ ID NO: 317) |
| AAVS1_T3 | OT-2 | 3 | CTGAGAATGGTGGTCTCTAG |
| | | | (SEQ ID NO: 318) |
| AAVS1_T3 | OT-3 | 3 | GTGTGAATGGTGCTTCCTGG |
| | | | (SEQ ID NO: 319) |
| AAVS1_T3 | OT-4 | 3 | GTGGGAATGGCGCCTCCTAG |
| | | | (SEQ ID NO: 320) |
| AAVS1_T3 | OT-5 | 3 | CTGAGAATGGTGCTTTCTAG |
| | | | (SEQ ID NO: 321) |
| AAVS1_T3 | OT-6 | 3 | GATGAAATGGTGCTTCCTAG |
| | | | (SEQ ID NO: 322) |
| AAVS1_T3 | OT-7 | 3 | GTGAGAATGGTTCTGTCTAG |
| | | | (SEQ ID NO: 323) |
| AAVS1_T4 | OT-0 | 2 | GGGTTGTCCAGAAAAACGGT |
| | | | (SEQ ID NO: 324) |
| AAVS1_T4 | OT-1 | 2 | GAGGGTTGTCCAGAAAAAGG |
| | | | (SEQ ID NO: 325) |
| AAVS1_T4 | OT-2 | 3 | GGGGTGCTCCAGAAAAACGT |
| | | | (SEQ ID NO: 326) |
| AAVS1_T4 | OT-3 | 3 | GGGTTCTCCAGAAAAACGGA |
| | | | (SEQ ID NO: 327) |
| AAVS1_T4 | OT-4 | 3 | GGGTGTTGCCCAGAAAAAGG |
| | | | (SEQ ID NO: 328) |
| AAVS1_T4 | OT-5 | 3 | GGTGTTGTCCAGAAAGACGT |
| | | | (SEQ ID NO: 329) |
| AAVS1_T4 | OT-6 | 3 | AGGGTTGTCCCAGAAAAAGG |
| | | | (SEQ ID NO: 330) |
| AAVS1_T4 | OT-7 | 3 | TGGGTTGTATAGAAAAACGG |
| | | | (SEQ ID NO: 331) |
| AAVS1_T5 | OT-0 | 2 | ACTGGCCCTGGCTTTGGCAG |
| | | | (SEQ ID NO: 332) |
| AAVS1_T5 | OT-1 | 2 | AACTGGCCCTGGCTCTGGCC |
| | | | (SEQ ID NO: 333) |
| AAVS1_T5 | OT-2 | 3 | CAATGCCCCTGGCTTTGGCA |
| | | | (SEQ ID NO: 334) |
| AAVS1_T5 | OT-3 | 3 | AAACTGGCCCTGGCTCTGGC |
| | | | (SEQ ID NO: 335) |
| AAVS1_T5 | OT-4 | 3 | ACTGACCCTGGCTTATGGCA |
| | | | (SEQ ID NO: 336) |
| AAVS1_T5 | OT-5 | 3 | CCCTGGCTCTGGCTTTGGCA |
| | | | (SEQ ID NO: 337) |
| AAVS1_T5 | OT-6 | 3 | AATGGCCACTGGATTTGGCA |
| | | | (SEQ ID NO: 338) |
| AAVS1_T5 | OT-7 | 3 | AACTGTCCCTGGGCTTTGCA |
| | | | (SEQ ID NO: 339) |
| AAVS1_T6 | OT-0 | 2 | TAGCCTCTCCCGCTCTGGTT |
| | | | (SEQ ID NO: 340) |
| AAVS1_T6 | OT-1 | 3 | GCAGCCACTCCCTCTCTGGT |
| | | | (SEQ ID NO: 341) |
| AAVS1_T6 | OT-2 | 3 | GTAGACTCTCTCTCTCTGGT |
| | | | (SEQ ID NO: 342) |
| AAVS1_T6 | OT-3 | 3 | AGTGCCTCTCCTGCTCTGGT |
| | | | (SEQ ID NO: 343) |
| AAVS1_T6 | OT-4 | 3 | GTTAGCCTCTCCCTCTCTGT |
| | | | (SEQ ID NO: 344) |
| AAVS1_T6 | OT-5 | 3 | GAGTTCTCTCCCGCTCTGGT |
| | | | (SEQ ID NO: 345) |
| AAVS1_T6 | OT-6 | 3 | TAGCCTCACCTCGCTCTGGT |
| | | | (SEQ ID NO: 346) |
| AAVS1_T6 | OT-7 | 3 | GTAGTCCTCTCCCTCTCTGG |
| | | | (SEQ ID NO: 347) |
| AAVS1_T7 | OT-0 | 2 | GGGCAGTGGTTGGTGAGCAT |
| | | | (SEQ ID NO: 348) |
| AAVS1_T7 | OT-1 | 2 | GGGAGTGGTTGGTCATGCAT |
| | | | (SEQ ID NO: 349) |
| AAVS1_T7 | OT-2 | 2 | GGTAAGTGCTTGGTCAGCAT |
| | | | (SEQ ID NO: 350) |
| AAVS1_T7 | OT-3 | 2 | GGAAGTGGTTGGTCAGCATG |
| | | | (SEQ ID NO: 351) |
| AAVS1_T7 | OT-4 | 3 | GGGAAATGGTTTGTCAGCAG |
| | | | (SEQ ID NO: 352) |
| AAVS1_T7 | OT-5 | 3 | GGGAAGTGATTAGGTCACAT |
| | | | (SEQ ID NO: 353) |
| AAVS1_T7 | OT-6 | 3 | GGGAAGTTTTTGGTCATCAT |
| | | | (SEQ ID NO: 354) |
| AAVS1_T7 | OT-7 | 3 | GGAAAGTTGTTGGGCAGCAT |
| | | | (SEQ ID NO: 355) |
| AAVS1_T8 | OT-0 | 2 | AGCGGGTAGGGAAGGGTCTT |
| | | | (SEQ ID NO: 356) |
| AAVS1_T8 | OT-1 | 2 | GCGGGTATGGGAAGGGCTTT |
| | | | (SEQ ID NO: 357) |
| AAVS1_T8 | OT-2 | 3 | ACGCAGGATGGGAAGGGCTT |
| | | | (SEQ ID NO: 358) |
| AAVS1_T8 | OT-3 | 3 | AGCGGCTATGGGGAGGCCTT |
| | | | (SEQ ID NO: 359) |
| AAVS1_T8 | OT-4 | 3 | AGTAGGTATGGGAAGGGCCT |
| | | | (SEQ ID NO: 360) |
| AAVS1_T8 | OT-5 | 3 | AGAGGGTATGGGAATGGCTA |
| | | | (SEQ ID NO: 361) |
| AAVS1_T8 | OT-6 | 3 | AGCAGGTGAGGGAAGGGCTT |
| | | | (SEQ ID NO: 362) |
| AAVS1_T8 | OT-7 | 3 | AGTGGGTCTGGGTAGGGCTT |
| | | | (SEQ ID NO: 363) |
| EMX1 T1 | OT-0 | 2 | GGCGCAGGGCCACCTGGACC |
| | | | (SEQ ID NO: 364) |
| EMX1_T1 | OT-1 | 3 | GGGGCAGGGCCACCTGTAGC |
| | | | (SEQ ID NO: 365) |
| EMX1_T1 | OT-2 | 3 | GTGCCCAGGGCCACCCGGAC |
| | | | (SEQ ID NO: 366) |
| EMX1_T1 | OT-3 | 3 | GTTGCGCAGGGCCACTGGAC |
| | | | (SEQ ID NO: 367) |
| EMX1_T1 | OT-4 | 3 | GGGCGTGGGGCAACCTGGAC |
| | | | (SEQ ID NO: 368) |
| EMX1_T1 | OT-5 | 3 | GGCGCAGGGCACACCGGGAC |
| | | | (SEQ ID NO: 369) |
| EMX1_T1 | OT-6 | 3 | GGGCACAGGGGCCACCTGGA |
| | | | (SEQ ID NO: 370) |
| EMX1_T1 | OT-7 | 3 | AGGCACAGGGCCACTTGGAC |
| | | | (SEQ ID NO: 371) |
| EMX1_T2 | OT-0 | 2 | GATGGCGACTTCAGGCACAG |
| | | | (SEQ ID NO: 372) |
| EMX1 T2 | OT-1 | 3 | GATAGGCGATTTCAGGCACA |
| | | | (SEQ ID NO: 373) |
| EMX1_T2 | OT-2 | 3 | GGATGGCGACTGTAAGGCAA |
| | | | (SEQ ID NO: 374) |
| EMX1 T2 | OT-3 | 3 | GGATGGCGAATACAGGAACA |
| | | | (SEQ ID NO: 375) |
| EMX1 T2 | OT-4 | 3 | GCAGGGCGACTTCAGACACA |
| | | | (SEQ ID NO: 376) |
| EMX1 T2 | OT-5 | 3 | GGATGGCGAATTCAGTCACT |
| | | | (SEQ ID NO: 377) |
| EMX1 T2 | OT-6 | 4 | TGATGGAGAAATCAGGCACA |
| | | | (SEQ ID NO: 378) |
| EMX1 T2 | OT-7 | 4 | GGATGGGGCCTCCAGGCTCA |
| | | | (SEQ ID NO: 379) |
| EMX1_T3 | OT-0 | 2 | ATGTGACTTGAGCCCAAAGG |
| | | | (SEQ ID NO: 380) |
| EMX1_T3 | OT-1 | 2 | ATGTGATTTTTGCCCAAAGG |
| | | | (SEQ ID NO: 381) |
| EMX1 T3 | OT-2 | 2 | TGTGATTGATGCCCAAAGGT |
| | | | (SEQ ID NO: 382) |
| EMX1_T3 | OT-3 | 3 | ATGTAATTATGCCCCAAAGG |
| | | | (SEQ ID NO: 383) |
| EMX1_T3 | OT-4 | 3 | ATGTGATTGTTGACCAAATG |
| | | | (SEQ ID NO: 384) |
| EMX1_T3 | OT-5 | 3 | CTGTGATAGAAGCCCAAAGG |
| | | | (SEQ ID NO: 385) |
| EMX1_T3 | OT-6 | 3 | ATATGATAGATGCCCAAATG |
| | | | (SEQ ID NO: 386) |
| EMX1_T3 | OT-7 | 3 | ATTTGATGGAAGCCCAAAGG |
| | | | (SEQ ID NO: 387) |
| EMX1 T4 | OT-0 | 2 | GTGGAGTCCTGGAGTCATGG |
| | | | (SEQ ID NO: 388) |
| EMX1 T4 | OT-1 | 2 | GGGAGGCCTGGAGTCATGGC |
| | | | (SEQ ID NO: 389) |
| EMX1 T4 | OT-2 | 2 | GTGGGAGGCCTGGAGCATGG |
| | | | (SEQ ID NO: 390) |
| EMX1 T4 | OT-3 | 2 | GGGGAGGGTTGGAGTCATGG |
| | | | (SEQ ID NO: 391) |
| EMX1 T4 | OT-4 | 3 | GGGGAGGCCTGGAGATGATG |
| | | | (SEQ ID NO: 392) |
| EMX1 T4 | OT-5 | 3 | GGGAGGCCTCAGAGTCATGG |
| | | | (SEQ ID NO: 393) |
| EMX1 T4 | OT-6 | 3 | GGGAGGCCTTGGAATCATGG |
| | | | (SEQ ID NO: 394) |
| EMX1 T4 | OT-7 | 3 | GTGGAGTGCCTGGAGCATGG |
| | | | (SEQ ID NO: 395) |
| EMX1 T5 | OT-0 | 2 | TTATTATTCCCATGGGAAGG |
| | | | (SEQ ID NO: 396) |
| EMX1_T5 | OT-1 | 2 | TATTATTCCCATATTGAAGG |
| | | | (SEQ ID NO: 397) |
| EMX1_T5 | OT-2 | 2 | ATTATTCCCATAGGGAAGGG |
| | | | (SEQ ID NO: 398) |
| EMX1_T5 | OT-3 | 2 | TATTAATCCTATAGGGAAGG |
| | | | (SEQ ID NO: 399) |
| EMX1 T5 | OT-4 | 3 | TATTATGCCCATAGAGAAGA |
| | | | (SEQ ID NO: 400) |
| EMX1_T5 | OT-5 | 3 | TATTATTCTCAAAGGGGAGG |
| | | | (SEQ ID NO: 401) |
| EMX1_T5 | OT-6 | 3 | TATTTTTCCCATAGTGAAGA |
| | | | (SEQ ID NO: 402) |
| EMX1_T5 | OT-7 | 3 | TATTGTTCTCATAGAGAAGG |
| | | | (SEQ ID NO: 403) |
| EMX1_T6 | OT-0 | 2 | AGCCAGTGTTGCTAGTCAAG |
| | | | (SEQ ID NO: 404) |
| EMX1_T6 | OT-1 | 3 | GTAGCCGTGTTGCTAGTGAA |
| | | | (SEQ ID NO: 405) |
| EMX1 T6 | OT-2 | 3 | GAGCCACTGTTGGTACTCAA |
| | | | (SEQ ID NO: 406) |
| EMX1 T6 | OT-3 | 3 | GAGGCAGTGTTGCATAGCAA |
| | | | (SEQ ID NO: 407) |
| EMX1_T6 | OT-4 | 3 | GAGACAGIGTGGCAAGTCAA |
| | | | (SEQ ID NO: 408) |
| EMX1 T6 | OT-5 | 3 | CAGCCATTGTTGCTAGGCAA |
| | | | (SEQ ID NO: 409) |
| EMX1 T6 | OT-6 | 3 | TGCCAGTGTTGCTAAGTCAA |
| | | | (SEQ ID NO: 410) |
| EMX1 T6 | OT-7 | 3 | GAGCCACTGTTGAAAGTCAA |
| | | | (SEQ ID NO: 411) |
| EMX1 T7 | OT-0 | 3 | AGCCAGGGACTTTTCAGGGC |
| | | | (SEQ ID NO: 412) |
| EMX1_T7 | OT-1 | 3 | TGCAGGGGACTATTCAGAGA |
| | | | (SEQ ID NO: 413) |
| EMX1 T7 | OT-2 | 3 | AGCCAGGGACTTTTCAGGGC |
| | | | (SEQ ID NO: 414) |
| EMX1 T7 | OT-3 | 3 | ATCAAGGCACAATTCAGGGA |
| | | | (SEQ ID NO: 415) |
| EMX1 T7 | OT-4 | 3 | AGGAAGGGACTCTTCAGAGA |
| | | | (SEQ ID NO: 416) |
| EMX1 T7 | OT-5 | 3 | AGCAAGGGCCTTTCCAGGGA |
| | | | (SEQ ID NO: 417) |
| EMX1 T7 | OT-6 | 3 | AGCAAGTGATATTCAGGAGA |
| | | | (SEQ ID NO: 418) |
| EMX1 T7 | OT-7 | 3 | AGCAGGGGACATTCCAGGGA |
| | | | (SEQ ID NO: 419) |
| EMX1_T8 | OT-0 | 2 | AAATTGAGCAATCTACCCTG |
| | | | (SEQ ID NO: 420) |
| EMX1 T8 | OT-1 | 3 | AACTATTGAGCAATCTCCCT |
| | | | (SEQ ID NO: 421) |
| EMX1 T8 | OT-2 | 3 | AAAATTGAGTAATTTTCCCT |
| | | | (SEQ ID NO: 422) |
| EMX1 T8 | OT-3 | 3 | AAAATTGACCAATTCTACCC |
| | | | (SEQ ID NO: 423) |
| EMX1_T8 | OT-4 | 3 | ACAGTTGAGCATTCTACCCT |
| | | | (SEQ ID NO: 424) |
| EMX1_T8 | OT-5 | 3 | AAAGTTAGCAACTCTACCCT |
| | | | (SEQ ID NO: 425) |
| EMX1 T8 | OT-6 | 3 | AAAAATAAGCAATCTGCCCT |
| | | | (SEQ ID NO: 426) |
| EMX1_T8 | OT-7 | 3 | AATAATTGGCAATCTACTCT |
| | | | (SEQ ID NO: 427) |
| VEGFA_T1 | OT-0 | 2 | GGGGGTGACCGCCGGAGCGC |
| | | | (SEQ ID NO: 428) |
| VEGFA_T1 | OT-1 | 3 | TGGGGGAGCCGCCCGGAGCG |
| | | | (SEQ ID NO: 429) |
| VEGFA_T1 | OT-2 | 3 | TGGAGGTGACCGCCTGGGCG |
| | | | (SEQ ID NO: 430) |
| VEGFA T1 | OT-3 | 3 | TGGGGGTGACAGCTGGTGCG |
| | | | (SEQ ID NO: 431) |
| VEGFA T1 | OT-4 | 4 | TGGGGGTGGCAGCTGGACCG |
| | | | (SEQ ID NO: 432) |
| VEGFA T1 | OT-5 | 4 | TGCGGGTGTCCGCCTGGACG |
| | | | (SEQ ID NO: 433) |
| VEGFA_T1 | OT-6 | 4 | TTGGGGTGACAGCCGGCGGG |
| | | | (SEQ ID NO: 434) |
| VEGFA T1 | OT-7 | 4 | TTGGGGTGACCACAGGAGCA |
| | | | (SEQ ID NO: 435) |
| VEGFA T2 | OT-0 | 2 | ATCCTCCACCAGTCATGGTG |
| | | | (SEQ ID NO: 436) |
| VEGFA_T2 | OT-1 | 3 | AATCCTCCCACAGTCCTGGT |
| | | | (SEQ ID NO: 437) |
| VEGFA_T2 | OT-2 | 3 | ATCCATCCACCAGCCATGGT |
| | | | (SEQ ID NO: 438) |
| VEGFA_T2 | OT-3 | 3 | AATCCTCCAACAGCCCTGGT |
| | | | (SEQ ID NO: 439) |
| VEGFA_T2 | OT-4 | 3 | AATCTTCCACACAGTCTGGT |
| | | | (SEQ ID NO: 440) |
| VEGFA_T2 | OT-5 | 3 | AATCCTCCACCTGTCTTGGC |
| | | | (SEQ ID NO: 441) |
| VEGFA_T2 | OT-6 | 3 | AATCCATCCACCATTCATGG |
| | | | (SEQ ID NO: 442) |
| VEGFA_T2 | OT-7 | 3 | AAGTCCTCACCAGTCATTGT |
| | | | (SEQ ID NO: 443) |
| VEGFA_T3 | OT-0 | 2 | TTGACATTGTCCACACCTGG |
| | | | (SEQ ID NO: 444) |
| VEGFA_T3 | OT-1 | 2 | GTTGACATTGACCACAGCTG |
| | | | (SEQ ID NO: 445) |
| VEGFA_T3 | OT-2 | 3 | GTTGACATGTCCAGCAGCTG |
| | | | (SEQ ID NO: 446) |
| VEGFA_T3 | OT-3 | 3 | GTGACACTTATCCACACCTG |
| | | | (SEQ ID NO: 447) |
| VEGFA_T3 | OT-4 | 3 | GTTGACTATTTTCCAACCTG |
| | | | (SEQ ID NO: 448) |
| VEGFA_T3 | OT-5 | 3 | TTTAACATTTTCCACACCTG |
| | | | (SEQ ID NO: 449) |
| VEGFA_T3 | OT-6 | 3 | GTTGACATTGCCCAGATCTG |
| | | | (SEQ ID NO: 450) |
| VEGFA_T3 | OT-7 | 3 | GAAAACATTGTCCACACCTG |
| | | | (SEQ ID NO: 451) |
| VEGFA_T4 | OT-0 | 2 | TGAAATCTAGTGAGGCTCTG |
| | | | (SEQ ID NO: 452) |
| VEGFA_T4 | OT-1 | 2 | GGAAATCCAGTGAGGCTCTG |
| | | | (SEQ ID NO: 453) |
| VEGFA_T4 | OT-2 | 2 | TGAAATCTAGTGAGGCTCTG |
| | | | (SEQ ID NO: 454) |
| VEGFA_T4 | OT-3 | 2 | GAAATCTATTGAGGCTCTGG |
| | | | (SEQ ID NO: 455) |
| VEGFA_T4 | OT-4 | 3 | AAAAATCTATTGAGGCTGTG |
| | | | (SEQ ID NO: 456) |
| VEGFA_T4 | OT-5 | 3 | GGATATCTATTAGTGCTCTG |
| | | | (SEQ ID NO: 457) |
| VEGFA_T4 | OT-6 | 3 | GGAAATATTTAGAGGCTCTG |
| | | | (SEQ ID NO: 458) |
| VEGFA_T4 | OT-7 | 3 | TGAAATCTAGTGGGGCTCTG |
| | | | (SEQ ID NO: 459) |
| VEGFA_T5 | OT-0 | 2 | TAAACTCTCCATGGACCAGG |
| | | | (SEQ ID NO: 460) |
| VEGFA_T5 | OT-1 | 3 | TTAAAATCTCCATGACCATG |
| | | | (SEQ ID NO: 461) |
| VEGFA_T5 | OT-2 | 3 | TTAATCTCTCCATGGAGCAC |
| | | | (SEQ ID NO: 462) |
| VEGFA_T5 | OT-3 | 3 | TTAAACTCTCTCATGGATCA |
| | | | (SEQ ID NO: 463) |
| VEGFA_T5 | OT-4 | 3 | GTTAAACTCTGCATGGACAG |
| | | | (SEQ ID NO: 464) |
| VEGFA_T5 | OT-5 | 3 | GTAAACTCTCCCTTGACCAG |
| | | | (SEQ ID NO: 465) |
| VEGFA_T5 | OT-6 | 3 | TTAAATCTCCATGAGACAAG |
| | | | (SEQ ID NO: 466) |
| VEGFA_T5 | OT-7 | 3 | TTAAACTCTACAGGACCAAG |
| | | | (SEQ ID NO: 467) |
| VEGFA_T6 | OT-0 | 2 | GCCCATGCTGGGGAACAAGG |
| | | | (SEQ ID NO: 468) |
| VEGFA_T6 | OT-1 | 2 | CCCATACTGGGGACCAAGGA |
| | | | (SEQ ID NO: 469) |
| VEGFA_T6 | OT-2 | 2 | GTCCATACTGGGAACCAAGG |
| | | | (SEQ ID NO: 470) |
| VEGFA_T6 | OT-3 | 3 | GCCCATGACAGGGGCCAAGG |
| | | | (SEQ ID NO: 471) |
| VEGFA_T6 | OT-4 | 3 | CCACAGACTGGGGACCAAGG |
| | | | (SEQ ID NO: 472) |
| VEGFA_T6 | OT-5 | 3 | GCCCATACTCGGGGACCTGG |
| | | | (SEQ ID NO: 473) |
| VEGFA_T6 | OT-6 | 3 | TCCCATAGTGGAGACCAAGG |
| | | | (SEQ ID NO: 474) |
| VEGFA_T6 | OT-7 | 3 | TACCATACTGGGGACCCAGG |
| | | | (SEQ ID NO: 475) |
| VEGFA_T7 | OT-0 | 2 | CCGTAACCCTTCGTGGGTAG |
| | | | (SEQ ID NO: 476) |
| VEGFA_T7 | OT-1 | 3 | GCTGTAATCCCTCGTGGGTA |
| | | | (SEQ ID NO: 477) |
| VEGFA_T7 | OT-2 | 3 | GCCTAACCCTGTTGTGGGTA |
| | | | (SEQ ID NO: 478) |
| VEGFA_T7 | OT-3 | 3 | GCCTAACCCTATCGTGGTTA |
| | | | (SEQ ID NO: 479) |
| VEGFA_T7 | OT-4 | 4 | GACGGAATCCTTCGTGAGTA |
| | | | (SEQ ID NO: 480) |
| VEGFA_T7 | OT-5 | 4 | GCCGTAAGCCCTTCGAGGTC |
| | | | (SEQ ID NO: 481) |
| VEGFA_T7 | OT-6 | 4 | GCGCAAAACCCTTCGTGGGT |
| | | | (SEQ ID NO: 482) |
| VEGFA T7 | OT-7 | 4 | GCCATAACCCTACGGGGTGA |
| | | | (SEQ ID NO: 483) |
| VEGFA_T8 | OT-0 | 2 | GCTCTCGTGGGGGTGGGTCA |
| | | | (SEQ ID NO: 484) |
| VEGFA_T8 | OT-1 | 2 | AGCTCTGTGGGGGTGGGATC |
| | | | (SEQ ID NO: 485) |
| VEGFA_T8 | OT-2 | 3 | GGCTCTCATGGGGGTGTGTC |
| | | | (SEQ ID NO: 486) |
| VEGFA_T8 | OT-3 | 3 | AGCCTGGTGGGGGTGAGGTC |
| | | | (SEQ ID NO: 487) |
| VEGFA_T8 | OT-4 | 3 | AGGCTCCGTGGGTGTGGGTC |
| | | | (SEQ ID NO: 488) |
| VEGFA_T8 | OT-5 | 3 | AGCTCTGTGGGGGTGAGGGC |
| | | | (SEQ ID NO: 489) |
| VEGFA_T8 | OT-6 | 3 | GGCTCTGTGGGGGTGGGATC |
| | | | (SEQ ID NO: 490) |
| VEGFA_T8 | OT-7 | 3 | AGCTCTCGGGGGGTGTGGGC |
| | | | (SEQ ID NO: 491) |

RNA guide sequences corresponding to each target site, as shown in Table 24, were prepared as described in Example 11. HEK293T cells were plated into wells of a 96-well plate, and each well was transfected with a Cas12i2 plasmid and an RNA guide sequence, as described in Example 11.

Samples were collected and prepared for NGS as described in Example 11. Primer3 software was used to identify primer pairs for each non-target site, centering the non-target sequence within a 120-140 base pair amplicon for targeted amplicon sequencing in edited and negative control (effector only) samples. NGS analysis of the on-target amplicons, non-target amplicons, and unedited control samples was then completed. To minimize technical variation, input material for each on-target and all non-targets corresponding to that on-target was taken from the same harvested well. Background corrected non-target activity rates were generated using maximum likelihood estimation (MLE), which estimates the non-target indel level for a sample given the indel level of its paired negative control sample.

On-target editing was defined by an indel level above background (>0.5% in this assay) at a target sequence. Off-target editing was defined by an indel level above background (>0.2% after MLE correction in this assay) at one or more of the corresponding non-target sequences. Indel levels for each on-target sequence and non-target sequence were calculated for the variant Cas12i2 binary complexes.

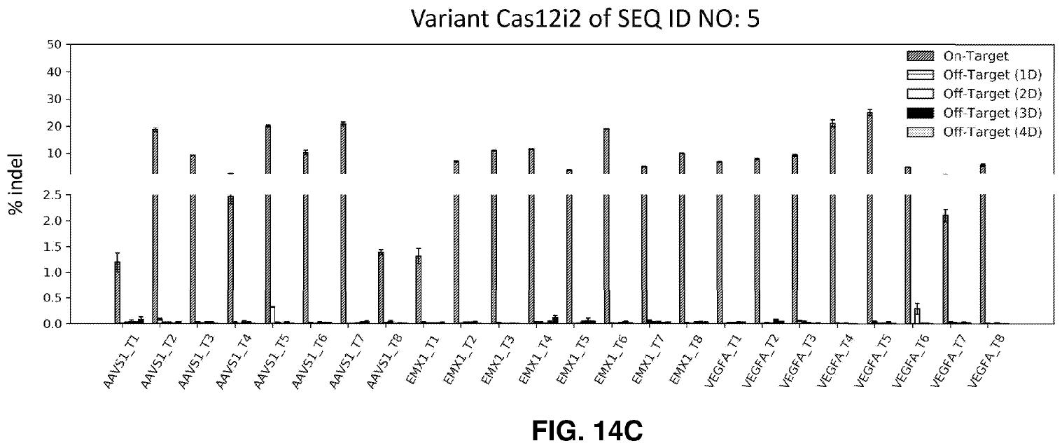

FIG. 14A, FIG. 14B, and FIG. 14C show on-target indel percentages on the AAVS1, EMX1, and VEGFA loci of Table 24 using the Cas12i2 variant of SEQ ID NO: 3, Cas12i2 variant of SEQ ID NO: 4, and Cas12i2 variant of SEQ ID NO: 5, respectively. FIG. 14A, FIG. 14B, and FIG. 14C further show indel percentages on the non-target loci of Table 25. Some off-target enzymatic activity by each of the Cas12i2 variants was observed at various non-target loci, such as at non-target loci corresponding to AAVS1_T5 (edit distance of 2 and/or 3) and VEGFA_T6 (edit distance of 2). However, overall, minimal off-target activity was observed across each of the non-target loci, and enzymatic activity at off-target loci corresponding to particular on-target loci (e.g., AAVS1_T4, AAVS1_T7, AAVS1_T8, EMX1_T1, EMX1_T2, EMX1_T3, EMX1_T5, EMX1_T6, EMX1_T7, VEGFA_T4, VEGFA_T5, VEGFA_T7, and VEGFA_T8) was determined to be absent or negligible. Therefore, each of the Cas12i2 variants exhibited higher on-target activity than off-target activity at multiple loci.

These methods are repeated with wild-type Cas12i2 binary complexes (e.g., parent binary complexes). A variant Cas12i2 binary complex having decreased enzymatic activity at a non-target locus exhibits lower indel percentages at a non-target locus compared to a parent binary complex. A variant Cas12i2 binary complex having increased enzymatic activity at an on-target locus exhibits higher indel percentages at the target locus compared to a parent binary complex. Therefore, this Example shows how enzymatic activity of wild-type Cas12i2 and variant Cas12i2 at on-target and non-target loci can be measured and compared.

### Example 15—Determination of Off-Target Binding Activity by Cas12i2 Variants

This Example describes methods for assessing and comparing binding of a Cas12i2 (wild-type and variant Cas12i2) binary complex to on-target and non-target sequences.

dsDNA substrates corresponding to the on-target sequences and non-target sequences from Example 14 are generated and labeled as described in Example 7. Cas12i2 RNPs targeting each of the on-target sequences of Example 14 are then prepared according to the method of Example 7. Apo reactions (Cas12i2 without RNA guide) are prepared in the same manner, making up the volume of RNA guide with H₂O.

Each RNP or Apo sample is separately incubated with an on-target dsDNA substrate and a non-target dsDNA substrate for 1 hour at 37° C. as described in Example 7. Bromophenol blue is added to the reactions, which are then loaded onto a 6% DNA Retardation Gel (Thermofisher), and the gel is imaged on a Licor Odyssey CLx.

In this assay, the rate at which DNA migrates through the gel is determined by its size. A DNA only sample is able to migrate a particular distance. However, if an RNP binds to the DNA, a band that represents a larger, less mobile DNA complex appears, which is “upshifted” on the gel. Therefore, the “unbound dsDNA” and the “bound dsDNA” bands in the gel are identified for each RNP, wherein the “bound dsDNA” migrate less than the “unbound dsDNA.”

A variant Cas12i2 RNP having increased on-target binding exhibits increased binding to a dsDNA target sequence, as evidenced by a more intense upshifted band compared to a parent RNP. A variant Cas12i2 RNP having decreased non-target binding exhibits decreased binding to a dsDNA non-target sequence, as evidenced by a less intense or missing upshifted band compared to a parent RNP.

Therefore, this Example shows how binding activity of wild-type Cas12i2 and variant Cas12i2 binary complexes on on-target and non-target substrates can be measured and compared.

### Example 16—Methods for Determination of Variant Cas12i2 Off-Target Cleavage Sites

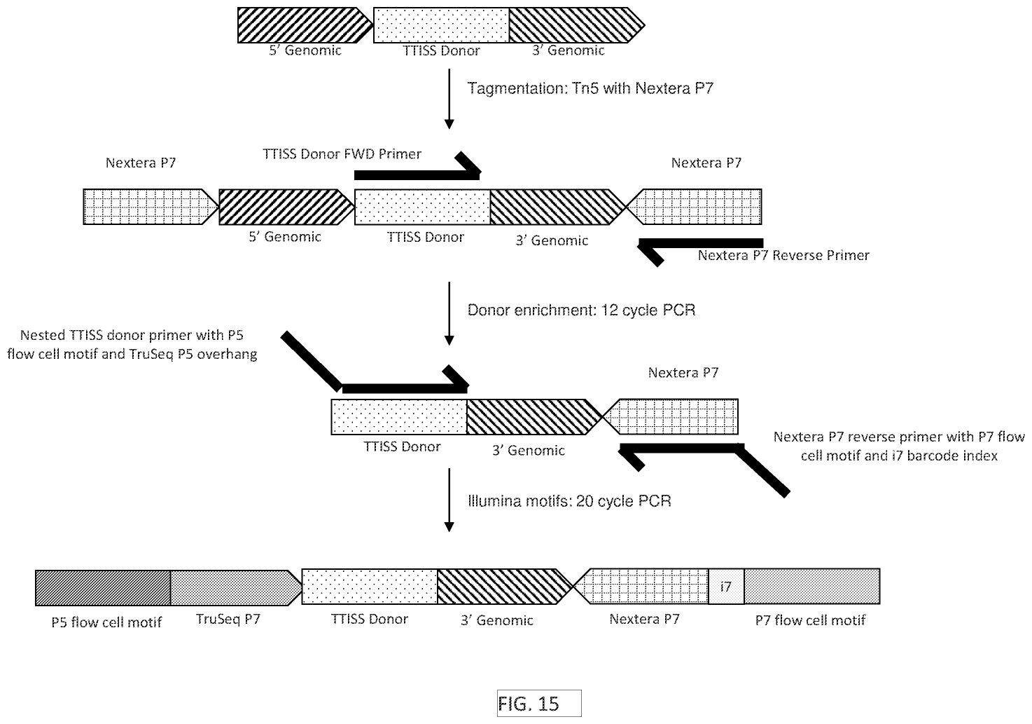

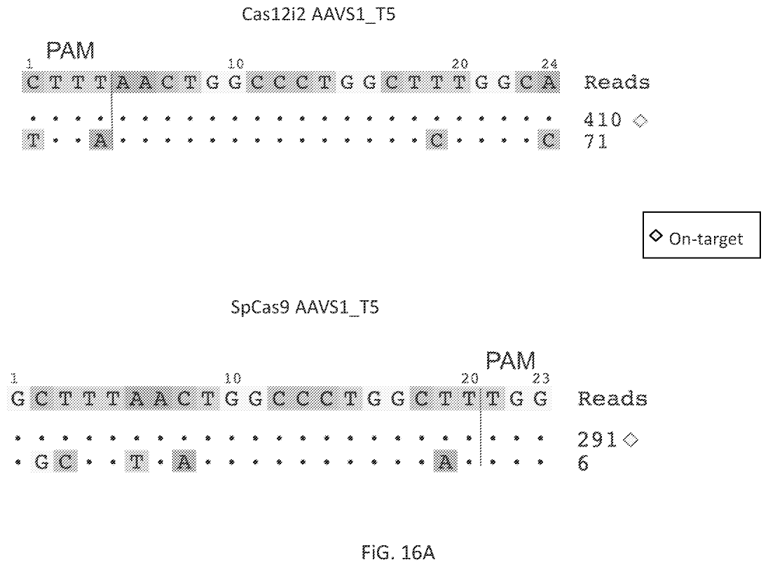

In this Example, a modified version of the tagmentation-based tag integration site sequencing (TTISS) method described in Schmid-Burgk et al., Technology 78(4): 794-800 (2000) was used to analyze Cas12i2 variant specificity and activity. A schematic of the method described herein is shown in FIG. 15. The TTISS donor oligonucleotides and primers in Table XX were synthesized by Integrated DNA Technologies (Coralville, IA). “/5phos/” indicates a 5′ phosphorylation site, “3ddC/” indicates a 3′ dideoxycytidine, and “*” indicates a phosphorothioate linkage.

TABLE 26
TTISS method sequences.

| | |
|---|---|
| Donor_Sense | /5phos/G*T*TGTGAGCAAGGGCGAGGAGGATAACGCCTCTCTCCCA |
| | GCGACT*A*T (SEQ ID NO: 513) |
| Donor_Antisense | /5phos/A*T*AGTCGCTGGGAGAGAGGCGTTATCCTCCTCGCCCTTG |
| | CTCACA*A*C (SEQ ID NO: 514) |
| Transposon_ME | /5Phos/CTGTCTCTTATACA/3ddC/ (SEQ ID NO: 515) |
| Transposon_Read_2 | GTCTCGTGGGCTCGGAGATGTGTATAAGAGACAG (SEQ ID NO: |
| | 516) |
| PCR_Fwd_1 | GTCGCTGGGAGAGAGGCGTTATC (SEQ ID NO: 517) |
| Read_2_Priming_ | GTCTCGTGGGCTCGGAGATGTGT (SEQ ID NO: 518) |
| Sequence | |
| PCR2_ | AATGATACGGCGACCACCGAGATCTACACTATAGCCTACACTCTTTCC |
| StaggeredPrimer1 | CTACACGACGCTCTTCCGATCTTTATCCTCCTCGCCCTTGCTCAC |
| | (SEQ ID NO: 519) |
| PCR2_ | AATGATACGGCGACCACCGAGATCTACACTATAGCCTACACTCTTTCC |
| StaggeredPrimer2 | CTACACGACGCTCTTCCGATCTGTTATCCTCCTCGCCCTTGCTCA |
| | (SEQ ID NO: 520) |
| PCR2_ | AATGATACGGCGACCACCGAGATCTACACTATAGCCTACACTCTTTCC |
| StaggeredPrimer3 | CTACACGACGCTCTTCCGATCTCGTTATCCTCCTCGCCCTTGCTC |
| | (SEQ ID NO: 521) |
| PCR2_ | AATGATACGGCGACCACCGAGATCTACACTATAGCCTACACTCTTTCC |
| StaggeredPrimer4 | CTACACGACGCTCTTCCGATCTGCGTTATCCTCCTCGCCCTTGCT |
| | (SEQ ID NO: 522) |
| PCR2_ | AATGATACGGCGACCACCGAGATCTACACTATAGCCTACACTCTTTCC |
| StaggeredPrimer5 | CTACACGACGCTCTTCCGATCTGGCGTTATCCTCCTCGCCCTTGC |
| | (SEQ ID NO: 523) |
| PCR2_ | AATGATACGGCGACCACCGAGATCTACACTATAGCCTACACTCTTTCC |
| StaggeredPrimer6 | CTACACGACGCTCTTCCGATCTAGGCGTTATCCTCCTCGCCCTTG |
| | (SEQ ID NO: 524) |
| PCR2_ | AATGATACGGCGACCACCGAGATCTACACTATAGCCTACACTCTTTCC |
| StaggeredPrimer7 | CTACACGACGCTCTTCCGATCTGAGGCGTTATCCTCCTCGCCCTT |
| | (SEQ ID NO: 525) |
| PCR2_ | AATGATACGGCGACCACCGAGATCTACACTATAGCCTACACTCTTTCC |
| StaggeredPrimer8 | CTACACGACGCTCTTCCGATCTAGAGGCGTTATCCTCCTCGCCCT |
| | (SEQ ID NO: 526) |
| PCR2_ | AATGATACGGCGACCACCGAGATCTACACTATAGCCTACACTCTTTCC |
| StaggeredPrimer9 | CTACACGACGCTCTTCCGATCTGAGAGGCGTTATCCTCCTCGCCC |
| | (SEQ ID NO: 527) |
| PCR2_ | AATGATACGGCGACCACCGAGATCTACACTATAGCCTACACTCTTTCC |
| StaggeredPrimer10 | CTACACGACGCTCTTCCGATCTAGAGAGGCGTTATCCTCCTCGCC |
| | (SEQ ID NO: 528) |
| PCR2_ | AATGATACGGCGACCACCGAGATCTACACTATAGCCTACACTCTTTCC |
| StaggeredPrimer11 | CTACACGACGCTCTTCCGATCTGAGAGAGGCGTTATCCTCCTCGC |
| | (SEQ ID NO: 529) |
| PCR2_ | AATGATACGGCGACCACCGAGATCTACACTATAGCCTACACTCTTTCC |
| StaggeredPrimer12 | CTACACGACGCTCTTCCGATCTGGAGAGAGGCGTTATCCTCCTCG |
| | (SEQ ID NO: 530) |
| PCR_rev_BC1 | CAAGCAGAAGACGGCATACGAGATCGAGTAATGTCTCGTGGGCTCGGA |
| | GATGTGT (SEQ ID NO: 531) |
| PCR_rev_BC2 | CAAGCAGAAGACGGCATACGAGATTCTCCGGAGTCTCGTGGGCTCGGA |
| | GATGIGT (SEQ ID NO: 532) |
| PCR_rev_BC3 | CAAGCAGAAGACGGCATACGAGATAATGAGCGGTCTCGTGGGCTCGGA |
| | GATGIGT (SEQ ID NO: 533) |
| PCR_rev_BC4 | CAAGCAGAAGACGGCATACGAGATGGAATCTCGTCTCGTGGGCTCGGA |
| | GATGIGT (SEQ ID NO: 534) |
| PCR_rev_BC5 | CAAGCAGAAGACGGCATACGAGATTTCTGAATGTCTCGTGGGCTCGGA |
| | GATGIGT (SEQ ID NO: 535) |
| PCR_rev_BC6 | CAAGCAGAAGACGGCATACGAGATACGAATTCGTCTCGTGGGCTCGGA |
| | GATGIGT (SEQ ID NO: 536) |
| PCR_rev_BC7 | CAAGCAGAAGACGGCATACGAGATAGCTTCAGGTCTCGTGGGCTCGGA |
| | GATGTGT (SEQ ID NO: 537) |
| PCR_rev_BC8 | CAAGCAGAAGACGGCATACGAGATGCGCATTAGTCTCGTGGGCTCGGA |
| | GATGTGT (SEQ ID NO: 538) |
| PCR_rev_BC9 | CAAGCAGAAGACGGCATACGAGATCATAGCCGGTCTCGTGGGCTCGGA |
| | GATGIGT (SEQ ID NO: 539) |
| PCR_rev_BC10 | CAAGCAGAAGACGGCATACGAGATTTCGCGGAGTCTCGTGGGCTCGGA |
| | GATGTGT (SEQ ID NO: 540) |
| PCR_rev_BC11 | CAAGCAGAAGACGGCATACGAGATGCGCGAGAGTCTCGTGGGCTCGGA |
| | GATGTGT (SEQ ID NO: 541) |
| PCR_rev_BC12 | CAAGCAGAAGACGGCATACGAGATCTATCGCTGTCTCGTGGGCTCGGA |
| | GATGIGT (SEQ ID NO: 542) |
| PCR rev_BC13 | CAAGCAGAAGACGGCATACGAGATTGTAGTGCGTCTCGTGGGCTCGGA |
| | GATGIGT (SEQ ID NO: 543) |
| PCR_rev_BC14 | CAAGCAGAAGACGGCATACGAGATGCGTCGACGTCTCGTGGGCTCGGA |
| | GATGTGT (SEQ ID NO: 544) |
| PCR_rev_BC15 | CAAGCAGAAGACGGCATACGAGATGGTCTTCTGTCTCGTGGGCTCGGA |
| | GATGIGT (SEQ ID NO: 545) |
| PCR_rev_BC16 | CAAGCAGAAGACGGCATACGAGATAAATGTCCGTCTCGTGGGCTCGGA |
| | GATGIGT (SEQ ID NO: 546) |
| PCR_rev_BC17 | CAAGCAGAAGACGGCATACGAGATGTTGAAACGTCTCGTGGGCTCGGA |
| | GATGIGT (SEQ ID NO: 547) |
| PCR_rev_BC18 | CAAGCAGAAGACGGCATACGAGATTCTTTACGGTCTCGTGGGCTCGGA |
| | GATGIGT (SEQ ID NO: 548) |
| PCR_rev_BC19 | CAAGCAGAAGACGGCATACGAGATATGCCTGGGTCTCGTGGGCTCGGA |
| | GATGIGT (SEQ ID NO: 549) |
| PCR_rev_BC20 | CAAGCAGAAGACGGCATACGAGATCAATAAGGGTCTCGTGGGCTCGGA |
| | GATGIGT (SEQ ID NO: 550) |
| PCR_rev_BC21 | CAAGCAGAAGACGGCATACGAGATCGCCGTAAGTCTCGTGGGCTCGGA |
| | GATGTGT (SEQ ID NO: 551) |
| PCR rev_BC22 | CAAGCAGAAGACGGCATACGAGATTAAGGCTTGTCTCGTGGGCTCGGA |
| | GATGIGT (SEQ ID NO: 552) |
| PCR_rev_BC23 | CAAGCAGAAGACGGCATACGAGATTTGCTGCCGTCTCGTGGGCTCGGA |
| | GATGTGT (SEQ ID NO: 553) |
| PCR_rev_BC24 | CAAGCAGAAGACGGCATACGAGATCTCAATGTGTCTCGTGGGCTCGGA |
| | GATGTGT (SEQ ID NO: 554) |

PCR_fwd_2_stagger_mix (1 μM) was prepared by adding equal volumes of PCR2_StaggeredPrimer1 through PCR2_Staggeredprimer12 and diluting 10-fold in 10 mM Tris-HCl.

Double-stranded TTISS donor oligos were prepared by mixing equimolar amounts of Donor_Sense and Donor_Antisense oligos and mixing 45 μL of the equimolar mixture with 5 μL of IDT duplex buffer (Integrated DNA Technologies) in a 96-well PCR plate. Oligos were then duplexed by placing the plate in a thermal cycler and running a program to heat samples to 95° C. and subsequently ramp temperature to 25° C. by running 700 6-second cycles in which the temperature decreased by 0.1° C. for each cycle.

Transposon DNA was prepared by combining 332 μL each of 100 μM Transposon_ME and 100 μM of Transposon_Read_2 oligos with 80 μL of 10× annealing buffer (300 mM NaCl, 1.5 mM EDTA, 15 mM HCl), and 40 μL of Ultrapure Water (Invitrogen). The mixture was distributed across 8 wells of a 96-well PCR plate. The transposon DNA was annealed by placing the PCR plate in a thermal cycler and running a program to heat samples to 95° C. for 3 minutes and subsequently ramp temperature to 25° C. by running 700 6-second cycles in which the temperature decreased by 0.1° C. for each cycle.

Upon completion of the annealing step, transposon DNA was then diluted to 100 ng/μL. Next, 375 μL 100 ng/μL transposon DNA was mixed with 375 μL glycerol and 750 μL EZ-Tn5 Transposase (Lucigen). The solutions were then mixed by vortexing and incubated at room temperature for 30 minutes to form the loaded Tn5 transposome. Transposome was stored at −20° C. until ready for use.

Prior to transfection, HEK 293T cells were plated in 24-well plates (Corning) at a density of 125,000 cells per well in 500 μL D10 media (DMEM containing GlutaMax and pyruvate (Gibco) supplemented with 10% Premium Heat-inactivated FBS (Corning) and Penicillin/Streptomycin antibiotic (Hyclone)). Cells were cultured overnight in an incubator set to 37° C. and 5% CO₂.

Cells were transfected approximately 15 hours after plating. For each well to be transfected, 500 ng donor oligo at ˜1.25 ng/μL, 375 ng Cas12i2 effector plasmid (Cas12i2 variant of SEQ ID NO: 4) at 1000 ng/μL, and 125 ng RNA guide plasmid at 100 ng/μL were added to Opti-MEM media (Gibco) to a final volume of 125 μL to form Opti-1 solution. The RNA guide and target sequences are shown in Table 27; SpCas9 was used as a control. In a separate vessel, 122.5 μL Opti-MEM media was mixed with 2.5 μL GeneJuice transfection reagent (Millipore-Sigma) to form Opti-2 solution. Opti-2 solution was incubated at room temperature for 5 minutes. After the Opti-2 incubation, Opti-1 and Opti-2 were mixed together and incubated at room temperature for 5-15 minutes. After incubation, the Opti-1+Opti-2 solution was added dropwise to a single well of a 24-well plate. Cells were returned to the incubator for approximately 72 h.

TABLE 27
Target and crRNA sequences for TTISS.

| Target Locus | Target | crRNA | Effector |
|---|---|---|---|
| AAVS1_T1 | TGTCCCCCCAAGTTTTGG | AGAAATCCGTCTTTCATTGACGGTGT | Variant |
| | AC | CCCCCCAAGTTTTGGAC (SEQ ID | Cas12i2 |
| | (SEQ ID NO: 252) | NO: 253) | |
| AAVS1_T3 | GTGAGAATGGTGCGTCCT | AGAAATCCGTCTTTCATTGACGGGTG | Variant |
| | AG | AGAATGGTGCGTCCTAG (SEQ ID | Cas12i2 |
| | (SEQ ID NO: 256) | NO: 257) | |
| AAVS1_T5 | AACTGGCCCTGGCTTTGG | AGAAATCCGTCTTTCATTGACGGAAC | Variant |
| | CA | TGGCCCTGGCTTTGGCA (SEQ ID | Cas12i2 |
| | (SEQ ID NO: 260) | NO: 261) | |
| AAVS1_T6 | GTAGCCTCTCCCGCTCTG | AGAAATCCGTCTTTCATTGACGGGTA | Variant |
| | GT | GCCTCTCCCGCTCTGGT (SEQ ID | Cas12i2 |
| | (SEQ ID NO: 262) | NO: 263) | |
| AAVS1_T7 | GGGAAGTGGTTGGTCAGC | AGAAATCCGTCTTTCATTGACGGGGG | Variant |
| | AT | AAGTGGTTGGTCAGCAT (SEQ ID | Cas12i2 |
| | (SEQ ID NO: 264) | NO: 265) | |
| AAVS1_T8 | AGCGGGTATGGGAAGGGC | AGAAATCCGTCTTTCATTGACGGAGC | Variant |
| | TT | GGGTATGGGAAGGGCTT (SEQ ID | Cas12i2 |
| | (SEQ ID NO: 266) | NO: 267) | |
| EMX1_T1 | GGGCGCAGGGCCACCTGG | AGAAATCCGTCTTTCATTGACGGGGG | Variant |
| | AC | CGCAGGGCCACCTGGAC (SEQ ID | Cas12i2 |
| | (SEQ ID NO: 268) | NO: 269) | |
| EMX1 T2 | GGATGGCGACTTCAGGCA | AGAAATCCGTCTTTCATTGACGGGGA | Variant |
| | CA | TGGCGACTTCAGGCACA (SEQ ID | Cas12i2 |
| | (SEQ ID NO: 270) | NO: 271) | |
| EMX1_T4 | GGGGAGGCCTGGAGTCAT | AGAAATCCGTCTTTCATTGACGGGGG | Variant |
| | GG | GAGGCCTGGAGTCATGG (SEQ ID | Cas12i2 |
| | (SEQ ID NO: 274) | NO: 275) | |
| EMX1_T6 | GAGCCAGTGTTGCTAGTC | AGAAATCCGTCTTTCATTGACGGGAG | Variant |
| | AA | CCAGTGTTGCTAGTCAA (SEQ ID | Cas12i2 |
| | (SEQ ID NO: 278) | NO: 279) | |
| EMX1 T7 | AGCAAGGGACTATTCAGG | AGAAATCCGTCTTTCATTGACGGAGC | Variant |
| | GA | AAGGGACTATTCAGGGA (SEQ ID | Cas12i2 |
| | (SEQ ID NO: 280) | NO: 281) | |
| EMX1_T8 | AAAATTGAGCAATCTACC | AGAAATCCGTCTTTCATTGACGGAAA | Variant |
| | CT | ATTGAGCAATCTACCCT (SEQ ID | Cas12i2 |
| | (SEQ ID NO: 282) | NO: 283) | |
| VEGFA_T1 | TGGGGGTGACCGCCGGAG | AGAAATCCGTCTTTCATTGACGGTGG | Variant |
| | CG | GGGTGACCGCCGGAGCG (SEQ ID | Cas12i2 |
| | (SEQ ID NO: 284) | NO: 285) | |
| VEGFA_T2 | AATCCTCCACCAGTCATG | AGAAATCCGTCTTTCATTGACGGAAT | Variant |
| | GT | CCTCCACCAGTCATGGT (SEQ ID | Cas12i2 |
| | (SEQ ID NO: 286) | NO: 287) | |
| VEGFA_T3 | GTTGACATTGTCCACACC | AGAAATCCGTCTTTCATTGACGGGTT | Variant |
| | TG | GACATTGTCCACACCTG (SEQ ID | Cas12i2 |
| | (SEQ ID NO: 288) | NO: 289) | |
| VEGFA_T5 | TTAAACTCTCCATGGACC | AGAAATCCGTCTTTCATTGACGGTTA | Variant |
| | AG | AACTCTCCATGGACCAG (SEQ ID | Cas12i2 |
| | (SEQ ID NO: 292) | NO: 293) | |
| VEGFA_T6 | GCCCATACTGGGGACCAA | AGAAATCCGTCTTTCATTGACGGGCC | Variant |
| | GG | CATACTGGGGACCAAGG (SEQ ID | Cas12i2 |
| | (SEQ ID NO: 294) | NO: 295) | |
| VEGFA_T7 | GCCGTAACCCTTCGTGGG | AGAAATCCGTCTTTCATTGACGGGCC | Variant |
| | TA | GTAACCCTTCGTGGGTA (SEQ ID | Cas12i2 |
| | (SEQ ID NO: 296) | NO: 297) | |
| AAVS1_T1 | GCTTTTGTCCCCCCAAGT | GCTTTTGTCCCCCCAAGTTTGTTTTA | SpCas9 |
| | TT (SEQ ID NO: | GAGCTAGAAATAGCAAGTTAAAATAA | |
| | 555) | GGCTAGTCCGTTATCAACTTGAAAAA | |
| | | GTGGCACCGAGTCGGTGC (SEQ ID | |
| | | NO: 556) | |
| AAVS1_T3 | TTGTGAGAATGGTGCGTC | TTGTGAGAATGGTGCGTCCTGTTTTA | SpCas9 |
| | CT (SEQ ID NO: | GAGCTAGAAATAGCAAGTTAAAATAA | |
| | 557) | GGCTAGTCCGTTATCAACTTGAAAAA | |
| | | GTGGCACCGAGTCGGTGC (SEQ ID | |
| | | NO: 558) | |
| AAVS1_T5 | GCTTTAACTGGCCCTGGC | GCTTTAACTGGCCCTGGCTTGTTTTA | SpCas9 |
| | TT (SEQ ID NO: | GAGCTAGAAATAGCAAGTTAAAATAA | |
| | 559) | GGCTAGTCCGTTATCAACTTGAAAAA | |
| | | GTGGCACCGAGTCGGTGC (SEQ ID | |
| | | NO: 560) | |
| AAVS1_T6 | CTTTGTAGCCTCTCCCGC | CTTTGTAGCCTCTCCCGCTCGTTTTA | SpCas9 |
| | TC (SEQ ID NO: | GAGCTAGAAATAGCAAGTTAAAATAA | |
| | 561) | GGCTAGTCCGTTATCAACTTGAAAAA | |
| | | GTGGCACCGAGTCGGTGC (SEQ ID | |
| | | NO: 562) | |
| AAVS1_T7 | TGGGAAGTGGTTGGTCAG | TGGGAAGTGGTTGGTCAGCAGTTTTA | SpCas9 |
| | CA (SEQ ID NO: | GAGCTAGAAATAGCAAGTTAAAATAA | |
| | 563) | GGCTAGTCCGTTATCAACTTGAAAAA | |
| | | GTGGCACCGAGTCGGTGC (SEQ ID | |
| | | NO: 564) | |
| AAVS1_T8 | GACTTTAGCGGGTATGGG | GACTTTAGCGGGTATGGGAAGTTTTA | SpCas9 |
| | AA (SEQ ID NO: | GAGCTAGAAATAGCAAGTTAAAATAA | |
| | 565) | GGCTAGTCCGTTATCAACTTGAAAAA | |
| | | GTGGCACCGAGTCGGTGC (SEQ ID | |
| | | NO: 566) | |
| EMX1_T1 | TCTTTGGGCGCAGGGCCA | TCTTTGGGCGCAGGGCCACCGTTTTA | SpCas9 |
| | CC (SEQ ID NO: | GAGCTAGAAATAGCAAGTTAAAATAA | |
| | 567) | GGCTAGTCCGTTATCAACTTGAAAAA | |
| | | GTGGCACCGAGTCGGTGC (SEQ ID | |
| | | NO: 568) | |
| EMX1_T2 | TGGATGGCGACTTCAGGC | TGGATGGCGACTTCAGGCACGTTTTA | SpCas9 |
| | AC (SEQ ID NO: | GAGCTAGAAATAGCAAGTTAAAATAA | |
| | 569) | GGCTAGTCCGTTATCAACTTGAAAAA | |
| | | GTGGCACCGAGTCGGTGC (SEQ ID | |
| | | NO: 570) | |
| EMX1_T4 | TTTGGGGAGGCCTGGAGT | TTTGGGGAGGCCTGGAGTCAGTTTTA | SpCas9 |
| | CA (SEQ ID NO: | GAGCTAGAAATAGCAAGTTAAAATAA | |
| | 571) | GGCTAGTCCGTTATCAACTTGAAAAA | |
| | | GTGGCACCGAGTCGGTGC (SEQ ID | |
| | | NO: 572) | |
| EMX1_T6 | GAGCCAGTGTTGCTAGTC | GAGCCAGTGTTGCTAGTCAAGTTTTA | SpCas9 |
| | AA (SEQ ID NO: | GAGCTAGAAATAGCAAGTTAAAATAA | |
| | 573) | GGCTAGTCCGTTATCAACTTGAAAAA | |
| | | GTGGCACCGAGTCGGTGC (SEQ ID | |
| | | NO: 574) | |
| EMX1_T7 | CTTTAGCAAGGGACTATT | CTTTAGCAAGGGACTATTCAGTTTTA | SpCas9 |
| | CA (SEQ ID NO: | GAGCTAGAAATAGCAAGTTAAAATAA | |
| | 575) | GGCTAGTCCGTTATCAACTTGAAAAA | |
| | | GIGGCACCGAGTCGGTGC (SEQ ID | |
| | | NO: 576) | |
| EMX1_T8 | TAAAATTGAGCAATCTAC | TAAAATTGAGCAATCTACCCGTTTTA | SpCas9 |
| | CC (SEQ ID NO: | GAGCTAGAAATAGCAAGTTAAAATAA | |
| | 577) | GGCTAGTCCGTTATCAACTTGAAAAA | |
| | | GTGGCACCGAGTCGGTGC (SEQ ID | |
| | | NO: 578) | |
| VEGFA_T1 | TGGGGGTGACCGCCGGAG | TGGGGGTGACCGCCGGAGCGGTTTTA | SpCas9 |
| | CG (SEQ ID NO: | GAGCTAGAAATAGCAAGTTAAAATAA | |
| | 579) | GGCTAGTCCGTTATCAACTTGAAAAA | |
| | | GTGGCACCGAGTCGGTGC (SEQ ID | |
| | | NO: 580) | |
| VEGFA_T2 | CTTTAATCCTCCACCAGT | CTTTAATCCTCCACCAGTCAGTTTTA | SpCas9 |
| | CA (SEQ ID NO: | GAGCTAGAAATAGCAAGTTAAAATAA | |
| | 581) | GGCTAGTCCGTTATCAACTTGAAAAA | |
| | | GTGGCACCGAGTCGGTGC (SEQ ID | |
| | | NO: 582) | |
| VEGFA_T3 | TTGTTGACATTGTCCACA | TTGTTGACATTGTCCACACCGTTTTA | SpCas9 |
| | CC (SEQ ID NO: | GAGCTAGAAATAGCAAGTTAAAATAA | |
| | 583) | GGCTAGTCCGTTATCAACTTGAAAAA | |
| | | GTGGCACCGAGTCGGTGC (SEQ ID | |
| | | NO: 584) | |
| VEGFA_T5 | TTTTAAACTCTCCATGGA | TTTTAAACTCTCCATGGACCGTTTTA | SpCas9 |
| | CC (SEQ ID NO: | GAGCTAGAAATAGCAAGTTAAAATAA | |
| | 585) | GGCTAGTCCGTTATCAACTTGAAAAA | |
| | | GTGGCACCGAGTCGGTGC (SEQ ID | |
| | | NO: 586) | |
| VEGFA_T6 | TTTGCCCATACTGGGGAC | TTTGCCCATACTGGGGACCAGTTTTA | SpCas9 |
| | CA (SEQ ID NO: | GAGCTAGAAATAGCAAGTTAAAATAA | |
| | 587) | GGCTAGTCCGTTATCAACTTGAAAAA | |
| | | GTGGCACCGAGTCGGTGC (SEQ ID | |
| | | NO: 588) | |
| VEGFA_T7 | GCTTTGCCGTAACCCTTC | GCTTTGCCGTAACCCTTCGTGTTTTA | SpCas9 |
| | GT (SEQ ID NO: | GAGCTAGAAATAGCAAGTTAAAATAA | |
| | 589) | GGCTAGTCCGTTATCAACTTGAAAAA | |
| | | GTGGCACCGAGTCGGTGC (SEQ ID | |
| | | NO: 590) | |

Cells were then dissociated from the plate by removing media, washing once with 200 μL PBS (Gibco), adding 50 μL of TrypLE dissociation reagent (Gibco), and incubating at 37° C. for 5 minutes. Cells were then resuspended by adding 200 μL of D10 media and mixing well. Resuspended cells were then transferred to a 96-well PCR plate and spun down at 400× g for 10 minutes. The supernatant was removed and cell pellets were stored at −20° C. until DNA extraction.

DNA was extracted from cells by resuspending pellet in 50 μL lysis buffer (1 mM CaCl2, 3 mM MgCl2, 1 mM EDTA, 1% Triton X-100 (Sigma), 8 U/mL Proteinase K (NEB), 10 mM Tris-HCl) and incubating resuspension at 65° C. for 10 minutes. Next gDNA was purified using the Zymo gDNA clean and concente-5 kit (Zymo Research) following the manufacturer's instructions and eluting in 35 μL of 10 mM Tris-HCl. gDNA was visualized on a gel and quantified by the Qubit high-sensitivity dsDNA kit (Invitrogen) following the manufacturer's instructions. gDNA extracts were then normalized to 50 ng/μL in 10 mM Tris-HCl.

Genomic DNA was tagmented by first preparing a solution 24 μL transposome, 6 μL EZ-Tn5 10× Reaction Buffer (Lucigen), 24 μL purified and diluted genomic DNA, and 6 μL of water. This solution was mixed well and incubated at 37° C. for two hours. Following the incubation, 6 μL of Ez-Tn5 10× stop solution was mixed into the reaction. The reaction+stop solution mixture was then heated at 70° C. for 10 minutes. The stopped reaction was then purified using the Zymo gDNA clean and concente-5 kit by following the manufacturer's instructions and eluting in 50 μL 10 mM Tris-HCl.

Round 1 PCR was performed using a KOD HotStart PCR Kit (Millipore Sigma) with the following conditions per reaction: 5 μL 10× Buffer for KOD Hot Start DNA Polymerase, 3 μL 25 mM MgSO4, 5 μL dNTPs (2 mM each), 1.5 μL PCR_fwd_1 (1 μM), 1.5 μL Transposon_Read_2 (1 μM), 1 μL KOD Hot Start DNA Polymerase (1.0 U/μL), 15 μL tagmented and purified gDNA, and 18 μL Ultrapure water. All reagents were mixed well, and the reaction was carried out following the manufacturer's instructions using an annealing temperature of 60° C. and an extension time of 60 seconds.

Round 2 PCR was performed with the following conditions per reaction: 5 μL 10×Buffer for KOD Hot Start DNA Polymerase, 3 μL 25 mM MgSO4, 5 μL dNTPs (2 mM each), PCR_fwd_2_stagger_mix (10 μM), 1.5 μL of one reverse barcoding primer (PCR_rev_BC1 through PCR_rev_BC24) (1 μM), 1 μL KOD Hot Start DNA Polymerase (1.0 U/μL), 3 μL PCR 1 product, and 30 μL Ultrapure water. All reagents were mixed well, and the reaction was carried out following the manufacturer's instructions using an annealing temperature of 65° C. and an extension time of 60 seconds.

The 50 μL PCR2 reaction was then purified by a 0.8× SPRI cleanup using Clean-NGS beads (Bulldog Bio). SPRI reaction was eluted in 25 μL 10 mM Tris-HCl. Cleaned reactions were pooled and the pool was loaded onto a 2% E-gel EX (Invitrogen), which was then run for 10 minutes on an E-gel base. The resulting smear was then excised from 250-1000 bp with a razor and the excised library was purified with a Gel Extraction kit (Qiagen) following the manufacturer's instructions and eluting in 50 μL. A second library purification was performed using the Clean and Concentrate-5 kit (Zymo Research) following the manufacturer's instructions and eluting in 20 μL.

The library was then quantified by the Qubit high-sensitivity dsDNA kit and normalized to 3 nM using an estimated average library size of 400 bp. The normalized library was mixed with 3 nM PhiX (Illumina) so the library:PhiX ratio was 4:1. The final library:PhiX mix was then loaded onto an Illumina NextSeq 550 High-output kit at a final concentration of 2.0 pM. Read lengths were set to 75/8/0/25 R1/I1/I2/R2.

After sequencing, the TTISS data was analyzed to identify on and off-target sites. The raw sequencing data was first demultiplexed and converted to paired-end fastq files using Illumina's bcl2fastq2 software. Reads that did not begin with the dsODN primer sequence or had low sequencing quality were filtered out, and the remaining reads were truncated to 25 bp of genomic DNA in the forward direction and 15 bp of genomic DNA in the reverse direction. The truncated reads were then aligned to the GRCh38 human reference genome using bowtie2, allowing for a maximum fragment length of 1000 bp (Langmead & Salzberg, Nature Methods 9:357-9 (2012). The alignments were filtered to only include read pairs that mapped properly, and PCR duplicate reads were marked using SAMtools (Li et al., Bioinformatics 35(16): 2078-9 (2009). All 100 bp genomic windows with at least two unique aligned reads were identified as possible integration sites, and for each window, a cut site was predicted based on the frequency and positional distribution of its aligned reads. Each window was then searched for any sequences within an edit distance of 6 from the target sequence(s) used for the sample, and the putative cut site for the sequence was determined. SpCas9 cuts −4 bp from the 3′ end of the target, Cas12i2 cuts 3 bp from the 3′ end of the target. Windows without any putative on/off-target sequences were removed, as were windows where the putative on/off-target sequence's cut site was over 8 bp from the window's predicted cut site. Windows that appeared in negative control samples were also removed to avoid common break sites. The remaining windows, their on/off-target sequence, and relevant metrics such as the count of read aligned to the window and the edit distance between the identified on/off-target and the target sequence were then output.

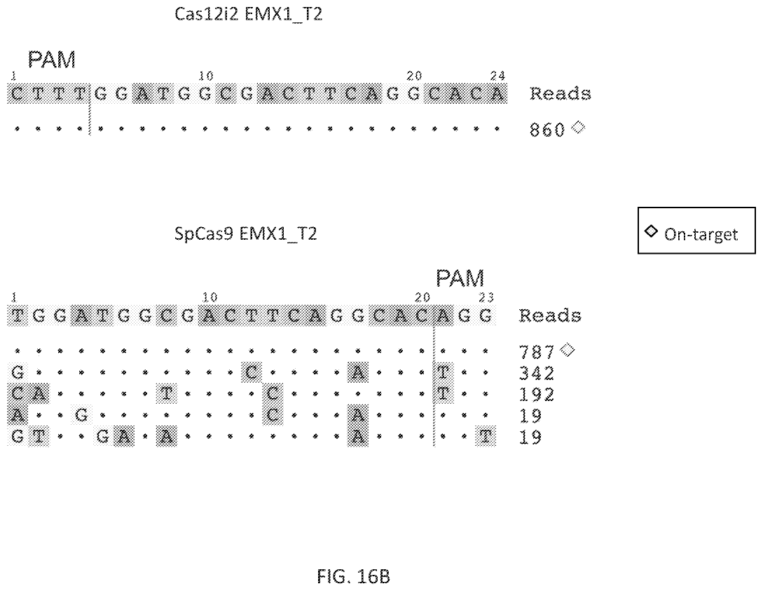

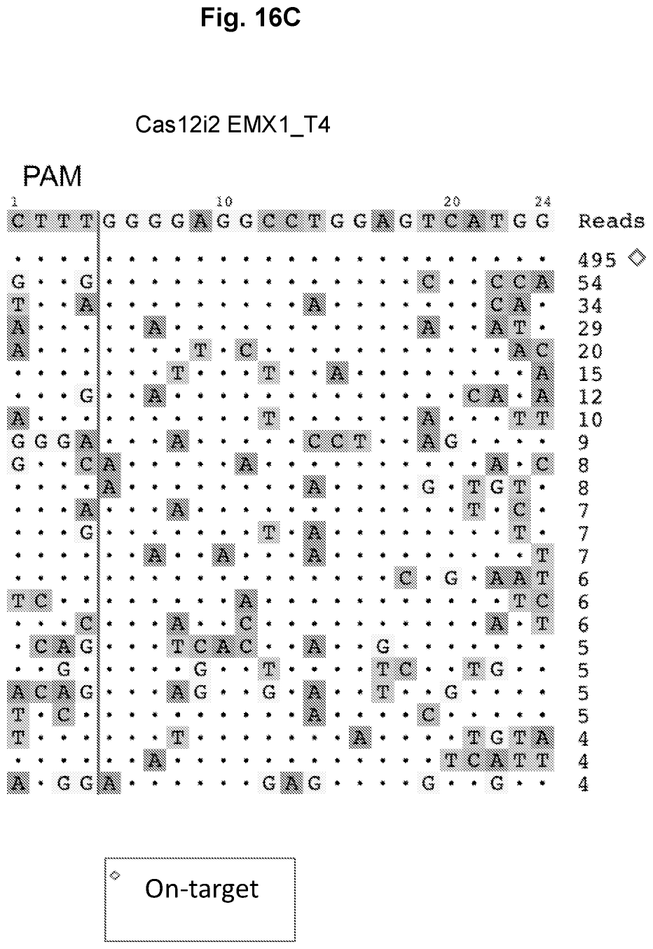

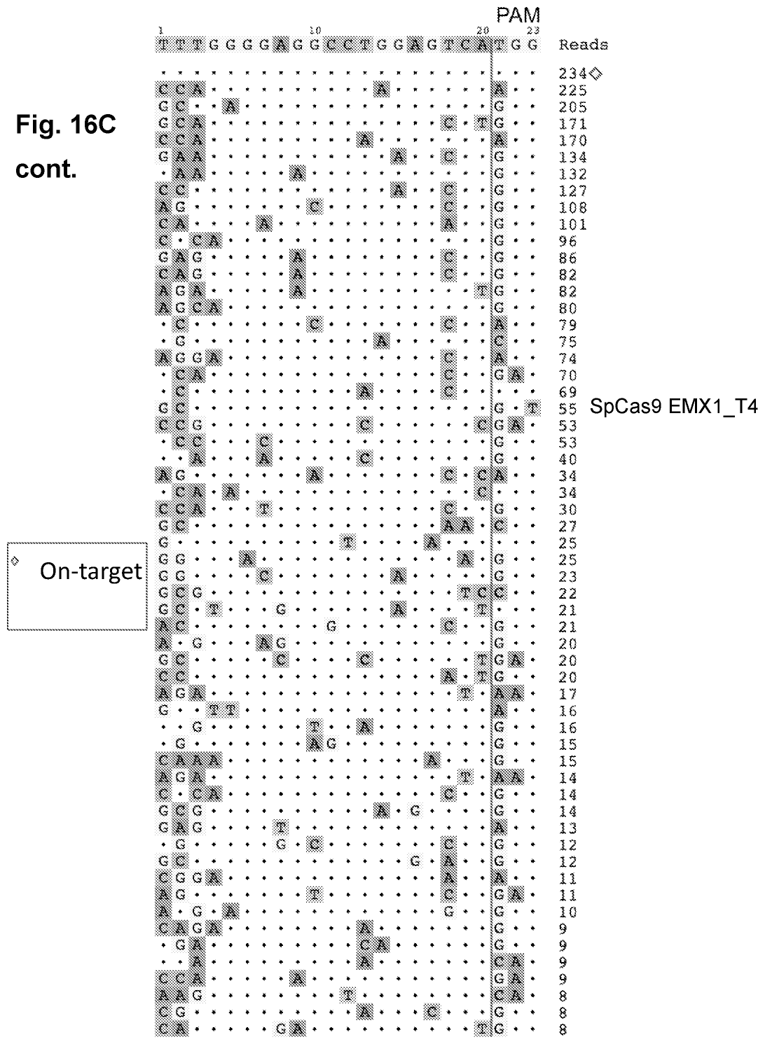

Table 28 shows the on-target reads and number of off-targets that were measured for each Cas12i2 and SpCas9 target. “On-target reads” refer to the number of unique reads mapping to 100 bp window around the on-target site. “Number of off-targets” refers to the number of sites in the genome that had at least two unique reads map within a 100 bp window around the site and contained a putative off-target sequence (e.g., a sequence within an edit distance of 6 from the guide sequence and with a predicted cut site within 8 bp of the cut site predicted from the distribution of reads mapping to the site). “Highest off-target reads” refers to the number of reads for the off-target site with the highest number of reads. FIG. 16A, FIG. 16B, FIG. 16C, and FIG. 16D are schematics of on-target and off-target reads for variant Cas12i2 of SEQ ID NO: 4 and SpCas9 at the target EMX1_T2. Each base in a read that corresponds with (e.g., is identical to) the on-target sequence is indicated with a dot, and each base in a read that does not correspond with (e.g., is not identical to) the on-target sequence is indicated with the sequenced base.

TABLE 28
On-target and off-target reads.

| | | | Cas12i2 | | | SpCas9 |
|---|---|---|---|---|---|---|
| | Cas12i2 | Cas12i2 | Highest | SpCas9 | SpCas9 | Highest |
| Target | On-target | Number | off-target | On-target | Number | off-target |
| Locus | reads | off-targets | reads | reads | off-targets | reads |
| AAVS1_T1 | 289 | 0 | 0 | 79 | 0 | 0 |
| AAVS1_T3 | 251 | 1 | 4 | 747 | 1 | 14 |
| AAVS1_T5 | 410 | 1 | 71 | 291 | 1 | 6 |
| AAVS1_T6 | 507 | 2 | 4 | 674 | 1 | 9 |
| AAVS1_T7 | 725 | 2 | 12 | 87 | 1 | 4 |
| AAVS1_T8 | 66 | 1 | 13 | 864 | 1 | 29 |
| EMX1_T1 | 259 | 22 | 14 | 241 | 4 | 119 |
| EMX1_T2 | 860 | 0 | 0 | 787 | 4 | 342 |
| EMX1_T4 | 495 | 23 | 54 | 234 | 81 | 225 |
| EMX1_T6 | 522 | 0 | 0 | 329 | 0 | 0 |
| EMX1_T7 | 357 | 5 | 37 | 171 | 0 | 0 |
| EMX1_T8 | 274 | 3 | 24 | 713 | 2 | 7 |
| VEGFA_T1 | 389 | 4 | 7 | 388 | 5 | 112 |
| VEGFA_T2 | 118 | 1 | 14 | 180 | 2 | 9 |
| VEGFA_T3 | 523 | 0 | 0 | 874 | 0 | 0 |
| VEGFA_T5 | 465 | 0 | 0 | 444 | 0 | 0 |
| VEGFA_T6 | 217 | 2 | 14 | 431 | 4 | 41 |
| VEGFA_T7 | 272 | 0 | 0 | 1215 | 0 | 0 |

The data in Table 28 and FIG. 16A, FIG. 16B, FIG. 16C, and FIG. 16D show that the variant Cas12i2 of SEQ ID NO: 4 demonstrated significantly fewer off-target reads than on-target reads. In contract, SpCas9 produced several off-targets with high read counts, suggesting that SpCas9 had higher off-target editing efficiency compared to variant Cas12i2. As shown in FIG. 16B, each Cas12i2 read for EMX1_T2 was an on-target read. However, SpCas9 reads showed multiple EMX1_T2 off-target sequences with high read counts. As shown in FIG. 16C, SpCas9 off-target reads exceeded on-target reads for EMX1_T4. See also the “highest off-target read” columns in Table 28. These conclusions are also supported by deep sequencing of select off-target sits where the maximum off-target indel rate was 2.6% for variant Cas12i2 and 77.7% for SpCas9.

The TTISS method further demonstrated that variant Cas12i2 prefers a 5′-NTTN-3′ PAM sequence. Off-targets identified with non-canonical PAM sequences occurred with low read counts and high edit distances between the guide and off-target, suggesting that those ready were likely false positives instead of true off-target sites.

This Example shows that variant Cas12i2 of SEQ ID NO: 4 exhibits high on-target selectivity and activity. The off-target binding/activity by SpCas9 exceeds that of variant Cas12i2 of SEQ ID NO: 4.

### Example 17—Targeting of Mammalian Genes by Cas12i2 Variants

This Example describes indel assessment on multiple targets using wild-type Cas12i2 and Cas12i2 variants introduced into mammalian cells by transient transfection.

Cas12i2 engineered variants in this Example were cloned into the pcda3.1 backbone (Invitrogen) on top of the previously engineered Cas12i2 variant of SEQ ID NO: 5, which has the following mutations: D581R, 1926R, V1030G, and S1046G. The plasmids were ordered from Genewiz as midi-preps normalized to 1 μg/μL in 10 T buffer. The crRNA sequences, as shown in Table 14, were ordered as circularized plasmid midi-preps and normalized to 100 ng/μL in 10 T buffer.

TABLE 29
crRNA and Target Sequences for
Transient Transfection.

| Target | crRNA sequence | Target Sequence |
|---|---|---|
| AAVS1_T6 | AGAAATCCGTCTTTCATTGACGG | GTAGCCTCTCCCGCTC |
| | GTAGCCTCTCCCGCTCTGGT | TGGT (SEQ ID NO: |
| | (SEQ ID NO: 591) | 592) |
| EMX1 T2 | AGAAATCCGTCTTTCATTGACGG | GGATGGCGACTTCAGG |
| | GGATGGCGACTTCAGGCACA | CACA (SEQ ID NO: |
| | (SEQ ID NO: 593) | 594) |
| EMX1_T6 | AGAAATCCGTCTTTCATTGACGG | GAGCCAGTGTTGCTAG |
| | GAGCCAGTGTTGCTAGTCAA | TCAA |
| | (SEQ ID NO: 595) | (SEQ ID NO: 596) |
| EMX1_T7 | AGAAATCCGTCTTTCATTGACGG | AGCAAGGGACTATTCA |
| | AGCAAGGGACTATTCAGGGA | GGGA (SEQ ID NO: |
| | (SEQ ID NO: 597) | 598) |
| VEGFA_T5 | AGAAATCCGTCTTTCATTGACGG | TTAAACTCTCCATGGA |
| | TTAAACTCTCCATGGACCAG | CCAG (SEQ ID NO: |
| | (SEQ ID NO: 599) | 600) |
| VEGFA T7 | AGAAATCCGTCTTTCATTGACGG | GCCGTAACCCTTCGTG |
| | GCCGTAACCCTTCGTGGGTA | GGTA |
| | (SEQ ID NO: 601) | (SEQ ID NO: 602) |

Approximately 16-18 hours prior to transfection, 100 μL of 25,000 HEK293T cells in DMEM/10% FBS+Pen/Strep were plated into each well of a 96-well plate. On the day of transfection, the cells were 70-90% confluent. For each well to be transfected, a mixture of 0.5 μL of Lipofectamine 2000 and 9.5 μL of Opti-MEM was prepared and then incubated at room temperature for 5-20 minutes (Solution 1). After incubation, the lipofectamine:OptiMEM mixture was added to a separate mixture containing 182 ng of effector plasmid and 14 ng of crRNA and water up to 10 μL (Solution 2). The solution 1 and solution 2 mixtures were mixed by pipetting up and down and then incubated at room temperature for 25 minutes. Following incubation, 20 μL of the Solution 1 and Solution 2 mixture were added dropwise to each well of a 96 well plate containing the cells. 72 hours post transfection, cells were trypsinized by adding 10 μL of TrypLE to the center of each well and incubated for approximately 5 minutes. 100 μL of D10 media was then added to each well and mixed to resuspend cells. The cells were then spun down at 500 g for 10 minutes, and the supernatant was discarded. QuickExtract buffer was added to ⅕ the amount of the original cell suspension volume. Cells were incubated at 65° C. for 15 minutes, 68° C. for 15 minutes, and 98° C. for 10 minutes.

Samples for Next Generation Sequencing were prepared by two rounds of PCR. The first round (PCR1) was used to amplify specific genomic regions depending on the target. PCR1 products were purified by column purification. Round 2 PCR (PCR2) was done to add Illumina adapters and indexes. Reactions were then pooled, loaded onto a 2% E-gel EX for 10 minutes and gel extracted. Sequencing runs were done with a 150 cycle NextSeq v2.5 mid or high output kit.

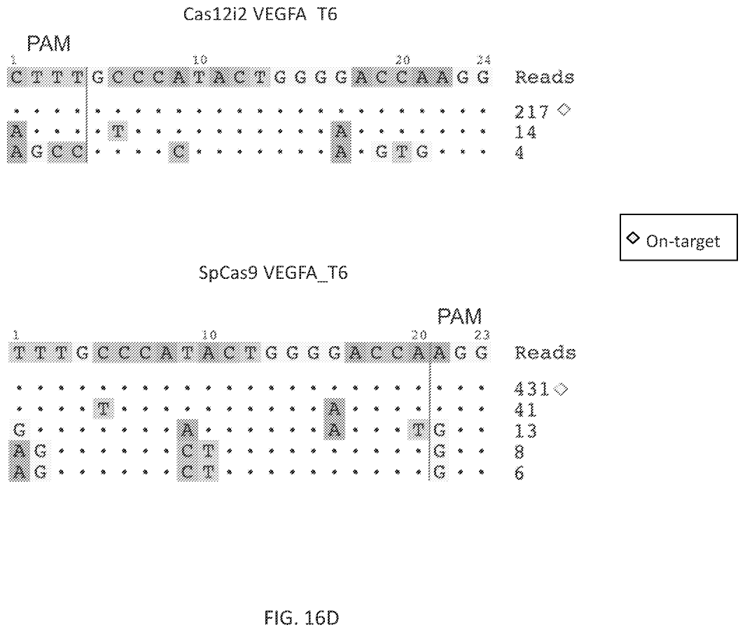

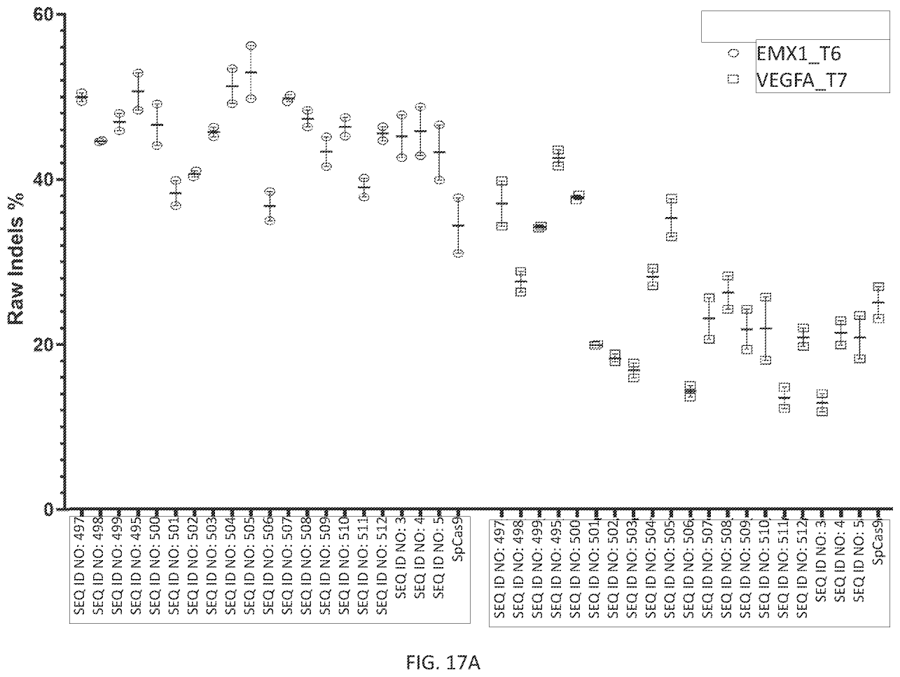

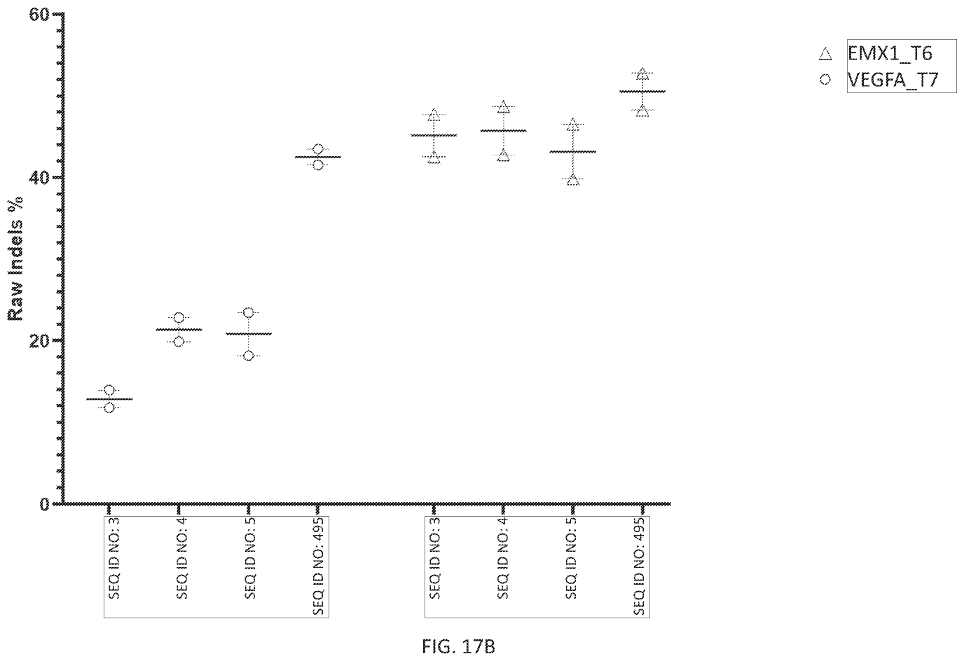

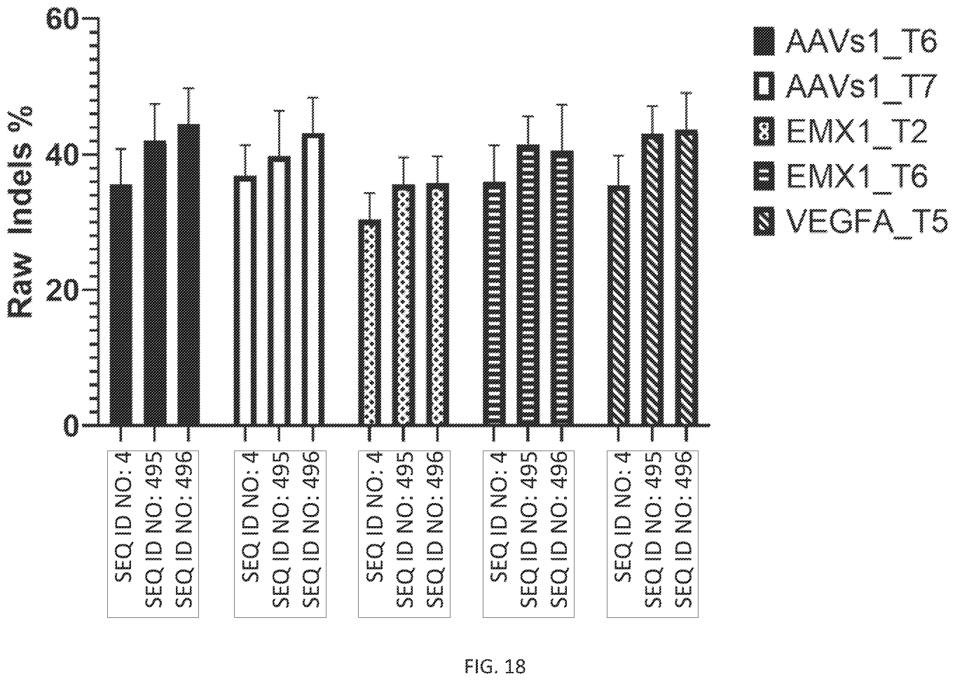

Edited targets were defined as targets that showed indel levels above background (>1.0% in this assay) and greater than 10,000 read counts by Next Generation Sequencing. FIG. 17A shows indels induced in EMX1_T6 and VEGFA_T7 by several engineered Cas12i2 variants. FIG. 17B shows indels induced in EMX1_T6 and VEGFA_T7 by the Cas12i2 variants of SEQ ID NOs: 3-5 and 495. FIG. 18 shows indels induced in AAVS1_T6, AAVS1_T7, EMX1_T2, EMX1_T6, and VEGFA_T5 by the Cas12i2 variants of SEQ ID NOs: 4, 495, and 496.

This Example shows that the Cas12i2 variants of SEQ ID NO: 495 and SEQ ID NO: 496 induced increased indel levels at multiple target sites.

What is claimed is:

1. A variant Cas12i2 polypeptide comprising a sequence having at least 95% identity to a sequence set forth in any one of SEQ ID NOs: 3 to 146 or any one of SEQ ID NOs: 495 to 512.

2. The variant Cas12i2 polypeptide of claim 1, comprising the sequence set forth in SEQ ID NO: 4.

3. The variant Cas12i2 polypeptide of claim 1 or 2, comprising the sequence set forth in SEQ ID NO: 5.

4. The variant Cas12i2 polypeptide of claim 1, comprising the sequence set forth in SEQ ID NO: 495.

5. The variant Cas12i2 polypeptide of claim 1, comprising the sequence set forth in SEQ ID NO: 496.

6. The variant Cas12i2 polypeptide of any one of claims 1 to 5, further comprising a substitution of Table 2.

7. The variant Cas12i2 polypeptide of any one of claims 1 to 6, wherein the variant Cas12i2 polypeptide is a variant of a Cas12i2 polypeptide comprising the sequence set forth in SEQ ID NO: 2.

8. A composition comprising a variant Cas12i2 polypeptide of any one of claims 1 to 7, wherein the composition further comprises an RNA guide or a nucleic acid encoding the RNA guide, wherein the RNA guide comprises a direct repeat sequence and a spacer sequence.

9. The composition of claim 8, wherein the direct repeat sequence comprises a nucleotide sequence with at least 95% sequence identity to any one of SEQ ID NO: 492-494.

10. The composition of claim 9, wherein the direct repeat sequence comprises a nucleotide sequence set forth in any one of SEQ ID NO: 492-494.

11. The composition of any one of claims 8 to 10, wherein the spacer sequence comprises between about 11 and about 50 nucleotides.

12. The composition of claim 11, wherein the spacer sequence comprises between about 15 and about 35 nucleotides.

13. The composition of any one of claims 8 to 12, wherein the spacer sequence binds to a target nucleic acid sequence, and wherein the target nucleic acid sequence is adjacent to a 5′-NTTN-3′ sequence.

14. The variant Cas12i2 polypeptide of any one of claims 1 to 7, or the composition of any one of any one of claims 8 to 13, wherein the variant Cas12i2 polypeptide further comprises at least one nuclear localization signal (NLS), at least one nuclear export signal (NES), or at least one NLS and at least one NES.

15. The variant Cas12i2 polypeptide of any one of claims 1 to 7 or 14, or the composition of any one of claims 8 to 14, wherein the variant Cas12i2 polypeptide further comprises a peptide tag, a fluorescent protein, a base-editing domain, a DNA methylation domain, a histone residue modification domain, a localization factor, a transcription modification factor, a light-gated control factor, a chemically inducible factor, or a chromatin visualization factor.

16. The composition of any one of claims 8 to 15, wherein the composition is present in a delivery system comprising a nanoparticle, a liposome, an exosome, a microvesicle, or a gene-gun.

17. A nucleic acid molecule encoding a variant Cas12i2 polypeptide of any one of claims 1 to 7, 14, or 15.

18. A cell comprising the composition or variant Cas12i2 polypeptide of any previous claim.

19. The cell of claim 18, wherein the cell is a eukaryotic cell or a prokaryotic cell.

20. The cell of claim 18 or 19, wherein the cell is a mammalian cell or a plant cell.

21. The cell of any one of claims 18 to 20, wherein the cell is a human cell.

22. A composition or formulation comprising the variant Cas12i2 polypeptide of any one of claims 1 to 7, 14, or 15 and optionally an RNA guide and/or a cell.

23. A composition comprising a variant Cas12i2 polypeptide and an RNA guide, wherein the Cas12i2 variant polypeptide and the RNA guide form a complex, and wherein the variant Cas12i2 polypeptide exhibits increased complex formation with the RNA guide as compared to a parent polypeptide.

24. A composition comprising a variant Cas12i2 polypeptide and an RNA guide, wherein the Cas12i2 variant polypeptide and the RNA guide form a complex, and wherein the Cas12i2 variant polypeptide exhibits increased binding affinity to the RNA guide, as compared to a parent polypeptide.

25. A composition comprising a variant Cas12i2 polypeptide and an RNA guide, wherein the Cas12i2 variant polypeptide and the RNA guide form a complex, and the Cas12i2 variant polypeptide and the RNA guide exhibit increased protein-RNA interactions, as compared to a parent polypeptide and the RNA guide.

26. The composition of any one of claims 23 to 25, wherein the variant Cas12i2 polypeptide exhibits increased complex formation, increased binding affinity to the RNA guide and/or increased stability over a range of temperatures, e.g., 20° C. to 65° C.

27. The composition of any one of claims 23 to 26, wherein the variant Cas12i2 polypeptide exhibits increased complex formation, increased binding affinity to the RNA guide and/or increased stability over a range of incubation times.

28. The composition of any one of claims 23 to 27, wherein the variant Cas12i2 polypeptide exhibits increased complex formation, increased binding affinity to the RNA guide and/or increased stability in a buffer having a pH in a range of about 7.3 to about 8.6.

29. The composition of any one of claims 23 to 28, wherein the variant Cas12i2 polypeptide exhibits increased complex formation, increased binding affinity to the RNA guide and/or increased stability when a Tₘ value of the variant binary complex is at least 8° C. greater than the Tₘ value of the parent complex.

30. The composition of any one of claims 23 to 29, wherein the parent polypeptide comprises the amino acid sequence of SEQ ID NO: 2.

31. The composition of any one of claims 23 to 30, wherein the variant Cas12i2 polypeptide exhibits equivalent to or greater enzymatic activity than the parent polypeptide.

32. The composition of claim 31, wherein the equivalent to or greater enzymatic activity occurs at a temperature range from about 20° C. to about 90° C.

33. The composition of any one of claims 23 to 32, wherein the variant Cas12i2 polypeptide exhibits increased stability and/or protein-RNA interactions.

34. The composition of any one of claims 23 to 33, wherein the variant Cas12i2 polypeptide further lacks enzymatic activity.

35. The composition of claim 34, wherein the variant Cas12i2 polypeptide further exhibits increased enzymatic activity.

36. The composition of any one of claims 23 to 35, wherein the variant Cas12i2 polypeptide exhibits increased RNA guide complex formation, RNA guide binding activity and/or RNA guide binding specificity.

37. The composition of any one of claims 23 to 36, wherein the variant Cas12i2 polypeptide exhibits altered on-target binding.

38. The composition of any one of claims 23 to 37, wherein the variant Cas12i2 polypeptide exhibits altered off-target binding.

39. A composition comprising a variant Cas12i2 polypeptide and an RNA guide, wherein the Cas12i2 variant polypeptide and the RNA guide form a variant binary complex, and wherein the variant binary complex exhibits decreased complex dissociation than a complex formed by a parent polypeptide and the RNA guide.

40. A composition comprising a variant Cas12i2 polypeptide and an RNA guide, wherein the Cas12i2 variant polypeptide and the RNA guide form a variant binary complex, and wherein the RNA guide exhibits decreased dissociation from the variant Cas12i2 polypeptide than an RNA guide of a parent complex.

41. A composition comprising a variant Cas12i2 polypeptide and an RNA guide, wherein the Cas12i2 variant polypeptide and the RNA guide form a variant binary complex, and wherein the variant binary complex exhibits increased stability than a complex formed by a parent polypeptide and the RNA guide.

42. The composition of any one of claims 39 to 41, wherein the variant binary complex exhibits increased ternary complex formation, increased binding affinity to the target nucleic acid and/or increased stability over a range of temperatures, e.g., 20° C. to 65° C.

43. The composition of any one of claims 39 to 42, wherein the variant binary complex exhibits increased stability over a range of incubation times.

44. The composition of any one of claims 39 to 43, wherein the variant binary complex exhibits increased stability in a buffer having a pH in a range of about 7.3 to about 8.6.

45. The composition of any one of claims 39 to 44, wherein the variant binary complex exhibits increased ternary complex formation, increased binding affinity to the target nucleic acid and/or increased stability hen a Tₘ value of the variant binary complex is at least 8° C. greater than the Tₘ value of the parent complex.

46. The composition of any one of claims 39 to 45, wherein the parent polypeptide comprises the amino acid sequence of SEQ ID NO: 2.

47. The composition of any one of claims 39 to 46, wherein the variant Cas12i2 polypeptide exhibits equivalent or greater enzymatic activity than the parent polypeptide.

48. The composition of claim 47, wherein the equivalent or greater enzymatic activity occurs at a temperature range from about 20° C. to about 90° C.

49. The composition of any one of claims 39 to 48, wherein the variant Cas12i2 polypeptide exhibits increased stability and/or protein-RNA interactions.

50. The composition of any one of claims 39 to 49, wherein the variant Cas12i2 polypeptide further lacks enzymatic activity.

51. The composition of any one of claims 39 to 49, wherein the variant Cas12i2 polypeptide further exhibits increased enzymatic activity.

52. The composition of any one of claims 39 to 51, wherein the variant Cas12i2 polypeptide exhibits increased RNA guide complex formation, RNA guide binding activity and/or RNA guide binding specificity.

53. The composition of any one of claims 39 to 52, wherein the variant Cas12i2 polypeptide exhibits altered on-target binding.

54. The composition of any one of claims 39 to 53, wherein the variant Cas12i2 polypeptide exhibits altered off-target binding.

55. A method of complexing the variant Cas12i2 polypeptide with the RNA guide, e.g., RNA guide, of any one of claims 39 to 54.

56. A composition comprising a variant Cas12i2 polypeptide, an RNA guide and a target nucleic acid, wherein the variant Cas12i2 polypeptide and the RNA guide form a variant binary complex, and wherein the variant binary complex exhibits increased ternary complex formation with the target nucleic acid as compared to a parent binary complex.

57. A composition comprising a variant Cas12i2 polypeptide, an RNA guide and a target nucleic acid, wherein the Cas12i2 variant polypeptide and the RNA guide form a variant binary complex, and wherein the variant binary complex exhibits increased binding affinity to the target nucleic acid, as compared to a parent binary complex.

58. A composition comprising a variant Cas12i2 polypeptide, an RNA guide and target nucleic acid, wherein the Cas12i2 variant polypeptide and the RNA guide form a variant binary complex, and wherein the variant binary complex exhibit increased protein-RNA interactions as compared to a parent binary complex.

59. A composition comprising a variant Cas12i2 polypeptide, an RNA guide and target nucleic acid, wherein the Cas12i2 variant polypeptide and the RNA guide form a variant binary complex, and wherein the variant binary complex exhibit increased protein-DNA interactions as compared to a parent binary complex.

60. The composition of any one of claims 56 to 59, wherein the variant binary complex exhibits increased ternary complex formation, increased binding affinity to the target nucleic acid and/or increased stability over a range of temperatures, e.g., 20° C. to 65° C.

61. The composition of any one of claims 56 to 60, wherein the variant binary complex exhibits increased ternary complex formation, increased binding affinity to the target nucleic acid and/or increased stability over a range of incubation times.

62. The composition of any one of claims 56 to 61, wherein the variant binary complex exhibits increased ternary complex formation, increased binding affinity to the target nucleic acid and/or increased stability in a buffer having a pH in a range of about 7.3 to about 8.6.

63. The composition of any one of claims 56 to 62, wherein the variant binary complex exhibits increased ternary complex formation, increased binding affinity to the target nucleic acid and/or increased stability when a Tₘ value of the binary complex is at least 8° C. greater than the Tₘ value of the parent binary complex.

64. The composition of any one of claims 56 to 63, wherein the parent polypeptide comprises the amino acid sequence of SEQ ID NO: 2.

65. The composition of any one of claims 56 to 64, wherein the variant binary complex exhibits equivalent to or greater enzymatic activity than the parent binary complex.

66. The composition of claim 65, wherein the equivalent to or greater enzymatic activity occurs at a temperature range from about 20° C. to about 90° C.

67. The composition of any one of claims 56 to 66, wherein the variant binary complex exhibits increased stability and/or protein-RNA interactions.

68. The composition of any one of claims 56 to 67, wherein the variant binary complex exhibits increased stability and/or protein-DNA interactions.

69. The composition of any one of claims 56 to 68, wherein the variant binary complex further lacks enzymatic activity.

70. The composition of any one of claims 56 to 68, wherein the variant binary complex further exhibits increased enzymatic activity.

71. The composition of any one of claims 56 to 70, wherein the variant Cas12i2 polypeptide exhibits increased RNA guide complex formation, RNA guide binding activity and/or RNA guide binding specificity.

72. The composition of any one of claims 56 to 71, wherein the variant binary complex exhibits increased target nucleic acid complex formation, target nucleic acid activity and/or target nucleic acid specificity.

73. The composition of any one of claims 56 to 72, wherein the variant binary complex exhibits altered on-target binding.

74. The composition of any one of claims 56 to 73, wherein the variant binary complex exhibits altered off-target binding

75. A composition comprising a variant Cas12i2 polypeptide, an RNA guide and target nucleic acid, wherein the Cas12i2 variant polypeptide and the RNA guide form a variant binary complex, wherein the variant binary complex and target nucleic acid form a variant ternary complex, and wherein the variant ternary complex exhibits decreased complex dissociation than a parent ternary complex.

76. A composition comprising a variant Cas12i2 polypeptide, an RNA guide and target nucleic acid, wherein the Cas12i2 variant polypeptide and the RNA guide form a variant binary complex, wherein the variant binary complex and target nucleic acid form a variant ternary complex, and wherein the target nucleic acid exhibits decreased dissociation from the variant ternary complex than a parent ternary complex.

77. A composition comprising a variant Cas12i2 polypeptide, an RNA guide and target nucleic acid, wherein the Cas12i2 variant polypeptide and the RNA guide form a variant binary complex, wherein the variant binary complex and target nucleic acid form a variant ternary complex, and wherein the variant ternary complex exhibits increased stability than a parent ternary complex.

78. The composition of any one of claims 75 to 77, wherein the variant ternary complex exhibits increased stability over a range of temperatures, e.g., 20° C. to 65° C.

79. The composition of any one of claims 75 to 78, wherein the variant ternary complex exhibits increased stability over a range of incubation times.

80. The composition of any one of claims 75 to 79, wherein the variant ternary complex exhibits increased stability in a buffer having a pH in a range of about 7.3 to about 8.6.

81. The composition of any one of claims 75 to 80, wherein the variant ternary complex exhibits increased stability hen a Tₘ value of the variant ternary complex is at least 8° C. greater than the Tₘ value of the parent ternary complex.

82. The composition of any one of claims 75 to 81, wherein the parent polypeptide comprises the amino acid sequence of SEQ ID NO: 2.

83. The composition of any one of claims 75 to 82, wherein the variant Cas12i2 polypeptide exhibits equivalent or greater enzymatic activity than the parent polypeptide.

84. The composition of claim 83, wherein the equivalent or greater enzymatic activity occurs at a temperature range from about 20° C. to about 90° C.

85. The composition of any one of claims 75 to 84, wherein the variant Cas12i2 polypeptide exhibits increased stability and/or protein-RNA interactions.

86. The composition of any one of claims 75 to 85, wherein the variant binary complex exhibits increased stability and/or protein-DNA interactions.

87. The composition of any one of claims 75 to 86, wherein the variant ternary complex exhibits increased stability.

88. The composition of any one of claims 75 to 87, wherein the variant binary complex further lacks enzymatic activity.

89. The composition of any one of claims 75 to 87, wherein the variant binary complex further exhibits increased enzymatic activity.

90. The composition of any one of claims 75 to 89, wherein the variant Cas12i2 polypeptide exhibits increased RNA guide complex formation, RNA guide binding activity and/or RNA guide binding specificity.

91. The composition of any one of claims 75 to 90, wherein the variant binary complex exhibits increased target nucleic acid complex formation, target nucleic acid binding activity and/or target nucleic acid binding specificity.

92. The composition of any one of claims 75 to 91, wherein the variant binary complex exhibits altered on-target binding.

93. The composition of any one of claims 75 to 92, wherein the variant binary complex exhibits altered off-target binding.

94. A method of complexing the variant binary complex with the target nucleic acid, e.g., DNA, of any one of claims 75 to 93.

95. A composition comprising a variant Cas12i2 polypeptide and an RNA guide, wherein the variant Cas12i2 polypeptide and the RNA guide form a variant binary complex, and wherein the variant binary complex exhibits increased binding affinity to a target nucleic acid as compared to a parent binary complex.

96. A composition comprising a variant Cas12i2 polypeptide and an RNA guide, wherein the Cas12i2 variant polypeptide and the RNA guide form a variant binary complex, and wherein the variant binary complex exhibits increased target binding affinity to a target locus of a target nucleic acid as compared to a parent binary complex.

97. The composition of any one of claims 95 to 96, wherein the variant binary complex exhibits increased ternary complex formation and/or increased stability over a range of temperatures, e.g., 20° C. to 65° C.

98. The composition of any one of claims 95 to 97, wherein the variant binary complex exhibits increased ternary complex formation and/or increased stability over a range of incubation times.

99. The composition of any one of claims 95 to 98, wherein the variant binary complex exhibits increased ternary complex formation and/or increased stability in a buffer having a pH in a range of about 7.3 to about 8.6.

100. The composition of any one of claims 95 to 99, wherein the variant binary complex exhibits increased ternary complex formation and/or increased stability when a Tₘ value of the binary complex is at least 8° C. greater than the Tₘ value of the parent binary complex.

101. The composition of any one of claims 95 to 100, wherein the parent polypeptide comprises the amino acid sequence of SEQ ID NO: 2.

102. The composition of any one of claims 95 to 101, wherein the variant binary complex exhibits equivalent to or greater enzymatic activity than the parent binary complex.

103. The composition of claim 102, wherein the equivalent to or greater enzymatic activity occurs at a temperature range from about 20° C. to about 90° C.

104. The composition of any one of claims 95 to 103, wherein the variant binary complex exhibits increased stability and/or protein-RNA interactions.

105. The composition of any one of claims 95 to 104, wherein the variant binary complex exhibits increased stability and/or protein-DNA interactions.

106. The composition of any one of claims 95 to 105, wherein the variant binary complex further lacks enzymatic activity.

107. The composition of any one of claims 95 to 105, wherein the variant binary complex further exhibits increased enzymatic activity.

108. The composition of any one of claims 95 to 107, wherein the variant Cas12i2 polypeptide exhibits increased RNA guide complex formation, RNA guide binding activity and/or RNA guide binding specificity.

109. The composition of any one of claims 95 to 108, wherein the variant binary complex exhibits increased target nucleic acid complex formation, target nucleic acid activity and/or target nucleic acid specificity.

110. The composition of any one of claims 95 to 109, wherein the variant binary complex exhibits altered on-target binding.

111. The composition of any one of claims 95 to 110, wherein the variant binary complex exhibits altered off-target binding.

112. A composition comprising a plurality of variant Cas12i2 polypeptides and two or more distinct RNA guides, wherein the distinct RNA guides individually form variant binary complexes with separate variant Cas12i2 polypeptides, and wherein the variant binary complexes specifically bind with two or more target loci of a target nucleic acid.

113. A composition comprising a plurality of variant Cas12i2 polypeptides and two or more distinct RNA guides, wherein the distinct RNA guides individually form variant binary complexes with separate variant Cas12i2 polypeptides, and wherein the variant binary complexes exhibit increased on-target binding of two or more target loci of a target nucleic acid as compared to parent binary complexes.

114. A composition comprising a plurality of variant Cas12i2 polypeptides and two or more distinct RNA guides, wherein the distinct RNA guides individually form variant binary complexes with separate variant Cas12i2 polypeptides, and wherein the variant binary complexes exhibit increased on-target binding with two or more target loci of a target nucleic acid as compared to parent binary complexes.

115. A composition comprising a plurality of variant Cas12i2 polypeptides and two or more distinct RNA guides, wherein the distinct RNA guides individually form variant binary complexes with separate variant Cas12i2 polypeptides, and wherein the variant binary complexes exhibit on-target ternary complex formation with two or more target loci of a target nucleic acid.

116. A composition comprising a plurality of variant Cas12i2 polypeptides and two or more distinct RNA guides, wherein the distinct RNA guides individually form variant binary complexes with separate variant Cas12i2 polypeptides, and wherein the variant binary complexes exhibit increased ternary complex formation with two or more target loci of a target nucleic acid as compared to parent binary complexes.

117. The composition of any one of claims 112 to 116, wherein the variant binary complex exhibits increased ternary complex formation with the target nucleic acid and/or increased stability over a range of temperatures, e.g., 20° C. to 65° C.

118. The composition of any one of claims 112 to 117, wherein the variant binary complex exhibits increased ternary complex formation with the target nucleic acid and/or increased stability over a range of incubation times.

119. The composition of any one of claims 112 to 118, wherein the variant binary complex exhibits increased ternary complex formation with the target nucleic acid and/or increased stability in a buffer having a pH in a range of about 7.3 to about 8.6.

120. The composition of any one of claims 112 to 119, wherein the variant binary complex exhibits increased ternary complex formation with the target nucleic acid and/or increased stability when a Tₘ value of the binary complex is at least 8° C. greater than the Tₘ value of the parent binary complex.

121. The composition of any one of claims 112 to 120, wherein the parent polypeptide comprises the amino acid sequence of SEQ ID NO: 2.

122. The composition of any one of claims 112 to 121, wherein the variant Cas12i2 polypeptide exhibits equivalent or greater enzymatic activity than the parent polypeptide.

123. The composition of claim 122, wherein the equivalent or greater enzymatic activity occurs at a temperature range from about 20° C. to about 90° C.

124. The composition of any one of claims 112 to 123, wherein the variant Cas12i2 polypeptide exhibits increased stability and/or protein-RNA interactions.

125. The composition of any one of claims 112 to 124, wherein the variant binary complex exhibits increased stability and/or protein-DNA interactions.

126. The composition of any one of claims 112 to 125, wherein the variant binary complex further lacks enzymatic activity.

127. The composition of any one of claims 112 to 125, wherein the variant binary complex further exhibits increased enzymatic activity.

128. The composition of any one of claims 112 to 127, wherein the variant Cas12i2 polypeptide exhibits increased RNA guide complex formation, RNA guide binding activity and/or RNA guide binding specificity.

129. The composition of any one of claims 112 to 128, wherein the variant binary complex exhibits increased target nucleic acid ternary complex formation, target nucleic acid binding affinity and/or target nucleic acid binding specificity.

130. The composition of any one of claims 112 to 129, wherein the variant binary complex exhibits altered on-target binding.

131. The composition of any one of claims 112 to 130, wherein the variant binary complex exhibits altered off-target binding.

132. A method of complexing the variant binary complex with the target nucleic acid, e.g., DNA, of any one of claims 112 to 131.

133. A composition comprising a variant Cas12i2 polypeptide and an RNA guide, wherein the Cas12i2 variant polypeptide and the RNA guide form a variant binary complex, and wherein the variant binary complex exhibits increased on-target binding affinity to a target locus of a target nucleic acid as compared to a parent binary complex.

134. A composition comprising a variant Cas12i2 polypeptide and an RNA guide, wherein the variant Cas12i2 polypeptide and the RNA guide form a variant binary complex, and wherein the variant binary complex exhibits decreased binding affinity to a non-target locus of a target nucleic acid as compared to a parent binary complex.

135. A composition comprising a variant Cas12i2 polypeptide and an RNA guide, wherein the variant Cas12i2 polypeptide and the RNA guide form a variant binary complex, and wherein the variant binary complex exhibits increased activity at an on-target locus of a target nucleic acid as compared to a parent binary complex.

136. A composition comprising a variant Cas12i2 polypeptide and an RNA guide, wherein the variant Cas12i2 polypeptide and the RNA guide form a variant binary complex, and wherein the variant binary complex exhibits decreased activity at a non-target locus of a target nucleic acid as compared to a parent binary complex.

137. The composition of any one of claims 133 to 136, wherein the variant binary complex exhibits increased ternary complex formation at the target locus and/or increased stability over a range of temperatures, e.g., 20° C. to 65° C.

138. The composition of any one of claims 133 to 137, wherein the variant binary complex exhibits increased ternary complex formation at the target locus and/or increased stability over a range of incubation times.

139. The composition of any one of claims 133 to 138, wherein the variant binary complex exhibits increased ternary complex formation at the target locus and/or increased stability in a buffer having a pH in a range of about 7.3 to about 8.6.

140. The composition of any one of claims 133 to 139, wherein the variant binary complex exhibits increased ternary complex formation at the target locus and/or increased stability when a Tₘ value of the binary complex is at least 8° C. greater than the Tₘ value of the parent binary complex.

141. The composition of any one of claims 133 to 140, wherein the parent binary complex comprises a parent polypeptide that comprises the amino acid sequence of SEQ ID NO: 2.

142. The composition of claim 141, wherein the variant Cas12i2 polypeptide exhibits equivalent or greater enzymatic activity than the parent polypeptide.

143. The composition of claim 142, wherein the equivalent or greater enzymatic activity occurs at a temperature range from about 20° C. to about 90° C.

144. The composition of any one of claims 133 to 143, wherein the variant binary complex exhibits increased stability and/or protein-RNA interactions.

145. The composition of any one of claims 133 to 144, wherein the variant binary complex exhibits increased stability and/or protein-DNA interactions.

146. The composition of any one of claims 133 to 145, wherein the variant Cas12i2 polypeptide exhibits increased binary complex formation, RNA guide binding activity and/or RNA guide binding specificity.

147. A composition comprising a plurality of variant Cas12i2 polypeptides and two or more distinct RNA guides, wherein the distinct RNA guides individually form a plurality of variant binary complexes with separate variant Cas12i2 polypeptides, and wherein the plurality of variant binary complexes exhibit increased on-target binding to two or more target loci of a target nucleic acid as compared to a plurality of parent binary complexes.

148. A composition comprising a plurality of variant Cas12i2 polypeptides and two or more distinct RNA guides, wherein the distinct RNA guides individually form a plurality of variant binary complexes with separate variant Cas12i2 polypeptides, and wherein the plurality of variant binary complexes exhibit decreased off-target binding to two or more non-target loci of a target nucleic acid as compared to a plurality of parent binary complexes.

149. A composition comprising a plurality of variant Cas12i2 polypeptides and two or more distinct RNA guides, wherein the distinct RNA guides individually form a plurality of variant binary complexes with separate variant Cas12i2 polypeptides, and wherein the plurality of variant binary complexes exhibit increased on-target activity at two or more target loci of a target nucleic acid as compared to a plurality of parent binary complexes.

150. A composition comprising a plurality of variant Cas12i2 polypeptides and two or more distinct RNA guides, wherein the distinct RNA guides individually form a plurality of variant binary complexes with separate variant Cas12i2 polypeptides, and wherein the plurality of variant binary complexes exhibit decreased off-target activity at two or more non-target loci of a target nucleic acid as compared to a plurality of parent binary complexes.

151. The composition of any one of claims 147 to 150, wherein the plurality of variant binary complexes exhibit increased ternary complex formation at the target loci of the target nucleic acid and/or increased stability over a range of temperatures, e.g., 20° C. to 65° C.

152. The composition of any one of claims 147 to 151, wherein the plurality of variant binary complexes exhibit increased ternary complex formation at the target loci of the target nucleic acid and/or increased stability over a range of incubation times.

153. The composition of any one of claims 147 to 152, wherein the plurality of variant binary complexes exhibit increased ternary complex formation at the target loci the target nucleic acid and/or increased stability in a buffer having a pH in a range of about 7.3 to about 8.6.

154. The composition of any one of claims 147 to 153, wherein the plurality of variant binary complexes exhibit increased ternary complex formation at the target loci of the target nucleic acid and/or increased stability when a Tₘ value of the binary complex is at least 8° C. greater than the Tₘ value of the parent binary complex.

155. The composition of any one of claims 147 to 154, wherein the plurality of parent binary complexes comprise a parent polypeptide that comprises the amino acid sequence of SEQ ID NO: 2.

156. The composition of any one of claims 147 to 155, wherein the variant Cas12i2 polypeptide exhibits equivalent or greater enzymatic activity than the parent polypeptide.

157. The composition of claim 156, wherein the equivalent or greater enzymatic activity occurs at a temperature range from about 20° C. to about 90° C.

158. The composition of any one of claims 147 to 157, wherein the variant Cas12i2 polypeptide exhibits increased stability and/or protein-RNA interactions.

159. The composition of any one of claims 147 to 158, wherein the plurality of variant binary complexes exhibit increased stability and/or protein-DNA interactions.

160. The composition of any one of claims 147 to 159, wherein the variant Cas12i2 polypeptide exhibits increased binary complex formation, RNA guide binding activity and/or RNA guide binding specificity.

161. A method of complexing the variant binary complex with the target nucleic acid, e.g., DNA, of any one of claims 147 to 160.

162. A method of complexing the plurality of variant binary complexes with the target nucleic acid, e.g., DNA, of any one of claims 147 to 161.

163. The composition of any one of claims 23 to 54, 56 to 93, 95 to 131, and 133 to 160, wherein the variant Cas12i2 polypeptide comprises at least one of a D581, G624, F626, D835, L836, P868, S879, D911, 1926, V1020, V1030, E1035, and 51046 substitution of amino acid sequence of SEQ ID NO: 2.

164. The composition of any one of claims 23 to 54, 56 to 93, 95 to 131, 133 to 160, and 163, wherein the variant Cas12i2 polypeptide comprises at least one of a D581G, D581R, G624R, F626G, F626R, D835G, D835R, L836G, L836R, P868G, P868R, P868T, S879G, S879R, D911G, D911R, 1926G, 1926R, V1020G, V1020R, V1030G, V1030R, E1035G, E1035R, S1046G, and S1046R substitution of amino acid sequence of SEQ ID NO: 2.

165. The composition of any one of claims 23 to 54, 56 to 93, 95 to 131, 133 to 160, 163, and 164, wherein the variant Cas12i2 polypeptide comprises at least one of a D581R, G624R, F626R, P868T, D911R, I926R, V1030G, E1035R, and S1046G substitution of amino acid sequence of SEQ ID NO: 2.

166. The composition of any one of claims 23 to 54, 56 to 93, 95 to 131, 133 to 160, and 163 to 165, wherein the variant Cas12i2 polypeptide comprises at least one substitution listed in Table 1.

167. The composition of any one of claims 23 to 54, 56 to 93, 95 to 131, 133 to 160, and 163 to 166, wherein the variant Cas12i2 polypeptide comprises any one amino acid sequence of SEQ ID NO: 3 to 146 or any one amino acid sequence of SEQ ID NOs: 495 to 512.

168. The composition of any one of claims 23 to 54, 56 to 93, 95 to 131, 133 to 160, and 163 to 167, wherein the variant Cas12i2 polypeptide comprises at least one of an epitope peptide, nuclear localization signal, and nuclear export signal.

169. The composition of any one of claims 23 to 54, 56 to 93, 95 to 131, 133 to 160, and 163 to 168, wherein the RNA guide comprises a DNA targeting sequence.

170. The composition of claim 169, wherein the DNA targeting sequence is an RNA guide.

171. The composition of claim 169 or 170, wherein the DNA targeting sequence is between 13 to 30 nucleotides.

172. The composition of any one of claims 23 to 54, 56 to 93, 95 to 131, 133 to 160, and 163 to 171, wherein the RNA guide comprises a direct repeat sequence linked to a DNA targeting sequence.

173. The composition of any one of claims 23 to 54, 56 to 93, 95 to 131, 133 to 160, and 163 to 172, further comprising a target nucleic acid.

174. The composition of claim 173, wherein the target nucleic acid is present in a cell.

175. The composition of any one of claims 23 to 54, 56 to 93, 95 to 131, 133 to 160, and 163 to 174, wherein the variant Cas12i2 polypeptide and RNA guide are encoded in a vector, e.g., expression vector.

176. A cell comprising the composition of any one of claims 23 to 54, 56 to 93, 95 to 131, 133 to 160, and 163 to 175.

177. A method of expressing the vector of claim 175.

178. A method of producing the composition of any one of claims 23 to 54, 56 to 93, 95 to 131, 133 to 160, and 163 to 175.

179. A method of delivering the composition of any one of claims 23 to 54, 56 to 93, 95 to 131, 133 to 160, and 163 to 175.

180. A kit or system comprising a composition of any one of claims 23 to 54, 56 to 93, 95 to 131, 133 to 160, and 163 to 175 or one or more component thereof.

181. The composition of any one of claims 23 to 54, 56 to 93, 95 to 131, 133 to 160, and 163 to 175, wherein the RNA guide comprises or consists of, or about, 43 nucleotides.

182. The composition of any one of claims 23 to 54, 56 to 93, 95 to 131, 133 to 160, 163 to 175, and 181, wherein the RNA guide is a tracr-less RNA guide.

183. The composition of any one of claims 23 to 54, 56 to 93, 95 to 131, 133 to 160, 163 to 175, 181, and 182, wherein the variant Cas12i2 polypeptide further exhibits about 40× greater enzymatic activity than parent polypeptide.

184. The composition of any one of claims 23 to 54, 56 to 93, 95 to 131, 133 to 160, 163 to 175, and 181 to 183, wherein the variant Cas12i2 polypeptide exhibits increased on-target specificity as compared to the parent polypeptide.

185. The composition of any one of claims 23 to 54, 56 to 93, 95 to 131, 133 to 160, 163 to 175, and 181 to 184, wherein the variant Cas12i2 polypeptide exhibits decreased off-target specificity as compared to the parent polypeptide.

186. The composition of any one of claims 23 to 54, 56 to 93, 95 to 131, 133 to 160, 163 to 175, and 181 to 185, wherein the variant Cas12i2 polypeptide selectively induces a deletion adjacent to a 5′-NTTN-3′ sequence, wherein N is any nucleotide.

187. The composition of claim 186, wherein the deletion is downstream of the 5′-NTTN-3′ sequence.

188. The composition of claim 186 or 187, wherein the parent polypeptide does not induce the deletion.

189. The composition of any one of claims 186 to 188, wherein the length of the deletion is greater than the length of a Cas9 polypeptide-induced deletion.

190. The composition of any one of claims 186 to 189, wherein the deletion is in a gene of a cell.

191. The composition of any one of claims 186 to 190, wherein the deletion is up to about 40 nucleotides in length.

192. The composition of any one of claims 186 to 191, wherein the deletion is from about 4 nucleotides to 40 nucleotides in length.

193. The composition of any one of claims 186 to 192, wherein the deletion is from about 4 nucleotides to 25 nucleotides in length.

194. The composition of any one of claims 186 to 193, wherein the deletion is from about 10 nucleotides to 25 nucleotides in length.

195. The composition of any one of claims 186 to 194, wherein the deletion is from about 10 nucleotides to 15 nucleotides in length.

196. The composition of any one of claims 186 to 195, wherein the deletion starts within about 5 nucleotides to about 15 nucleotides of the 5′-NTTN-3′ sequence.

197. The composition of any one of claims 186 to 196, wherein the deletion starts within about 5 nucleotides to about 10 nucleotides of the 5′-NTTN-3′ sequence.

198. The composition of any one of claims 186 to 197, wherein the deletion starts within about 10 nucleotides to about 15 nucleotides of the 5′-NTTN-3′ sequence.

199. The composition of any one of claims 186 to 198, wherein the deletion starts within about 5 nucleotides to about 15 nucleotides downstream of the 5′-NTTN-3′ sequence.

200. The composition of any one of claims 186 to 199, wherein the deletion starts within about 5 nucleotides to about 10 nucleotides downstream of the 5′-NTTN-3′ sequence.

201. The composition of any one of claims 186 to 200, wherein the deletion starts within about 10 nucleotides to about 15 nucleotides downstream of the 5′-NTTN-3′ sequence.

202. The composition of any one of claims 186 to 201, wherein the deletion ends within about 20 nucleotides to about 30 nucleotides of the 5′-NTTN-3′ sequence.

203. The composition of any one of claims 186 to 202, wherein the deletion ends within about 20 nucleotides to about 25 nucleotides of the 5′-NTTN-3′ sequence.

204. The composition of any one of claims 186 to 203, wherein the deletion ends within about 25 nucleotides to about 30 nucleotides of the 5′-NTTN-3′ sequence.

205. The composition of any one of claims 186 to 204, wherein the deletion ends within about 20 nucleotides to about 30 nucleotides downstream of the 5′-NTTN-3′ sequence.

206. The composition of any one of claims 186 to 205, wherein the deletion ends within about 20 nucleotides to about 25 nucleotides downstream of the 5′-NTTN-3′ sequence.

207. The composition of any one of claims 186 to 206, wherein the deletion ends within about 25 nucleotides to about 30 nucleotides downstream of the 5′-NTTN-3′ sequence.

208. The composition of any one of claims 186 to 207, wherein the deletion starts within about 5 nucleotides to about 15 nucleotides downstream of the 5′-NTTN-3′ sequence and ends within about 20 nucleotides to about 30 nucleotides downstream of the 5′-NTTN-3′ sequence.

209. The composition of any one of claims 186 to 208, wherein the deletion starts within about 5 nucleotides to about 15 nucleotides downstream of the 5′-NTTN-3′ sequence and ends within about 20 nucleotides to about 25 nucleotides downstream of the 5′-NTTN-3′ sequence.

210. The composition of any one of claims 186 to 209, wherein the deletion starts within about 5 nucleotides to about 15 nucleotides downstream of the 5′-NTTN-3′ sequence and ends within about 25 nucleotides to about 30 nucleotides downstream of the 5′-NTTN-3′ sequence.

211. The composition of any one of claims 186 to 210, wherein the deletion starts within about 5 nucleotides to about 10 nucleotides downstream of the 5′-NTTN-3′ sequence and ends within about 20 nucleotides to about 30 nucleotides downstream of the 5′-NTTN-3′ sequence.

212. The composition of any one of claims 186 to 211, wherein the deletion starts within about 5 nucleotides to about 10 nucleotides downstream of the 5′-NTTN-3′ sequence and ends within about 20 nucleotides to about 25 nucleotides downstream of the 5′-NTTN-3′ sequence.

213. The composition of any one of claims 186 to 212, wherein the deletion starts within about 5 nucleotides to about 10 nucleotides downstream of the 5′-NTTN-3′ sequence and ends within about 25 nucleotides to about 30 nucleotides downstream of the 5′-NTTN-3′ sequence.

214. The composition of any one of claims 186 to 213, wherein the deletion starts within about 10 nucleotides to about 15 nucleotides downstream of the 5′-NTTN-3′ sequence and ends within about 20 nucleotides to about 30 nucleotides downstream of the 5′-NTTN-3′ sequence.

215. The composition of any one of claims 186 to 214, wherein the deletion starts within about 10 nucleotides to about 15 nucleotides downstream of the 5′-NTTN-3′ sequence and ends within about 20 nucleotides to about 25 nucleotides downstream of the 5′-NTTN-3′ sequence.

216. The composition of any one of claims 186 to 215, wherein the deletion starts within about 10 nucleotides to about 15 nucleotides downstream of the 5′-NTTN-3′ sequence and ends within about 25 nucleotides to about 30 nucleotides downstream of the 5′-NTTN-3′ sequence.

217. The composition of any one of claims 186 to 216, wherein the 5′-NTTN-3′ sequence is 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′, wherein Y is C or T, B is any nucleotide except for A, D is any nucleotide except for C, and R is A or G.

218. The composition of any one of claims 186 to 217, wherein the 5′-NTTN-3′ sequence is 5′-CTTT-3′, 5′-CTTC-3′, 5′-GTTT-3′, 5′-GTTC-3′, 5′-TTTC-3′, 5′-GTTA-3′, or 5′-GTTG-3′.

219. The composition of any one of claims 186 to 218, wherein the deletion is in an exon of the gene, e.g., B2M, TRAC, PDCD1.

220. The composition of any one of claims 186 to 219, wherein the deletion overlaps with a mutation in the gene.

221. The composition of any one of claims 186 to 220, wherein the deletion overlaps with an insertion in the gene.

222. The composition of any one of claims 186 to 221, wherein the deletion removes a repeat expansion of the gene.

223. The composition of any one of claims 186 to 222, wherein the deletion disrupts one or both alleles of the gene.

224. The composition of any one of claims 186 to 223, wherein the deletion is induced in a eukaryotic cell or a prokaryotic cell.

225. The composition of any one of claims 186 to 224, wherein the deletion is induced in an animal cell, a plant cell, or a fungal cell or the cell is derived from an animal cell, a plant cell, or a fungal cell.

226. The composition of any one of claims 186 to 225, wherein the deletion is induced in a mammalian cell or derived from a mammalian cell.

227. The composition of any one of claims 186 to 226, wherein the deletion is induced in a human cell or derived from a human cell.

228. The composition of any one of claims 186 to 227, wherein the deletion is induced in a primary cell.

229. The composition of any one of claims 186 to 228, wherein the deletion is induced in a cell line.

230. The composition of any one of claims 186 to 229, wherein the deletion is induced in a T cell.

231. The composition of any one of claims 186 to 230, wherein the deletion is induced in a stem cell (e.g., a totipotent/omnipotent stem cell, a pluripotent stem cell, a multipotent stem cell, an oligopotent stem cell, or an unipotent stem cell), a differentiated cell, or a terminally differentiated cell.

232. The composition of any one of claims 186 to 231, wherein 2 or more (e.g., multiplexed targeted deletions) deletions are induced.

233. A method of obtaining a deletion in a cell, wherein the method comprises contacting the variant Cas12i2 polypeptide or complex of any one of claims 1 to 16, 22 to 54, 56 to 93, 95 to 131, 133 to 160, 163 to 175, and 181 to 232 with DNA in the cell.

234. A composition or formulation comprising the variant Cas12i2 polypeptide of any one of claims 1 to 16, 22 to 54, 56 to 93, 95 to 131, 133 to 160, 163 to 175, and 181 to 232, an RNA guide, and a cell.

235. A method of producing the composition of any previous claim.

236. A method of complexing a variant Cas12i2 polypeptide of any previous claim with an RNA guide, such as an RNA guide of any previous claim.

237. A method of complexing a variant binary complex of any previous claim with a target nucleic acid.

238. A method of delivering the composition of any previous claim.

239. A composition comprising a variant Cas12i2 polypeptide, wherein the variant Cas12i2 polypeptide comprises a substitution that increases interactions between the variant Cas12i2 polypeptide and a nucleic acid, as compared to a parent polypeptide.

240. The composition of claim 239, wherein the variant Cas12i2 polypeptide comprises the sequence set forth in SEQ ID NO: 4.

241. The composition of claim 239, wherein the variant Cas12i2 polypeptide comprises the sequence set forth in any one of SEQ ID NO: 3, SEQ ID NO: 5, SEQ ID NO: 495, and SEQ ID NO: 496.

242. The composition of claim 239, wherein the interactions are electrostatic interactions.

243. The composition of claim 239, wherein the interactions are non-specific interactions.

244. The composition of claim 239, wherein the interactions are aromatic, hydrophobic, Van der Waals, and/or cation-pi interactions.

245. The composition of claim 239, wherein the substitution is in or adjacent to a nucleic acid interface.

246. The composition of any one of claims 239 to 245, wherein the nucleic acid is an RNA guide comprising a direct repeat sequence and a spacer sequence.

247. The composition of claim 246, wherein the direct repeat sequence comprises a nucleotide sequence with at least 95% sequence identity to any one of SEQ ID NO: 492-494.

248. The composition of claim 246, wherein the direct repeat sequence comprises a nucleotide sequence set forth in any one of SEQ ID NO: 492-494.

249. The composition of any one of claims 246 to 248, wherein the substitution increases interactions between the variant Cas12i2 polypeptide and the direct repeat sequence.

250. The composition of any one of claims 239 to 249, wherein the substitution increases binary complex formation, as compared to a parent polypeptide.

251. The composition of any one of claims 239 to 250, wherein a binary complex comprising the variant Cas12i2 polypeptide exhibits increased stability, as compared to a parent binary complex.

252. The composition of any one of claims 239 to 251, wherein the substitution is an arginine, lysine, glutamine, asparagine, histidine, tyrosine, or serine substitution.

253. The composition of any one of claims 239 to 252, wherein the substitution is in an RNA binding interface.

254. The composition of any one of claims 239 to 253, wherein the substitution is a substitution in the Wedge domain or Rec2 domain.

255. The composition of any one of claims 239 to 254, wherein the substitution is a substitution listed in Table 4.

256. The composition of any one of claims 239 to 254, wherein the variant Cas12i2 polypeptide comprises a sequence having at least 95% identity to a sequence set forth in any one of SEQ ID NOs: 3-146 or in any one of SEQ ID NOs: 495-512.

257. The composition of any one of claims 239 to 254, wherein the variant Cas12i2 polypeptide comprises a sequence having at least 95% identity to a sequence set forth in any one of SEQ ID NOs: 3-146 or in any one of SEQ ID NOs: 495-512 and further having a substitution listed in Table 4.

258. The composition of any one of claims 239 to 254, wherein the variant Cas12i2 polypeptide comprises a sequence set forth in any one of SEQ ID NOs: 3-146 or in any one of SEQ ID NOs: 495-512.

259. The composition of any one of claims 239 to 254, wherein the variant Cas12i2 polypeptide comprises a sequence set forth in any one of SEQ ID NOs: 3-146 or in any one of SEQ ID NOs: 495-512 and further has a substitution listed in Table 4.

260. The composition of any one of claims 246 and 249 to 254, wherein the direct repeat sequence is set forth in SEQ ID NO: 492 and the variant Cas12i2 polypeptide comprises a sequence set forth in SEQ ID NO: 4.

261. The composition of any one of claims 246 and 249 to 254, wherein the direct repeat sequence is set forth in SEQ ID NO: 492 and the variant Cas12i2 polypeptide comprises the sequence set forth in any one of SEQ ID NO: 3, SEQ ID NO: 5, SEQ ID NO: 495, and SEQ ID NO: 496.

262. The composition of any one of claims 239 to 245, wherein the nucleic acid is a target nucleic acid.

263. The composition of any one of claims 239 to 245 and 262, wherein the nucleic acid is double-stranded DNA.

264. The composition of claim 263, wherein the substitution increases interactions between the variant Cas12i2 polypeptide and double-stranded DNA.

265. The composition of claim 263 or claim 264 wherein the double-stranded DNA comprises a PAM sequence.

266. The composition of any one of claims 239 to 245 and 262 to 265, wherein the substitution increases ternary complex formation, as compared to a parent polypeptide.

267. The composition of any one of claims 239 to 245 and 262 to 266, wherein a ternary complex comprising the variant Cas12i2 polypeptide exhibits increased stability, as compared to a parent ternary complex.

268. The composition of any one of claims 239 to 245 and 262 to 267, wherein the substitution is an arginine, lysine, glutamine, asparagine, histidine, or serine substitution.

269. The composition of any one of claims 239 to 245 and 262 to 268, wherein the substitution is in a double-stranded DNA binding interface.

270. The composition of any one of claims 239 to 245 and 262 to 269, wherein the substitution is a substitution in the Rec1 domain, PI domain, or Wedge domain.

271. The composition of any one of claims 239 to 245 and 262 to 270, wherein the substitution is a substitution listed in Table 5.

272. The composition of any one of claims 239 to 245 and 262 to 270, wherein the variant Cas12i2 polypeptide comprises a sequence having at least 95% identity to a sequence set forth in any one of SEQ ID NOs: 3-146 or in any one of SEQ ID NOs: 495-512.

273. The composition of any one of claims 239 to 245 and 262 to 270, wherein the variant Cas12i2 polypeptide comprises a sequence having at least 95% identity to a sequence set forth in any one of SEQ ID NOs: 3-146 or in any one of SEQ ID NOs: 495-512 and further having a substitution listed in Table 5.

274. The composition of any one of claims 239 to 245 and 262 to 270, wherein the variant Cas12i2 polypeptide comprises a sequence set forth in any one of SEQ ID NOs: 3-146 or in any one of SEQ ID NOs: 495-512.

275. The composition of any one of claims 239 to 245 and 262 to 270, wherein the variant Cas12i2 polypeptide comprises a sequence set forth in any one of SEQ ID NOs: 3-146 or in any one of SEQ ID NOs: 495-512 and further has a substitution listed in Table 5.

276. The composition of any one of claims 239 to 245, wherein the nucleic acid is single-stranded DNA.

277. The composition of claim 276, wherein the single-stranded DNA comprises a non-target strand.

278. The composition of claim 276, wherein the single-stranded DNA comprises a target strand.

279. The composition of any one of claims 239 to 245 and 276 to 278, wherein the substitution increases ternary complex formation, as compared to a parent polypeptide.

280. The composition of any one of claims 239 to 245 and 276 to 279, wherein a ternary complex comprising the variant Cas12i2 polypeptide exhibits increased stability, as compared to a parent ternary complex.

281. The composition of any one of claims 239 to 245 and 276 to 280, wherein the substitution is an arginine, lysine, glutamine, asparagine, histidine, or alanine substitution.

282. The composition of any one of claims 239 to 245 and 276 to 281, wherein the substitution is in a single-stranded DNA binding interface.

283. The composition of any one of claims 239 to 245 and 276 to 282, wherein the substitution is a substitution in the PI domain, Rec1 domain, Wedge domain, RuvC domain, Rec2 domain, or Nuc domain.

284. The composition of any one of claims 239 to 245 and 276 to 283, wherein the substitution is a substitution listed in Table 6.

285. The composition of any one of claims 239 to 245 and 276 to 283, wherein the variant Cas12i2 polypeptide comprises a sequence having at least 95% identity to a sequence set forth in any one of SEQ ID NOs: 3-146 or in any one of SEQ ID NOs: 495-512.

286. The composition of any one of claims 239 to 245 and 276 to 283, wherein the variant Cas12i2 polypeptide comprises a sequence having at least 95% identity to a sequence set forth in any one of SEQ ID NOs: 3-146 or in any one of SEQ ID NOs: 495-512 and further having a substitution listed in Table 6.

287. The composition of any one of claims 239 to 245 and 276 to 283, wherein the variant Cas12i2 polypeptide comprises a sequence set forth in any one of SEQ ID NOs: 3-146 or in any one of SEQ ID NOs: 495-512.

288. The composition of any one of claims 239 to 245 and 276 to 283, wherein the variant Cas12i2 polypeptide comprises a sequence set forth in any one of SEQ ID NOs: 3-146 or in any one of SEQ ID NOs: 495-512 and further has a substitution listed in Table 6.

289. The composition of any one of claims 239 to 245, wherein the substitution increases interactions between the variant Cas12i2 polypeptide and a DNA/RNA hybrid molecule.

290. The composition of claim 289, wherein the DNA/RNA hybrid molecule is a heteroduplex comprising a spacer sequence of an RNA guide and a target strand.

291. The composition of any one of claims 239 to 245 and 289 to 290, wherein the substitution stabilizes the heteroduplex.

292. The composition of any one of claims 239 to 245 and 289 to 291, wherein the substitution increases ternary complex formation, as compared to a parent polypeptide.

293. The composition of any one of claims 239 to 245 and 289 to 292, wherein a ternary complex comprising the variant Cas12i2 polypeptide exhibits increased stability, as compared to a parent ternary complex.

294. The composition of any one of claims 239 to 245 and 289 to 293, wherein the substitution is an arginine, lysine, glutamine, asparagine, histidine, or serine substitution.

295. The composition of any one of claims 239 to 245 and 289 to 294, wherein the substitution is a substitution in the Rec1 domain, PI domain, Rec2 domain, or RuvC2 motif.

296. The composition of any one of claims 239 to 245 and 289 to 295, wherein the substitution is a substitution listed in Table 7.

297. The composition of any one of claims 239 to 245 and 289 to 295, wherein the variant Cas12i2 polypeptide comprises a sequence having at least 95% identity to a sequence set forth in any one of SEQ ID NOs: 3-146 or in any one of SEQ ID NOs: 495-512.

298. The composition of any one of claims 239 to 245 and 289 to 295, wherein the variant Cas12i2 polypeptide comprises a sequence having at least 95% identity to a sequence set forth in any one of SEQ ID NOs: 3-146 or in any one of SEQ ID NOs: 495-512 and further having a substitution listed in Table 7.

299. The composition of any one of claims 239 to 245 and 289 to 295, wherein the variant Cas12i2 polypeptide comprises a sequence set forth in any one of SEQ ID NOs: 3-146 or in any one of SEQ ID NOs: 495-512.

300. The composition of any one of claims 239 to 245 and 289 to 295, wherein the variant Cas12i2 polypeptide comprises a sequence set forth in any one of SEQ ID NOs: 3-146 or in any one of SEQ ID NOs: 495-512 and further has a substitution listed in Table 7.

301. The composition of any one of claims 239 to 245, wherein the substitution increases interactions between the variant Cas12i2 polypeptide and bases of (a) a double-stranded DNA duplex and/or (b) a heteroduplex comprising a spacer sequence of an RNA guide and a target strand.

302. The composition of claim 301, wherein the double-stranded DNA duplex comprises a PAM sequence.

303. The composition of any one of claims 239 to 245 and 301 to 302, wherein the interactions are aromatic, hydrophobic, Van der Waals, and/or cation-pi interactions.

304. The composition of any one of claims 239 to 245 and 301 to 303, wherein the substitution stabilizes the R-loop.

305. The composition of any one of claims 239 to 245 and 301 to 304, wherein the substitution increases ternary complex formation, as compared to a parent polypeptide.

306. The composition of any one of claims 239 to 245 and 301 to 305, wherein a ternary complex comprising the variant Cas12i2 polypeptide exhibits increased stability, as compared to a parent ternary complex.

307. The composition of any one of claims 239 to 245 and 301 to 306, wherein the substitution is an arginine, lysine, tryptophan, phenylalanine, tyrosine, methionine, histidine, glutamine, threonine, or valine substitution.

308. The composition of any one of claims 239 to 245 and 301 to 307, wherein the substitution is a substitution in the Wedge domain, Rec1 domain, or RuvC domain.

309. The composition of any one of claims 239 to 245 and 301 to 308, wherein the substitution is a substitution listed in Table 8.

310. The composition of any one of claims 239 to 245 and 301 to 308, wherein the variant Cas12i2 polypeptide comprises a sequence having at least 95% identity to a sequence set forth in any one of SEQ ID NOs: 3-146 or in any one of SEQ ID NOs: 495-512.

311. The composition of any one of claims 239 to 245 and 301 to 308, wherein the variant Cas12i2 polypeptide comprises a sequence having at least 95% identity to a sequence set forth in any one of SEQ ID NOs: 3-146 or in any one of SEQ ID NOs: 495-512 and further having a substitution listed in Table 8.

312. The composition of any one of claims 239 to 245 and 301 to 308, wherein the variant Cas12i2 polypeptide comprises a sequence set forth in any one of SEQ ID NOs: 3-146 or in any one of SEQ ID NOs: 495-512.

313. The composition of any one of claims 239 to 245 and 301 to 308, wherein the variant Cas12i2 polypeptide comprises a sequence set forth in any one of SEQ ID NOs: 3-146 or in any one of SEQ ID NOs: 495-512 and further has a substitution listed in Table 8.

314. The composition of any one of claims 239 to 313, wherein the parent polypeptide comprises the amino acid sequence of SEQ ID NO: 2.

315. The composition of any one of claims 239 to 314, wherein the variant Cas12i2 polypeptide exhibits increased enzymatic activity, as compared to a parent polypeptide.

316. The composition of any one of claims 239 to 315, wherein the composition further comprises an RNA guide comprising a direct repeat sequence and a spacer sequence.

317. The composition of claim 316, wherein the direct repeat sequence comprises a nucleotide sequence with at least 95% sequence identity to any one of SEQ ID NO: 492-494.

318. The composition of claim 316, wherein the direct repeat sequence comprises a nucleotide sequence set forth in any one of SEQ ID NO: 492-494.

319. The composition of claim 316, wherein the direct repeat sequence is set forth in SEQ ID NO: 492 and the variant Cas12i2 polypeptide comprises a sequence set forth in SEQ ID NO: 4.

320. The composition of claim 316, wherein the direct repeat sequence is set forth in SEQ ID NO: 492 and the variant Cas12i2 polypeptide comprises the sequence set forth in any one of SEQ ID NO: 3, SEQ ID NO: 5, SEQ ID NO: 495, and SEQ ID NO: 496.

321. A cell comprising the composition of any one of claims 239 to 320.

322. The cell of claim 321, wherein the composition does not substantially affect viability of the cell.

323. A composition comprising a variant Cas12i2 polypeptide, wherein the variant Cas12i2 polypeptide comprises a substitution that increases flexibility of the variant Cas12i2 polypeptide during DNA binding, as compared to a parent polypeptide.

324. The composition of claim 323, wherein the variant Cas12i2 polypeptide comprises the sequence set forth in SEQ ID NO: 4.

325. The composition of claim 323, wherein the Cas12i2 polypeptide comprises the sequence set forth in any one of SEQ ID NO: 3, SEQ ID NO: 5, SEQ ID NO: 495, and SEQ ID NO: 496.

326. The composition of any one of claims 323 to 325, wherein the substitution increases binding of the variant Cas12i2 polypeptide to DNA.

327. The composition of any one of claims 323 to 326, wherein the substitution increases binding of the variant Cas12i2 polypeptide to double-stranded DNA.

328. The composition of any one of claims 323 to 327, wherein the substitution increases binding of the variant Cas12i2 polypeptide to single-strand DNA.

329. The composition of any one of claims 323 to 328, wherein the substitution increases ternary complex formation, as compared to a parent polypeptide.

330. The composition of any one of claims 323 to 329, wherein a ternary complex comprising the variant Cas12i2 polypeptide exhibits increased stability, as compared to a parent ternary complex.

331. The composition of any one of claims 323 to 330, wherein the substitution is a substitution of a bulky amino acid to an amino acid with a smaller side chain.

332. The composition of any one of claims 323 to 331, wherein the substitution is an alanine, valine, glycine, or serine substitution.

333. The composition of any one of claims 323 to 332, wherein the substitution is in the Helical II domain of the variant Cas12i2 polypeptide.

334. The composition of any one of claims 323 to 333, wherein the substitution is a substitution listed in Table 9.

335. The composition of any one of claims 323 to 332, wherein the variant Cas12i2 polypeptide comprises a sequence having at least 95% identity to a sequence set forth in any one of SEQ ID NOs: 3-146 or in any one of SEQ ID NOs: 495-512.

336. The composition of any one of claims 323 to 332, wherein the variant Cas12i2 polypeptide comprises a sequence having at least 95% identity to a sequence set forth in any one of SEQ ID NOs: 3-146 or in any one of SEQ ID NOs: 495-512 and further having a substitution listed in Table 9.

337. The composition of any one of claims 323 to 332, wherein the variant Cas12i2 polypeptide comprises a sequence set forth in any one of SEQ ID NOs: 3-146 or in any one of SEQ ID NOs: 495-512.

338. The composition of any one of claims 323 to 332, wherein the variant Cas12i2 polypeptide comprises a sequence set forth in any one of SEQ ID NOs: 3-146 or in any one of SEQ ID NOs: 495-512 and further has a substitution listed in Table 9.

339. A composition comprising a variant Cas12i2 polypeptide, wherein the variant Cas12i2 polypeptide comprises a substitution that stabilizes a domain-domain interface that forms during ternary complex formation, as compared to a parent polypeptide.

340. The composition of claim 339, wherein the variant Cas12i2 polypeptide comprises the sequence set forth in SEQ ID NO: 4.

341. The composition of claim 339, wherein the variant Cas12i2 polypeptide comprises the sequence set forth in any one of SEQ ID NO: 3, SEQ ID NO: 5, SEQ ID NO: 495, and SEQ ID NO: 496.

342. The composition of any one of claims 339 to 341, wherein the domain-domain interface forms when single-stranded DNA contacts an active site of the variant Cas12i2 polypeptide.

343. The composition of any one of claims 339 to 342, wherein the domain-domain interface is a Helical II domain-Nuc domain interface.

344. The composition of any one of claims 339 to 343, wherein the substitution increases ternary complex formation, as compared to a parent polypeptide.

345. The composition of any one of claims 339 to 344, wherein a ternary complex comprising the variant Cas12i2 polypeptide exhibits increased stability, as compared to a parent ternary complex.

346. The composition of any one of claims 339 to 345, wherein the substitution is an aspartic acid, glutamic acid, arginine, or lysine substitution.

347. The composition of any one of claims 339 to 346, wherein the substitution is a substitution listed in Table 10.

348. The composition of any one of claims 339 to 346, wherein the variant Cas12i2 polypeptide comprises a sequence having at least 95% identity to a sequence set forth in any one of SEQ ID NOs: 3-146 or in any one of SEQ ID NOs: 495-512.

349. The composition of any one of claims 339 to 346, wherein the variant Cas12i2 polypeptide comprises a sequence having at least 95% identity to a sequence set forth in any one of SEQ ID NOs: 3-146 or in any one of SEQ ID NOs: 495-512 and further having a substitution listed in Table 10.

350. The composition of any one of claims 339 to 346, wherein the variant Cas12i2 polypeptide comprises a sequence set forth in any one of SEQ ID NOs: 3-146 or in any one of SEQ ID NOs: 495-512.

351. The composition of one of claims 339 to 346, wherein the variant Cas12i2 polypeptide comprises a sequence set forth in any one of SEQ ID NOs: 3-146 or in any one of SEQ ID NOs: 495-512 and further has a substitution listed in Table 10.

352. The composition of any one of claims 323 to 351, wherein the parent polypeptide comprises the amino acid sequence of SEQ ID NO: 2.

353. The composition of any one of claims 323 to 352, wherein the variant Cas12i2 polypeptide exhibits increased enzymatic activity, as compared to a parent polypeptide.

354. The composition of any one of claims 323 to 353, wherein the composition further comprises an RNA guide comprising a direct repeat sequence and a spacer sequence.

355. The composition of claim 354, wherein the direct repeat sequence comprises a nucleotide sequence with at least 95% sequence identity to any one of SEQ ID NO: 492-494.

356. The composition of claim 354, wherein the direct repeat sequence comprises a nucleotide sequence set forth in any one of SEQ ID NO: 492-494.

357. The composition of claim 354, wherein the direct repeat sequence is set forth in SEQ ID NO: 492 and the variant Cas12i2 polypeptide comprises a sequence set forth in SEQ ID NO: 4.

358. The composition of claim 354, wherein the direct repeat sequence is set forth in SEQ ID NO: 492 and the variant Cas12i2 polypeptide comprises the sequence set forth in any one of SEQ ID NO: 3, SEQ ID NO: 5, SEQ ID NO: 495, and SEQ ID NO: 496.

359. A cell comprising the composition of any one of claims 323 to 358.

360. The cell of claim 359, wherein the composition does not substantially affect viability of the cell.

361. A composition comprising a variant Cas12i2 polypeptide, wherein the variant Cas12i2 polypeptide comprises a substitution that increases on-target specificity of the variant Cas12i2 polypeptide, as compared to a parent polypeptide.

362. The composition of claim 361, wherein the variant Cas12i2 polypeptide comprises the sequence set forth in SEQ ID NO: 4.

363. The composition of claim 361, wherein the variant Cas12i2 polypeptide comprises the sequence set forth in any one of SEQ ID NO: 3, SEQ ID NO: 5, SEQ ID NO: 495, and SEQ ID NO: 496.

364. The composition of any of claims 361 to 363, wherein the substitution increases on-target DNA binding.

365. The composition of any one of claims 361 to 364, wherein the substitution decreases off-target DNA binding.

366. The composition of any one of claims 361 to 365, wherein the substitution increases on-target ternary complex formation, as compared to a parent polypeptide.

367. The composition of any one of claims 361 to 366, wherein an on-target ternary complex comprising the variant Cas12i2 polypeptide exhibits increased stability, as compared to a parent ternary complex.

368. The composition of any one of claims 361 to 367, wherein the substitution is a substitution of an amino acid contacting the spacer sequence of an RNA guide.

369. The composition of any one of claims 361 to 368, wherein the substitution is a substitution of a bulky amino acid to an amino acid with a smaller side chain.

370. The composition of any one of claims 361 to 369, wherein the substitution is an alanine, serine, valine, glutamine, or asparagine substitution.

371. The composition of any one of claims 361 to 370, wherein the substitution is a substitution in the Wedge domain, Rec1 domain, Rec2 domain, or RuvC domain.

372. The composition of any one of claims 361 to 371, wherein the substitution is a substitution in the Helical II domain.

373. The composition of any one of claims 361 to 372, wherein the substitution is a substitution listed in Table 11.

374. The composition of any one of claims 361 to 372, wherein the variant Cas12i2 polypeptide comprises a sequence having at least 95% identity to a sequence set forth in any one of SEQ ID NOs: 3-146 or in any one of SEQ ID NOs: 495-512.

375. The composition of any one of claims 361 to 372, wherein the variant Cas12i2 polypeptide comprises a sequence having at least 95% identity to a sequence set forth in any one of SEQ ID NOs: 3-146 or in any one of SEQ ID NOs: 495-512 and further having a substitution listed in Table 11.

376. The composition of any one of claims 361 to 372, wherein the variant Cas12i2 polypeptide comprises a sequence set forth in any one of SEQ ID NOs: 3-146 or in any one of SEQ ID NOs: 495-512.

377. The composition of any one of claims 361 to 372, wherein the variant Cas12i2 polypeptide comprises a sequence set forth in any one of SEQ ID NOs: 3-146 or in any one of SEQ ID NOs: 495-512 and further has a substitution listed in Table 11.

378. The composition of any one of claims 361 to 367, wherein the substitution decreases a catalysis rate (Kcat) of the variant Cas12i2 polypeptide.

379. The composition of any one of claims 361 to 367 and 378, wherein the substitution is an alanine, serine, threonine, valine, leucine, methionine, asparagine, or isoleucine substitution.

380. The composition of any one of claims 361 to 367 and 378 to 379, wherein the substitution is a substitution in the Wedge domain, Rec1 domain, Rec2 domain, or RuvC domain.

381. The composition of any one of claims 361 to 367 and 378 to 380, wherein the substitution is a substitution in the RuvC domain.

382. The composition of any one of claims 361 to 367 and 378 to 381, wherein the substitution is a substitution listed in Table 12.

383. The composition of any one of claims 361 to 367 and 378 to 381, wherein the variant Cas12i2 polypeptide comprises a sequence having at least 95% identity to a sequence set forth in any one of SEQ ID NOs: 3-146 or in any one of SEQ ID NOs: 495-512.

384. The composition of any one of claims 361 to 367 and 378 to 381, wherein the variant Cas12i2 polypeptide comprises a sequence having at least 95% identity to a sequence set forth in any one of SEQ ID NOs: 3-146 or in any one of SEQ ID NOs: 495-512 and further having a substitution listed in Table 12.

385. The composition of any one of claims 361 to 367 and 378 to 381, wherein the variant Cas12i2 polypeptide comprises a sequence set forth in any one of SEQ ID NOs: 3-146 or in any one of SEQ ID NOs: 495-512.

386. The composition of any one of claims 361 to 367 and 378 to 381, wherein the variant Cas12i2 polypeptide comprises a sequence set forth in any one of SEQ ID NOs: 3-146 or in any one of SEQ ID NOs: 495-512 and further has a substitution listed in Table 12.

387. The composition of one of claims 361 to 386, wherein the variant Cas12i2 polypeptide exhibits increased on-target enzymatic activity, as compared to a parent polypeptide.

388. The composition of any one of claims 361 to 387, wherein the variant Cas12i2 polypeptide exhibits decreased off-target enzymatic activity, as compared to a parent polypeptide.

389. The composition of any one of claims 361 to 388, wherein the variant Cas12i2 polypeptide exhibits off-target editing that is no more than 10% of the on-target editing.

390. The composition of any one of claims 361 to 388, wherein the variant Cas12i2 polypeptide exhibits off-target editing that is no more than 5% of the on-target editing.

391. The composition of any one of claims 361 to 390, wherein the composition further comprises an RNA guide comprising a direct repeat sequence and a spacer sequence.

392. The composition of claim 391, wherein the direct repeat sequence comprises a nucleotide sequence with at least 95% sequence identity to any one of SEQ ID NO: 492-494.

393. The composition of claim 391, wherein the direct repeat sequence comprises a nucleotide sequence set forth in any one of SEQ ID NO: 492-494.

394. The composition of claim 391, wherein the direct repeat sequence is set forth in SEQ ID NO: 492 and the variant Cas12i2 polypeptide comprises a sequence set forth in SEQ ID NO: 4.

395. The composition of claim 391, wherein the direct repeat sequence is set forth in SEQ ID NO: 492 and the variant Cas12i2 polypeptide comprises the sequence set forth in any one of SEQ ID NO: 3, SEQ ID NO: 5, SEQ ID NO: 495, and SEQ ID NO: 496.

396. The composition of any one of claims 365 to 395, wherein the variant Cas12i2 polypeptide exhibits a higher ratio of on-target binding to off-target binding, as compared to a Cas9 polypeptide.

397. The composition of any one of claims 365 to 396, wherein the variant Cas12i2 polypeptide exhibits a higher ratio of on-target activity to off-target activity, as compared to a Cas9 polypeptide.

398. The composition of any one of claims 365 to 397, wherein the variant Cas12i2 polypeptide exhibits a higher ratio of on-target editing to off-target editing, as compared to a Cas9 polypeptide.

399. The composition of any one of claims 365 to 395, wherein the variant Cas12i2 polypeptide exhibits less off-target binding, as compared to a Cas9 polypeptide.

400. The composition of any one of claims 365 to 396, wherein the variant Cas12i2 polypeptide exhibits less off-target activity, as compared to a Cas9 polypeptide.

401. The composition of any one of claims 365 to 397, wherein the variant Cas12i2 polypeptide exhibits less off-target editing, as compared to a Cas9 polypeptide.

402. The composition of any one of claims 365 to 401, wherein off-target binding, off-target activity, and/or off-target editing by the variant Cas12i2 polypeptide is at least 10% less than off-target binding, off-target activity, and/or off-target editing by a Cas9 polypeptide.

403. The composition of any one of claims 365 to 402, wherein off-target binding, off-target activity, and/or off-target editing by the variant Cas12i2 polypeptide is at least 20% less than off-target binding, off-target activity, and/or off-target editing by a Cas9 polypeptide.

404. The composition of any one of claims 365 to 403, wherein off-target binding, off-target activity, and/or off-target editing by the variant Cas12i2 polypeptide is at least 30% less than off-target binding, off-target activity, and/or off-target editing by a Cas9 polypeptide.

405. The composition of any one of claims 365 to 404, wherein off-target binding, off-target activity, and/or off-target editing by the variant Cas12i2 polypeptide is at least 40% less than off-target binding, off-target activity, and/or off-target editing by a Cas9 polypeptide.

406. The composition of any one of claims 365 to 405, wherein off-target binding, off-target activity, and/or off-target editing by the variant Cas12i2 polypeptide is at least 50% less than off-target binding, off-target activity, and/or off-target editing by a Cas9 polypeptide.

407. A cell comprising the composition of any one of claims 365 to 406.

408. The cell of claim 407, wherein the composition does not substantially affect viability of the cell.
